(19) United States
(12) Patent Application Publication
(10) Pub. No.: US 2022/0107328 A1
(43) Pub. Date: Apr. 7, 2022

(54) METHODS OF TREATING LIVER DISEASES

(71) Applicant: Camp4 Therapeutics Corporation, Cambridge, MA (US)

(72) Inventors: David A. Bumcrot, Belmont, MA (US); Alfica Sehgal, Cambridge, MA (US); Alla A. Sigova, Newton, MA (US); Brian Elliott Schwartz, Somerville, MA (US); Gavin Whissell, North Reading, MA (US); Iris Grossman, Cambridge, MA (US); Vaishnavi Rajagopal, Andover, MA (US); Cynthia Marie Smith, Boston, MA (US); Mario Esteban Contreras Gamboa, Boston, MA (US)

(21) Appl. No.: 17/268,431

(22) PCT Filed: Aug. 14, 2019
(86) PCT No.: PCT/US19/46556

§ 371 Date: Feb. 12, 2021

Related U.S. Application Data
(60) Provisional application No. 62/805,516, filed on Feb. 14, 2019.
(60) Provisional application No. 62/795,397, filed on Jan. 22, 2019.
(60) Provisional application No. 62/789,469, filed on Jan. 7, 2019.
(60) Provisional application No. 62/718,607, filed on Aug. 14, 2018.

(30) Foreign Application Priority Data
Aug. 14, 2018 (US) PCT/US2018/046634

(51) Int. Cl. G01N 33/68; A61K 31/23; A61K 31/5377; A61K 31/55; A61P 1/16; G01N 33/92

(52) U.S. Cl. CPC G01N 33/6893; A61K 31/23; A61K 31/5377; A61K 31/55; G01N 2800/52; G01N 33/92; G01N 2333/91057; G01N 2500/04; G01N 2800/08; A61P 1/16

(57) ABSTRACT

Provided herein are methods and compositions for the treating a patient with one or more conditions associated with PNPLA3, such as nonalcoholic fatty liver disease (NAFLD), nonalcoholic steatohepatitis (NASH), and/or alcoholic liver disease (ALD). Methods and compositions are also provided for modulating the expression of the PNPLA3 gene in a cell by altering gene signaling networks. Companion diagnostic methods, compositions and kits are also provided.

### CROSS REFERENCE TO RELATED APPLICATIONS

This application claims the benefit of International Application No. PCT/US2018/046634 filed on Aug. 14, 2018; U.S. Provisional Application No. 62/718,607, filed Aug. 14, 2018; U.S. Provisional Application No. 62/789,469, filed Jan. 7, 2019; U.S. Provisional Application No. 62/795,397, filed Jan. 22, 2019; and U.S. Provisional Application No. 62/805,516, filed Feb. 14, 2019; each of which are hereby incorporated by reference in their entirety.

### SEQUENCE LISTING

The present application is being filed along with a Sequence Listing in electronic format. The Sequence Listing file, entitled CTC_009WO_Sequence_listing.txt, was created on Aug. 7, 2019, and is 31,330 bytes in size. The information in electronic format of the Sequence Listing is incorporated herein by reference in its entirety.

### BACKGROUND

Nonalcoholic fatty liver disease (NAFLD) is one of the most common hepatic disorders worldwide. In the United States, it affects an estimated 80 to 100 million people. NAFLD occurs in every age group but especially in people in their 40s and 50s. NAFLD is a buildup of excessive fat in the liver that can lead to liver damage resembling the damage caused by alcohol abuse, but this occurs in people who drink little to no alcohol. The condition is also associated with adverse metabolic consequences, including increased abdominal fat, poor ability to use the hormone insulin, high blood pressure and high blood levels of triglycerides.

In some cases, NAFLD leads to inflammation of the liver, referred to as non-alcoholic steatohepatitis (NASH). NASH is a progressive liver disease characterized by fat accumulation in the liver leading to liver fibrosis. About 20 percent of people with NASH will progress to fibrosis. NASH affects approximately 26 million people in the United States. With continued inflammation, fibrosis spreads to take up more and more liver tissue, leading to liver cancer and/or end-stage liver failure in most severe cases. NASH is highly correlated to obesity, diabetes and related metabolic disorders. Genetic and environmental factors also contribute to the development of NASH.

Currently, no drug treatment exists for NAFLD or NASH. The condition is primarily managed in early stages through lifestyle modification (e.g., physical exercise, weight loss, and healthy diet) which may encounter poor adherence. Losing weight addresses the conditions that contribute to nonalcoholic fatty liver disease. Weight-loss surgery is also an option for those who need to lose a great deal of weight. Anti-diabetic medication, vitamins or dietary supplements can be useful for controlling the condition. For those who have cirrhosis due to NASH, liver transplantation may be an option. This is the 3^{rd} most common reason for liver transplants in the US and is projected to become most common reason in three years.

Alcoholic liver disease (ALD) accounts for the majority of chronic liver diseases in Western countries. It encompasses a spectrum of liver manifestations of alcohol overconsumption, including fatty liver, alcoholic hepatitis, and alcoholic cirrhosis. Alcoholic liver cirrhosis is the most advanced form of ALD and is one of the major causes of liver failure, hepatocellular carcinoma and liver-related mortality causes. Restricting alcohol intake is the primary treatment for ALD. Other treatment options include supportive care (e.g., healthy diet, vitamin supplements), use of corticosteroids, and sometimes liver transplantation.

Therefore, there is a need for developing effective therapeutics for the treatment of NAFLD, NASH and/or ALD.

### SUMMARY

Provided herein are compositions and methods for the diagnosis and treatment of a disease or disorder associated with Patatin-like phospholipase domain-containing protein 3 (PNPLA3), such as NAFLD, NASH and ALD. Such treatments are directed to modulating the gene expression regulation of the PNPLA3 gene (e.g., via altering a gene signaling network), thereby altering the expression of PNPLA3.

Provided herein are methods of treating a subject in need thereof with a Patatin-like phospholipase domain-containing protein 3 (PNPLA3)-targeted therapy comprising obtaining or having obtained a dataset comprising genomic data from a biological sample obtained from the subject; determining or having determined the presence or absence of a G allele at SNP rs738409 in the dataset; identifying or having identified the subject as eligible for the PNPLA3-targeted treatment based on the presence of the G allele at SNP rs738409; and administering to the subject an effective amount of a compound capable of reducing the expression of the PNPLA3 gene, wherein the compound capable of reducing the expression of the PNPLA3 gene comprises an mTOR inhibitor that does not inhibit the PI3K pathway.

In some embodiments, the determining step comprises detecting the allele using a method selected from the group consisting of: mass spectroscopy, oligonucleotide microarray analysis, allele-specific hybridization, allele-specific PCR, and nucleic acid sequencing.

In another aspect, provided herein are methods of treating a subject in need thereof with a PNPLA3-targeted therapy comprising obtaining or having obtained a dataset comprising proteomic data from a biological sample obtained from the subject; determining or having determined the presence or absence of a mutant PNPLA3 protein carrying the I148M mutation in the dataset; identifying or having identified the subject as eligible for the PNPLA3-targeted treatment based on the presence of the mutant PNPLA3 protein carrying the I148M mutation; and administering to the subject an effective amount of a compound capable of reducing the expression of the PNPLA3 gene, wherein the compound capable of reducing the expression of the PNPLA3 gene comprises an mTOR inhibitor that does not inhibit the PI3K pathway.

In some embodiments, the determining step comprises detecting the mutant protein using mass spectroscopy. In some embodiments, the biological sample is a biopsy sample.

In some embodiments, the mTOR inhibitor does not inhibit PI3Kβ activity. In some embodiments, the mTOR inhibitor does not inhibit DNA-PK. In some embodiments, the mTOR inhibitor is OSI-027. In some embodiments, the mTOR inhibitor comprises an mTORC2 inhibitor. In some embodiments, mTORC2 inhibitor comprises a RICTOR inhibitor. In some embodiments, the RICTOR inhibitor is JR-AB2-011.

In some embodiments, the administration of the compound capable of reducing the expression of the PNPLA3 gene does not induce hyperinsulinemia in the subject. In some embodiments, the administration of the compound capable of reducing the expression of the PNPLA3 gene does not induce hyperglycemia in the subject.

In some embodiments, the compound capable of reducing the expression of the PNPLA3 gene is selected from the group consisting of OSI-027, WYE-125132, CC-223, Everolimus, Palomid 529 (P529), GDC-0349, Torin 1, PP242, WAY600, CZ415, INK128, TAK659, AZD-8055, Deforolimus, and JR-AB2-011.

In some embodiments, the compound comprises one or more small interfering RNA (siRNA) targeting one or more genes selected from the group consisting of RICTOR, mTOR, Deptor, AKT, mLST8, mSIN1, and Protor. In some embodiments, the one or more small interfering RNA (siRNA) targets RICTOR.

In some embodiments, the subject is homozygous for the G allele at SNP rs738409. In some embodiments, the subject is heterozygous for the G allele at SNP rs738409. In some embodiments, the subject is homozygous for the mutant PNPLA3 protein carrying the I148M mutation. In some embodiments, the subject is heterozygous for the mutant PNPLA3 protein carrying the I148M mutation.

In some embodiments, the expression of the PNPLA3 gene is reduced by at least about 30%. In some embodiments, the expression of the PNPLA3 gene is reduced by at least about 50%. In some embodiments, the expression of the PNPLA3 gene is reduced by at least about 70%. In some embodiments, the reduction is determined in a population of test subjects and the amount of reduction is determined by reference to a matched control population.

In some embodiments, the expression of the PNPLA3 gene is reduced in the liver of the subject. In some embodiments, the expression of the PNPLA3 gene is reduced in the hepatocytes of the subject. In some embodiments, the expression of the PNPLA3 gene is reduced in the hepatic stellate cells of the subject. In some embodiments, the expression of the PNPLA3 gene is reduced in the hepatocytes and hepatic stellate cells of the subject.

In some embodiments, the method further comprises assessing or having assessed a hepatic triglyceride content in the subject. In some embodiments, the assessing or having assessed step comprises using a method selected from the group consisting of liver biopsy, liver ultrasonography, computer-aided tomography (CAT) and nuclear magnetic resonance (NMR). In some embodiments, the assessing or having assessed step comprises proton magnetic resonance spectroscopy (¹H-MRS). In some embodiments, the subject is eligible for treatment based on a hepatic triglyceride content greater than 5.5% volume/volume.

In another aspect, provided herein are methods of reducing the lipid content in cells in a subject, comprising the steps of: obtaining or having obtained a biological sample from the subject; determining or having determined in the biological sample the amount of lipid content; and administering an effective amount of a compound capable of reducing the expression of the PNPLA3 gene.

In some embodiments, the method further comprises assessing the hepatic triglyceride in the subject. In some embodiments, the assessing step comprises using a method selected from the group consisting of liver biopsy, liver ultrasonography, computer-aided tomography (CAT) and nuclear magnetic resonance (NMR).

In some embodiments, the lipid content is in hepatocytes. In some embodiments, the lipid content is in hepatic stellate cells. In some embodiments, the lipid content is in a population of hepatocytes and hepatic stellate cells.

In some embodiments, the compound comprises an mTOR inhibitor. In some embodiments, the compound comprises OSI-027, or a derivative or an analog thereof. In some embodiments, the mTOR inhibitor comprises an mTORC2 inhibitor. In some embodiments, the mTORC2 inhibitor comprises a RICTOR inhibitor.

In some embodiments, the RICTOR inhibitor is JR-AB2-011, or a derivative or an analog thereof. In some embodiments, the compound comprises PF-04691502, or a derivative or an analog thereof. In some embodiments, the compound capable of reducing the expression of the PNPLA3 gene comprises at least one selected from the group consisting of OSI-027, PF-04691502, Momelotinib, WYE-125132, CC-223, Everolimus, Palomid 529 (P529), GDC-0349, Torin 1, PP242, WAY600, CZ415, INK128, TAK659, AZD-8055, Deforolimus, and JR-AB2-011.

In some embodiments, the compound comprises one or more small interfering RNA (siRNA) targeting one or more genes selected from the group consisting of JAK1, JAK2, mTOR, RICTOR, Deptor, AKT, mLST8, mSIN1, and Protor. In some embodiments, the one or more small interfering RNA (siRNA) targets RICTOR. In some embodiments, the one or more small interfering RNA (siRNA) targets mTOR.

In some embodiments, the expression of the PNPLA3 gene is reduced by at least about 30%. In some embodiments, the expression of the PNPLA3 gene is reduced by at least about 50%. In some embodiments, the expression of the PNPLA3 gene is reduced by at least about 70%.

In another aspect, provided herein are methods of identifying a compound that reduces PNPLA3 gene expression comprising providing a candidate compound; assaying the candidate compound for at least two of the activities selected from the group consisting of: mTOR inhibitory activity, mTORC2 inhibitory activity, PI3K inhibitory activity, PI3Kβ inhibitory activity, DNA-PK inhibitory activity, ability to induce hyperinsulinemia, ability to induce hyperglycemia, and PNPLA3 gene expression inhibitory activity; and identifying the candidate compound as the compound based on results of the two or more assays that indicate the candidate compound has two or more desirable properties.

In some embodiments, the desirable properties are selected from the group consisting of: mTOR inhibitory activity, lack of PI3K inhibitory activity, lack of PI3Kβ inhibitory activity, lack of DNA-PK inhibitory activity, lack of ability to induce hyperinsulinemia, lack of ability to induce hyperglycemia, and PNPLA3 gene expression inhibitory activity. In some embodiments, mTOR inhibitory activity comprises inhibition of mTORC2 activity. In some embodiments, the mTOR inhibitory activity is mTORC1 and mTOR2 inhibitory activity. In some embodiments, the PI3K inhibitory activity is PI3Kβ inhibitory activity.

In some embodiments, the assaying step comprises assaying for at least three of the activities. In some embodiments, the assaying step comprises assaying for at least four of the activities. In some embodiments, the assaying step comprises assaying for at least five of the activities.

In some embodiments, the at least two assays of step (b) comprise assays for mTOR inhibitory activity and PI3K inhibitory activity. In some embodiments, the at least two assays of step (b) comprise assays for mTORC2 inhibitory activity and PI3Kβ inhibitory activity. In some embodiments, the at least three assays of step (b) comprise assays for mTOR inhibitory activity, PI3K inhibitory activity, and ability to induce hyperinsulinemia. In some embodiments, the at least four assays of step (b) comprise mTOR inhibitory activity, PI3K inhibitory activity, ability to induce hyperinsulinemia, and DNA-PK inhibitory activity.

In some embodiments, the assay is a biochemical assay. In some embodiments, the assay is in a cell. In some embodiments, the cell is an animal cell or a human cell. In some embodiments, the cell is a wild type cell. In some embodiments, the cell comprises the G allele at SNP rs738409 of the PNPLA3 gene or a mutant I148M PNPLA3 protein. In some embodiments, the cell is homozygous for the G allele at SNP rs738409. In some embodiments, the cell is heterozygous for the G allele at SNP rs738409. In some embodiments, the cell is homozygous for the mutant PNPLA3 protein carrying the I148M mutation. In some embodiments, the cell is heterozygous for the mutant PNPLA3 protein carrying the I148M mutation.

In some embodiments, assaying the PNPLA3 gene expression comprises a method selected from the group consisting of: mass spectroscopy, oligonucleotide microarray analysis, allele-specific hybridization, allele-specific PCR, and nucleic acid sequencing.

In some embodiments, the expression of the PNPLA3 gene is reduced by at least about 30%. In some embodiments, the expression of the PNPLA3 gene is reduced by at least about 50%. In some embodiments, the expression of the PNPLA3 gene is reduced by at least about 70%. In some embodiments, the reduction is determined in a population of cells and the amount of reduction is determined by reference to a matched control cell population.

### BRIEF DESCRIPTION OF THE DRAWINGS

The foregoing and other objects, features and advantages will be apparent from the following description of particular embodiments of the invention, as illustrated in the accompanying drawings. The drawings are not necessarily to scale; emphasis instead being placed upon illustrating the principles of various embodiments of the invention.

FIG. 1 illustrates the packaging of chromosomes in a nucleus, the localized topological domains into which chromosomes are organized, insulated neighborhoods in TADs and finally an example of an arrangement of a signaling center(s) around a particular disease gene.

FIG. 2A illustrates a linear arrangement of the CTCF boundaries of an insulated neighborhood. FIG. 2B illustrates a 3D arrangement of the CTCF boundaries of an insulated neighborhood.

FIG. 3A illustrates tandem insulated neighborhoods and gene loops formed in such insulated neighborhoods. FIG. 3B illustrates tandem insulated neighborhoods and gene loops formed in such insulated neighborhoods.

FIG. 4 illustrates the concept of an insulated neighborhood contained within a larger insulated neighborhood and the signaling which may occur in each.

FIG. 5 illustrates the components of a signaling center; including transcriptional factors, signaling proteins, and/or chromatin regulators.

FIG. 6 shows the dose response curve of Momelotinib in primary human hepatocytes.

FIG. 7 shows the dose response curve of Momelotinib in hepatic stellate cells.

FIG. 8 shows the dose response curve of Momelotinib in HepG2 cells.

FIG. 9 shows the effect of Momelotinib treatment on PNPLA3 expression in mouse liver.

FIG. 10 shows the effect of WYE-125132 treatment on COL1A1 expression in mouse liver.

FIG. 11A shows the effects of OSI-027 and PF-04691502 on PNPLA3 expression in multiple homozygous M/M human hepatocyte donors. FIG. 11B shows the effects of OSI-027 and PF-04691502 on PNPLA3 expression in multiple heterozygous I/M human hepatocyte donors. FIG. 11C shows the effects of OSI-027 and PF-04691502 on PNPLA3 expression in multiple homozygous I/I human hepatocyte donors.

FIG. 12A shows the effects of OSI-027 and PF-04691502 on PNPLA3 expression in homozygous I/I human stellate cells. FIG. 12B shows the effects of OSI-027 and PF-04691502 on PNPLA3 expression in homozygous M/M human stellate cells.

FIG. 13 shows the dose response effects of OSI-027 and PF-04691502 on primary human hepatocytes.

FIG. 14A shows the effects of OSI-027 and PF-04691502 on lipid content in primary human hepatocytes. FIG. 14B shows the effects of OSI-027 and PF-04691502 on lipid content in primary human hepatocytes.

FIG. 15A shows the effect of OSI-027 on triglyceride content in HepG2 cells. FIG. 15B shows the effect of OSI-027 on triglyceride content (nmol/μg protein) in HepG2 cells.

FIG. 16A shows the effects of OSI-027 and PF-04691502 on PNPLA3 liver mRNA levels in vivo at 12 hrs post dosing. FIG. 16B shows the effects of OSI-027 and PF-04691502 on PNPLA3 liver mRNA levels in vivo at 6 hrs post dosing.

FIG. 17A shows the effects of OSI-027 on PNPLA3 liver mRNA levels in vivo at 6 hrs post dosing. FIG. 17B shows the effects of OSI-027 on PNPLAS liver mRNA levels in vivo at 6 hrs post dosing. FIG. 17C shows the effects of OSI-027 on COL1A1 liver mRNA levels in vivo at 6 hrs post dosing. FIG. 17D show the effects of OSI-027 on PCSK9 liver mRNA levels in vivo at 6 hrs post dosing. FIG. 17E show the effects of OSI-027 on ANGPTL3 liver mRNA levels in vivo at 6 hrs post dosing.

FIG. 18A shows the effects of PF-04691502 on PNPLA3 liver mRNA levels in vivo at 6 hrs post dosing. FIG. 18B shows the effects of PF-04691502 on PNPLAS liver mRNA levels in vivo at 6 hrs post dosing. FIG. 18C shows the effects of PF-04691502 on COL1A1 liver mRNA levels in vivo at 6 hrs post dosing. FIG. 18D shows the effects of PF-04691502 on PCSK9 liver mRNA levels in vivo at 6 hrs post dosing. FIG. 18E shows the effects of PF-04691502 on ANGPTL3 liver mRNA levels in vivo at 6 hrs post dosing.

FIG. 19A shows the effects of LY2157299 on PNPLA3 liver mRNA levels in vivo at 6 hrs post dosing. FIG. 19B shows the effects of LY2157299 on PNPLAS liver mRNA levels in vivo at 6 hrs post dosing. FIG. 19C shows the effects of LY2157299 on COL1A1 liver mRNA levels in vivo at 6 hrs post dosing. FIG. 19D shows the effects of LY2157299 on PCSK9 liver mRNA levels in vivo at 6 hrs post dosing. FIG. 19E shows the effects of LY2157299 on ANGPTL3 liver mRNA levels in vivo at 6 hrs post dosing.

FIG. 20 shows gene circuitry mapping of the PNPLA3 gene. The top section shows the HiChIP chromatin mapping, the bottom section shows a comparison of the HiChIP, ChIP-seq, ATAC-seq, and RNA-seq mapping of the PNPLA3 gene.

FIG. 21 shows a diagram of the known and newly identified PNPLA3 transcription factors.

FIG. 22 shows a diagram of the pathways that contribute to PNPLA3 expression as identified by gene circuitry mapping.

FIG. 23 shows the relative PNPLA3 mRNA levels in human hepatocytes after treatment with the indicated siRNA.

FIGS. 24A show that Momelotinib reduces chromatin accessibility of the PNPLA3 gene. FIG. 24B provides a diagram of the PNPLA3 chromatin mapping with the primer locations.

FIG. 25 shows the effects of Momelotinib on PNPLA3 expression in a dose-dependent manner in primary hepatocytes regardless of the PNPLA3 allele status of the cells.

FIG. 26 shows the effects of Momelotinib on PNPLA3 liver mRNA levels in vivo.

FIG. 27 provides the total triglyceride (nmol) amount in HepG2 after treatment with OSI-027.

FIG. 28 shows the relative PNPLA3 mRNA levels in human hepatocytes after treatment with the indicated compounds.

FIG. 29A show the relative PNPLA3 mRNA in mouse samples before re-analysis of OSI-027 treated mice. FIG. 2B show the relative PNPLA3 mRNA in mouse samples after re-analysis of OSI-027 treated mice. FIG. 29C show the relative PNPLA3 mRNA in mouse samples before re-analysis of PF-04691502 treated mice. FIG. 29D show the relative PNPLA3 mRNA in mouse samples after re-analysis of PF-04691502 treated mice.

FIG. 30A shows that treatment of hepatocyte cell line Yecuris RMG with the momelotinib metabolite M21 reduced PNPLA3 mRNA expression. FIG. 30B shows that treatment of hepatocyte cells line HU4282 with the momelotinib metabolite M21 reduced PNPLA3 mRNA expression. FIG. 30C shows that treatment of hepatocyte cells lines ST1 and ST8 with the momelotinib metabolite M21 reduced PNPLA3 mRNA expression.

FIG. 31A shows PNPLA3 expression in hepatocytes after treatment with OSI-027 with and without mTOR siRNA knockdown. FIG. 31B shows PNPLA3 expression in hepatocytes after treatment with PF-04691502 with and without mTOR siRNA knockdown.

FIG. 32 shows the effects of mTOR inhibitors on COL1A1, PNPLA3, MMP2, TIM2, TGFB1, COL1A2, and ACTA2 expression.

FIG. 33 shows the effects of TGF-β pathway inhibitors on PNPLA3 mRNA expression in primary human hepatocytes.

FIG. 34 shows the effects of BMP pathway inhibitors on PNPLA3 mRNA expression in primary human hepatocytes.

FIG. 35A shows TGFβ-ligand induces expression of PNPLA3 in a dose dependent manner. FIG. 35B shows TGFβ-ligand induces expression of COL1A1 in a dose dependent manner.

FIG. 36 shows PNPLA3 expression in hepatocytes after treatment with LY2157299 and TGFβ-ligand.

FIG. 37 shows PNPLA3 expression in stellate cells after treatment with the indicated compounds and TGFβ-ligand.

FIG. 38 shows relative PNPLA3 mRNA expression in hepatocytes after siRNA knockdown of mTOR or PRKDC (DNA-PK).

FIG. 39A shows the relative amount of PNPLA3 mRNA compared to GUSB after OSI-027 treatment in cells that were pretreated with mTOR and AKT3 siRNA or control siRNA. FIG. 39B shows the relative amount of PNPLA3 mRNA compared to GUSB after PF-04691502 treatment in cells that were pretreated with mTOR and AKT3 siRNA or control siRNA.

FIG. 40A shows the relative amounts of PNPLA3 mRNA normalized to GUSB expression and indicated phosphorylated protein as compared to total protein in hepatocytes after treatment with PF-04691502. FIG. 40B shows the relative amounts of PNPLA3 mRNA normalized to GUSB expression and indicated phosphorylated protein as compared to total protein in hepatocytes after treatment with OSI-027. FIG. 40C shows the relative amounts of PNPLA3 mRNA normalized to GUSB expression and indicated phosphorylated protein as compared to total protein in hepatocytes after treatment with CH5132799. FIG. 40D shows the relative amounts of PNPLA3 mRNA normalized to GUSB expression and indicated phosphorylated protein as compared to total protein in hepatocytes after treatment with Rapamycin. FIG. 40E shows the relative amounts of PNPLA3 mRNA normalized to GUSB expression and amount of indicated phosphorylated protein as compared to total protein in hepatocytes after treatment with Alpelisib (BYL719).

FIG. 41A shows PNPLA3 liver mRNA levels in mice after OSI-027 treatment. FIG. 41B shows PNPLA3 liver mRNA levels in mice after PF-04691502 treatment.

FIG. 42A shows the serum glucose levels in mice after OSI-027 or PF-04691502 treatment. FIG. 42B shows the serum insulin levels in mice after OSI-027 or PF-04691502 treatment.

### DETAILED DESCRIPTION

### I. Introduction

Provided herein are compositions and methods for the treatment of liver diseases in humans. In particular, the invention relates to the use of compounds that modulate Patatin-like phospholipase domain-containing protein 3 (PNPLA3) for the treatment of PNPLA3-related diseases, e.g., nonalcoholic fatty liver disease (NAFLD), nonalcoholic steatohepatitis (NASH) and/or alcoholic liver disease (ALD).

Also provided herein are methods that embrace the alteration, perturbation and ultimate regulated control of gene signaling networks (GSNs). Such gene signaling networks include genomic signaling centers found within insulated neighborhoods of the genomes of biological systems. Compounds modulating PNPLA3 expression may act through modulating one or more gene signaling networks.

As used herein, a “gene signaling network” or “GSN” comprises the set of biomolecules associated with any or all of the signaling events from a particular gene, e.g., a gene-centric network. As there are over 20,000 protein-coding genes in the human genome, there are at least this many gene signaling networks. And to the extent some genes are non-coding genes, the number increases greatly. Gene signaling networks differ from canonical signaling pathways which are mapped as standard protein cascades and feedback loops.

Traditionally, signaling pathways have been identified using standard biochemical techniques and, for the most part, are linear cascades with one protein product signaling the next protein product-driven event in the cascade. While these pathways may bifurcate or have feedback loops, the focus has been almost exclusively at the protein level.

Gene signaling networks (GSNs) of the present invention represent a different paradigm to defining biological signaling—taking into account protein-coding and nonprotein-coding signaling molecules, genomic structure, chromosomal occupancy, chromosomal remodeling, the status of the biological system and the range of outcomes associated with the perturbation of any biological systems comprising such gene signaling networks.

Genomic architecture, while not static, plays an important role in defining the framework of the GSNs of the present invention. Such architecture includes the concepts of chromosomal organization and modification, topologically associated domains (TADs), insulated neighborhoods (INs), genomic signaling centers (GSCs), signaling molecules and their binding motifs or sites, and of course, the genes encoded within the genomic architecture.

The present invention, by elucidating a more definitive set of connectivities of the GSNs associated with the PNPLA3 gene, provides a fine-tuned mechanism to address PNPLA3-related diseases, including NAFLD, NASH, and/or ALD.

### Genomic Architecture

Cells control gene expression using thousands of elements that link cellular signaling to the architecture of the genome. Genomic system architecture includes regions of DNA, RNA transcripts, chromatin remodelers, and signaling molecules.

### Chromosomes

Chromosomes are the largest subunit of genome architecture that contain most of the DNA in humans. Specific chromosome structures have been observed to play important roles in gene control, as described in Hnisz et al., Cell 167, Nov. 17, 2016, which is hereby incorporated by reference in its entirety. The “non-coding regions” including introns provide protein binding sites and other regulatory structures, while the exons encode for proteins such as signaling molecules (e.g., transcription factors), that interact with the non-coding regions to regulate gene expression. DNA sites within non-coding regions on the chromosome also interact with each other to form looped structures. These interactions form a chromosome scaffold that is preserved through development and plays an important role in gene activation and repression. Interactions rarely occur among chromosomes and are usually within the same domain of a chromosome.

In situ hybridization techniques and microscopy have revealed that each interphase chromosomes tends to occupy only a small portion of the nucleus and does not spread throughout this organelle. See, Cremer and Cremer, Cold Spring Harbor Perspectives in Biology 2, a003889, 2010, which is hereby incorporated by reference in its entirety. This restricted surface occupancy area might reduce interactions between chromosomes.

### Topologically Associating Domains (TADs)

Topologically Associating Domains (TADs), alternatively known as topological domains, are hierarchical units that are subunits of the mammalian chromosome structure. See, Dixon et al., Nature, 485(7398):376-80, 2012; Filippova et al., Algorithms for Molecular Biology, 9:14, 2014; Gibcus and Dekker Molecular Cell, 49(5):773-82, 2013; Naumova et al., Science, 42(6161):948-53, 2013; which are hereby incorporated by reference in their entireties. TADs are megabase-sized chromosomal regions that demarcate a microenvironment that allows genes and regulatory elements to make productive DNA-DNA contacts. TADs are defined by DNA-DNA interaction frequencies. The boundaries of TADs consist of regions where relatively fewer DNA-DNA interactions occur, as described in Dixon et al., Nature, 485(7398):376-80, 2012; Nora et al., Nature, 485(7398):381-5, 2012; which are hereby incorporated by reference in their entirety. TADs represent structural chromosomal units that function as gene expression regulators.

TADs may contain about 7 or more protein-coding genes and have boundaries that are shared by the different cell types. See, Smallwood et al., Current Opinion in Cell Biology, 25(3):387-94, 2013, which is hereby incorporated by reference in its entirety. Some TADs contain active genes and others contain repressed genes, as the expression of genes within a single TAD is usually correlated. See, Cavalli et al., Nature Structural & Molecular Biology, 20(3):290-9, 2013, which is hereby incorporated by reference in its entirety. Sequences within a TAD find each other with high frequency and have concerted, TAD-wide histone chromatin signatures, expression levels, DNA replication timing, lamina association, and chromocenter association. See, Dixon et al., Nature, 485(7398):376-80, 2012; Le Dily et al., Genes Development, 28:2151-62, 2014; Dixon et al., Nature, 485(7398):376-80, 2012; Wijchers, Genome Research, 25:958-69, 2015, which are hereby incorporated by reference in their entireties.

Gene loops and other structures within TADs influence the activities of transcription factors (TFs), cohesin, and 11-zinc finger protein (CTCF), a transcriptional repressor. See, Baranello et al., Proceedings of the National Academy of Sciences, 111(3):889-9, 2014, which is hereby incorporated by reference in its entirety. The structures within TADs include cohesin-associated enhancer-promoter loops that are produced when enhancer-bound TFs bind cofactors, for example Mediator, that, in turn, bind RNA polymerase II at promoter sites. See, Lee and Young, Cell, 152(6):1237-51, 2013; Lelli et al., 2012; Roeder, Annual Reviews Genetics 46:43-68, 2005; Spitz and Furlong, Nature Reviews Genetics, 13(9):613-26, 2012; Dowen et al., Cell, 159(2): 374-387, 2014; Lelli et al., Annual Review of Genetics, 46:43-68, 2012, which are hereby incorporated by reference in their entireties. The cohesin-loading factor Nipped-B-like protein (NIPBL) binds Mediator and loads cohesin at these enhancer-promoter loops. See, Kagey et al., Nature, 467(7314):430-5, 2010, which is hereby incorporated by reference in its entirety.

TADs have similar boundaries in all human cell types examined and constrain enhancer-gene interactions. See, Dixon et al., Nature, 518:331-336, 2015; Dixon et al., Nature, 485:376-380, 2012, which are hereby incorporated by reference in their entirety. This architecture of the genome helps explain why most DNA contacts occur within the TADs and enhancer-gene interactions rarely occur between chromosomes. However, TADs provide only partial insight into the molecular mechanisms that influence specific enhancer-gene interactions within TADs.

Long-range genomic contacts segregate TADs into an active and inactive compartment. See, Lieberman-Aiden et al., Science, 326:289-93, 2009, which is hereby incorporated by reference in its entirety. The loops formed between TAD boundaries seem to represent the longest-range contacts that are stably and reproducibly formed between specific pairs of sequences. See, Dixon et al., Nature, 485(7398):376-80, 2012, which is hereby incorporated by reference in its entirety.

In some embodiments, the methods of the present invention are used to alter gene expression from genes located in a TAD. In some embodiments, TAD regions are modified to alter gene expression of a non-canonical pathway as defined herein or as definable using the methods described herein.

### Insulated Neighborhoods

As used herein, an “insulated neighborhood” (IN) is defined as a chromosome structure formed by the looping of two interacting sites in the chromosome sequence. These interacting sites may comprise CCCTC-Binding factor (CTCF). These CTCF sites are often co-occupied by cohesin. The integrity of these cohesin-associated chromosome structures affects the expression of genes in the insulated neighborhood as well as those genes in the vicinity of the insulated neighborhoods. A “neighborhood gene” is a gene localized within an insulated neighborhood. Neighborhood genes may be coding or non-coding.

Insulated neighborhood architecture is defined by at least two boundaries which come together, directly or indirectly, to form a DNA loop. The boundaries of any insulated neighborhood comprise a primary upstream boundary and a primary downstream boundary. Such boundaries are the outermost boundaries of any insulated neighborhood. Within any insulated neighborhood loop, however, secondary loops may be formed. Such secondary loops, when present, are defined by secondary upstream boundaries and secondary downstream boundaries, relative to the primary insulated neighborhood. Where a primary insulated neighborhood contains more than one internal loop, the loops are numbered relative to the primary upstream boundary of the primary loop, e.g., the secondary loop (first loop within the primary loop), the tertiary loop (second loop within the primary loop), the quaternary loop (the third loop within the primary loop) and so on.

Insulated neighborhoods may be located within topologically associated domains (TADs) and other gene loops. Largest insulated neighborhoods may be TADs. TADs are defined by DNA-DNA interaction frequencies, and average 0.8 Mb, contain approximately 7 protein-coding genes and have boundaries that are shared by the different cell types of an organism. According to Dowen, the expression of genes within a TAD is somewhat correlated, and thus some TADs tend to have active genes and others tend to have repressed genes. See Dowen et al., Cell. 2014 Oct. 9; 159(2): 374-387, which is hereby incorporated by reference herein in its entirety.

Insulated neighborhoods may exist as contiguous entities along a chromosome or may be separated by non-insulated neighborhood sequence regions. Insulated neighborhoods may overlap linearly only to be defined once the DNA looping regions have been joined. While insulated neighborhoods may comprise 3-12 genes, they may contain, 1, 2, 3, 4, 5, 6, 7, 8, 9, 10, 11, 12, 13 or more genes.

A “minimal insulated neighborhood” is an insulated neighborhood having at least one neighborhood gene and associated regulatory sequence region (RSRs) or regions which facilitate the expression or repression of the neighborhood gene such as a promoter and/or enhancer and/or repressor region, and the like. It is contemplated that in some instances regulatory sequence regions may coincide or even overlap with an insulated neighborhood boundary. Regulatory sequence regions, as used herein, include but are not limited to regions, sections, sites or zones along a chromosome whereby interactions with signaling molecules occur in order to alter expression of a neighborhood gene. As used herein, a “signaling molecule” is any entity, whether protein, nucleic acid (DNA or RNA), organic small molecule, lipid, sugar or other biomolecule, which interacts directly, or indirectly, with a regulatory sequence region on a chromosome. Regulatory sequence regions (RSRs) may also refer to a portion of DNA that functions as a binding site for a GSC.

One category of specialized signaling molecules are transcription factors. “Transcription factors” are those signaling molecules which alter, whether to increase or decrease, the transcription of a target gene, e.g., a neighborhood gene.

According to the present invention, neighborhood genes may have any number of upstream or downstream genes along the chromosome. Within any insulated neighborhood, there may be one or more, e.g., one, two, three, four or more, upstream and/or downstream neighborhood genes relative to the primary neighborhood gene. A “primary neighborhood gene” is a gene which is most commonly found within a specific insulated neighborhood along a chromosome. An upstream neighborhood gene of a primary neighborhood gene may be located within the same insulated neighborhood as the primary neighborhood gene. A downstream neighborhood gene of a primary neighborhood gene may be located within the same insulated neighborhood as the primary neighborhood gene.

The present invention provides methods of altering the penetrance of a gene or gene variant. As used herein, “penetrance” is the proportion of individuals carrying a particular variant of a gene (e.g., mutation, allele or generally a genotype, whether wild type or not) that also exhibits an associated trait (phenotype) of that variant gene. In some situations of disease, penetrance of a disease-causing mutation measured as the proportion of individuals with the mutation who exhibit clinical symptoms. Consequently, penetrance of any gene or gene variant exists on a continuum.

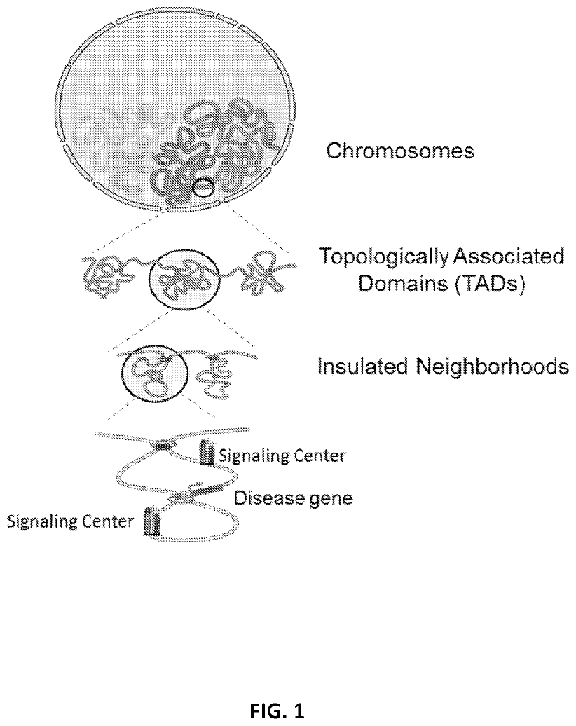

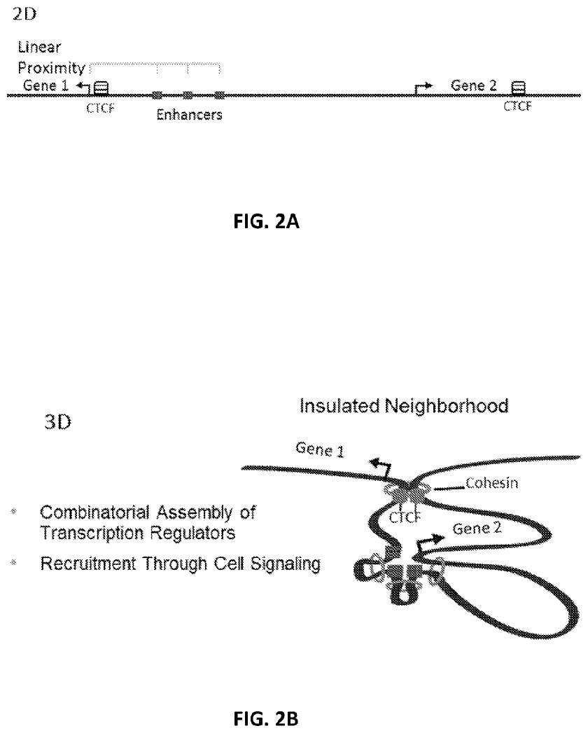

Insulated neighborhoods are functional units that may group genes under the same control mechanism, which are described in Dowell et al., Cell, 159: 374-387 (2014), which is hereby incorporated by reference in its entirety. Insulated neighborhoods provide the mechanistic background for higher-order chromosome structures, such as TADs which are shown in FIG. 1. Insulated neighborhoods are chromosome structures formed by the looping of the two interacting CTCF sites co-occupied by cohesin as shown in FIG. 2B. The integrity of these structures is important for proper expression of local genes. Generally, 1 to 10 genes are clustered in each neighborhood with a median number of 3 genes within each one. The genes controlled by the same insulated neighborhood are not readily apparent from a two-dimensional view of DNA. In humans, there are about 13,801 insulated neighborhoods in a size range of 25 kb-940 kb with a median size of 1861 b. Insulated neighborhoods are conserved among different cell types. Smaller INs that occur within a bigger IN are referred to as nested insulated neighborhoods (NINs). TADs can consist of a single IN as shown in FIG. 1, or one IN and one NIN and two NINs as shown in FIG. 2B.

As used herein, the term “boundary” refers to a point, limit, or range indicating where a feature, element, or property ends or begins. Accordingly, an “insulated neighborhood boundary” refers to a boundary that delimits an insulated neighborhood on a chromosome. According to the present invention, an insulated neighborhood is defined by at least two insulated neighborhood boundaries, a primary upstream boundary and a primary downstream boundary. The “primary upstream boundary” refers to the insulated neighborhood boundary located upstream of a primary neighborhood gene. The “primary downstream boundary” refers to the insulated neighborhood boundary located downstream of a primary neighborhood gene. Similarly, when secondary loops are present as shown in FIG. 2B, they are defined by secondary upstream and downstream boundaries. A “secondary upstream boundary” is the upstream boundary of a secondary loop within a primary insulated neighborhood, and a “secondary downstream boundary” is the downstream boundary of a secondary loop within a primary insulated neighborhood. The directionality of the secondary boundaries follows that of the primary insulated neighborhood boundaries.

Components of an insulated neighborhood boundary may comprise the DNA sequences at the anchor regions and associated factors (e.g., CTCF, cohesin) that facilitate the looping of the two boundaries. The DNA sequences at the anchor regions may contain at least one CTCF binding site. Experiments using the ChIP-exo technique revealed a 52 bb CTCF binding motif containing four CTCF binding modules (see FIG. 1, Ong and Corces, Nature reviews Genetics, 12:283-293, 2011, which is incorporated herein by reference in its entirety). The DNA sequences at the insulated neighborhood boundaries may contain insulators. In some cases, insulated neighborhood boundaries may also coincide or overlap with regulatory sequence regions, such as enhancer-promoter interaction sites.

In some embodiments of the present invention, disrupting or altering an insulated neighborhood boundary may he accomplished by altering specific DNA sequences (e.g., CTCF binding sites) at the boundaries. For example, existing CTCF binding sites at insulated neighborhood boundaries may be deleted, mutated, or inverted. Alternatively, new CTCF binding sites may be introduced to form new insulated neighborhoods. In other embodiments, disrupting or altering an insulated neighborhood boundary may be accomplished by altering the histone modification (e.g., methylation, demethylation) at the boundaries. In other embodiments, disrupting or altering an insulated neighborhood boundary may be accomplished by altering (e.g., blocking) the binding of CTCF and/or cohesin to the boundaries. In cases where insulated neighborhood boundaries coincide or overlap with regulatory sequence regions, disrupting or altering an insulated neighborhood boundary may be accomplished by altering the regulatory sequence regions (RSR) or the binding of the RSR-associated signaling molecules.

Controlling Expression from Insulated Neighborhoods: Signaling Centers

Historically, the term “signaling center” has been used to describe a group of cells responding to changes in the cellular environment. See, Guger et at. Developmental Biology 172: 115-125 (1995), which is incorporated by reference herein in its entirety. Similarly, the term “signaling center”, as used herein, refers to a defined region of a living organism that interacts with a defined set of biomolecules, such as signaling proteins or signaling molecules (e.g., transcription factors) to regulate gene expression in a context-specific manner.

Specifically, the term “genomic signaling center”, i.e., a “signaling center”, as used herein, refers to regions within insulated neighborhoods that include regions capable of binding context-specific combinatorial assemblies of signaling molecules/signaling proteins that participate in the regulation of the genes within that insulated neighborhood or among more than one insulated neighborhood.

Signaling centers have been discovered to regulate the activity of insulated neighborhoods. These regions control which genes are expressed and the level of expression in the human genome. Loss of the structural integrity of signaling centers contributes to deregulation of gene expression and potentially causing disease.

Signaling centers include enhancers bound by a highly context-specific combinatorial assemblies of transcription factors. These factors are recruited to the site through cellular signaling. Signaling centers include multiple genes that interact to form a three-dimensional transcription factor hub macrocomplex. Signaling centers are generally associated with one to four genes in a loop organized by biological function.

The compositions of each signaling center has a unique composition including the assemblies of transcription factors, the transcription apparatus, and chromatin regulators. Signaling centers are highly context specific, permitting drugs to control response by targeting signaling pathways.

Multiple signaling centers may interact to control the different combinations of genes within the same insulated neighborhood.

### Binding Sites for Signaling Molecules

A series of consensus binding sites, or binding motifs for binding sites, for signaling molecules has been identified by the present inventors. These consensus sequences reflect binding sites along a chromosome, gene, or polynucleotide for signaling molecules or for complexes which include one or more signaling molecules.

In some embodiments, binding sites are associated with more than one signaling molecule or complex of molecules.

### Enhancers

Enhancers are gene regulatory elements that control cell type specific gene expression programs in humans. See, Buecker and Wysocka, Trends in genetics: TIG 28, 276-284, 2012; Heinz etal., Nature reviews Molecular Cell Biology, 16:144-154, 2015; Levine etal., Cell, 157:13-25, 2014; 0 ng and Corces, Nature reviews Genetics, 12:283-293, 2011; Ren and Yue, Cold Spring Harbor symposia on quantitative biology, 80:17-26, 2015, which are hereby incorporated by reference in their entireties. Enhancers are segments of DNA that are generally a few hundred base pairs in length that may be occupied by multiple transcription factors that recruit co-activators and RNA polymerase II to target genes. See, Bulger and Groudine, Cell, 144:327-339, 2011; Spitz and Furlong, Nature reviews Genetics, 13:613-626, 2012; Tjian and Maniatis, Cell, 77:5-8, 1994, which are hereby incorporated by reference in their entireties. Enhancer RNA molecules transcribed from these regions of DNA also “trap” transcription factors capable of binding DNA and RNA. A region with more than one enhancer is a “super-enhancer.”

Insulated neighborhoods provide a microenvironment for specific enhancer-gene interactions that are vital for both normal gene activation and repression. Transcriptional enhancers control over 20,000 protein-coding genes to maintain cell type-specific gene expression programs in all human cells. Tens of thousands of enhancers are estimated to be active in any given human cell type. See, ENCODE Project Consortium et al., Nature, 489, 57-74, 2012; Roadmap Epigenomics et al., Nature, 518, 317-330, 2015, which are hereby incorporated by reference in their entirety. Enhancers and their associated factors can regulate expression of genes located upstream or downstream by looping to the promoters of these genes. Cohesin ChIA-PET studies carried out to gain insight into the relationship between transcriptional control of cell identity and control of chromosome structure reveal that majority of the super-enhancers and their associated genes occur within large loops that are connected through interacting CTCF-sites co-occupied by cohesin. Such super-enhancer domains (SD) usually contain one super-enhancer that loops to one gene within the SD and the SDs appear to restrict super-enhancer activity to genes within the SD. The correct association of super-enhancers and their target genes in insulated neighborhoods is highly vital because the mis-targeting of a single super-enhancer is sufficient to cause disease. See Groschel et al., Cell, 157(2):369-81, 2014.

Most of the disease-associated non-coding variation occurs in the vicinity of enhancers and hence might impact these enhancer target genes. Therefore, deciphering the features conferring specificity to enhancers is important for modulatory gene expression. See, Ernst et al., Nature, 473, 43-49, 2011; Farh et al., Nature, 518, 337-343,2015; Hnisz et al., Cell, 155, 934-947, 2013; Maurano et al., Science, 337, 1190-1195, 2012, which are hereby incorporated by reference in their entirety. Studies suggest that some of the specificity of enhancer-gene interactions may be due to the interaction of DNA binding transcription factors at enhancers with specific partner transcription factors at promoters. See, Butler and Kadonaga, Genes & Development, 15, 2515-2519, 2001; Choi and Engel, Cell, 55, 17-26, 1988; Ohtsuki et al., Genes & Development, 12, 547-556, 1998, which are hereby incorporated by reference in their entireties. DNA sequences in enhancers and in promoter-proximal regions bind to a variety of transcription factors expressed in a single cell. Diverse factors bound at these two sites interact with large cofactor complexes and interact with one another to produce enhancer-gene specificity. See, Zabidi et al., Nature, 518:556-559, 2015, which is hereby incorporated by reference in its entirety.

In some embodiments, enhancer regions may be targeted to alter or elucidate gene signaling networks (GSNs).

### Insulators

Insulators are regulatory elements that block the ability of an enhancer to activate a gene when located between them and contribute to specific enhancer-gene interactions. See, Chung et al., Cell 74:505-514, 1993; Geyer and Corces, Genes & Development 6:1865-1873, 1992; Kellum and Schedl, Cell 64:941-950, 1991; Udvardy et al., Journal of molecular biology 185:341-358, 1985, which are hereby incorporated by reference in their entirety. Insulators are bound by the transcription factor CTCF but not all CTCF sites function as insulators. See, Bell et al., Cell 98: 387-396, 1999; Liu et al., Nature biotechnology 33:198-203, 2015, which are hereby incorporated by reference in their entireties. The features that distinguish the subset of CTCF sites that function as insulators have not been previously understood.

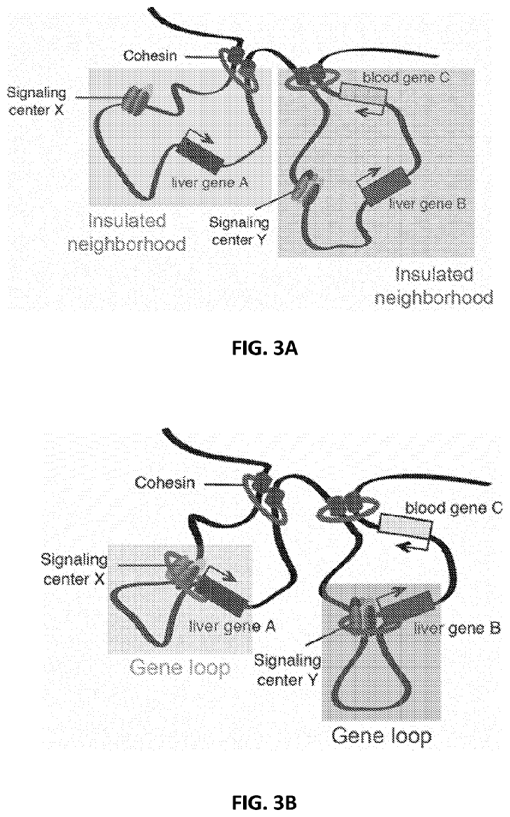

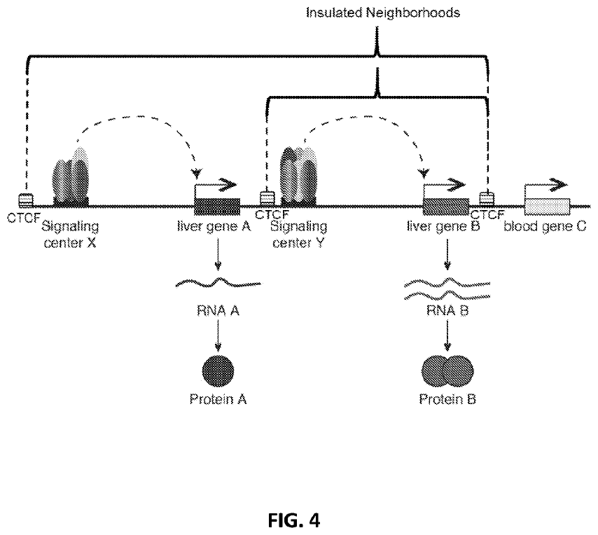

Genome-wide maps of the proteins that bind enhancers, promoters and insulators, together with knowledge of the physical contacts that occur between these elements provide further insight into understanding of the mechanisms that generate specific enhancer-gene interactions. See, Chepelev et al., Cell research, 22:490-503, 2012; DeMare et al., Genome Research, 23:1224-1234, 2013; Dowen et al., Cell, 159:374-387, 2014; Fullwood et al., Genes & Development 6:1865-1873, 2009; Handoko et al., Nature genetics 43:630-638, 2011; Phillips-Cremins et al., Cell, 153:1281-1295, 2013; Tang et al., Cell 163:1611-1627, 2015, which are hereby incorporated by reference in their entirety. Enhancer-bound proteins are constrained such that they tend to interact only with genes within these CTCF-CTCF loops. The subset of CTCF sites that form these loop anchors thus function to insulate enhancers and genes within the loop from enhancers and genes outside the loop, as shown in FIG. 3B. In some embodiments, insulator regions may be targeted to alter or elucidate gene signaling networks (GSNs).

### Cohesin and CTCF Associated Loops and Anchor Sites/Regions

CTCF interactions link sites on the same chromosome forming loops, which are generally less than 1 Mb in length. Transcription occurs both within and outside the loops, but the nature of this transcription differs between the two regions. Studies show that enhancer-associated transcription is more prominent within the loops. Thus, the insulator state is enriched specifically at the CTCF loop anchors. CTCF loops thus either enclose gene poor regions, with a tendency for genes to be centered within the loops or leave out gene dense regions outside the CTCF loops. FIG. 2A and FIG. 2B compare the linear to the 3-dimensional (3D) conformation of the loops.

CTCF loops exhibit reduced exon density relative to their flanking regions. Gene ontology analysis reveals that genes located within CTCF loops are enriched for response to stimuli and for extracellular, plasma membrane and vesicle cellular localizations. On the other hand, genes present within the flanking regions just outside the loops exhibit an expression pattern similar to housekeeping genes i.e. these genes are on average more highly expressed than the loop-enclosed genes, are less cell-line specific in their expression pattern, and have less variation in their expression levels across cell lines. See Oti et al., BMC Genomics, 17:252, 2016, which is hereby incorporated by reference in its entirety.

Anchor regions are binding sites for CTCF that influence conformation of an insulated neighborhood. Deletion of anchor sites may result in activation of genes that are usually transcriptionally silent, thereby resulting in a disease phenotype. In fact, somatic mutations are common in loop anchor sites of oncogene-associated insulated neighborhoods. The CTCF DNA-binding motif of the loop anchor region has been observed to be the most altered human transcription-factor binding sequence of cancer cells. See, Hnisz et al., Cell 167, Nov. 17, 2016, which is incorporated by reference in its entirety.

Anchor regions have been observed to be largely maintained during cell development, and are especially conserved in the germline of humans and primates. In fact, the DNA sequence of anchor regions are more conserved in CTCF anchor regions than at CTCF binding sites that are not part of an insulated neighborhood. Therefore, cohesin may be used as a target for ChIA-PET to identify locations of both.

Cohesin also becomes associated with CTCF-bound regions of the genome, and some of these cohesin-associated CTCF sites facilitate gene activation while others may function as insulators. See, Dixon et al., Nature, 485(7398):376-80, 2012; Parelho et al., Cell, 132(3):422-33, 2008; Phillips-Cremins and Corces, Molecular Cell, 50(4):461-74, 2013); Seitan et al., Genome Research, 23(12):2066-77, 2013; Wendt et al., Nature, 451(7180):796-801, 2008), which are hereby incorporated by reference in their entireties. Cohesin and CTCF are associated with large loop substructures within TADs, and cohesin and Mediator are associated with smaller loop structures that form within CTCF-bounded regions. See, de Wit et al., Nature, 501(7466):227-31, 2013; Cremins et al., Cell, 153(6):1281-95, 2013; Sofueva et al., EMBO, 32(24):3119-29, 2013, which are hereby incorporated by reference in their entireties. In some embodiments, cohesin and CTCF associated loops and anchor sites/regions may be targeted to alter or elucidate gene signaling networks (GSNs).

Genetic Variants

Genetic variations within signaling centers are known to contribute to disease by disrupting protein binding on chromosomes, such as described in Hnisz et al., Cell 167, Nov. 17, 2016, which is hereby incorporated by reference in its entirety. Variations of the sequence of CTCF anchor regions of insulated neighborhood boundary sites that interfere with formation of insulated neighborhoods are observed to result in dysregulation of gene activation and repression. CTCF malfunctions caused by various genetic and epigenetic mechanisms may lead to pathogenesis. Therefore, in some embodiments, it is beneficial to alter any one or more gene signaling networks (GSNs) associated with such variant-driven etiology in order to effect one or more positive treatment outcomes.

Single Nucleotide Polymorphisms (SNPs)

94.2% of SNPs occur in non-coding regions, which include enhancer regions. In some embodiments, SNPs are altered in order to study and/or alter the signaling from one or more GSN.

Signaling Molecules

Signaling molecules include any protein that functions in cellular signaling pathways, whether canonical or the gene signaling network pathways defined herein or capable of being defined using the methods described herein. Transcription factors are a subset of signaling molecules. Certain combinations of signaling and master transcription factors associate to an enhancer region to influence expression of a gene. Master transcription factors direct transcription factors in specific tissues. For example, in blood, GATA transcription factors are master transcription factors that direct TCF7L2 of the Wnt cellular signaling pathway. In the liver, HNF4A is a master transcription factor to direct SMAD in lineage tissues and patterns.

Transcriptional regulation allows controlling how often a given gene is transcribed. Transcription factors alter the rate at which transcripts are produced by making conditions for transcription initiation more or less favorable. A transcription factor selectively alters a signaling pathway which in turn affects the genes controlled by a genomic signaling center. Genomic signaling centers are components of transcriptional regulators. In some embodiments, signaling molecules may be used, or targeted in order to elucidate or alter the signaling of gene signaling networks of the present invention.

Table 22 of International Application No. PCT/US18/31056, which is hereby incorporated by reference in its entirety, provides a list of signaling molecules including those which act as transcription factors (TF) and/or chromatin remodeling factors (CR) that function in various cellular signaling pathways. The methods described herein may be used to inhibit or activate the expression of one or more signaling molecules associated with the regulatory sequence region of the primary neighborhood gene encoded within an insulated neighborhood. The methods may thus alter the signaling signature of one or more primary neighborhood genes which are differentially expressed upon treatment with the therapeutic agent compared to an untreated control.

### Transcription Factors

Transcription factors generally regulate gene expression by binding to enhancers and recruiting coactivators and RNA polymerase II to target genes. See Whyte et al., Cell, 153(2): 307-319, 2013, which is incorporated by reference in its entirety. Transcription factors bind “enhancers” to stimulate cell-specific transcriptional program by binding regulatory elements distributed throughout the genome.

There are about 1800 known transcription factors in the human genome. There are epitopes on the DNA of the chromosomes that provide binding sites for proteins or nucleic acid molecules such as ribosomal RNA complexes. Master regulators direct a combination of transcription factors through cell signaling above and DNA below. These characteristics allow for determination of the location of the next signaling center. In some embodiments, transcription factors may be used or targeted, to alter or elucidate the gene signaling networks of the present invention.

### Master Transcription Factors

Master transcription factors bind and establish cell-type specific enhancers. Master transcription factors recruit additional signaling proteins, such as other transcription factors, to enhancers to form signaling centers. An atlas of candidate master TFs for 233 human cell types and tissues is described in D'Alessio et al., Stem Cell Reports 5, 763-775 (2015), which is hereby incorporated by reference in its entirety. In some embodiments, master transcription factors may be used or targeted, to alter or elucidate the gene signaling networks of the present invention.

### Signaling Transcription Factors

Signaling transcription factors are transcription factors, such as homeoproteins, that travel between cells as they contain protein domains that allow them to do the so. Homeoproteins such as Engrailed, Hoxa5, Hoxb4, Hoxc8, Emx1, Emx2, Otx2 and Pax6 are able to act as signaling transcription factors. The homeoprotein Engrailed possesses internalization and secretion signals that are believed to be present in other homeoproteins as well. This property allows homeoproteins to act as signaling molecules in addition to being transcription factors. Homeoproteins lack characterized extracellular functions leading to the perception that their paracrine targets are intracellular. The ability of homeoproteins to regulate transcription and, in some cases, translation is most likely to affect paracrine action. See Prochiantz and Joliot, Nature Reviews Molecular Cell Biology, 2003. In some embodiments, signaling transcription factors may be used or targeted, to alter or elucidate the gene signaling networks of the present invention.

### Chromatin Modifications

Chromatin remodeling is regulated by over a thousand proteins that are associated with histone modification. See, Ji et al., PNAS, 112(12):3841-3846(2015), which is hereby incorporated by reference in its entirety. Chromatin regulators are specific sets of proteins associated with genomic regions marked with modified histones. For example, histones may be modified at certain lysine residues: H3K20me3, H3K27ac, H3K4me3, H3K4me1, H3K79me2, H3K36me3, H3K9me2, and H3K9me3. Certain histone modifications mark regions of the genome that are available for binding by signaling molecules. For example, previous studies have observed that active enhancer regions include nucleosomes with H3K27ac, and active promoters include nucleosomes with H3K27ac. Further, transcribed genes include nucleosomes with H3K79me2. ChIP-MS may be performed to identify chromatin regulator proteins associated with specific histone modification. ChIP-seq with antibodies specific to certain modified histones may also be used to identify regions of the genome that are bound by signaling molecules. In some embodiments, chromatin modifying enzymes or proteins may be used or targeted, to alter or elucidate the gene signaling networks of the present invention.

RNAs Derived from Regulatory Sequence Regions

Many active regulatory sequence regions (RSRs), such as regions from enhancers, signaling centers, and promoters of protein-coding genes, are known to produce non-coding RNAs. Transcripts produced at or in the vicinity of active regulatory sequence regions have been implicated in transcription regulation of nearby genes. Recent reports have demonstrated that enhancer-associated RNAs (eRNAs) are strong indicators of enhancer activity (See Li et al., Nat Rev Genet. 2016 April; 17(4):207-23, which is hereby incorporated by reference in its entirety). Further, non-coding RNAs from active regulatory sequence regions have been shown to be involved in facilitating the binding of transcription factors to these regions (Sigova et al., Science. 2015 Nov. 20; 350(6263):978-81, which is hereby incorporated by reference in its entirety). This suggests that such RNAs may be important for the assembly of signaling centers and regulation of neighborhood genes. In some embodiments, RNAs derived from regulatory sequence regions of the PNPLA3 gene may be used or targeted to alter or elucidate the gene signaling networks of the present invention.

In some embodiments, RNAs derived from regulatory sequence regions may be an enhancer-associated RNA (eRNA). In some embodiments, RNAs derived from regulatory sequence regions may be a promoter-associated RNA, including but not limited to, a promoter upstream transcript (PROMPT), a promoter-associated long RNA (PALR), and a promoter-associated small RNA (PASR). In further embodiments, RNAs derived from regulatory sequence regions may include but are not limited to transcription start sites (TSS)-associated RNAs (TSSa-RNAs), transcription initiation RNAs (tiRNAs), and terminator-associated small RNAs (TASRs).

In some embodiments, RNAs derived from regulatory sequence regions may be long non-coding RNAs (lncRNAs) (i.e., >200 nucleotides). In some embodiments, RNAs derived from regulatory sequence regions may be intermediate non-coding RNAs. (i.e., about 50 to 200 nucleotides). In some embodiments, RNAs derived from regulatory sequence regions may be short non-coding RNAs (i.e., about 20 to 50 nucleotides).

In some embodiments, eRNAs that may be modulated by methods and compounds described herein may be characterized by one or more of the following features: (1) transcribed from regions with high levels of monomethylation on lysine 4 of histone 3 (H3K4me1) and low levels of trimethylation on lysine 4 of histone 3 (H3K4me3); (2) transcribed from genomic regions with high levels of acetylation on lysine 27 of histone 3 (H3K27ac); (3) transcribed from genomic regions with low levels of trimethylation on lysine 36 of histone 3 (H3K36me3); (4) transcribed from genomic regions enriched for RNA polymerase II (Pol II); (5) transcribed from genomic regions enriched for transcriptional co-regulators, such as the p300 co-activator; (6) transcribed from genomic regions with low density of CpG island; (7) their transcription is initiated from Pol II-binding sites and elongated bidirectionally; (8) evolutionarily conserved DNA sequences encoding eRNAs; (9) short half-life; (10) reduced levels of splicing and polyadenylation, (11) dynamically regulated upon signaling; (12) positively correlated to levels of nearby mRNA expression; (13) extremely high tissue specificity; (14) preferentially nuclear and chromatin-bound; and/or (15) degraded by the exosome.

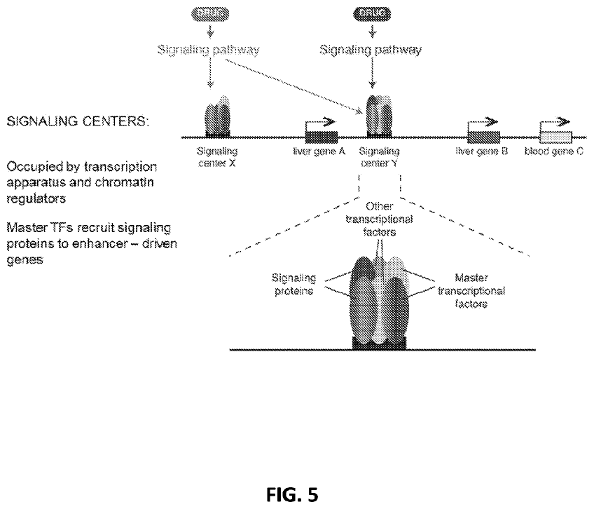

Exemplary eRNAs include those described in Djebali et al., Nature. 2012 Sep. 6; 489(7414) (for example, Supplementary data file for FIG. 5a) and Andersson et al., Nature. 2014 Mar. 27; 507(7493):455-461 (for example, Supplementary Tables S3, S12, S13, S15, and 16), which are herein incorporated by reference in their entirety.

In some embodiments, promoter-associated RNAs that may be modulated by methods or compounds described herein may be characterized by one or more of the following features: (1) transcribed from regions with high levels of H3K4me1 and low to medium levels of H3K4me3; (2) transcribed from genomic regions with high levels of H3K27ac; (3) transcribed from genomic regions with no or low levels of H3K36me3; (4) transcribed from genomic regions enriched for RNA polymerase II (Pol II); (5) transcribed from genomic regions with high density of CpG island; (6) their transcription is initiated from Pol II-binding sites and elongated in the opposite direction from the sense strand (that is, mRNAs) or bidirectionally; (7) short half-life; (8) reduced levels of splicing and polyadenylation; (9) preferentially nuclear and chromatin-bound; and/or (10) degraded by the exosome.

In some embodiments, compositions and methods described herein may be used to modulate RNAs derived from regulatory sequence regions to alter or elucidate the gene signaling networks of the present invention. In some embodiments, methods and compounds described herein may be used to inhibit the production and/or function of an RNA derived from regulatory sequence regions. In some embodiments, a hybridizing oligonucleotide such as an siRNA or an antisense oligonucleotide may be used to inhibit the activity of the RNA of interest via RNA interference (RNAi), or RNase H-mediated cleavage, or physically block binding of various signaling molecules to the RNA. Exemplary hybridizing oligonucleotide may include those described in U.S. Pat No. 9,518,261 and PCT Publication No. WO 2014/040742, which are hereby incorporated by reference in their entirety. The hybridizing oligonucleotide may be provided as a chemically modified or unmodified RNA, DNA, locked nucleic acids (LNA), or a combination of RNA and DNA, a nucleic acid vector encoding the hybridizing oligonucleotide, or a virus carrying such vector. In other embodiments, genome editing tools such as CRISPR/Cas9 may be used to delete specific DNA elements in the regulatory sequence regions that control the transcription of the RNA or degrade the RNA itself. In other embodiments, genome editing tools such as a catalytically inactive CRISPR/Cas9 may be used to bind to specific elements in the regulatory sequence regions and block the transcription of the RNA of interest. In further embodiments, bromodomain and extra-terminal domain (BET) inhibitors (e.g., JQ1, I-BET) may be used to reduce RNA transcription through inhibition of histone acetylation by BET protein Brd4.

In alterative embodiments, methods and compounds described herein may be used to increase the production and/or function of an RNA derived from regulatory sequence regions. In some embodiments, an exogenous synthetic RNA that mimic the RNA of interest may be introduced into the cell. The synthetic RNA may be provided as an RNA, a nucleic acid vector encoding the RNA, or a virus carrying such vector. In other embodiments, genome editing tools such as CRISPR/Cas9 may be used to tether an exogenous synthetic RNA to specific sites in the regulatory sequence regions. Such RNA may be fused to the guide RNA of the CRISPR/Cas9 complex.

In some embodiments, modulation of RNAs derived from regulatory sequence regions increases the expression of the PNPLA3 gene. In some embodiments, modulation of RNAs derived from regulatory sequence regions reduces the expression of the PNPLA3 gene.

In some embodiments, RNAs modulated by compounds described herein include RNAs derived from regulatory sequence regions of the PNPLA3 in a liver cell (e.g., a hepatocyte or a stellate cell).

Perturbation of Genomic Systems

Behavior of one or more components of the gene signaling networks (GSNs), genomic signaling centers (GSCs), and/or insulated neighborhoods (INs) related to PNPLA3 as described herein may be altered by contacting the systems containing such features with a perturbation stimulus. Potential stimuli may include exogenous biomolecules such as small molecules, antibodies, proteins, peptides, lipids, fats, nucleic acids, and the like or environmental stimuli such as radiation, pH, temperature, ionic strength, sound, light and the like.

The present invention serves, not only as a discovery tool for the elucidation of better defined gene signaling networks (GSNs) and consequently a better understanding of biological systems. The present invention allows the ability to properly define gene signaling for PNPLA3 at the gene level in a manner which allows the prediction, a priori, of potential treatment outcomes, the identification of novel compounds or targets which may have never been implicated in the treatment of a PNPLA3-related disease or condition, reduction or removal of one or more treatment liabilities associated with new or known drugs such as toxicity, poor half-life, poor bioavailability, lack of or loss of efficacy or pharmacokinetic or pharmacodynamic risks.

Treatment of disease by altering gene expression of canonical cellular signaling pathways has been shown to be effective. Even small changes in gene expression may have a significant impact on disease. For example, changes in signaling centers leading to signaling pathways affecting cell suicide suppression are associated with disease. The present invention, by elucidating a more definitive set of connectivities of the GSNs provides a fine-tuned mechanism to address disease, including genetic diseases. A method of treating a disease may include modifying a signaling center that is involved in a gene associated with that disease. Such genes may not presently be associated with the disease except as is elucidated using the methods described herein.

A perturbation stimulus may be a small molecule, a known drug, a biological, a vaccine, an herbal preparation, a hybridizing oligonucleotide (e.g., siRNA and antisense oligonucleotide), a gene or cell therapy product, or other treatment product.

In some embodiments, methods of the present invention include applying a perturbation stimulus to perturb GSNs, genomic signaling centers, and/or insulated neighborhoods associated with the PNPLA3 gene. Perturbation stimuli that causes changes in PNPLA3 expression may inform the connectivities of the associated GSNs and provide potential targets and/or treatments for PNPLA3-related disorders.

### Downstream Targets

In certain embodiments, a stimulus is administered that targets a downstream product of a gene of a gene signaling network. Alternatively, the stimulus disrupts a gene signaling network that affects downstream expression of at least one downstream target. In some embodiments, the gene is PNPLA3.

mRNA

Perturbation of a single or multiple gene signaling network (GSN) associated with a single insulated neighborhood or across multiple insulated neighborhoods can affect the transcription of a single gene or a multiple set of genes by altering the boundaries of the insulated neighborhood due to loss of anchor sites comprising cohesins. Specifically, perturbation of a GSC may also affect the transcription of a single gene or a multiple set of genes. Perturbation stimuli may result in the modification of the RNA expression and/or the sequences in the primary transcript within the mRNA, i.e. the exons or the RNA sequences between the exons that are removed by splicing, i.e. the introns. Such changes may consequently alter the members of the set of signaling molecules within the gene signaling network of a gene, thereby defining a variant of the gene signaling network.

### Proteins

Perturbation of a single or multiple gene signaling networks associated with a single insulated neighborhood or across multiple insulated neighborhoods can affect the translation of a single gene or a multiple set of genes that are part of the genomic signaling center, as well as those downstream to the genomic signaling center. Specifically, perturbation of a genomic signaling center may affect translation. Perturbation may result in the inhibition of the translated protein.

### Nearest Neighbor Gene

Perturbation stimuli may cause interactions with signaling molecules to occur in order to alter expression of the nearest primary neighborhood gene that may be located upstream or downstream of the primary neighborhood gene. Neighborhood genes may have any number of upstream or downstream genes along the chromosome. Within any insulated neighborhood, there may be one or more, e.g., one, two, three, four or more, upstream and/or downstream neighborhood genes relative to the primary neighborhood gene. A “primary neighborhood gene” is a gene which is most commonly found within a specific insulated neighborhood along a chromosome. An upstream neighborhood gene of a primary neighborhood gene may be located within the same insulated neighborhood as the primary neighborhood gene. A downstream neighborhood gene of a primary neighborhood gene may be located within the same insulated neighborhood as the primary neighborhood gene.

### Canonical Cell Signaling Pathways

It is understood that there may be some overlap between the canonical pathways detailed in the art and the gene signaling networks (GSNs) defined herein.

Whereas canonical pathways permit a certain degree of promiscuity of members across pathways (cross talk), gene signaling networks (GSN) of the invention are defined at the gene level and characterized based on any number of stimuli or perturbation to the cell, tissue, organ or organ system expressing that gene. Hence the nature of a GSN is both structurally (e.g., the gene) and situationally (e.g., the function, e.g., expression profile) defined. And while two different gene signaling networks may share members, they are still unique in that the nature of the perturbation can distinguish them. Hence, the value of GSNs in the elucidation of the function of biological systems in support of therapeutic research and development.

It should be understood that it is not intended that no connection ever be made between canonical pathways and gene signaling networks; in fact, the opposite is the case. In order to bridge the two signaling paradigms for further scientific insights, it will be instructive to compare the canonical signaling pathway paradigm with the gene signaling networks of the present invention.

In some embodiments, methods of the present invention involve altering the Janus kinases (JAK)/signal transducers and activators of transcription (STAT) pathway. The JAK/STAT pathway is the major mediator for a wide array of cytokines and growth factors. Cytokines are regulatory molecules that coordinate immune responses. JAKs are a family of intracellular, nonreceptor tyrosine kinases that are typically associated with cell surface receptors such as cytokine receptors. Mammals are known to have 4 JAKs: JAK1, JAK2, JAK3, and Tyrosine kinase 2 (TYK2). Binding of cytokines or growth factors to their respective receptors at the cell surface initiates trans-phosphorylation of JAKs, which activates downstream STATs. STATs are latent transcription factors that reside in the cytoplasm until activated. There are seven mammalian STATs: STAT1, STAT2, STAT3, STAT4, STAT5 (STAT5A and STAT5B), and STAT6. Activated STATs translocate to the nucleus where they complex with other nuclear proteins and bind to specific sequences to regulate the expression of target genes. Thus, the JAK/STAT pathway provides a direct mechanism to translate an extracellular signal into a transcriptional response. Target genes regulated by JAK/STAT pathway are involved in immunity, proliferation, differentiation, apoptosis and oncogenesis. Activation of JAKs may also activate the phosphatidylinositol 3-kinase (PI3K) and mitogen-activated protein kinase (MAPK) pathways.

In some embodiments, methods of the present invention involve altering the p53 mediated apoptosis pathway. Tumor protein p53 regulates the cell cycle and hence functions as a tumor suppressor to prevent cancer. p53 plays an important role in apoptosis, inhibition of angiogenesis and genomic stability by activating DNA repair proteins, arresting cell growth though holding the cell cycle and initiating apoptosis. p53 becomes activated in response to DNA damage, osmotic shock, oxidative stress or other myriad stressors. Activated p53 activates the expression of several genes by binding DNA including p21. p21 binds to the G1-S/CDK complexes which is an important molecule for the G1/S transition, then causes cell cycle arrest. p53 promotes apoptosis through two major apoptotic pathways: extrinsic pathway and intrinsic pathways. The extrinsic pathway involves activation of particular cell-surface death receptors that belong to the tumor necrosis factor (TNF) receptor family and, through the formation of the death-inducing signaling complex (DISC), leads to a cascade of activation of caspases, including Caspase8 and Caspase3, which in turn induce apoptosis. In the intrinsic pathway, p53 participates interacts with the multidomain members of the Bcl-2 family (e.g., Bcl-2, Bcl-xL) to induce mitochondrial outer membrane permeabilization.

In some embodiment, methods of the present invention involve altering the phosphoinositide 3-kinase (PI3K)/Akt signaling pathway. The PI3K/Akt signaling pathway plays a critical role in regulating various cellular functions including metabolism, growth, proliferation, survival, transcription and protein synthesis. The signaling cascade is activated by receptor tyrosine kinases, integrins, B and T cell receptors, cytokine receptors, G-protein-coupled receptors and other stimuli that induce production of phosphatidylinositol (3,4,5) trisphosphates (PIP3) by PI3K. the serine/threonine kinase Akt (also known as protein kinase B or PKB) interacts with these phospholipids, causing its translocation to the inner membrane, where it is phosphorylated and activated by pyruvate dehydrogenase kinases PDK1 and PDK2. Activated Akt modulates the function of numerous substrates involved in the regulation of cell survival, cell cycle progression and cellular growth.

In some embodiment, methods of the present invention involve altering the spleen tyrosine kinase (Syk)-dependent signaling pathway. Syk is a protein tyrosine kinase associated with various inflammatory cells, including macrophages. Syk plays a key role in the signaling of activating Fc receptors and the B-cell receptor (BCR). When Fc-receptors for IgG I, IIA, and IIIA bind to their ligands, the receptor complex becomes activated and triggers the phosphorylation of the immunoreceptor-activating motifs (ITAMs). This activates various genes, which leads to a cytoskeletal rearrangement that mediates phagocytosis in cells of the monocyte/macrophage lineage. Because Syk plays an important role in Fc receptor-mediated signal transduction and inflammatory propagation, it is considered a good target for the inhibition of various autoimmune conditions, such as rheumatoid arthritis and lymphoma.

In some embodiment, methods of the present invention involve altering the insulin like growth factor 1 receptor (IGF-1R)/insulin receptor (InsR) signaling pathway. Insulin-like growth factor 1 (IGF-1) controls many biological processes such as cellular metabolism, proliferation, differentiation, and apoptosis. These effects are mediated through ligand activation of the tyrosine kinase activity intrinsic to their receptors IGF-1R. InsR substrates 1 and 2 (IRS1 and IRS2) are key signaling intermediates, and their known downstream effectors are PI3K/AKT and MAPK/ERK1. The consequence of signaling results in a temporal transcriptional response leading to a wide range of biological processes including cell proliferation and survival.

In some embodiment, methods of the present invention involve altering the Fms-like Tyrosine Kinase-3 (FLT3) signaling pathway. FLT3, also known as FLK2 (Fetal Liver Kinase-2) and STK1 (human Stem Cell Kinase-1) is a cytokine receptor which belongs to the receptor tyrosine kinase class III. It is expressed on the surface of many hematopoietic progenitor cells. Signaling of FLT3 is important for the normal development of hematopoietic stem cells and progenitor cells. Binding of FLT3 ligand to FLT3 triggers the PI3K and RAS pathways, leading to increased cell proliferation and the inhibition of apoptosis.

In some embodiment, methods of the present invention involve altering the Hippo signaling pathway. The Hippo signaling pathway plays an important role in tissue regeneration, stem cell self-renewal and organ size control. It controls organ size in animals through the regulation of cell proliferation and apoptosis. The Mammalian Sterile 20-like kinases (MST1 and MST2) are key components of the Hippo signaling pathway in mammals.

In some embodiments, methods of the present invention involve altering the mammalian Target Of Rapamycin (mTOR) pathway. The mTOR pathway is a central regulator of cell metabolism, growth, proliferation and survival. mTOR is an atypical serine/threonine kinase that is present in two distinct complexes: mTOR complex 1 (mTORC1) and mTORC2. mTORC1 functions as a nutrient/energy/redox sensor and controls protein synthesis. It senses and integrates diverse nutritional and environmental cues, including growth factors, energy levels, cellular stress, and amino acids. mTORC2 has been shown to function as an important regulator of the actin cytoskeleton. In addition, mTORC2 is also involved in the activation of IGF-IR and InsR. Aberrant mTOR signaling is linked to many human diseases including cancer, cardiovascular disease, and diabetes. mTORC1 comprises the mTOR protein, the Raptor protein subunit, the mLST8 protein subunit, the Deptor protein subunit, and the PRAS40 protein subunit. mTORC2 comprises the mTOR protein, the Deptor and mLST8 protein subunits, the RICTOR protein subunit, the Protor protein subunit, and the mSIN1 protein subunit. mTORC2 lacks the Raptor protein subunit, while mTORC1 lacks the RICTOR protein subunit.

In some embodiments, methods of the present invention involve altering the Glycogen synthase kinase 3 (GSK3) pathway. GSK3 is a constitutively active, highly conserved serine/threonine protein kinase involved in numerous cellular functions including glycogen metabolism, gene transcription, protein translation, cell proliferation, apoptosis, immune response, and microtubule stability. GSK3 participates in a variety of signaling pathways, including cellular responses to WNT, growth factors, insulin, Reelin, receptor tyrosine kinases (RTK), Hedgehog pathways, and G-protein-coupled receptors (GPCR). GSK3 is localized predominantly in the cytoplasm but its subcellular localization is changed in response to stimuli.

In some embodiments, methods of the present invention involve altering the transforming growth factor-beta (TGF-beta)/SMAD signaling pathway. TGF-beta/SMAD signaling pathway is involved in many biological processes in both the adult organism and the developing embryo including cell growth, cell differentiation, apoptosis, cellular homeostasis and other cellular functions. TGF-beta superfamily ligands include Bone morphogenetic proteins (BMPs), Growth and differentiation factors (GDFs), Anti mullerian hormone (AMH), Activin, Nodal and TGF-beta. They act via specific receptors activating multiple intracellular pathways resulting in phosphorylation of receptor-regulated SMAD proteins that associate with the common mediator, SMAD4. Such complex translocates to the nucleus, binds to DNA and regulates transcription of many genes. BMPs may cause the transcription of mRNAs involved in osteogenesis, neurogenesis, and ventral mesoderm specification. TGF-betas may cause the transcription of mRNAs involved in apoptosis, extracellular matrix neogenesis and immunosuppression. It is also involved in G1 arrest in the cell cycle. Activin may cause the transcription of mRNAs involved in gonadal growth, embryo differentiation and placenta formation. Nodal may cause the transcription of mRNAs involved in left and right axis specification, mesoderm and endoderm induction. The roles of TGF-beta superfamily members are reviewed in Wakefield et al., Nature Reviews Cancer 13(5):328-41, which is hereby incorporated by reference in its entirety.

In some embodiments, methods of the present invention involve altering the nuclear factor-kappa B (NF-κB) signaling pathway. NF-κB is a transcription factor found in all cell types and is involved in cellular responses to stimuli such as stress and cytokines. NF-κB signaling plays an important role in inflammation, the innate and adaptive immune response and stress. In unstimulated cells NF-κB dimers are sequestered inactively in the cytoplasm by a protein complex called inhibitor of kappa B (IκB). Activation of NF-κB occurs via degradation of IκB, a process that is initiated by its phosphorylation by IκB kinase (IKK). This enables the active NF-κB transcription factor subunits to translocate to the nucleus and induce target gene expression. NF-κB activation turns on expression of the IκBα gene, forming a negative feedback loop. Dysregulation of NF-κB signaling can lead to inflammatory and autoimmune diseases and cancer. The role of NF-κB pathway in inflammation is reviewed in Lawrence, Cold Spring Harb Perspect Biol. 2009; 1(6): a001651, which is hereby incorporated by reference in its entirety.

### II. Features and Properties of the Patatin-Like Phospholipase Domain-Containing Protein 3 (PNPLA3) Gene

In some embodiments, methods of the present invention involve modulating the expression of the Patatin-like phospholipase domain-containing protein 3 (PNPLA3) gene. PNPLA3 may also be referred to as Adiponutrin, Calcium-Independent Phospholipase A2-Epsilon, Acylglycerol O-Acyltransferase, Patatin-Like Phospholipase Domain-Containing Protein 3, Patatin-Like Phospholipase Domain Containing 3, Chromosome 22 Open Reading Frame 20, IPLA(2)Epsilon, IPLA2epsilon, IPLA2-Epsilon, C22orf20, ADPN, EC 2.7.7.56, EC 4.2.3.4, EC 3.1.1.3, and EC 2.3.1.-. PNPLA3 has a cytogenetic location of 22₈13.31 and the genomic coordinate are on Chromosome 22 on the forward strand at position 43,923,739-43,964,488. PNPLAS (ENSG00000100341) is the gene upstream of PNPLA3 on the forward strand and SAMM50 (ENSG00000100347) is the gene downstream of PNPLA3 on the forward strand. PNPLA3 has a NCBI gene ID of 80339, Uniprot ID of Q9NST1 and Ensembl Gene ID of ENSG00000100344. The nucleotide sequence of PNPLA3 is shown in SEQ ID NO: 1.

In some embodiments, methods of the present invention involve altering the composition and/or the structure of the insulated neighborhood containing the PNPLA3 gene. The present inventors have identified the insulated neighborhood containing the PNPLA3 gene in primary human hepatocytes. The insulated neighborhood that contains the PNPLA3 gene is on chromosome 22 at position 43,782,676-45,023,137 with a size of approximately 1,240 kb. The number of signaling centers within the insulated neighborhood is 12. The insulated neighborhood contains PNPLA3 and 7 other genes, namely MPPED1, EFCAB6, SULT4A1, PNPLAS, SAMM50, PARVB, and PARVG. The chromatin marks, or chromatin-associated proteins, identified at the insulated neighborhood include H3k27ac, BRD4, p300, H3K4me1 and H3K4me3. Transcription factors involved in the insulated neighborhood include HNF3b, HNF4a, HNF4, HNF6, Myc, ONECUT2 and YY1. Signaling proteins involved in the insulated neighborhood include TCF4, HIF1a, HNF1, ERa, GR, JUN, RXR, STAT3, VDR, NF-κB, SMAD2/3, STAT1, TEAD1, p53, SMAD4, and FOS. Any components of these signaling centers and/or signaling molecules, or any regions within or near the insulated neighborhood, may be targeted or altered to change the composition and/or structure of the insulated neighborhood, thereby modulating the expression of PNPLA3.

PNPLA3 encodes a lipid droplet-associated, carbohydrate-regulated lipogenic and/or lipolytic enzyme. PNPLA3 is predominantly expressed in liver (hepatocytes and hepatic stellate cells) and adipose tissue. Hepatic stellate cells (HSCs, also called perisinusoidal cells or Ito cells) are contractile cells that reside between the hepatocytes and small blood vessels in the liver. HSCs have been identified as the main matrix-producing cells in the process of liver fibrosis. PNPLA3 is known to be involved in various metabolic pathways, such as glycerophospholipid biosynthesis, triacylglycerol biosynthesis, adipogenesis, and eicosanoid synthesis.

Variations in PNPLA3 are associated with metabolic disorders such as nonalcoholic fatty liver disease, nonalcoholic steatohepatitis, hepatic steatosis, alcoholic liver disease, alcoholic liver cirrhosis, alcoholic steatosis, liver cancer, lipid storage disease, obesity and other inherited metabolic disorders. Any one or more of these disorders may be treated using the compositions and methods described herein.

A polymorphic variation rs738409 C/G of PNPLA3, encoding for the isoleucine to methionine substitution at residue 148 (I148M), has been linked to NAFLD, hepatic steatosis and nonalcoholic steatohepatitis (NASH) as well as its pathobiological sequelae fibrosis, cirrhosis, and hepatocellular cancer (Krawczyk M et al., Semin Liver Dis. 2013 November; 33(4):369-79, which is hereby incorporated by reference in its entirety). The rs738409 C/G allele in PNPLA3 was first reported to be strongly associated with increased hepatic fat levels (P=5.9×10⁻¹⁰) and with hepatic inflammation (P=3.7×10⁻⁴) (Romeo et al., Nat Genet. 2008 December; 40(12):1461-5, which is hereby incorporated by reference in its entirety). Research suggests that the altered protein leads to increased production and decreased breakdown of fats in the liver. PNPLA3 I148M enhances steatosis by impairing the liberation of triglycerides from lipid droplets (Trépo E et al., J Hepatol. 2016 August; 65(2):399-412, which is hereby incorporated by reference in its entirety). Recent data also suggests that PNPLA3 I148M protein evades degradation and accumulates on lipid droplets (BasuRay et al., Hepatology. 2017 October; 66(4):1111-1124, which is hereby incorporated by reference in its entirety). I148M variant is associated with NAFLD in both adults and in children, but is predominant in women, not in men. The specific mechanism of the PNPLA3 I148M variant in the development and progression of NAFLD is still not clear. However, it is thought that the PNPLA3 I148M variant may promote the development of fibrogenesis by activating the hedgehog signaling pathway, which, in turn, leads to the activation and proliferation of hepatic stellate cells, and excessive generation and deposition of intrahepatic extracellular matrix (Chen L Z, et al., World J Gastroenterol. 2015 Jan. 21; 21(3): 794-802, which is hereby incorporated by reference in its entirety).

The I148M variant has also been correlated with alcoholic liver disease and clinically evident alcoholic cirrhosis (Tian et al., Nature Genetics 42,21-23 (2010), which is hereby incorporated by reference in its entirety). Moreover, it has been identified as a prominent risk factor for hepatocellular carcinoma in patients with alcoholic cirrhosis (Nischalke et al., PLoS One. 2011; 6(11):e27087, which is hereby incorporated by reference in its entirety).

The I148M variant also influences insulin secretion levels and obesity. In obese subjects the body mass index and waist are higher in carriers of the variant allele (Johansson L E et al., Eur J Endocrinol. 2008 November; 159(5):577-83, which is hereby incorporated by reference in its entirety). The I148M carriers display decreased insulin secretion in response to oral glucose tolerance test. I148M allele carriers are seemingly more insulin resistant at a lower body mass index.

The mutated PNPLA3 protein is not accessible by traditional antibody or small molecule approaches and its expression across hepatocytes and stellate cells leads to significant delivery challenges for oligo modality. This present invention provides novel treatment options for targeting PNPLA3 by altering the expression level of the mutant PNPLA3.

In some embodiments, methods of the present invention involve modulating the expression of the Collagen Type I Alpha 1 Chain (COL1A1) gene. COL1A1 is a member of group I collagen (fibrillar forming collagen). Activation of Hepatic stellate cells (HSCs) in damaged liver leads to secretion of collagen (such as COL1A1) and formation of scar tissue, which contribute to chronic fibrosis or cirrhosis. Expression of PNPLA3 increases during the early phases of activation and remains elevated in fully activated HSCs. Emerging evidence suggests that PNPLA3 is involved in HSC activation and its genetic variant I148M potentiates pro-fibrogenic features such as increased pro-inflammatory cytokine secretion. Reduction of PNPLA3 has been reported to affect the fibrotic phenotype in HSCs including COL1A1 levels (Bruschi et al., Hepatology. 2017; 65(6):1875-1890, the content of which is hereby incorporated by reference in its entirety).

In some embodiments, methods of the present invention involve modulating the expression of the Patatin-like phospholipase domain-containing protein 5 (PNPLAS) gene. PNPLAS, also known as GS2-like protein, is a member of the patatin-like phospholipase family. Inventors of the present invention discovered that PNPLAS is located in the same insulated neighborhood as PNPLA3 in primary hepatocytes and responds to compound treatment similarly to PNPLA3. In fact, PNPLA3 was reported to be qualitatively expressed and regulated in a manner similar to PNPLA5 in mice (Lake et al., J Lipid Res. 2005; 46(11):2477-87, the content of which is hereby incorporated by reference in its entirety). Lake et al. also observed that PNPLA3 expression was undetectable in the liver of C57Bl/6J mice under both fasting and fed conditions, but was strongly induced in the liver of ob/ob mice, suggesting a role in hepatic lipogenesis.

In some embodiments, methods of the present invention involve modulating the expression of the Hydroxysteroid 17-Beta Dehydrogenase 13 (HSD17B13) gene. SNPs in HSD17B13 such as rs72613567:TA have been reported to be significantly associated with histological features of chronic liver diseases including nonalcoholic steatohepatitis. RNA sequencing-based expression analysis revealed that HSD17B13 rs72613567:TA was associated with decreased PNPLA3 messenger RNA (mRNA) expression in an allele dose-dependent manner. See, Abul-Husn et al., N Engl J Med 2018;378:1096-106, the content of which is hereby incorporated by reference in its entirety.

### III. Compositions and Methods

The present invention provides compositions and methods for modulating the expression of PNPLA3 to treat one or more PNPLA3-related disorders. Any one of the compositions and methods described herein may be used to treat a PNPLA3-related disorder in a subject. In some embodiments, a combination of the compositions and methods described herein may be used to treat a PNPLA3-related disorder.

As used herein, the term “PNPLA3-related disorder” refers to any disorder, disease, or state that is associated with the expression of the PNPLA3 gene and/or function of the PNPLA3 gene product (e.g., mRNA, protein). Such disorders include but are not limited to nonalcoholic fatty liver disease (NAFLD), nonalcoholic steatohepatitis (NASH), hepatic steatosis, alcoholic liver disease (ALD), alcoholic liver cirrhosis, liver cancer, lipid storage disease, obesity, and other inherited metabolic disorders. In some embodiments, the PNPLA3-related disorder is NAFLD. In some embodiments, the PNPLA3-related disorder is NASH. In some embodiments, the PNPLA3-related disorder is ALD, including alcoholic liver cirrhosis.

As used herein, the term “PNPLA3-targeted therapy” refers to any treatment method involving administering to a subject or a cell a compound that has direct or indirect effect in modulating the expression of PNPLA3.

The terms “subject” and “patient” are used interchangeably herein and refer to an animal to whom treatment with the compositions according to the present invention is provided. In some embodiments, the subject is a mammal. In some embodiments, the subject is a human.

In some embodiments, subjects or patients may have been diagnosed with or have symptoms for a PNPLA3-related disorder, e.g., NAFLD, NASH, and/or ALD. In other embodiments, subjects or patients may be susceptible to a PNPLA3-related disorder, e.g., NAFLD, NASH, and/or ALD. Subjects or patients may have dysregulated expression of the PNPLA3 gene and/or abnormal function of the PNPLA3 protein. Subjects or patients may carry mutations within or near the PNPLA3 gene. In some embodiments, subjects or patients may carry the mutation I148M in the PNPLA3 gene. Subjects or patients carry one or two I148M alleles of the PNPLA3 gene.

In some embodiments, compositions and methods of the present invention may be used to decrease expression of the PNPLA3 gene in a cell or a subject. Changes in gene expression may be assessed at the RNA level or protein level by various techniques known in the art and described herein, such as RNA-seq, qRT-PCR, Western Blot, or enzyme-linked immunosorbent assay (ELISA). Changes in gene expression may be determined by comparing the level of PNPLA3 expression in the treated cell or subject to the level of expression in an untreated or control cell or subject.

In some embodiments, compositions and methods of the present invention cause reduction in the expression of a PNPLA3 gene as measured in a cell-based assay of cells exposed to the compound at a level corresponding to the plasma level achieved at steady state in a subject dosed with the effective amount as compared to cells exposed to a placebo. In some embodiments, the cells are homozygous for the wild type PNPLA3 gene. In some embodiments, the cells are heterozygous for the wild type and the mutant I148M PNPLA3 gene. In some embodiments, the cells are homozygous for the mutant I148M PNPLA3 gene.

In some embodiments, compositions and methods of the present invention cause reduction in the expression of a PNPLA3 gene on average in a population administered the compound as compared to control subjects administered a placebo.

In some embodiments, compositions and methods of the present invention cause reduction in the expression of a PNPLA3 gene in a subject as compared to pre-dosing PNPLA3 gene expression levels in the subject.

In some embodiments, the expression of the PNPLA3 gene is decreased by at least about 10%, at least about 20%, at least about 30%, at least about 40%, at least about 50%, from about 25% to about 50%, from about 40% to about 60%, from about 50% to about 70%, from about 60% to about 80%, more than 80%, or even more than 90%, 95% or 99% as compared to the PNPLA3 expression in an untreated cell, untreated subject, or untreated population. In some embodiments, the administration of a compound reduces the expression of the PNPLA3 gene in a cell in vivo or in vitro by at least about 1%, 5%, 10%, 15%, 20%, 25%, 30%, 35%, 40%, 45%, 50%, 55%, 60%, 65%, 70%, 75%, 80%, 85%, 90%, 95%, or 99% as compared to the PNPLA3 expression in an untreated cell, untreated subject, or untreated population. In some embodiments, the reduced expression is in a cell in a subject.

In some embodiments, reduction in PNPLA3 expression induced by compositions and methods of the present invention may be sufficient to prevent or alleviate at least one or more signs or symptoms of NAFLD, NASH, and/or ALD.

### Small Molecules

In some embodiments, compounds used to modulate PNPLA3 gene expression may include small molecules. As used herein, the term “small molecule” refers to any molecule having a molecular weight of 5000 Daltons or less. In certain embodiments, at least one small molecule compounds described herein is applied to a genomic system to alter the boundaries of an insulated neighborhood and/or disrupt signaling centers, thereby modulating the expression of PNPLA3.

A small molecule screen may be performed to identify small molecules that act through signaling centers of an insulated neighborhood to alter gene signaling networks which may modulate expression of a select group of disease genes. For example, known signaling agonists/antagonists may be administered. Credible hits are identified and validated by the small molecules that are known to work through a signaling center and modulate expression of the target gene PNPLA3.

In some embodiments, small molecule compounds capable of modulating PNPLA3 expression include but are not limited to Amuvatinib, BMS-754807, BMS-986094, LY294002, Momelotinib, Pacritinib, R788, WYE-125132, XMU-MP-1 or derivatives or analogs thereof. Any one or more of such compounds may be administered to a subject to treat a PNPLA3-related disorder, e.g., NAFLD, NASH, and/or ALD. Amuvatinib

In some embodiments, compounds capable of modulating the expression of the PNPLA3 gene may include Amuvatinib, or a derivative or an analog thereof. Amuvatinib, also known as MP-470, or HPK 56, is an orally bioavailable synthetic carbothioamide with potential antineoplastic activity. It has a CAS number of 850879-09-3 and PubChem Compound ID of 11282283. The structure of Amuvatinib is shown below.

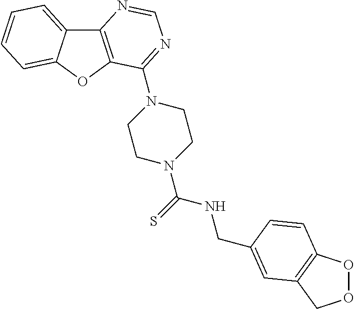 [SMILES: S=C(NCc1ccc2c(c1)COO2)N1CCN(c2ncnc3c2oc2ccccc23)CC1]

Amuvatinib is a potent and multi-targeted inhibitor of stem cell growth factor receptor (SCFR or c-Kit), Platelet-derived growth factor receptor alpha (PDGFRα), and FLT3 with IC₅₀ of 10 nM, 40 nM, and 81 nM, respectively. Amuvatinib also inhibits clinically mutant forms of c-Kit, PDGFRα, and FLT3, which are often associated with cancer. Mechanistically, Amuvatinib inhibits tyrosine kinase receptor c-Kit through occupying its ATP binding domain and disrupts DNA repair through suppression of DNA repair protein Rad51 as well as synergistic effects in combination with DNA damage-inducing agents. Amuvatinib exhibits antitumor activity against several human cancer cell lines, such as GIST-48 human cell line.

Amuvatinib is currently in Phase 1/2 clinical trials as single agent or in combination with chemotherapies to treat solid tumors.

BMS-754807

In some embodiments, compounds capable of modulating the expression of the PNPLA3 gene may include BMS-754807, or a derivative or an analog thereof. BMS-754807 is a reversible, orally available dual inhibitor of the insulin-like growth factor 1 receptor (IGF-1R)/insulin receptor (InsR) family kinases. It has a CAS number of 001350-96-4 and PubChem Compound ID of 329774351. The structure of BMS-754807 is shown below.

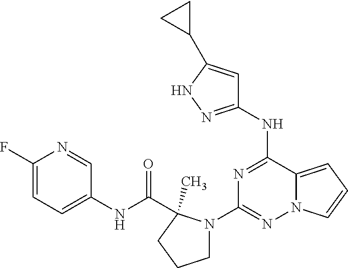 [SMILES: C[C@@]1(C(=O)Nc2ccc(F)nc2)CCCN1c1nc(Nc2cc(C3CC3)[nH]n2)c2cccn2n1]

BMS-754807 inhibits IGF-1R and InsR with IC₅₀ of 1.8 nM and 1.7 nM, respectively. It has minimal effect against an array of other tyrosine and serine/threonine kinases (Wittman et al., Journal of Medicinal Chemistry 52, 7630-7363 (2009), which is hereby incorporated by reference in its entirety). BMS-754807 acts as a reversible ATP-competitive antagonist of IGF-1R by restricting the catalytic domain of the IGF-1R. BMS-754807 inhibits tumor growth in multiple xenograft tumor models (e.g., epithelial, mesenchymal, and hematopoietic). Combination studies with BMS-754807 have been done on multiple human tumor cell types and mouse models, and showed synergies (combination index, <1.0) when combined with cytotoxic, hormonal, and targeted agents. See, Awasthi et al., Molecular Cancer Therapeutics 11(12), 2644-2653 (2012); Carboni et al., Mol Cancer Ther. 2009 December; 8(12):3341-9; which are hereby incorporated by reference in their entirety.

BMS-986094

In some embodiments, compounds capable of modulating the expression of the PNPLA3 gene may include BMS-986094, or a derivative or an analog thereof. BMS-986094, also known as INX-08189, INX-189, or IDX-189, is a prodrug of a guanosine nucleotide analogue (2′-C-methylguanosine). It has a CAS number of 1234490-83-5 and PubChem Compound ID of 46700744. The structure of BMS-986094 is shown below.

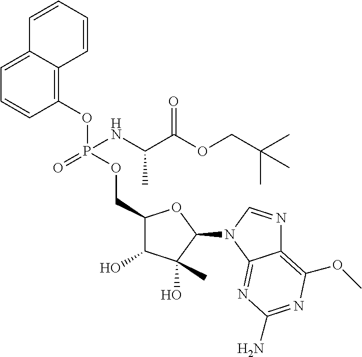 [SMILES: COc1nc(N)nc2c1ncn2[C@@H]1O[C@H](COP(=O)(N[C@@H](C)C(=O)OCC(C)(C)C)Oc2cccc3ccccc23)[C@@H](O)[C@@]1(C)O]

BMS-986094 is an RNA-directed RNA polymerase (NSSB) inhibitor originally developed by Inhibitex (acquired by Bristol-Myers Squibb in 2012). It was in phase II clinical trials for the treatment of hepatitis C virus infection. However, the study was discontinued due to unexpected cardiac and renal adverse events.

LY294002

In some embodiments, compounds capable of modulating the expression of the PNPLA3 gene may include LY294002, or a derivative or an analog thereof. LY294002, also known as 2-Morpholin-4-yl-8-phenylchromen-4-one, SF 1101, or NSC 697286, is a cell permeable, broad-spectrum inhibitor of Phosphatidylinositol-4,5-bisphosphate 3-kinases (PI3Ks). It has a CAS number of 154447-36-6 and PubChem Compound ID of 3973. The structure of LY294002 is shown below.

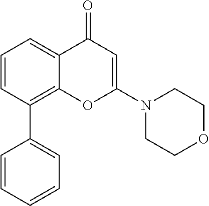 [SMILES: O=c1cc(N2CCOCC2)oc2c(-c3ccccc3)cccc12]

LY294002 inhibits PI3Kα/δ/β with IC₅₀ of 0.5 μM/0.57 μM/0.97 μM in cell-free assays, respectively. It acts as a competitor inhibitor of the ATP binding site of the PI3Ks. LY294002 does not affect the activities of EGF receptor kinase, MAP kinase, PKC, PI4-kinase, S6 kinase and c-Src even at 50 μM (Vlahos, C. J. et al. (1994) J Biol Chem 269, 5241-8, which is hereby incorporated by reference in its entirety). LY294002 has been shown to block PI3K-dependent Akt phosphorylation and kinase activity. It has also been established as an autophagy inhibitor that blocks autophagosome. Besides PI3Ks, LY294002 is a potent inhibitor of many other proteins, such as casein kinase II, and BET bromodomains.

Momelotinib

In some embodiments, compounds capable of modulating the expression of the PNPLA3 gene may include Momelotinib, or a derivative or an analog thereof. Momelotinib, also known as N-(cyanomethyl)-4-{2-[4-(morpholin-4-yl)anilino]pyrimidin-4-yl}benzamide, CYT-387, CYT-11387, or GS-0387, is an ATP-competitive inhibitor of Janus kinases JAK1 and JAK2. It has a CAS number of 1056634-68-4 and PubChem Compound ID of 25062766. The structure of Momelotinib is shown below.

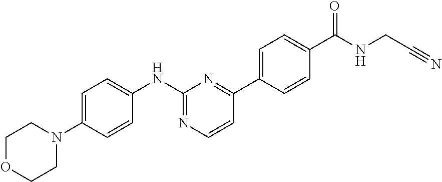 [SMILES: N#CCNC(=O)c1ccc(-c2ccnc(Nc3ccc(N4CCOCC4)cc3)n2)cc1]

Momelotinib inhibits JAK1 and JAK2 with IC₅₀ of 11 nM and 18 nM, respectively (Pardanani A, et al. Leukemia, 2009, 23(8), 1441-1445, which is hereby incorporated by reference in its entirety). The activity is significantly less towards other kinases, including JAK3 (IC₅₀=160 nM). Inhibition of JAK1/2 activation leads to inhibition of the JAK/STAT signaling pathway, and hence the induction of apoptosis. Momelotinib shows antiproliferative effects in IL-3 stimulated Ba/F3 cells. It also causes the inhibition of cell proliferation in several cell lines constitutively activated by JAK2 or MPL signaling, including Ba/F3-MPLW515L cells, CHRF-288-11 cells and Ba/F3-TEL-JAK2 cells. In a murine myeloproliferative neoplasms model, Momelotinib induces hematologic responses and restores physiologic levels of inflammatory cytokines (Tyner J W, et al. Blood, 2010, 115(25), 5232-5240, which is hereby incorporated by reference in its entirety).

Momelotinib is also known to inhibit a spectrum of other kinases including TYK2 with IC₅₀ of ˜20 nM, and CDK2, JNK1, PKD3, PKCu, ROCK2 and TBK1 with IC₅₀ of less than 100 nM (Tyner J W, et al. Blood, 2010, 115(25), 5232-5240, which is hereby incorporated by reference in its entirety). TBK1 has been linked to the mTOR pathway. It was recently demonstrated that Momelotinib also inhibits BMPR kinase activin A receptor, type I (ACVR1), which is also called activin receptor-like kinase-2 (ALK2), with IC₅₀ of 8 nM (Asshoff M et al., Blood 2017 129:1823-1830, which is hereby incorporated by reference in its entirety). ACVR1 is known to be involved in the TGF-beta/SMAD signaling pathway.

Momelotinib is being developed by Gilead Sciences in a Phase III trial for the treatment of pancreatic and non-small cell lung cancers, and myeloproliferative disorders (including myelofibrosis, essential thrombocythemia and polycythemia vera).

Pacritinib

In some embodiments, compounds capable of modulating the expression of the PNPLA3 gene may include Pacritinib, or a derivative or an analog thereof. Pacritinib, also known as SB1518, is an oral tyrosine kinase inhibitor developed by CTi BioPharma. It has a CAS number of 937272-79-2 and PubChem Compound ID of 46216796. The structure of Pacritinib is shown below.

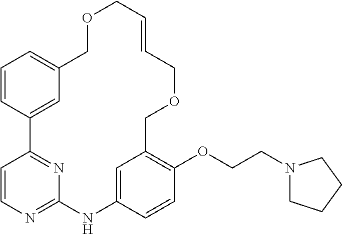 [SMILES: C1=C/COCc2cc(ccc2OCCN2CCCC2)Nc2nccc(n2)-c2cccc(c2)COC/1]

Pacritinib is known to inhibit Janus Associated Kinase 2 (JAK2) and FMS-like tyrosine kinase 3 (FLT3) with reported IC₅₀ values of 23 nM and 22 nM in cell-free assays, respectively. The JAK family of enzymes is a family of intracellular, nonreceptor tyrosine kinases that transduce cytokine-mediated signals via the JAK/STAT pathway. Pacritinib has potent effects on cellular JAK/STAT pathways, inhibiting tyrosine phosphorylation on JAK2 (Y221) and downstream STATs. Pacritinib induces apoptosis, cell cycle arrest and antiproliferative effects in JAK2-dependent cells. Pacritinib also inhibits FLT3 phosphorylation and downstream STAT, MAPK and PI3K signaling. See William et al., J. Med. Chem., 2011, 54 (13), 4638-4658; Hart S et al., Leukemia, 2011, 25(11), 1751-1759; Hart S et al., Blood Cancer J, 2011, 1(11), e44; which are hereby incorporated by reference in their entirety.

Pacritinib has demonstrated encouraging results in Phase 1 and 2 studies for patients with myelofibrosis and may offer an advantage over other JAK inhibitors through effective treatment of symptoms while having less treatment-emergent thrombocytopenia and anemia than has been seen in currently approved and in-development JAK inhibitors.

Pifithrin-μ

In some embodiments, compounds capable of modulating the expression of the PNPLA3 gene may include Pifithrinμ, or a derivative or an analog thereof. Pifithrinμ, also known as 2-Phenylethynesulfonamide or PFT-μ, is an inhibitor of p53-mediated apoptosis. It has a CAS number of 64984-31-2 and PubChem Compound ID of 24724568. The structure of Pifithrin-μ, is shown below.

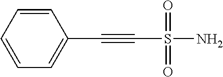 [SMILES: NS(=O)(=O)C#Cc1ccccc1]

Pifithrin-μ, interferes with p53 binding to mitochondria and inhibits rapid p53-dependent apoptosis of primary cell cultures of mouse thymocytes in response to gamma radiation (Strom E, et al. Nat Chem Biol. 2006, 2(9), 474-479, which is hereby incorporated by reference in its entirety). Pifithrin-μ, reduces the binding affinity of p53 to the anti-apoptotic proteins Bcl-xL and Bcl-2 at the mitochondria surface, while displaying no effect on the transactivational or cell cycle checkpoint control function of p53. Pifithrin-μ, protects mice from doses of gamma radiation that cause lethal hematopoietic syndrome. Pifithrin-μ reduces apoptosis triggered by nutlin-3, which inhibits MDM2/p53 binding and potentiates p53-mediated growth arrest and apoptosis (Vaseva et al., Cell Cycle 8(11), 1711-1719 (2009), which is hereby incorporated by reference in its entirety). Pifithrin-μ also interacts selectively with heat shock protein 70 (HSP70), leading to disruption of the association between HSP70 and several of its co-chaperones and substrate proteins (Leu et al., Molecular Cell 36(1), 15-27 (2009), which is hereby incorporated by reference in its entirety).

R788

In some embodiments, compounds capable of modulating the expression of the PNPLA3 gene may include R788, or a derivative or an analog thereof. R788, also known as fostamatinib disodium hexahydrate, tamatinib fosdium, NSC-745942; or R-935788, is an orally bioavailable inhibitor of the enzyme spleen tyrosine kinase (Syk). It has a CAS number of 1025687-58-4 and PubChem Compound ID of 25008120. The structure of R788 is shown below.

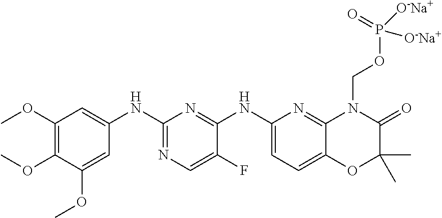

R788 is a methylene prodrug of active metabolite R406, which is an ATP-competitive inhibitor of Syk with IC₅₀ of 41 nM (Braselmann et al., J. Pharma. Exp. Ther. 2006, 319(3), 998-1008, which is hereby incorporated by reference in its entirety). R406 inhibits phosphorylation of Syk substrate linker for activation of T cells in mast cells and B-cell linker protein SLP65 in B cells. R406 is also a potent inhibitor of immunoglobulin E (IgE)- and IgG-mediated activation of Fc receptor signaling. R406 blocks Syk-dependent Fc receptor-mediated activation of monocytes/macrophages and neutrophils and B-cell receptor (BCR)-mediated activation of B lymphocytes. In a large panel of diffuse large B-cell lymphoma cell lines, R406 inhibited cellular proliferation with EC₅₀ values ranging from 0.8 to 8.1 uM (Chen L, et al. Blood, 2008, 111(4), 2230-2237, which is hereby incorporated by reference in its entirety). R788 was shown to effectively inhibit BCR signaling in vivo, reduce proliferation and survival of the malignant B cells, and significantly prolong survival in treated mice (Suljagic M, et al. Blood, 2010, 116(23), 4894-4905, which is hereby incorporated by reference in its entirety).

R788 was developed by Rigel Pharmaceuticals and is currently in clinical trials for several autoimmune diseases, including rheumatoid arthritis, autoimmune thrombocytopenia, autoimmune hemolytic anemia, IgA nephropathy, and lymphoma.

WYE-125132

In some embodiments, compounds capable of modulating the expression of the PNPLA3 gene may include WYE-125132, or a derivative or an analog thereof. WYE-125132, also known as WYE-132, is a highly potent, ATP-competitive mammalian Target Of Rapamycin (mTOR) inhibitor. It has a CAS number of 1144068-46-1 and PubChem Compound ID of 25260757. The structure of WYE-125132 is shown below.

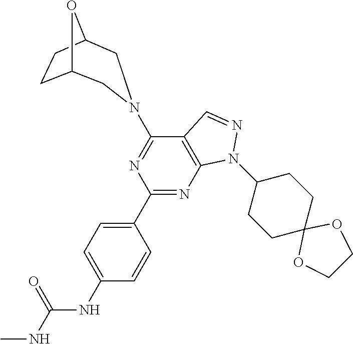 [SMILES: CNC(=O)Nc1ccc(-c2nc(N3CC4CCC(C3)O4)c3cnn(C4CCC5(CC4)OCCO5)c3n2)cc1]

WYE-125132 specifically inhibits mTOR with IC₅₀ of 0.19 nM. It is highly selective for mTOR versus PI3Ks or PI3K-related kinases hSMG1 and ATR. Unlike rapamycin, which inhibits mTOR through allosteric binding to mTOR complex 1 (mTORC1) only, WYE-132 inhibits both mTORC1 and mTORC2. WYE-132 shows anti-proliferative activity against a variety of tumor cell lines, including MDA361 breast, U87MG glioma, A549 and H1975 lung, as well as A498 and 786-O renal tumors. WYE-132 causes inhibition of protein synthesis and cell size, induction of apoptosis, and cell cycle progression.

XMU-MP-1

In some embodiments, compounds capable of modulating the expression of the PNPLA3 gene may include XMU-MP-1, or a derivative or an analog thereof. MU-MP-1, also known as AKOS030621725; ZINC498035595; CS-5818; or HY-100526, is a reversible, potent and selective inhibitor of Mammalian sterile 20-like kinases 1 and 2 (MST1/2). It has a CAS number of 2061980-01-4 and PubChem Compound ID of 121499143. The structure of XMU-MP-1 is shown below.

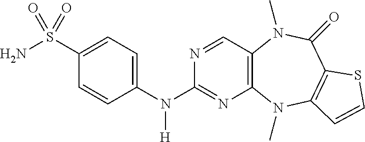 [SMILES: CN1C(=O)c2sccc2N(C)c2nc(Nc3ccc(S(N)(=O)=O)cc3)ncc21]

XMU-MP-1 inhibits MST1 and MST2 with IC₅₀ values of 71.1±12.9 nM and 38.1±6.9 nM, respectively. MST1 and MST2 are central components of the Hippo signaling pathway that play an important role in tissue regeneration, stem cell self-renewal and organ size control. Inhibition of MST1/2 kinase activities activates the downstream effector Yes-associated protein and leads to cell growth. XMU-MP-1 displays excellent in vivo pharmacokinetics and promotes mouse intestinal repair, as well as liver repair and regeneration, in both acute and chronic liver injury mouse models at a dose of 1 to 3 mg/kg via intraperitoneal injection. XMU-MP-1 treatment exhibited substantially greater repopulation rate of human hepatocytes in the Fah-deficient mouse model than in the vehicle-treated control, indicating that XMU-MP-1 treatment might facilitate human liver regeneration. See, Fan et al., Sci Transl Med. 2016, 8(352):352ra108, which is hereby incorporated by reference in its entirety.

OSI-027

In some embodiments, compounds capable of modulating the expression of the PNPLA3 gene include OSI-027, or a derivative or an analog thereof. OSI-027, also known as ASP4786, is a selective and potent dual inhibitor of mTORC1 and mTORC2. It has a CAS number of 936890-98-1 and PubChem Compound ID of 72698550. The structure of OSI-027 is shown below:

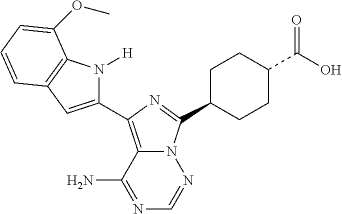 [SMILES: COc1cccc2cc(-c3nc([C@H]4CC[C@H](C(=O)O)CC4)n4ncnc(N)c34)[nH]c12]

OSI-027 inhibits mTORC1 and mTORC2 with IC₅₀ values of 22 nM and 65 nM, respectively. OSI-027 also inhibits mTOR signaling of phospho-4E-BP1 with an IC₅₀ of 1 μM and 4E-BP1, Akt, and S6 phosphorylation in vivo. OSI-027 shows anti-proliferative activity against a variety of tumor xenografts, including leukemia cell lines U937, KG-1, KBM-3B, ML-1, HL-60, and MEG-01, and breast cancer cells in vitro.

PF-04691502

In some embodiments, compounds capable of modulating the expression of the PNPLA3 gene include PF-04691502, or a derivative or an analog thereof. PF-04691502 is a PI3K(α/β/δ/γ) and mTOR dual inhibitor. It has a CAS number of 1013101-36-4 and PubChem Compound ID of 25033539. The structure of PF-04691502 is shown below:

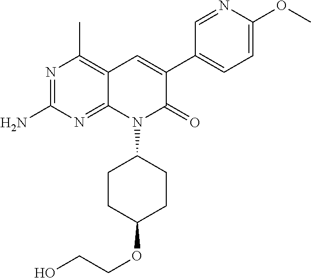 [SMILES: COc1ccc(-c2cc3c(C)nc(N)nc3n([C@H]3CC[C@H](OCCO)CC3)c2=O)cn1]

PF-04691502 inhibits mTORC1 with an IC₅₀ value of 32 nM and inhibits the activation of downstream mTOR and PI3K effectors including AKT, FKHRL1, PRAS40, p70S6K, 4EBP1, and S6RP. PF-04691502 shows anti-proliferative activity against a variety of non-small cell lung carcinoma xenografts.

LY2157299

In some embodiments, compounds capable of modulating the expression of the PNPLA3 gene include LY2157299, or a derivative or an analog thereof. LY2157299, also known as Galunisertib, is a TGFβ receptor I (TGFβRI) inhibitor. It has a CAS number of 700874-72-2 and PubChem Compound ID of 10090485. The structure of PF-04691502 is shown below:

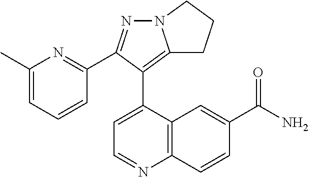 [SMILES: Cc1cccc(-c2nn3c(c2-c2ccnc4ccc(C(N)=O)cc24)CCC3)n1]

LY2157299 inhibits TGFβRI with IC₅₀ value of 56 nM and inhibits TGFβRI-induced Smad2 phosphorylation. LY2157299 stimulates hematopoiesis and angiogenesis in vitro and in vivo. LY2157299 shows anti-proliferative activity against Calu6 and MX1 xenografts in mice.

JR-AB2-011

In some embodiments, compounds capable of modulating the expression of the PNPLA3 gene include JR-AB2-011, or a derivative or an analog thereof. JR-AB2-011 is an mTORC2 inhibitor that blocks the interaction of mTOR and RICTOR. It has a CAS number of 329182-61-8. The structure of JR-AB2-011 is shown below:

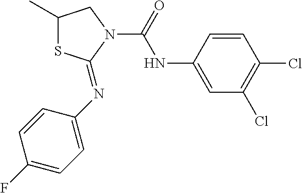 [SMILES: CC1CN(C(=O)Nc2ccc(Cl)c(Cl)c2)/C(=N/c2ccc(F)cc2)S1]

Other Compounds

In some embodiments, compounds for treatment of a PNPLA3-related disorder may include compounds that are also used to treat other liver diseases, disorders, or cancers. For example, the compound may be selected those contemplated for treatment of liver fibrosis, liver failure, liver cirrhosis, or liver cancer shown in WO 2016057278A1 such as aminopyridyloxypyrazole compounds that inhibit activity of transforming growth factor beta receptor 1 (TGF R1); WO 2003050129A1 such as LY582563; WO 1999050413A2 such as mFLINT; WO 2017007702A1 such as 4,4,4-trifluoro-N-((2S)-1-((9-methoxy-3,3-dimethyl-5-oxo-2,3,5,6-tetrahydro-1H-benzo[f]pyrrolo[1,2-a]azepin-6-yl)amino)-1-oxopropan-2-yl)butanamide or N-((2S)-1-((8,8-dimethyl-6-oxo-6,8,9,10-tetrahydro-5H-pyrido[3,2-f]pyrrolo[1,2-a]azepin-5-yl)amino)-1-oxopropan-2-yl)-4,4,4-trifluorobutanamide; WO 2016089670A1 such as N-(6-Fluoro-1-oxo-1,2-dihydroisoquinolin-7-yl)-5-[(3R)-3-hydroxypyrrolidin-1-yl]thiophene-2-sulfonamide; N-(6-Fluoro-1-oxo-1,2-dihydroisoquinolin-7-yl)-5-[(3S)-3-hydroxypyrrolidin-1-yl]thiophene-2-sulfonamide; 5-[(3S,4R)-3-Fluoro-4-hydroxy-pyrrolidin-1-yl]-N-(6-fluoro-1-oxo-1,2-dihydroisoquinolin-7-yl)thiophene-2-sulfonamide; 5-(3,3-Difluoro-(4R)-4-hydroxy-pyrrolidin-1-yl)-N-(6-fluoro-1-oxo-1,2-dihydroisoquinolin-7-yl)thiophene-2-sulfonamide; 5-(5,5-Dimethyl-6-oxo-1,4-dihydropyridazin-3-yl)-N-(6-fluoro-1-oxo-1,2-dihydroisoquinolin-7-yl)thiophene-2-sulfonamide; or N-(6-Fluoro-1-oxo-1,2-dihydroisoquinolin-7-yl)-54(IR,3R)-3-hydroxycyclopentyllthiophene-2-sulfonamide; or N-(6-Fluoro-1-oxo-1,2-dihydroisoquinolin-7-yl)-5-[(3R)-3-hydroxypyrrolidin-1-yl]thiophene-2-sulfonamide; WO 2015069512A1 such as 8-Methyl-2-[4-(pyrimidin-2-ylmethyl)piperazin-1-yl]-3,5,6,7-tetrahydropyrido[2,3-d]pyrimidin-4-one; 8-Methyl-2-[4-(1-pyrimidin-2-ylethyl)piperazin-1-yl]-3,5,6,7-tetrahydropyrido[2,3-d]pyrimidin-4-one; 2-[4-[(4-Chloropyrimidin-2-yl)methyl]piperazin-1-yl]-8-methyl-3, 5,6,7-tetrahydropyrido[2,3-d]pyrimidin-4-one; 2-[4-[(4-methoxypyrimidin-2-yl)methyl]piperazin-1-yl]-8-methyl-3, 5,6,7-tetrahydropyrido[2,3-d]pyrimidin-4-one; 2-[4-[(3-Bromo-2-pyridyl)methyl]piperazin-1-yl]-8-methyl-3,5,6,7-tetrahydropyrido[2,3-d]pyrimidin-4-one; 2-[4-[(3-Chloro-2-pyridyl)methyl]piperazin-1-yl]-8-methyl-3,5,6,7-tetrahydropyrido[2,3-d]pyrimidin-4-one; 2-[4-[(3-Fluoro-2-pyridyl)methyl]piperazin-1-yl]-8-methyl-3,5,6,7-tetrahydropyrido[2,3-d]pyrimidin-4-one; or 2-[[4-(8-Methyl-4-oxo-3,5,6,7-tetrahydropyrido[2,3-d]pyrimidin-2-yl)piperazin-1-yl]methyl]pyridine-3-carbonitrile; WO 2015054060A1 such as 2-hydroxy-2-methyl-N-[2-[2-(3-pyridyloxy)acetyl]-3,4-dihydro-1H-isoquinolin-6-yl]propane-1-sulfonamide or 2-methoxy-N-[2-[2-(3-pyridyloxy)acetyl]-3,4-dihydro-1H-isoquinolin-6-yl]ethanesulfonamide; WO 2013016081A1 such as 4,4,4-trifluoro-N-[(1S)-2-[[(7S)-5-(2-hydroxyethyl)-6-oxo-7H-pyrido[2,3-d][3]benzazepin-7-yl]amino]-1-methyl-2-oxo-ethyl]butanamide; WO 2012097039A1 such as 8-[5-(1-hydroxy-1-methylethyl)pyridin-3-yl]-1-[(2S)-2-methoxypropyl]-3-methyl-1,3-dihydro-2H-imidazo[4,5-c]quinolin-2-one; WO 2012064548A1 such as (R)-[5-(2-methoxy-6-methyl-pyridin-3-yl)-2H-pyrazol-3-yl]-[6-(piperidin-3-yloxy)-pyrazin-2-yl]-amine; WO 2010147917A1 such as 4-fluoro-N-methyl-N-(I-(4-(I-methyl-1H-pyrazol-5-yl)phthalazin-1-yl)piperidin-4-yl)-2-(trifluoromethyl)benzamide; U.S. Pat. No. 8,268,869B2 such as (E)-2-(4-(2-(5-(1-(3,5-dichloropyridin-4-yl)ethoxy)-1H-indazol-3-yl)vinyl)-1H-pyrazol-1-yl)ethanol or (R)-(E)-2-(4-(2-(5-(1-(3,5-dichloropyridin-4-yl)ethoxy)-1H-indazol-3-yl)vinyl)-1H-pyrazol-1-yl)ethanol; WO 2010077758A1 such as 5-(5-(2-(3-aminopropoxy)-6-methoxyphenyl)-1H-pyrazol-3-ylamino) pyrazine-2-carbonitrile; WO 2010074936A2 such as Enzastaurin; WO 2010056588A1 and WO 2010056620A1 such as tetrasubstituted pyridazines; WO 2010062507A1 such as 1,4-disubstituted phthalazines; WO 2009134574A2 such as disubstituted phthalazines; WO 1999052365A1 such as uinoxaline-5,8-dione derivatives as inhibitors of GTP binding to mutant Ras; U.S. Pat. No. 5,686,467A; U.S. Pat. No. 5,574,047A such as Raloxifene; and U.S. Pat. No. 6,124,311 such as a substituted indole, benzofuran, benzothiophene, naphthalene, or dihydronaphthalene; which are incorporated by reference herein in their entireties.

In some embodiments, compounds for treatment of a PNPLA3-related disorder may include compounds that inhibit the JAK/STAT pathway. In some embodiments, such compounds may be Janus kinase inhibitors, including but not limited to Ruxolitinib, Oclacitinib, Baricitinib, Filgotinib, Gandotinib, Lestaurtinib, PF-04965842, Upadacitinib, Cucurbitacin I, CHZ868, Fedratinib, AC430, AT9283, ati-50001 and ati-50002, AZ 960, AZD1480, BMS-911543, CEP-33779, Cerdulatinib (PRT062070, PRT2070), Curcumol, Decemotinib (VX-509), Fedratinib (SAR302503, TG101348), FLLL32, FM-381, GLPG0634 analogue, Go6976, JANEX-1 (WHI-P131), Momelotinib (CYT387), NVP-BSK805, Pacritinib (SB1518), Peficitinib (ASP015K, JNJ-54781532), PF-06651600, PF-06700841, R256 (AZD0449), Solcitinib (GSK2586184 or GLPG0778), S-Ruxolitinib (INCB018424), TG101209, Tofacitinib (CP-690550), WHI-P154, WP1066, XL019, ZM 39923 HCl, and those described herein.

In some embodiments, compounds for treatment of a PNPLA3-related disorder may include compounds that inhibit the mTOR pathway. In some embodiments, such compounds may be mTOR kinase inhibitors, including but not limited to Apitolisib (GDC-0980, RG7422), AZD8055, BGT226 (NVP-BGT226), CC-223, Chrysophanic Acid, CZ415, Dactolisib (BEZ235, NVP-BEZ235), Everolimus (RAD001), GDC-0349, Gedatolisib (PF-05212384, PKI-587), GSK1059615, INK 128 (MLN0128), KU-0063794, LY3023414, MHY1485, Omipalisib (GSK2126458, GSK458), OSI-027, Palomid 529 (P529), PF-04691502, PI-103, PP121, Rapamycin (Sirolimus), Ridaforolimus (Deforolimus, MK-8669), SF2523, Tacrolimus (FK506), Temsirolimus (CCI-779, NSC 683864), Torin 1, Torin 2, Torkinib (PP242), Vistusertib (AZD2014), Voxtalisib (SAR245409, XL765) Analogue, Voxtalisib (XL765, SAR245409), WAY-600, WYE-125132 (WYE-132), WYE-354, WYE-687, XL388, Zotarolimus (ABT-578), and those described herein.

In some embodiments, compounds for treatment of a PNPLA3-related disorder may include compounds that inhibit the Syk pathway. In some embodiments, such compounds may be Syk inhibitors, including but not limited to R788, tamatinib (R406), entospletinib (GS-9973), nilvadipine, TAK-659, BAY-61-3606, MNS (3,4-Methylenedioxy-β-nitrostyrene, MDBN), Piceatannol, PRT-060318, PRT062607 (P505-15, BIIB057), PRT2761, R09021, cerdulatinib, and those described herein. In some embodiments, such compounds may be Bruton's tyrosine kinase (BTK) inhibitors, including but not limited to ibrutinib, ONO-4059, ACP-196, and those described herein. In some embodiments, such compounds may be PI3K inhibitors, including but not limited to idelalisib, duvelisib, pilaralisib, TGR-1202, GS-9820, ACP-319, SF2523, and those described herein.

In some embodiments, compounds for treatment of a PNPLA3-related disorder may include compounds that inhibit the GSK3 pathway. In some embodiments, such compounds may be GSK3 inhibitors, including but not limited to BIO, AZD2858, 1-Azakenpaullone, AR-A014418, AZD1080, Bikinin, BIO-acetoxime, CHIR-98014, CHIR-99021 (CT99021), IM-12, Indirubin, LY2090314, SB216763, SB415286, TDZD-8, Tideglusib, TWS119, and those described herein.

In some embodiments, compounds for treatment of a PNPLA3-related disorder may include compounds that inhibit the TGF-beta/SMAD pathway. In some embodiments, such compounds may be ACVR1 inhibitors, including but not limited to Momelotinib, BML-275, DMH-1, Dorsomorphin, Dorsomorphin dihydrochloride, K 02288, LDN-193189, LDN-212854, and ML347. In some embodiments, such compounds may be SMAD3 inhibitors, including but not limited to SIS3. In some embodiments, such compounds may be SMAD4 inhibitors.

In some embodiments, compounds for treatment of a PNPLA3-related disorder may include compounds that inhibit the NF-κB pathway. In some embodiments, such compounds may include but not limited to ACHP, 10Z-Hymenialdisine, Amlexanox, Andrographolide, Arctigenin, Bay 11-7085, Bay 11-7821, Bengamide B, BI 605906, BMS 345541, Caffeic acid phenethyl ester, Cardamonin, C-DIM 12, Celastrol, CID 2858522, FPS ZM1, Gliotoxin, GSK 319347A, Honokiol, HU 211, IKK 16, IMD 0354, IP7e, IT 901, Luteolin, MG 132, ML 120B dihydrochloride, ML 130, Parthenolide, PF 184, Piceatannol, PR 39 (porcine), Pristimerin, PS 1145 dihydrochloride, PSI, Pyrrolidinedithiocarbamate ammonium, RAGE antagonist peptide, Ro 106-9920, SC 514, SP 100030, Sulfasalazine, Tanshinone IIA, TPCA-1, Withaferin A, Zoledronic Acid, and those described in Tables 1-3 in International Publication No. WO2008043157A1, the content of which is hereby incorporated by reference in its entirety.

### Polypeptides

In some embodiments, compounds for altering expression of the PNPLA3 gene comprise a polypeptide. As used herein, the term “polypeptide” refers to a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer or tetramer. They may also comprise single chain or multichain polypeptides and may be associated or linked. The term polypeptide may also apply to amino acid polymers in which one or more amino acid residues are an artificial chemical analog of a corresponding naturally occurring amino acid.

### Antibodies

In some embodiments, compounds for altering PNPLA3 expression comprise an antibody. In one embodiment, antibodies of the present invention comprising antibodies, antibody fragments, their variants or derivatives described herein are specifically immunoreactive with at least one molecule of the gene signaling network or networks associated with the insulated neighborhood which contain PNPLA3. Antibodies of the present invention comprising antibodies or fragments of antibodies may also bind to target sites on PNPLA3.

As used herein, the term “antibody” is used in the broadest sense and specifically covers various embodiments including, but not limited to monoclonal antibodies, polyclonal antibodies, multispecific antibodies (e.g. bispecific antibodies formed from at least two intact antibodies), and antibody fragments such as diabodies so long as they exhibit a desired biological activity. Antibodies are primarily amino-acid based molecules but may also comprise one or more modifications such as with sugar moieties.

“Antibody fragments” comprise a portion of an intact antibody, preferably comprising an antigen binding region thereof. Examples of antibody fragments include Fab, Fab′, F(ab′)₂, and Fv fragments; diabodies; linear antibodies; single-chain antibody molecules; and multispecific antibodies formed from antibody fragments. Papain digestion of antibodies produces two identical antigen-binding fragments, called “Fab” fragments, each with a single antigen-binding site. Also produced is a residual “Fc” fragment, whose name reflects its ability to crystallize readily. Pepsin treatment yields an F(ab′)₂ fragment that has two antigen-binding sites and is still capable of cross-linking antigen. Antibodies of the present invention may comprise one or more of these fragments. For the purposes herein, an “antibody” may comprise a heavy and light variable domain as well as an Fc region.

“Native antibodies” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain.

As used herein, the term “variable domain” refers to specific antibody domains that differ extensively in sequence among antibodies and are used in the binding and specificity of each particular antibody for its particular antigen. As used herein, the term “Fv” refers to antibody fragments which contain a complete antigen-recognition and antigen-binding site. This region consists of a dimer of one heavy chain and one light chain variable domain in tight, non-covalent association.

Antibody “light chains” from any vertebrate species can be assigned to one of two clearly distinct types, called kappa and lambda based on amino acid sequences of their constant domains. Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2.

“Single-chain Fv” or “scFv” as used herein, refers to a fusion protein of VH and VL antibody domains, wherein these domains are linked together into a single polypeptide chain. In some embodiments, the Fv polypeptide linker enables the scFv to form the desired structure for antigen binding.

The term “diabodies” refers to small antibody fragments with two antigen-binding sites, which fragments comprise a heavy chain variable domain V_{H} connected to a light chain variable domain V_{L} in the same polypeptide chain. By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA, 90:6444-6448 (1993), the contents of each of which are incorporated herein by reference in their entirety.

Antibodies of the present invention may be polyclonal or monoclonal or recombinant, produced by methods known in the art or as described in this application. The term “monoclonal antibody” as used herein refers to an antibody obtained from a population of substantially homogeneous cells (or clones), i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen.

The modifier “monoclonal” indicates the character of the antibody as being obtained from a substantially homogeneous population of antibodies, and is not to be construed as requiring production of the antibody by any particular method. The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from the hypervariable region from an antibody of the recipient are replaced by residues from the hypervariable region from an antibody of a non-human species (donor antibody) such as mouse, rat, rabbit or nonhuman primate having the desired specificity, affinity, and capacity.

The term “hypervariable region” when used herein in reference to antibodies refers to regions within the antigen binding domain of an antibody comprising the amino acid residues that are responsible for antigen binding. The amino acids present within the hypervariable regions determine the structure of the complementarity determining region (CDR). As used herein, the “CDR” refers to the region of an antibody that comprises a structure that is complimentary to its target antigen or epitope.

In some embodiments, the compositions of the present invention may be antibody mimetics. The term “antibody mimetic” refers to any molecule which mimics the function or effect of an antibody and which binds specifically and with high affinity to their molecular targets. As such, antibody mimics include nanobodies and the like.

In some embodiments, antibody mimetics may be those known in the art including, but are not limited to affibody molecules, affilins, affitins, anticalins, avimers, DARPins, Fynomers and Kunitz and domain peptides. In other embodiments, antibody mimetics may include one or more non-peptide region.

As used herein, the term “antibody variant” refers to a biomolecule resembling an antibody in structure and/or function comprising some differences in their amino acid sequence, composition or structure as compared to a native antibody.

The preparation of antibodies, whether monoclonal or polyclonal, is known in the art. Techniques for the production of antibodies are well known in the art and described, e.g. in Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999.

Antibodies of the present invention may be characterized by their target molecule(s), by the antigens used to generate them, by their function (whether as agonists or antagonists) and/or by the cell niche in which they function.

Measures of antibody function may be made relative to a standard under normal physiologic conditions, in vitro or in vivo. Measurements may also be made relative to the presence or absence of the antibodies. Such methods of measuring include standard measurement in tissue or fluids such as serum or blood such as Western blot, enzyme-linked immunosorbent assay (ELISA), activity assays, reporter assays, luciferase assays, polymerase chain reaction (PCR) arrays, gene arrays, Real Time reverse transcriptase (RT) PCR and the like.

Antibodies of the present invention exert their effects via binding (reversibly or irreversibly) to one or more target sites. While not wishing to be bound by theory, target sites which represent a binding site for an antibody, are most often formed by proteins or protein domains or regions. However, target sites may also include biomolecules such as sugars, lipids, nucleic acid molecules or any other form of binding epitope.

Alternatively, or additionally, antibodies of the present invention may function as ligand mimetics or nontraditional payload carriers, acting to deliver or ferry bound or conjugated drug payloads to specific target sites.

Changes elicited by antibodies of the present invention may result in a neomorphic change in the cell. As used herein, “a neomorphic change” is a change or alteration that is new or different. Such changes include extracellular, intracellular and cross cellular signaling.

In some embodiments, compounds or agents of the invention act to alter or control proteolytic events. Such events may be intracellular or extracellular.

Antibodies of the present invention, as well as antigens used to generate them, are primarily amino acid-based molecules. These molecules may be “peptides,” “polypeptides,” or “proteins.”

As used herein, the term “peptide” refers to an amino-acid based molecule having from 2 to 50 or more amino acids. Special designators apply to the smaller peptides with “dipeptide” referring to a two amino acid molecule and “tripeptide” referring to a three amino acid molecule. Amino acid based molecules having more than 50 contiguous amino acids are considered polypeptides or proteins.

The terms “amino acid” and “amino acids” refer to all naturally occurring L-alpha-amino acids as well as non-naturally occurring amino acids. Amino acids are identified by either the one-letter or three-letter designations as follows: aspartic acid (Asp:D), isoleucine (Ile:I), threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagines (Asn:N), where the amino acid is listed first followed parenthetically by the three and one letter codes, respectively.

In some embodiments, an antibody, such as those shown in WO 2007044411 and WO 2015100104A1, may be used to treat NASH.

### Hybridizing Oligonucleotides

In some embodiments, oligonucleotides, including those which function via a hybridization mechanism, whether single of double stranded such as antisense molecules, RNAi constructs (including siRNA, saRNA, microRNA, etc.), aptamers and ribozymes may be used to alter or as perturbation stimuli of the gene signaling networks associated with PNPLA3.

In some embodiments, hybridizing oligonucleotides (e.g., siRNA) may be used to knock down signaling molecules involved in the pathways regulating PNPLA3 expression such that PNPLA3 expression is reduced in the absence of the signaling molecule. For example, once a pathway is identified to positively regulate PNPLA3 expression, a component of the pathway (e.g., a receptor, a protein kinase, a transcription factor) may be knocked down with an RNAi agent (e.g., siRNA) to reduce the activation of PNPLA3 expression.

In some embodiments, the pathway targeted with a hybridizing oligonucleotide (e.g., siRNA) of the present invention to reduce PNPLA3 expression is the JAK/STAT pathway. In one embodiment, the hybridizing oligonucleotide (e.g., siRNA) is used to knock down JAK1. In one embodiment, the hybridizing oligonucleotide (e.g., siRNA) is used to knock down JAK2.

In some embodiments, the pathway targeted with a hybridizing oligonucleotide (e.g., siRNA) of the present invention to reduce PNPLA3 expression is the Syk pathway. In one embodiment, the hybridizing oligonucleotide (e.g., siRNA) is used to knock down SYK.

In some embodiments, the pathway targeted with a hybridizing oligonucleotide (e.g., siRNA) of the present invention to reduce PNPLA3 expression is the mTOR pathway. In one embodiment, the hybridizing oligonucleotide (e.g., siRNA) is used to knock down mTOR.

In some embodiments, the pathway targeted with a hybridizing oligonucleotide (e.g., siRNA) of the present invention to reduce PNPLA3 expression is the PDGFR pathway. In one embodiment, the hybridizing oligonucleotide (e.g., siRNA) is used to knock down PDGFRA. In one embodiment, the hybridizing oligonucleotide (e.g., siRNA) is used to knock down PDGFRB.

In some embodiments, the pathway targeted with a hybridizing oligonucleotide (e.g., siRNA) of the present invention to reduce PNPLA3 expression is the GSK3 pathway. In one embodiment, the hybridizing oligonucleotide (e.g., siRNA) is used to knock down GSK3.

In some embodiments, the pathway targeted with a hybridizing oligonucleotide (e.g., siRNA) of the present invention to reduce PNPLA3 expression is the TGF-beta/SMAD pathway. In one embodiment, the hybridizing oligonucleotide (e.g., siRNA) is used to knock down ACVR1. In another embodiment, the hybridizing oligonucleotide (e.g., siRNA) is used to knock down SMAD3. In yet another embodiment, the hybridizing oligonucleotide (e.g., siRNA) is used to knock down SMAD4.

In some embodiments, the pathway targeted with a hybridizing oligonucleotide (e.g., siRNA) of the present invention to reduce PNPLA3 expression is the NF-κB pathway. In one embodiment, the hybridizing oligonucleotide (e.g., siRNA) is used to knock down NF-κB.

In some embodiments, a hybridizing oligonucleotide (e.g., siRNA) of the present invention may target Hydroxysteroid 17-Beta Dehydrogenase 13 (HSD17B13) to reduce PNPLA3 expression.

In some embodiments, a hybridizing oligonucleotide as described above may be used together with another hybridizing oligonucleotide to target more than one components in the same pathway, or more than one components from different pathways, to reduce PNPLA3 expression. Such combination therapies may achieve additive or synergetic effects by simultaneously blocking multiple signaling molecules and/or pathways that positively regulate PNPLA3 expression.

As such oligonucleotides may also serve as therapeutics, their therapeutic liabilities and treatment outcomes may be ameliorated or predicted, respectively by interrogating the gene signaling networks of the invention.

### Genome Editing Approaches

In certain embodiments, expression of the PNPLA3 gene may be modulated by altering the chromosomal regions defining the insulated neighborhood(s) and/or genome signaling center(s) associated with PNPLA3. For example, PNPLA3 production may be reduced or eliminated by targeting any one of the members of the molecules of the gene signaling network or networks associated with the insulated neighborhood which contain PNPLA3.

Methods of altering the gene expression attendant to an insulated neighborhood include altering the signaling center (e.g. using CRISPR/Cas to change the signaling center binding site or repair/replace if mutated). These alterations may result in a variety of results including: activation of cell death pathways prematurely/inappropriately (key to many immune disorders), production of too little/much gene product (also known as the rheostat hypothesis), production of too little/much extracellular secretion of enzymes, prevention of lineage differentiation, switch of lineage pathways, promotion of stemness, initiation or interference with auto regulatory feedback loops, initiation of errors in cell metabolism, inappropriate imprinting/gene silencing, and formation of flawed chromatin states. Additionally, genome editing approaches including those well-known in the art may be used to create new signaling centers by altering the cohesin necklace or moving genes and enhancers.

In certain embodiments, genome editing approaches describe herein may include methods of using site-specific nucleases to introduce single-strand or double-strand DNA breaks at particular locations within the genome. Such breaks can be and regularly are repaired by endogenous cellular processes, such as homology-directed repair (HDR) and non-homologous end joining (NHEJ). HDR is essentially an error-free mechanism that repairs double-strand DNA breaks in the presence of a homologous DNA sequence. The most common form of HDR is homologous recombination. It utilizes a homologous sequence as a template for inserting or replacing a specific DNA sequence at the break point. The template for the homologous DNA sequence can be an endogenous sequence (e.g., a sister chromatid), or an exogenous or supplied sequence (e.g., plasmid or an oligonucleotide). As such, HDR may be utilized to introduce precise alterations such as replacement or insertion at desired regions. In contrast, NHEJ is an error-prone repair mechanism that directly joins the DNA ends resulting from a double-strand break with the possibility of losing, adding or mutating a few nucleotides at the cleavage site. The resulting small deletions or insertions (termed “Indels”) or mutations may disrupt or enhance gene expression. Additionally, if there are two breaks on the same DNA, NHEJ can lead to the deletion or inversion of the intervening segment. Therefore, NHEJ may be utilized to introduce insertions, deletions or mutations at the cleavage site.

CRISPR/Cas Systems

In certain embodiments, a CRISPR/Cas system may be used to delete CTCF anchor sites to modulate gene expression within the insulated neighborhood associated with that anchor site. See, Hnisz et al., Cell 167, Nov. 17, 2016, which is hereby incorporated by reference in its entirety. Disruption of the boundaries of insulated neighborhood prevents the interactions necessary for proper function of the associated signaling centers. Changes in the expression genes that are immediately adjacent to the deleted neighborhood boundary have also been observed due to such disruptions.

In certain embodiments, a CRISPR/Cas system may be used to modify existing CTCF anchor sites. For example, existing CTCF anchor sites may be mutated or inverted by inducing NHEJ with a CRISPR/Cas nuclease and one or more guide RNAs, or masked by targeted binding with a catalytically inactive CRISPR/Cas enzyme and one or more guide RNAs. Alteration of existing CTCF anchor sites may disrupt the formation of existing insulated neighborhoods and alter the expression of genes located within these insulated neighborhoods.

In certain embodiments, a CRISPR/Cas system may be used to introduce new CTCF anchor sites. CTCF anchor sites may be introduced by inducing HDR at a selected site with a CRISPR/Cas nuclease, one or more guide RNAs and a donor template containing the sequence of a CTCF anchor site. Introduction of new CTCF anchor sites may create new insulated neighborhoods and/or alter existing insulated neighborhoods, which may affect expression of genes that are located adjacent to these insulated neighborhoods.

In certain embodiments, a CRISPR/Cas system may be used to alter signaling centers by changing signaling center binding sites. For example, if a signaling center binding site contains a mutation that affects the assembly of the signaling center with associated transcription factors, the mutated site may be repaired by inducing a double strand DNA break at or near the mutation using a CRISPR/Cas nuclease and one or more guide RNAs in the presence of a supplied corrected donor template.

In certain embodiments, a CRISPR/Cas system may be used to modulate expression of neighborhood genes by binding to a region within an insulated neighborhood (e.g., enhancer) and block transcription. Such binding may prevent recruitment of transcription factors to signaling centers and initiation of transcription. The CRISPR/Cas system may be a catalytically inactive CRISPR/Cas system that do not cleave DNA.

In certain embodiments, a CRISPR/Cas system may be used to knockdown expression of neighborhood genes via introduction of short deletions in coding regions of these genes. When repaired, such deletions would result in frame shifts and/or introduce premature stop codons in mRNA produced by the genes followed by the mRNA degradation via nonsense-mediated decay. This may be useful for modulation of expression of activating and repressive components of signaling pathways that would result in decreased or increased expression of genes under control of these pathways including disease genes such as PNPLA3.

In other embodiments, a CRISPR/Cas system may also be used to alter cohesion necklace or moving genes and enhancers.

### CRISPR/Cas Enzymes

CRISPR/Cas systems are bacterial adaptive immune systems that utilize RNA-guided endonucleases to target specific sequences and degrade target nucleic acids. They have been adapted for use in various applications in the field of genome editing and/or transcription modulation. Any of the enzymes or orthologs known in the art or disclosed herein may be utilized in the methods herein for genome editing.

In certain embodiments, the CRISPR/Cas system may be a Type II CRISPR/Cas9 system. Cas9 is an endonuclease that functions together with a trans-activating CRISPR RNA (tracrRNA) and a CRISPR RNA (crRNA) to cleave double stranded DNAs. The two RNAs can be engineered to form a single-molecule guide RNA by connecting the 3′ end of the crRNA to the 5′ end of tracrRNA with a linker loop. Jinek et al., Science, 337(6096):816-821 (2012) showed that the CRISPR/Cas9 system is useful for RNA-programmable genome editing, and international patent application W02013/176772 provides numerous examples and applications of the CRISPR/Cas endonuclease system for site-specific gene editing, which are incorporated herein by reference in their entirety. Exemplary CRISPR/Cas9 systems include those derived from Streptococcus pyogenes, Streptococcus thermophilus, Neisseria meningitidis, Treponema denticola, Streptococcus aureas, and Francisella tularensis.

In certain embodiments, the CRISPR/Cas system may be a Type V CRISPR/Cpf1 system. Cpf1 is a single RNA-guided endonuclease that, in contrast to Type II systems, lacks tracrRNA. Cpf1 produces staggered DNA double-stranded break with a 4 or 5 nucleotide 5′ overhang. Zetsche et al. Cell. 2015 Oct. 22; 163(3):759-71 provides examples of Cpf1 endonuclease that can be used in genome editing applications, which is incorporated herein by reference in its entirety. Exemplary CRISPR/Cpf1 systems include those derived from Francisella tularensis, Acidaminococcus sp., and Lachnospiraceae bacterium.

In certain embodiments, nickase variants of the CRISPR/Cas endonucleases that have one or the other nuclease domain inactivated may be used to increase the specificity of CRISPR-mediated genome editing. Nickases have been shown to promote HDR versus NHEJ. HDR can be directed from individual Cas nickases or using pairs of nickases that flank the target area.

In certain embodiments, catalytically inactive CRISPR/Cas systems may be used to bind to target regions (e.g., CTCF anchor sites or enhancers) and interfere with their function. Cas nucleases such as Cas9 and Cpf1 encompass two nuclease domains. Mutating critical residues at the catalytic sites creates variants that only bind to target sites but do not result in cleavage. Binding to chromosomal regions (e.g., CTCF anchor sites or enhancers) may disrupt proper formation of insulated neighborhoods or signaling centers and therefore lead to altered expression of genes located adjacent to the target region.

In certain embodiments, a CRISPR/Cas system may include additional functional domain(s) fused to the CRISPR/Cas endonuclease or enzyme. The functional domains may be involved in processes including but not limited to transcription activation, transcription repression, DNA methylation, histone modification, and/or chromatin remodeling. Such functional domains include but are not limited to a transcriptional activation domain (e.g., VP64 or KRAB, SID or SID4X), a transcriptional repressor, a recombinase, a transposase, a histone remodeler, a DNA methyltransferase, a cryptochrome, a light inducible/controllable domain or a chemically inducible/controllable domain.

In certain embodiments, a CRISPR/Cas endonuclease or enzyme may be administered to a cell or a patient as one or a combination of the following: one or more polypeptides, one or more mRNAs encoding the polypeptide, or one or more DNAs encoding the polypeptide.

### Guide Nucleic Acid

In certain embodiments, guide nucleic acids may be used to direct the activities of an associated CRISPR/Cas enzymes to a specific target sequence within a target nucleic acid. Guide nucleic acids provide target specificity to the guide nucleic acid and CRISPR/Cas complexes by virtue of their association with the CRISPR/Cas enzymes, and the guide nucleic acids thus can direct the activity of the CRISPR/Cas enzymes.

In one aspect, guide nucleic acids may be RNA molecules. In one aspect, guide RNAs may be single-molecule guide RNAs. In one aspect, guide RNAs may be chemically modified.

In certain embodiments, more than one guide RNAs may be provided to mediate multiple CRISPR/Cas-mediated activities at different sites within the genome.

In certain embodiments, guide RNAs may be administered to a cell or a patient as one or more RNA molecules or one or more DNAs encoding the RNA sequences. Ribonucleoprotein complexes (RNPs)

In one embodiment, the CRISPR/Cas enzyme and guide nucleic acid may each be administered separately to a cell or a patient.

In another embodiment, the CRISPR/Cas enzyme may be pre-complexed with one or more guide nucleic acids. The pre-complexed material may then be administered to a cell or a patient. Such pre-complexed material is known as a ribonucleoprotein particle (RNP).

Zinc Finger Nucleases

In certain embodiments, genome editing approaches of the present invention involve the use of Zinc finger nucleases (ZFNs). Zinc finger nucleases (ZFNs) are modular proteins comprised of an engineered zinc finger DNA binding domain linked to a DNA-cleavage domain. A typical DNA-cleavage domain is the catalytic domain of the type II endonuclease FokI. Because FokI functions only as a dimer, a pair of ZFNs must are required to be engineered to bind to cognate target “half-site” sequences on opposite DNA strands and with precise spacing between them to allow the two enable the catalytically active FokI domains to dimerize. Upon dimerization of the FokI domain, which itself has no sequence specificity per se, a DNA double-strand break is generated between the ZFN half-sites as the initiating step in genome editing.

Transcription Activator-Like Effector Nucleases (TALENs)

In certain embodiments, genome editing approaches of the present invention involve the use of Transcription Activator-Like Effector Nucleases (TALENs). TALENs represent another format of modular nucleases which, similarly to ZFNs, are generated by fusing an engineered DNA binding domain to a nuclease domain, and operate in tandem to achieve targeted DNA cleavage. While the DNA binding domain in ZFN consists of Zinc finger motifs, the TALEN DNA binding domain is derived from transcription activator-like effector (TALE) proteins, which were originally described in the plant bacterial pathogen Xanthomonas sp. TALEs are comprised of tandem arrays of 33-35 amino acid repeats, with each repeat recognizing a single basepair in the target DNA sequence that is typically up to 20 bp in length, giving a total target sequence length of up to 40 bp. Nucleotide specificity of each repeat is determined by the repeat variable diresidue (RVD), which includes just two amino acids at positions 12 and 13. The bases guanine, adenine, cytosine and thymine are predominantly recognized by the four RVDs: Asn-Asn, Asn-Ile, His-Asp and Asn-Gly, respectively. This constitutes a much simpler recognition code than for zinc fingers, and thus represents an advantage over the latter for nuclease design. Nevertheless, as with ZFNs, the protein-DNA interactions of TALENs are not absolute in their specificity, and TALENs have also benefitted from the use of obligate heterodimer variants of the FokI domain to reduce off-target activity.

### Methods

Modulation of a chromatin binding protein, such as a transcription factor, can include one or more of: phosphorylation, de-phosphorylation, methylation, de-methylation, acetylation, de-acetylation, ubiquitination, de-ubiquitination, glycosylation, de-glyosylation, sumoylation, and de-sumoylation. The net effect of such modulation is to alter the function of the chromatin binding protein. Such alteration can include one or more of: increased or decreased binding to DNA, increased or decreased binding to one or more chromatin binding proteins, increased or decreased stability of the chromatin binding protein, or change in sub-cellular localization of the chromatin binding protein.

Gene circuitry mapping can be used to make novel connections between signaling pathways and genome-wide regulation of transcription, allowing for identification of druggable targets that are predicated to up- or down-regulate expression of disease-associated genes. The inventors have applied this gene circuitry mapping to identify drugging signaling pathways to modulate or reduce PNPLA3 transcription as therapeutic targets. Gene mapping utilizes four approaches: HiChIP, ATAC-Seq, ChIP-seq, and RNA-seq.

HiChIP is a technique that defines chromatin domains (insulated neighborhoods) and DNA-DNA interactions, such as enhancer-promoter interactions. ATAC-seq identifies open chromatin regions and activate enhancers. ChIP-seq reveals binding of transcription factors to DNA, modified histones, and chromatin-binding proteins genome wide. RNA-seq quantifies transcript levels of every gene.

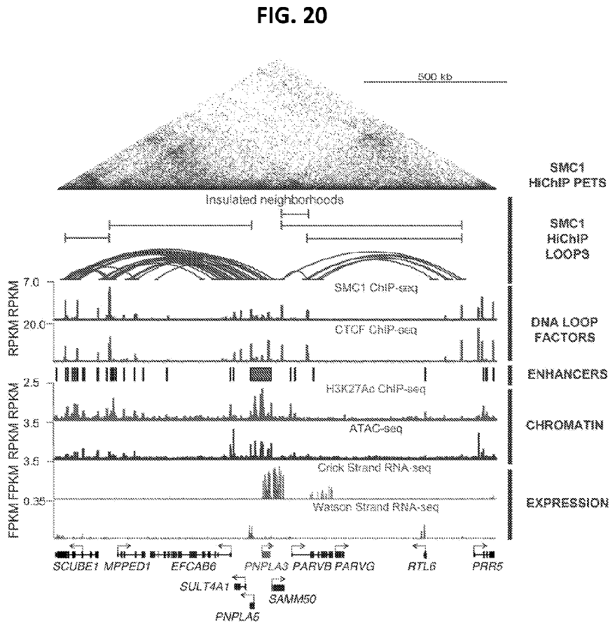

Using these gene mapping techniques showed PNPLA3 is insulated from neighboring domains and highlighted key enhancers that are likely to regulate expression. The gene mapping results are shown in FIG. 20. The top panel shows the results of the HiChIP mapping, while the bottom panel shows a comparison of the results with the additional mapping techniques.

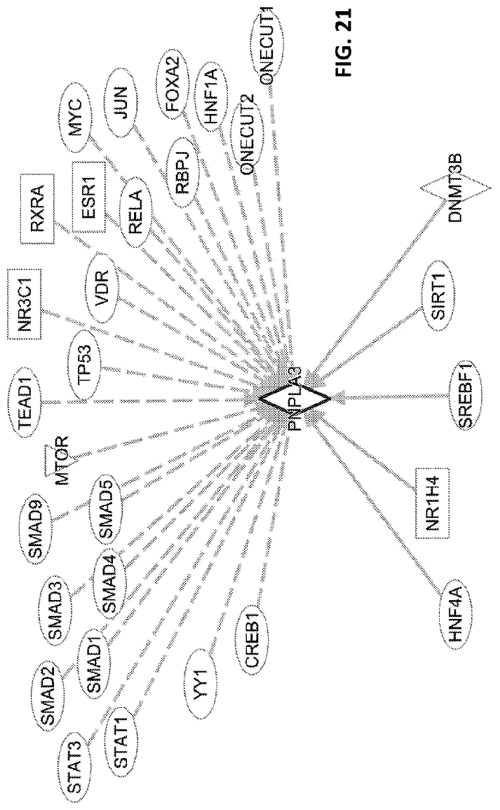

The ChIP-seq assay identified 16 new transcription factors, in addition to the previously reported transcription factors that bind the PNPLA3, as shown in FIG. 21. The gene circuitry mapping approach predicted multiple pathways with potential to regulate PNPLA3 expression.

### Diagnostic and Treatment Methods

In some embodiments, described herein are methods, compositions and kits for identifying a subject suitable for a PNPLA3-targeted treatment with the compositions and methods and administering a PNPLA3-targeted therapy.

In some embodiments, the methods for identifying a subject for the PNPLA3-targeted treatment includes the step of determining whether the subject has the mutation PNPLA3-I148M. Specifically, the genetic marker is a G allele at SNP rs738409 (c.444 C-G). The G allele frequency varies by ethnicity and is estimated to be about 0.57 in Latino, 0.38 in East Asian, 0.23 in European, 0.22 in South Asian, and 0.14 in African populations.

Genotyping for the PNPLA3-I148M variant may be carried out via any suitable methods known in the art. For example, a biological sample is obtained from the subject, and genomic DNA is isolated. The biological sample may be any material that can be used to determine a DNA profile such as blood, semen, saliva, urine, feces, hair, teeth, bone, tissue and cells. The gene variant may then be detected by methods such as, but not limited to, mass spectroscopy, oligonucleotide microarray analysis, allele-specific hybridization, allele-specific PCR, and/or sequencing. See U.S. Pat. No. 8,785,128, which is hereby incorporated by reference in its entirety.

Alternatively, the gene variant may also be detected by detecting the mutant PNPLA3 protein, e.g., with an antibody or any other binding molecules. An antibody binding assay, such as a Western blot or ELISA, may be performed. The mutant protein can also be detected using protein mass spectroscopy methods, including mass spectroscopy (MS), tandem mass spectroscopy (MS/MS), liquid chromatography-mass spectrometry (LC-MS) gas chromatography-mass spectrometry (GC-MS), or high performance liquid chromatography (HPLC) mass spectroscopy (LC-MS or LC-MS/MS). Any appropriate mass analyzer may be used, including, but not limited to, time-of-flight [TOF], orbitraps, quadrupoles and ion traps.

In some embodiments, the subject may have been biopsied or otherwise sampled prior to the diagnosis described herein. In that case, detection of the genetic marker of PNPLA3-I148M, whether DNA-based or protein-based, may be performed using the biopsy sample or any other biological sample already obtained from the subject.

In some embodiments, the presence of a PNPLA3 gene variant may be determined or already have been determined in the subject. Such determination or prior determination may be performed by a commercial or non-commercial third-party genetic test or genotyping kit. Commercial genotyping kits are available from a variety of vendors, including 23andMe, AncestryDNA, HelixDNA, Vitagene DNA Test, National Geographic DNA Test Kit: Geno2.0, and DNA Consultants. Determination or prior determination of the presence of a PNPLA3 gene variant may also be determined by a healthcare provider. In some embodiments, a biological sample is obtained from the subject and a dataset comprising the genomic or proteomic data from the biological sample is obtained.

In some embodiments, the methods for identifying a subject for the PNPLA3-targeted treatment may further include a step of measuring hepatic triglyceride in the subject. As a non-limiting example, the hepatic triglyceride content may be measured using proton magnetic resonance spectroscopy (¹H-MRS). Proton magnetic resonance spectroscopy allows for accurate, quantitative noninvasive assessment of tissue fat content.

In some embodiments, the methods for identifying a subject for the PNPLA3-targeted treatment may further include a step of determining if the subject has or is predisposed to having a PNPLA3-related disorder (e.g., NAFLD, NASH, and/or ALD). Such disorders may be assessed using conventional clinical diagnosis. For example, fatty liver or hepatic steatosis may be determined inter alia using computer-aided tomography (CAT) scan or nuclear magnetic resonance (NMR), such as proton magnetic resonance spectroscopy. Diagnosis is generally clinically defined as having hepatic triglyceride content greater than 5.5% volume/volume. Indicators of predisposition to fatty liver may include obesity, diabetes, insulin resistance, and alcohol ingestion.

In some embodiments, the methods may further include performing a liver biopsy, an imaging technique such as ultrasound, a liver function test, a fibrosis test, or any other techniques described in Yki-Jarvinen, H. Diabetologia (2016) 59: 1104; Madrazo Gastroenterol Hepatol (N Y). 2017 June; 13(6): 378-380, which are hereby incorporated by reference in their entirety.

In some embodiments, the diagnostic testing may be performed by others, such as a medical laboratory or clinical test provider.

In some embodiments, the methods may further include verifying the validity of the genotype and/or protein abnormality in silico.

In some embodiments, a targeted therapy is any therapy that directly or indirectly impacts PNPLA3 activity or expression. PNPLA3 gene expression can be measured via any known RNA, mRNA, or protein quantitative assay, including, but not limited to, as RNA-seq, quantitative reverse transcription PCR (qRT-PCR), RNA microarrays, fluorescent in situ hybridization (FISH), antibody binding, Western blotting, ELISA, or any other assay known in the art.

Non-human animal data, such as mouse in vivo data, showing the impact of small molecule inhibitors or RNAi knockdown of members of the multiple pathways that regulate PNPLA3 expression can be used as evidence that the therapy, when administered to a human, is a PNPLA3-targeted therapy. In addition, data obtained in human hepatocytes, including hepatocytes from humans who harbor the G allele at SNP rs738409, can be used to identify a therapy as a PNPLA3-targeted therapy.

In some embodiments, the PNPLA3 targeted therapy comprises an mTOR pathway inhibitor. The mTOR pathway comprises two signaling complexes, mTORC1 and mTORC2. The mTORC1 complex comprises mTOR, mLST8, PRAS40, Deptor, and Raptor. In contrast, the mTORC2 complex comprises mTOR, mLST8, mSIN1, Protor, Deptor, and RICTOR. Activation of the mTORC1 complex results in phosphorylation of p70^{S6K} (also called S6 Kinase, S6K or S6) and 4E-BP1, resulting in downstream gene transcription and translation. Activation of the mTORC2 complex results in phosphorylation and activation of the AKT, SGK1, NDRG1, and PKC proteins. mTORC2 phosphorylates AKT at serine 473 and Threonine 308. AKT also activates the mTORC1 complex. Direct or indirect inhibition includes, but is not limited to, inhibiting the catalytic activity of the mTOR kinase or inhibiting binding of substrate to the kinase.

In some embodiments, the mTOR inhibitor comprises an mTORC1 and mTORC2 inhibitor. In some embodiments, the mTOR inhibitor comprises an mTORC2 inhibitor. In some embodiments, the mTORC2 inhibitor comprises a RICTOR inhibitor.

Any appropriate method to measure inhibition of mTOR activity may be used. Such methods are well known in the art and include ELISAs or Western Blotting to measure the phosphorylation of mTOR substrates, such as S6K, AKT, SGK1, PKC, NDRG1, and/or 4EBP1, or any other mTOR substrate known in the art. ELISA kits for phosphorylated mTOR substrates are available from a variety of manufacturers, including MilliporeSigma, Cell Signaling, and Abcam. Antibodies for phosphorylated mTOR substrates are available from a variety of manufacturers, including Call Siganling, Abcam, and Santa Cruz Biotech.

In some embodiments, the PNPLA3 targeted therapy comprises an mTOR pathway inhibitor that does not inhibit phosphoinositide 3-kinases (PI3K, also known as phosphatidylinositol 3-kinase). PI3Ks are intracellular signaling molecules that phosporylate phosphatidylinositols (PIs). The PI3K family is divided into 3 classes based on primary structure, reulation and lipid substrate specificty: Class I, Class II, and Class III. Class I PI3Ks are heterodimeric molecules comprisng a regulatory subunit and a catalytic subunit. They catalyze the phosphorylation of phosphatidylinositol (4,5)-bisphosphate (PI(4,5)P2) into phosphatidylinositol (3,4,5)-trisphosphate (PI(3,4,5)P3) in vivo. Class IA PI3Ks comprise a p110α/β/δ catalytic subunit and a p85α/β, p55α/γ, or p50α regulatory subunit. PI3Kα, PI3Kβ, and PI3Kδ are all Class IA PI3Ks. Class IB PI3Ks comprise a p110γ catalytic subunit and a p101 regulatory subunit. PI3Kγ is a Class 1B PI3K. Class II PI3Ks comprise catalytic subunits only, termed C2α, C2β, and C2γ, which lack aspartic acid residues and catalyse the production of PI(3)P from PI and PI(3,4)P₂ from PI(4)P. Class III PI3Ks are heterodimers of a catlaytic subunit, Vps34, and regulator subunits (Vsp15/p150). Class III PI3Ks catalyze the production of only PI(3)P from PI.

Inhibitors that do not inhibit the PI3K pathway include mTOR inhibitors that do not directly or indirectly inhibit class I, class II, or class III PI3K proteins. In some embodiments, the mTOR inhibitors do not directly or indirectly inhibit class I, class II, or class III PI3K enzymatic activity. In some embodiments, the mTOR inhibitors do not directly or indirectly inhibit class I, class II, or class III PI3K protein stability or class I, class II, or class III PI3K gene expression. In some embodiments, the mTOR inhibitors do not directly or indirectly inhibit the catalytic subunits of the class I, class II, or class III PI3K proteins, or the regulatory subunits of the class I, class II, or class III PI3K proteins. Direct or indirect inhibition includes, but is not limited to, inhibiting the catalytic activity of the PI3 kinase or inhibiting binding of substrate to the kinase.

Methods of assessing PI3K activity in cells are known in the art and include ELISAs to measure the phosphorylation of PI3K substrates, such as PI, (PI(4,5)P2), or PI(3,4)P2. In addition, methods of assessing purified PI3K activity are also well known in the art and include monitoring of radioactive or fluorescent γ-ATP into PI3K substrates or ratiometric fluorescence superquenching (Stankewicz C, et al, Journal of Biomolecular Screening 11(4); 2006). Any appropriate method to measure PI3K activity may be used.

In some embodiments, the PNPLA3 targeted therapy comprises an mTOR pathway inhibitor that does not inhibit DNA-PK. DNA-PK is a member of the phosphatidylinositol 3-kinase-related kinases (PIKK) protein family, which is sometimes referred to as Class IV PI3K. DNA-PK is a heterodimer formed by the catalytic subunit DNA-PKcs and the autoimmune antigen Ku. DNA-PK phosphorylates p53, Akt/PKB, and CHK2, among other protein targets. Inhibitors that do not inhibit DNA-PK include inhibitors that do not directly or indirectly inhibit DNA-PK. In some embodiments, the mTOR inhibitors do not directly or indirectly inhibit DNA-PK enzymatic activity. In some embodiments, the mTOR inhibitors do not directly or indirectly inhibit DNA-PK protein stability or gene expression. In some embodiments, the mTOR inhibitors do not directly or indirectly inhibit the catalytic or regulatory subunits of DNA-PK. Direct or indirect inhibition includes, but is not limited to, inhibiting the catalytic activity of the DNA-PK kinase or inhibiting binding of substrate to the kinase.

In some embodiments, the PNPLA3 targeted therapy comprises an mTOR pathway inhibitor that does not inhibit PIP4K2C. PIP4K2C is a subunit of type-2 phosphatidylinositol-5-phosphate 4-kinase that converts phosphatidylinositol-5-phosphate (PI(5)P) to phosphatidylinositol-4,5-bisphosphate (PI(4,5)P2). Inhibitors that do not inhibit PIP4K2C include inhibitors that do not directly or indirectly inhibit PIP4K2C. In some embodiments, the mTOR inhibitors do not directly or indirectly inhibit PIP4K2C enzymatic activity. In some embodiments, the mTOR inhibitors do not directly or indirectly inhibit PIP4K2C protein stability or gene expression. In some embodiments, the mTOR inhibitors do not directly or indirectly inhibit the catalytic or regulatory subunits of PIP4K2C. Direct or indirect inhibition includes, but is not limited to, inhibiting the catalytic activity of the PIP4K2C kinase or inhibiting binding of substrate to the kinase.

In some embodiments, the compound capable of reducing the expression of the PNPLA3 gene does not induce hyperinsulinemia in the subject. Hyperinsulinemia is a higher than normal fasting insulin level in a subject's blood plasma. Reference ranges for hyperinsulinemia generally recite normal insulin levels under fasting conditions (8 hour fast) as less than 25 μU/L or less than 174 pmol/L. 30 minutes after a meal or glucose administration, a normal insulin level is 30-230 μU/L or 208-1597 pmol/L. One hour after a meal or glucose administration, a normal insulin level is 18-276 μU/L or 125-1917 pmol/L. Two hours after a meal or glucose administration, a normal insulin level is 16-166 μU/L or 111-1153 pmol/L. In some embodiments, hyperinsulinemia is an insulin level greater than 25 μU/L after an 8 hour fast. In some embodiments, hyperinsulinemia is an insulin level greater than 170 μU/L two hours after a meal or glucose administration.

In some embodiments, the compound capable of reducing the expression of the PNPLA3 gene does not induce hyperglycemia in the subject. Hyperglycemia is a higher than normal amount of glucose in a subject's blood plasma. Reference ranges for hyperglycemia generally recite blood sugar levels higher than 11.1 mmol/L or 200 mg/dL. A non-diabetic normal glucose level is generally considered to be under 140 mg/dL two hours after a meal. However, even consistent blood sugar levels between 5.6 and 7 mmol/l (100-126 mg/dL) can be considered slightly hyperglycemic. In some embodiments, a blood sugar level higher than 130 mg/dL after an 8 hour fast is a hyperglycemic level. In some embodiments, a blood sugar level higher than 180 mg/dL two hours after a meal is a hyperglycemic level.

### Kits

Further provided herein are compositions and kits for the detection of the genetic marker of PNPLA3-I148M, i.e., SNP rs738409, c.444 C-G. Such kits may include devices and instructions that a subject can use to obtain a sample, e.g., of buccal cells or blood, without the aid of a health care provider. The kit may also include a set of instructions and materials for preparing a tissue or cell sample and preparing nucleic acids (such as genomic DNA) from the sample.

In some embodiments, the invention provides compositions and kits comprising primers and primer pairs, which allow the specific amplification of the polynucleotides at the PNPLA3 SNP locus or any specific parts thereof, and/or probes that selectively or specifically hybridize to nucleic acid molecules at the PNPLA3 SNP locus or to any part thereof. Probes may be labeled with a detectable marker, such as, for example, a radioisotope, fluorescent compound, bioluminescent compound, a chemiluminescent compound, metal chelator or enzyme. Such probes and primers may be used to detect the presence of polynucleotides in a sample and as a means for detecting cell expressing proteins encoded by the polynucleotides. As will be understood by the skilled artisan, a great many different primers and probes may be prepared based on the sequence provided herein and used effectively to amplify, clone and/or determine the presence and/or levels of genomic DNAs.

In some embodiments, the kit may comprise reagents for detecting presence of a mutant PNPLA3 protein. Such reagents may be antibodies or other binding molecules that specifically bind to a mutant PNPLA3 protein. In some embodiments, such antibodies or binding molecules may be capable of distinguishing a structural variation to the protein as a result of polymorphism, and thus may be used for genotyping. The antibodies or binding molecules may be labeled with a detectable marker, such as, for example, a radioisotope, fluorescent compound, bioluminescent compound, a chemiluminescent compound, metal chelator or enzyme. Other reagents for performing binding assays, such as ELISA, may be included in the kit.

In some embodiments, the kits may further comprise a surface or substrate (such as a microarray) for capture probes for detecting of amplified nucleic acids. The kit may further comprise instructions for using the genetic marker to conduct a companion diagnostic test.

The kits may further comprise a carrier means being compartmentalized to receive in close confinement one or more container means such as vials, tubes, and the like, each of the container means comprising one of the separate elements to be used in the method. For example, one of the container means may comprise a probe that is or can be detectably labeled. Such probe may be a polynucleotide specific for the genetic marker. Where the kit utilizes nucleic acid hybridization to detect the target nucleic acid, the kit may also have containers containing nucleotide(s) for amplification of the target nucleic acid sequence and/or a container comprising a reporter-means, such as a biotin-binding protein, such as avidin or streptavidin, bound to a reporter molecule, such as an enzymatic, florescent, or radioisotope label.

The kit of the invention will typically comprise the container described above and one or more other containers comprising materials desirable from a commercial and user standpoint, including buffers, diluents, filters, needles, syringes, and package inserts with instructions for use. A label may be present on the container to indicate that the composition is used for a specific therapy or non-therapeutic application, and may also indicate directions for either in vivo or in vitro use, such as those described above.

The invention provides a variety of compositions suitable for use in performing methods of the invention, which may be used in kits. For example, the invention provides surfaces, such as arrays that can be used in such methods. In some embodiments, an array of the invention comprises individual or collections of nucleic acid molecules useful for detecting the genetic marker of the invention. For instance, an array of the invention may comprise a series of discretely placed individual nucleic acid oligonucleotides or sets of nucleic acid oligonucleotide combinations that are hybridizable to a sample comprising target nucleic acids, whereby such hybridization is indicative of genotypes of the genetic marker of the invention.

### IV. Formulations and Delivery

### Pharmaceutical Compositions

According to the present invention the compositions may be prepared as pharmaceutical compositions. It will be understood that such compositions necessarily comprise one or more active ingredients and, most often, a pharmaceutically acceptable excipient.

Relative amounts of the active ingredient, a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between .5 and 50%, between 1-30%, between 5-80%, at least 80% (w/w) active ingredient.

In some embodiments, the pharmaceutical compositions described herein may comprise at least one payload. As a non-limiting example, the pharmaceutical compositions may contain 1, 2, 3, 4 or 5 payloads.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds, including commercially relevant birds such as poultry, chickens, ducks, geese, and/or turkeys.

In some embodiments, compositions are administered to humans, human patients or subjects.

### Formulations

Formulations of the present invention can include, without limitation, saline, liposomes, lipid nanoparticles, polymers, peptides, proteins, cells transfected with viral vectors (e.g., for transfer or transplantation into a subject) and combinations thereof.

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general, such preparatory methods include the step of associating the active ingredient with an excipient and/or one or more other accessory ingredients.

Formulations of the compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/w) of the active ingredient. By way of example, the composition may comprise between 0.1% and 100%, e.g., between 0.5 and 50%, between 1-30%, between 5-80%, at least 80% (w/w) active ingredient.

### Excipients and Diluents

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

### Inactive Ingredients

In some embodiments, the pharmaceutical compositions formulations may comprise at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present invention may be approved by the US Food and Drug Administration (FDA).

In one embodiment, the pharmaceutical compositions comprise at least one inactive ingredient such as, but not limited to, 1,2,6-Hexanetriol; 1,2-Dimyristoyl-Sn-Glycero-3-(Phospho-S-(1-Glycerol)); 1,2-Dimyristoyl-Sn-Glycero-3-Phosphocholine; 1,2-Dioleoyl-Sn-Glycero-3-Phosphocholine; 1,2-Dipalmitoyl-Sn-Glycero-3-(Phospho-Rac-(1-Glycerol)); 1,2-Distearoyl-Sn-Glycero-3-(Phospho-Rac-(1-Glycerol)); 1,2-Distearoyl-Sn-Glycero-3-Phosphocholine; 1-O-Tolylbiguanide; 2-Ethyl-1,6-Hexanediol; Acetic Acid; Acetic Acid, Glacial; Acetic Anhydride; Acetone; Acetone Sodium Bisulfite; Acetylated Lanolin Alcohols; Acetylated Monoglycerides; Acetylcysteine; Acetyltryptophan, DL-; Acrylates Copolymer; Acrylic Acid-Isooctyl Acrylate Copolymer; Acrylic Adhesive 788; Activated Charcoal; Adcote 72A103; Adhesive Tape; Adipic Acid; Aerotex Resin 3730; Alanine; Albumin Aggregated; Albumin Colloidal; Albumin Human; Alcohol; Alcohol, Dehydrated; Alcohol, Denatured; Alcohol, Diluted; Alfadex; Alginic Acid; Alkyl Ammonium Sulfonic Acid Betaine; Alkyl Aryl Sodium Sulfonate; Allantoin; Allyl .Alpha.-Ionone; Almond Oil; Alpha-Terpineol; Alpha-Tocopherol; Alpha-Tocopherol Acetate, Dl-; Alpha-Tocopherol, Dl-; Aluminum Acetate; Aluminum Chlorhydroxy Allantoinate; Aluminum Hydroxide; Aluminum Hydroxide-Sucrose, Hydrated; Aluminum Hydroxide Gel; Aluminum Hydroxide Gel F 500; Aluminum Hydroxide Gel F 5000; Aluminum Monostearate; Aluminum Oxide; Aluminum Polyester; Aluminum Silicate; Aluminum Starch Octenylsuccinate; Aluminum Stearate; Aluminum Subacetate; Aluminum Sulfate Anhydrous; Amerchol C; Amerchol-Cab; Aminomethylpropanol; Ammonia; Ammonia Solution; Ammonia Solution, Strong; Ammonium Acetate; Ammonium Hydroxide; Ammonium Lauryl Sulfate; Ammonium Nonoxynol-4 Sulfate; Ammonium Salt Of C-12-C-15 Linear Primary Alcohol Ethoxylate; Ammonium Sulfate; Ammonyx; Amphoteric-2; Amphoteric-9; Anethole; Anhydrous Citric Acid; Anhydrous Dextrose; Anhydrous Lactose; Anhydrous Trisodium Citrate; Aniseed Oil; Anoxid Sbn; Antifoam; Antipyrine; Apaflurane; Apricot Kernel Oil Peg-6 Esters; Aquaphor; Arginine; Arlacel; Ascorbic Acid; Ascorbyl Palmitate; Aspartic Acid; Balsam Peru; Barium Sulfate; Beeswax; Beeswax, Synthetic; Beheneth-10; Bentonite; Benzalkonium Chloride; Benzenesulfonic Acid; Benzethonium Chloride; Benzododecinium Bromide; Benzoic Acid; Benzyl Alcohol; Benzyl Benzoate; Benzyl Chloride; Betadex; Bibapcitide; Bismuth Subgallate; Boric Acid; Brocrinat; Butane; Butyl Alcohol; Butyl Ester Of Vinyl Methyl Ether/Maleic Anhydride Copolymer (125000 Mw); Butyl Stearate; Butylated Hydroxyanisole; Butylated Hydroxytoluene; Butylene Glycol; Butylparaben; Butyric Acid; C20-40 Pareth-24; Caffeine; Calcium; Calcium Carbonate; Calcium Chloride; Calcium Gluceptate; Calcium Hydroxide; Calcium Lactate; Calcobutrol; Caldiamide Sodium; Caloxetate Trisodium; Calteridol Calcium; Canada Balsam; Caprylic/Capric Triglyceride; Caprylic/Capric/Stearic Triglyceride; Captan; Captisol; Caramel; Carbomer 1342; Carbomer 1382; Carbomer 934; Carbomer 934p; Carbomer 940; Carbomer 941; Carbomer 980; Carbomer 981; Carbomer Homopolymer Type B (Allyl Pentaerythritol Crosslinked); Carbomer Homopolymer Type C (Allyl Pentaerythritol Crosslinked); Carbon Dioxide; Carboxy Vinyl Copolymer; Carboxymethylcellulose; Carboxymethylcellulose Sodium; Carboxypolymethylene; Carrageenan; Carrageenan Salt; Castor Oil; Cedar Leaf Oil; Cellulose; Cellulose, Microcrystalline; Cerasynt-Se; Ceresin; Ceteareth-12; Ceteareth-15; Ceteareth-30; Cetearyl Alcohol/Ceteareth-20; Cetearyl Ethylhexanoate; Ceteth-10; Ceteth-2; Ceteth-20; Ceteth-23; Cetostearyl Alcohol; Cetrimonium Chloride; Cetyl Alcohol; Cetyl Esters Wax; Cetyl Palmitate; Cetylpyridinium Chloride; Chlorobutanol; Chlorobutanol Hemihydrate; Chlorobutanol, Anhydrous; Chlorocresol; Chloroxylenol; Cholesterol; Choleth; Choleth-24; Citrate; Citric Acid; Citric Acid Monohydrate; Citric Acid, Hydrous; Cocamide Ether Sulfate; Cocamine Oxide; Coco Betaine; Coco Diethanolamide; Coco Monoethanolamide; Cocoa Butter; Coco-Glycerides; Coconut Oil; Coconut Oil, Hydrogenated; Coconut Oil/Palm Kernel Oil Glycerides, Hydrogenated; Cocoyl Caprylocaprate; Cola Nitida Seed Extract; Collagen; Coloring Suspension; Corn Oil; Cottonseed Oil; Cream Base; Creatine; Creatinine; Cresol; Croscarmellose Sodium; Crospovidone; Cupric Sulfate; Cupric Sulfate Anhydrous; Cyclomethicone; Cyclomethicone/Dimethicone Copolyol; Cysteine; Cysteine Hydrochloride; Cysteine Hydrochloride Anhydrous; Cysteine, Dl-; D&C Red No. 28; D&C Red No. 33; D&C Red No. 36; D&C Red No. 39; D&C Yellow No. 10; Dalfampridine; Daubert 1-5 Pestr (Matte) 164z; Decyl Methyl Sulfoxide; Dehydag Wax Sx; Dehydroacetic Acid; Dehymuls E; Denatonium Benzoate; Deoxycholic Acid; Dextran; Dextran 40; Dextrin; Dextrose; Dextrose Monohydrate; Dextrose Solution; Diatrizoic Acid; Diazolidinyl Urea; Dichlorobenzyl Alcohol; Dichlorodifluoromethane; Dichlorotetrafluoroethane; Diethanolamine; Diethyl Pyrocarbonate; Diethyl Sebacate; Diethylene Glycol Monoethyl Ether; Diethylhexyl Phthalate; Dihydroxyaluminum Aminoacetate; Diisopropanolamine; Diisopropyl Adipate; Diisopropyl Dilinoleate; Dimethicone 350; Dimethicone Copolyol; Dimethicone Mdx4-4210; Dimethicone Medical Fluid 360; Dimethyl Isosorbide; Dimethyl Sulfoxide; Dimethylaminoethyl Methacrylate-Butyl Methacrylate-Methyl Methacrylate Copolymer; Dimethyldioctadecylammonium Bentonite; Dimethylsiloxane/Methylvinylsiloxane Copolymer; Dinoseb Ammonium Salt; Dipalmitoylphosphatidylglycerol, Dl-; Dipropylene Glycol; Disodium Cocoamphodiacetate; Disodium Laureth Sulfosuccinate; Disodium Lauryl Sulfosuccinate; Disodium Sulfosalicylate; Disofenin; Divinylbenzene Styrene Copolymer; Dmdm Hydantoin; Docosanol; Docusate Sodium; Duro-Tak 280-2516; Duro-Tak 387-2516; Duro-Tak 80-1196; Duro-Tak 87-2070; Duro-Tak 87-2194; Duro-Tak 87-2287; Duro-Tak 87-2296; Duro-Tak 87-2888; Duro-Tak 87-2979; Edetate Calcium Disodium; Edetate Disodium; Edetate Disodium Anhydrous; Edetate Sodium; Edetic Acid; Egg Phospholipids; Entsufon; Entsufon Sodium; Epilactose; Epitetracycline Hydrochloride; Essence Bouquet 9200; Ethanolamine Hydrochloride; Ethyl Acetate; Ethyl Oleate; Ethylcelluloses; Ethylene Glycol; Ethylene Vinyl Acetate Copolymer; Ethylenediamine; Ethylenediamine Dihydrochloride; Ethylene-Propylene Copolymer; Ethylene-Vinyl Acetate Copolymer (28% Vinyl Acetate); Ethylene-Vinyl Acetate Copolymer (9% Vinylacetate); Ethylhexyl Hydroxystearate; Ethylparaben; Eucalyptol; Exametazime; Fat, Edible; Fat, Hard; Fatty Acid Esters; Fatty Acid Pentaerythriol Ester; Fatty Acids; Fatty Alcohol Citrate; Fatty Alcohols; Fd&C Blue No. 1; Fd&C Green No. 3; Fd&C Red No. 4; Fd&C Red No. 40; Fd&C Yellow No. 10 (Delisted); Fd&C Yellow No. 5; Fd&C Yellow No. 6; Ferric Chloride; Ferric Oxide; Flavor 89-186; Flavor 89-259; Flavor Df-119; Flavor Df-1530; Flavor Enhancer; Flavor FIG. 827118; Flavor Raspberry Pfc-8407; Flavor Rhodia Pharmaceutical No. Rf 451; Fluorochlorohydrocarbons; Formaldehyde; Formaldehyde Solution; Fractionated Coconut Oil; Fragrance 3949-5; Fragrance 520a; Fragrance 6.007; Fragrance 91-122; Fragrance 9128-Y; Fragrance 93498g; Fragrance Balsam Pine No. 5124; Fragrance Bouquet 10328; Fragrance Chemoderm 6401-B; Fragrance Chemoderm 6411; Fragrance Cream No. 73457; Fragrance Cs-28197; Fragrance Felton 066m; Fragrance Firmenich 47373; Fragrance Givaudan Ess 9090/1c; Fragrance H-6540; Fragrance Herbal 10396; Fragrance Nj-1085; Fragrance P O Fl-147; Fragrance Pa 52805; Fragrance Pera Derm D; Fragrance Rbd-9819; Fragrance Shaw Mudge U-7776; Fragrance Tf 044078; Fragrance Ungerer Honeysuckle K 2771; Fragrance Ungerer N5195; Fructose; Gadolinium Oxide; Galactose; Gamma Cyclodextrin; Gelatin; Gelatin, Crosslinked; Gelfoam Sponge; Gellan Gum (Low Acyl); Gelva 737; Gentisic Acid; Gentisic Acid Ethanolamide; Gluceptate Sodium; Gluceptate Sodium Dihydrate; Gluconolactone; Glucuronic Acid; Glutamic Acid, Dl-; Glutathione; Glycerin; Glycerol Ester Of Hydrogenated Rosin; Glyceryl Citrate; Glyceryl Isostearate; Glyceryl Laurate; Glyceryl Monostearate; Glyceryl Oleate; Glyceryl Oleate/Propylene Glycol; Glyceryl Palmitate; Glyceryl Ricinoleate; Glyceryl Stearate; Glyceryl Stearate-Laureth-23; Glyceryl Stearate/Peg Stearate; Glyceryl Stearate/Peg-100 Stearate; Glyceryl Stearate/Peg-40 Stearate; Glyceryl Stearate-Stearamidoethyl Diethylamine; Glyceryl Trioleate; Glycine; Glycine Hydrochloride; Glycol Distearate; Glycol Stearate; Guanidine Hydrochloride; Guar Gum; Hair Conditioner (18n195-1m); Heptane; Hetastarch; Hexylene Glycol; High Density Polyethylene; Histidine; Human Albumin Microspheres; Hyaluronate Sodium; Hydrocarbon; Hydrocarbon Gel, Plasticized; Hydrochloric Acid; Hydrochloric Acid, Diluted; Hydrocortisone; Hydrogel Polymer; Hydrogen Peroxide; Hydrogenated Castor Oil; Hydrogenated Palm Oil; Hydrogenated Palm/Palm Kernel Oil Peg-6 Esters; Hydrogenated Polybutene 635-690; Hydroxide Ion; Hydroxyethyl Cellulose; Hydroxyethylpiperazine Ethane Sulfonic Acid; Hydroxymethyl Cellulose; Hydroxyoctacosanyl Hydroxystearate; Hydroxypropyl Cellulose; Hydroxypropyl Methylcellulose 2906; Hydroxypropyl-Beta-cyclodextrin; Hypromellose 2208 (15000 Mpa·S); Hypromellose 2910 (15000 Mpa·S); Hypromelloses; Imidurea; Iodine; Iodoxamic Acid; Iofetamine Hydrochloride; Irish Moss Extract; Isobutane; Isoceteth-20; Isoleucine; Isooctyl Acrylate; Isopropyl Alcohol; Isopropyl Isostearate; Isopropyl Myristate; Isopropyl Myristate-Myristyl Alcohol; Isopropyl Palmitate; Isopropyl Stearate; Isostearic Acid; Isostearyl Alcohol; Isotonic Sodium Chloride Solution; Jelene; Kaolin; Kathon Cg; Kathon Cg II; Lactate; Lactic Acid; Lactic Acid, Dl-; Lactic Acid, L-; Lactobionic Acid; Lactose; Lactose Monohydrate; Lactose, Hydrous; Laneth; Lanolin; Lanolin Alcohol-Mineral Oil; Lanolin Alcohols; Lanolin Anhydrous; Lanolin Cholesterols; Lanolin Nonionic Derivatives; Lanolin, Ethoxylated; Lanolin, Hydrogenated; Lauralkonium Chloride; Lauramine Oxide; Laurdimonium Hydrolyzed Animal Collagen; Laureth Sulfate; Laureth-2; Laureth-23; Laureth-4; Lauric Diethanolamide; Lauric Myristic Diethanolamide; Lauroyl Sarcosine; Lauryl Lactate; Lauryl Sulfate; Lavandula Angustifolia Flowering Top; Lecithin; Lecithin Unbleached; Lecithin, Egg; Lecithin, Hydrogenated; Lecithin, Hydrogenated Soy; Lecithin, Soybean; Lemon Oil; Leucine; Levulinic Acid; Lidofenin; Light Mineral Oil; Light Mineral Oil (85 Ssu); Limonene, (+/−)-; Lipocol Sc-15; Lysine; Lysine Acetate; Lysine Monohydrate; Magnesium Aluminum Silicate; Magnesium Aluminum Silicate Hydrate; Magnesium Chloride; Magnesium Nitrate; Magnesium Stearate; Maleic Acid; Mannitol; Maprofix; Mebrofenin; Medical Adhesive Modified 5-15; Medical Antiform A-F Emulsion; Medronate Disodium; Medronic Acid; Meglumine; Menthol; Metacresol; Metaphosphoric Acid; Methanesulfonic Acid; Methionine; Methyl Alcohol; Methyl Gluceth-10; Methyl Gluceth-20; Methyl Gluceth-20 Sesquistearate; Methyl Glucose Sesquistearate; Methyl Laurate; Methyl Pyrrolidone; Methyl Salicylate; Methyl Stearate; Methylboronic Acid; Methylcellulose (4000 Mpa·S); Methylcelluloses; Methylchloroisothiazolinone; Methylene Blue; Methylisothiazolinone; Methylparaben; Microcrystalline Wax; Mineral Oil; Mono And Diglyceride; Monostearyl Citrate; Monothioglycerol; Multisterol Extract; Myristyl Alcohol; Myristyl Lactate; Myristyl-.Gamma.-Picolinium Chloride; N-(Carbamoyl-Methoxy Peg-40)-1,2-Distearoyl-Cephalin Sodium; N,N-Dimethylacetamide; Niacinamide; Nioxime; Nitric Acid; Nitrogen; Nonoxynol Iodine; Nonoxynol-15; Nonoxynol-9; Norflurane; Oatmeal; Octadecene-1/Maleic Acid Copolymer; Octanoic Acid; Octisalate; Octoxynol-1; Octoxynol-40; Octoxynol-9; Octyldodecanol; Octylphenol Polymethylene; Oleic Acid; Oleth-10/Oleth-5; Oleth-2; Oleth-20; Oleyl Alcohol; Oleyl Oleate; Olive Oil; Oxidronate Disodium; Oxyquinoline; Palm Kernel Oil; Palmitamine Oxide; Parabens; Paraffin; Paraffin, White Soft; Parfum Creme 45/3; Peanut Oil; Peanut Oil, Refined; Pectin; Peg 6-32 Stearate/Glycol Stearate; Peg Vegetable Oil; Peg-100 Stearate; Peg-12 Glyceryl Laurate; Peg-120 Glyceryl Stearate; Peg-120 Methyl Glucose Dioleate; Peg-15 Cocamine; Peg-150 Distearate; Peg-2 Stearate; Peg-20 Sorbitan Isostearate; Peg-22 Methyl Ether/Dodecyl Glycol Copolymer; Peg-25 Propylene Glycol Stearate; Peg-4 Dilaurate; Peg-4 Laurate; Peg-40 Castor Oil; Peg-40 Sorbitan Diisostearate; Peg-45/Dodecyl Glycol Copolymer; Peg-5 Oleate; Peg-50 Stearate; Peg-54 Hydrogenated Castor Oil; Peg-6 Isostearate; Peg-60 Castor Oil; Peg-60 Hydrogenated Castor Oil; Peg-7 Methyl Ether; Peg-75 Lanolin; Peg-8 Laurate; Peg-8 Stearate; Pegoxol 7 Stearate; Pentadecalactone; Pentaerythritol Cocoate; Pentasodium Pentetate; Pentetate Calcium Trisodium; Pentetic Acid; Peppermint Oil; Perflutren; Perfume 25677; Perfume Bouquet; Perfume E-1991; Perfume Gd 5604; Perfume Tana 90/42 Scba; Perfume W-1952-1; Petrolatum; Petrolatum, White; Petroleum Distillates; Phenol; Phenol, Liquefied; Phenonip; Phenoxyethanol; Phenylalanine; Phenylethyl Alcohol; Phenylmercuric Acetate; Phenylmercuric Nitrate; Phosphatidyl Glycerol, Egg; Phospholipid; Phospholipid, Egg; Phospholipon 90g; Phosphoric Acid; Pine Needle Oil (Pinus Sylvestris); Piperazine Hexahydrate; Plastibase-50w; Polacrilin; Polidronium Chloride; Poloxamer 124; Poloxamer 181; Poloxamer 182; Poloxamer 188; Poloxamer 237; Poloxamer 407; Poly(Bis(P-Carboxyphenoxy)Propane Anhydride): Sebacic Acid; Poly(Dimethylsiloxane/Methylvinylsiloxane/Methylhydrogensiloxane) Dimethylvinyl Or Dimethylhydroxy Or Trimethyl Endblocked; Poly(Dl-Lactic-Co-Glycolic Acid), (50:50; Poly(Dl-Lactic-Co-Glycolic Acid), Ethyl Ester Terminated, (50:50; Polyacrylic Acid (250000 Mw); Polybutene (1400 Mw); Polycarbophil; Polyester; Polyester Polyamine Copolymer; Polyester Rayon; Polyethylene Glycol 1000; Polyethylene Glycol 1450; Polyethylene Glycol 1500; Polyethylene Glycol 1540; Polyethylene Glycol 200; Polyethylene Glycol 300; Polyethylene Glycol 300-1600; Polyethylene Glycol 3350; Polyethylene Glycol 400; Polyethylene Glycol 4000; Polyethylene Glycol 540; Polyethylene Glycol 600; Polyethylene Glycol 6000; Polyethylene Glycol 8000; Polyethylene Glycol 900; Polyethylene High Density Containing Ferric Oxide Black (<1%); Polyethylene Low Density Containing Barium Sulfate (20-24%); Polyethylene T; Polyethylene Terephthalates; Polyglactin; Polyglyceryl-3 Oleate; Polyglyceryl-4 Oleate; Polyhydroxyethyl Methacrylate; Polyisobutylene; Polyisobutylene (1100000 Mw); Polyisobutylene (35000 Mw); Polyisobutylene 178-236; Polyisobutylene 241-294; Polyisobutylene 35-39; Polyisobutylene Low Molecular Weight; Polyisobutylene Medium Molecular Weight; Polyisobutylene/Polybutene Adhesive; Polylactide; Polyols; Polyoxyethylene-Polyoxypropylene 1800; Polyoxyethylene Alcohols; Polyoxyethylene Fatty Acid Esters; Polyoxyethylene Propylene; Polyoxyl 20 Cetostearyl Ether; Polyoxyl 35 Castor Oil; Polyoxyl 40 Hydrogenated Castor Oil; Polyoxyl 40 Stearate; Polyoxyl 400 Stearate; Polyoxyl 6 And Polyoxyl 32 Palmitostearate; Polyoxyl Distearate; Polyoxyl Glyceryl Stearate; Polyoxyl Lanolin; Polyoxyl Palmitate; Polyoxyl Stearate; Polypropylene; Polypropylene Glycol; Polyquaternium-10; Polyquaternium-7 (70/30 Acrylamide/Dadmac; Polysiloxane; Polysorbate 20; Polysorbate 40; Polysorbate 60; Polysorbate 65; Polysorbate 80; Polyurethane; Polyvinyl Acetate; Polyvinyl Alcohol; Polyvinyl Chloride; Polyvinyl Chloride-Polyvinyl Acetate Copolymer; Polyvinylpyridine; Poppy Seed Oil; Potash; Potassium Acetate; Potassium Alum; Potassium Bicarbonate; Potassium Bisulfite; Potassium Chloride; Potassium Citrate; Potassium Hydroxide; Potassium Metabisulfite; Potassium Phosphate, Dibasic; Potassium Phosphate, Monobasic; Potassium Soap; Potassium Sorbate; Povidone Acrylate Copolymer; Povidone Hydrogel; Povidone K17; Povidone K25; Povidone K29/32; Povidone K30; Povidone K90; Povidone K90f; Povidone/Eicosene Copolymer; Povidones; Ppg-12/Smdi Copolymer; Ppg-15 Stearyl Ether; Ppg-20 Methyl Glucose Ether Distearate; Ppg-26 Oleate; Product Wat; Proline; Promulgen D; Promulgen G; Propane; Propellant A-46; Propyl Gallate; Propylene Carbonate; Propylene Glycol; Propylene Glycol Diacetate; Propylene Glycol Dicaprylate; Propylene Glycol Monolaurate; Propylene Glycol Monopalmitostearate; Propylene Glycol Palmitostearate; Propylene Glycol Ricinoleate; Propylene Glycol/Diazolidinyl Urea/Methylparaben/Propylparben; Propylparaben; Protamine Sulfate; Protein Hydrolysate; Pvm/Ma Copolymer; Quatemium-15; Quatemium-15 Cis-Form; Quaternium-52; Ra-2397; Ra-3011; Saccharin; Saccharin Sodium; Saccharin Sodium Anhydrous; Safflower Oil; Sd Alcohol 3a; Sd Alcohol 40; Sd Alcohol 40-2; Sd Alcohol 40b; Sepineo P 600; Serine; Sesame Oil; Shea Butter; Silastic Brand Medical Grade Tubing; Silastic Medical Adhesive, Silicone Type A; Silica, Dental; Silicon; Silicon Dioxide; Silicon Dioxide, Colloidal; Silicone; Silicone Adhesive 4102; Silicone Adhesive 4502; Silicone Adhesive Bio-Psa Q7-4201; Silicone Adhesive Bio-Psa Q7-4301; Silicone Emulsion; Silicone/Polyester Film Strip; Simethicone; Simethicone Emulsion; Sipon Ls 20np; Soda Ash; Sodium Acetate; Sodium Acetate Anhydrous; Sodium Alkyl Sulfate; Sodium Ascorbate; Sodium Benzoate; Sodium Bicarbonate; Sodium Bisulfate; Sodium Bisulfite; Sodium Borate; Sodium Borate Decahydrate; Sodium Carbonate; Sodium Carbonate Decahydrate; Sodium Carbonate Monohydrate; Sodium Cetostearyl Sulfate; Sodium Chlorate; Sodium Chloride; Sodium Chloride Injection; Sodium Chloride Injection, Bacteriostatic; Sodium Cholesteryl Sulfate; Sodium Citrate; Sodium Cocoyl Sarcosinate; Sodium Desoxycholate; Sodium Dithionite; Sodium Dodecylbenzenesulfonate; Sodium Formaldehyde Sulfoxylate; Sodium Gluconate; Sodium Hydroxide; Sodium Hypochlorite; Sodium Iodide; Sodium Lactate; Sodium Lactate, L-; Sodium Laureth-2 Sulfate; Sodium Laureth-3 Sulfate; Sodium Laureth-5 Sulfate; Sodium Lauroyl Sarcosinate; Sodium Lauryl Sulfate; Sodium Lauryl Sulfoacetate; Sodium Metabisulfite; Sodium Nitrate; Sodium Phosphate; Sodium Phosphate Dihydrate; Sodium Phosphate, Dibasic; Sodium Phosphate, Dibasic, Anhydrous; Sodium Phosphate, Dibasic, Dihydrate; Sodium Phosphate, Dibasic, Dodecahydrate; Sodium Phosphate, Dibasic, Heptahydrate; Sodium Phosphate, Monobasic; Sodium Phosphate, Monobasic, Anhydrous; Sodium Phosphate, Monobasic, Dihydrate; Sodium Phosphate, Monobasic, Monohydrate; Sodium Polyacrylate (2500000 Mw); Sodium Pyrophosphate; Sodium Pyrrolidone Carboxylate; Sodium Starch Glycolate; Sodium Succinate Hexahydrate; Sodium Sulfate; Sodium Sulfate Anhydrous; Sodium Sulfate Decahydrate; Sodium Sulfite; Sodium Sulfosuccinated Undecyclenic Monoalkylolamide; Sodium Tartrate; Sodium Thioglycolate; Sodium Thiomalate; Sodium Thiosulfate; Sodium Thiosulfate Anhydrous; Sodium Trimetaphosphate; Sodium Xylenesulfonate; Somay 44; Sorbic Acid; Sorbitan; Sorbitan Isostearate; Sorbitan Monolaurate; Sorbitan Monooleate; Sorbitan Monopalmitate; Sorbitan Monostearate; Sorbitan Sesquioleate; Sorbitan Trioleate; Sorbitan Tristearate; Sorbitol; Sorbitol Solution; Soybean Flour; Soybean Oil; Spearmint Oil; Spermaceti; Squalane; Stabilized Oxychloro Complex; Stannous 2-Ethylhexanoate; Stannous Chloride; Stannous Chloride Anhydrous; Stannous Fluoride; Stannous Tartrate; Starch; Starch 1500, Pregelatinized; Starch, Corn; Stearalkonium Chloride; Stearalkonium Hectorite/Propylene Carbonate; Stearamidoethyl Diethylamine; Steareth-10; Steareth-100; Steareth-2; Steareth-20; Steareth-21; Steareth-40; Stearic Acid; Stearic Diethanolamide; Stearoxytrimethylsilane; Steartrimonium Hydrolyzed Animal Collagen; Stearyl Alcohol; Sterile Water For Inhalation; Styrene/Isoprene/Styrene Block Copolymer; Succimer; Succinic Acid; Sucralose; Sucrose; Sucrose Distearate; Sucrose Polyesters; Sulfacetamide Sodium; Sulfobutylether .Beta.-Cyclodextrin; Sulfur Dioxide; Sulfuric Acid; Sulfurous Acid; Surfactol Qs; Tagatose, D-; Talc; Tall Oil; Tallow Glycerides; Tartaric Acid; Tartaric Acid, D1-; Tenox; Tenox-2; Tert-Butyl Alcohol; Tert-Butyl Hydroperoxide; Tert-Butylhydroquinone; Tetrakis(2-Methoxyisobutylisocyanide)Copper(I) Tetrafluoroborate; Tetrapropyl Orthosilicate; Tetrofosmin; Theophylline; Thimerosal; Threonine; Thymol; Tin; Titanium Dioxide; Tocopherol; Tocophersolan; Total parenteral nutrition, lipid emulsion; Triacetin; Tricaprylin; Trichloromonofluoromethane; Trideceth-10; Triethanolamine Lauryl Sulfate; Trifluoroacetic Acid; Triglycerides, Medium Chain; Trihydroxystearin; Trilaneth-4 Phosphate; Trilaureth-4 Phosphate; Trisodium Citrate Dihydrate; Trisodium Hedta; Triton 720; Triton X-200; Trolamine; Tromantadine; Tromethamine (TRIS); Tryptophan; Tyloxapol; Tyrosine; Undecylenic Acid; Union 76 Amsco-Res 6038; Urea; Valine; Vegetable Oil; Vegetable Oil Glyceride, Hydrogenated; Vegetable Oil, Hydrogenated; Versetamide; Viscarin; Viscose/Cotton; Vitamin E; Wax, Emulsifying; Wecobee Fs; White Ceresin Wax; White Wax; Xanthan Gum; Zinc; Zinc Acetate; Zinc Carbonate; Zinc Chloride; and Zinc Oxide.

Pharmaceutical composition formulations disclosed herein may include cations or anions. In one embodiment, the formulations include metal cations such as, but not limited to, Zn2+, Ca2+, Cu2+, Mn2+, Mg2+ and combinations thereof. As a non-limiting example, formulations may include polymers and complexes with a metal cation (See e.g., U.S. Pat. Nos. 6,265,389 and 6,555,525, each of which is herein incorporated by reference in its entirety).

Formulations of the invention may also include one or more pharmaceutically acceptable salts. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids.

Solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

### V. Administration and Dosing

### Administration

The terms “administering” and “introducing” are used interchangeable herein and refer to the delivery of the pharmaceutical composition into a cell or a subject. In the case of delivery to a subject, the pharmaceutical composition is delivered by a method or route that results in at least partial localization of the introduced cells at a desired site, such as hepatocytes, such that a desired effect(s) is produced.

In one aspect of the method, the pharmaceutical composition may be administered via a route such as, but not limited to, enteral (into the intestine), gastroenteral, epidural (into the dura matter), oral (by way of the mouth), transdermal, peridural, intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intravenous bolus, intravenous drip, intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection (into a pathologic cavity) intracavitary (into the base of the penis), intravaginal administration, intrauterine, extra-amniotic administration, transdermal (diffusion through the intact skin for systemic distribution), transmucosal (diffusion through a mucous membrane), transvaginal, insufflation (snorting), sublingual, sublabial, enema, eye drops (onto the conjunctiva), in ear drops, auricular (in or by way of the ear), buccal (directed toward the cheek), conjunctival, cutaneous, dental (to a tooth or teeth), electro-osmosis, endocervical, endosinusial, endotracheal, extracorporeal, hemodialysis, infiltration, interstitial, intra-abdominal, intra-amniotic, intra-articular, intrabiliary, intrabronchial, intrabursal, intracartilaginous (within a cartilage), intracaudal (within the cauda equine), intracisternal (within the cisterna magna cerebellomedularis), intracorneal (within the cornea), dental intracornal, intracoronary (within the coronary arteries), intracorporus cavernosum (within the dilatable spaces of the corporus cavernosa of the penis), intradiscal (within a disc), intraductal (within a duct of a gland), intraduodenal (within the duodenum), intradural (within or beneath the dura), intraepidermal (to the epidermis), intraesophageal (to the esophagus), intragastric (within the stomach), intragingival (within the gingivae), intraileal (within the distal portion of the small intestine), intralesional (within or introduced directly to a localized lesion), intraluminal (within a lumen of a tube), intralymphatic (within the lymph), intramedullary (within the marrow cavity of a bone), intrameningeal (within the meninges), intramyocardial (within the myocardium), intraocular (within the eye), intraovarian (within the ovary), intrapericardial (within the pericardium), intrapleural (within the pleura), intraprostatic (within the prostate gland), intrapulmonary (within the lungs or its bronchi), intrasinal (within the nasal or periorbital sinuses), intraspinal (within the vertebral column), intrasynovial (within the synovial cavity of a joint), intratendinous (within a tendon), intratesticular (within the testicle), intrathecal (within the cerebrospinal fluid at any level of the cerebrospinal axis), intrathoracic (within the thorax), intratubular (within the tubules of an organ), intratumor (within a tumor), intratympanic (within the aurus media), intravascular (within a vessel or vessels), intraventricular (within a ventricle), iontophoresis (by means of electric current where ions of soluble salts migrate into the tissues of the body), irrigation (to bathe or flush open wounds or body cavities), laryngeal (directly upon the larynx), nasogastric (through the nose and into the stomach), occlusive dressing technique (topical route administration which is then covered by a dressing which occludes the area), ophthalmic (to the external eye), oropharyngeal (directly to the mouth and pharynx), parenteral, percutaneous, periarticular, peridural, perineural, periodontal, rectal, respiratory (within the respiratory tract by inhaling orally or nasally for local or systemic effect), retrobulbar (behind the pons or behind the eyeball), intramyocardial (entering the myocardium), soft tissue, subarachnoid, subconjunctival, submucosal, topical, transplacental (through or across the placenta), transtracheal (through the wall of the trachea), transtympanic (across or through the tympanic cavity), ureteral (to the ureter), urethral (to the urethra), vaginal, caudal block, diagnostic, nerve block, biliary perfusion, cardiac perfusion, photopheresis and spinal.

Modes of administration include injection, infusion, instillation, and/or ingestion. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrastemal injection and infusion. In some examples, the route is intravenous. For the delivery of cells, administration by injection or infusion can be made.

The cells can be administered systemically. The phrases “systemic administration,” “administered systemically”, “peripheral administration” and “administered peripherally” refer to the administration other than directly into a target site, tissue, or organ, such that it enters, instead, the subject's circulatory system and, thus, is subject to metabolism and other like processes.

### Dosing

The term “effective amount” refers to the amount of the active ingredient needed to prevent or alleviate at least one or more signs or symptoms of a specific disease and/or condition, and relates to a sufficient amount of a composition to provide the desired effect. The term “therapeutically effective amount” therefore refers to an amount of active ingredient or a composition comprising the active ingredient that is sufficient to promote a particular effect when administered to a typical subject. An effective amount would also include an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate “effective amount” can be determined by one of ordinary skill in the art using routine experimentation.

The pharmaceutical, diagnostic, or prophylactic compositions of the present invention may be administered to a subject using any amount and any route of administration effective for preventing, treating, managing, or diagnosing diseases, disorders and/or conditions. The exact amount required will vary from subject to subject, depending on the species, age, and general condition of the subject, the severity of the disease, the particular composition, its mode of administration, its mode of activity, and the like. The subject may be a human, a mammal, or an animal. Compositions in accordance with the invention are typically formulated in unit dosage form for ease of administration and uniformity of dosage. It will be understood, however, that the total daily usage of the compositions of the present invention may be decided by the attending physician within the scope of sound medical judgment. The specific therapeutically effective, prophylactically effective, or appropriate diagnostic dose level for any particular individual will depend upon a variety of factors including the disorder being treated and the severity of the disorder; the activity of the specific payload employed; the specific composition employed; the age, body weight, general health, sex and diet of the patient; the time of administration, and route of administration; the duration of the treatment; drugs used in combination or coincidental with the active ingredient; and like factors well known in the medical arts.

In certain embodiments, pharmaceutical compositions in accordance with the present invention may be administered at dosage levels sufficient to deliver from about 0.01 mg/kg to about 100 mg/kg, from about 0.01 mg/kg to about 0.05 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic, effect.

The desired dosage of the composition present invention may be delivered only once, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. As used herein, a “split dose” is the division of “single unit dose” or total daily dose into two or more doses, e.g., two or more administrations of the “single unit dose”. As used herein, a “single unit dose” is a dose of any therapeutic administered in one dose/at one time/single route/single point of contact, i.e., single administration event.

### VI. Screening Methods

In another aspect, provided herein are methods to identify candidate compounds based on biochemical activity or activities as described elsewhere in the specification. In an embodiment, a candidate compound with mTOR inhibitory activity inhibits both the mTORC1 and mTORC2 complexes. In an embodiment, a candidate compound with mTORC2 inhibitory activity inhibits mTORC2 but not mTORC1. As shown in Examples 26 and 30, inhibition of mTORC1 alone via rapamycin treatment is insufficient to decrease PNPLA3 expression, while an mTORC1/mTORC2 inhibitor decreased PNPLA3 expression. Thus, inhibition of mTORC2, but not mTORC1, is necessary to decrease PNPLA3 expression. In an embodiment, a candidate compound selected for further study may thus inhibit either mTORC2 alone, or mTORC1 and mTORC2. A compound that has mTOR inhibitory activity can be a compound that was designed to inhibit mTOR or inhibit any other kinase, wherein the compound can be demonstrated to inhibit mTOR. In an embodiment, mTOR inhibitory activity comprises inhibiting mTOR kinase activity directly or indirectly. Direct or indirect inhibition includes, but is not limited to, inhibiting the catalytic activity of the kinase or inhibiting binding of substrate to the kinase.

In an aspect, provided herein are methods for identifying a compound that reduces PNPLA3 gene expression comprising providing a candidate compound; assaying the candidate compound for at least two of the activities selected from the group consisting of: mTOR inhibitory activity, mTORC2 inhibitory activity, PI3K inhibitory activity, PI3Kβ inhibitory activity, DNA-PK inhibitory activity, ability to induce hyperinsulinemia, ability to induce hyperglycemia, and PNPLA3 gene expression inhibitory activity; and identifying the candidate compound as the compound based on results of the two or more assays that indicate the candidate compound has two or more desirable properties. In some embodiments, the desirable properties are selected from the group consisting of: mTOR inhibitory activity, lack of PI3K inhibitory activity, lack of PI3Kβ inhibitory activity, lack of DNA-PK inhibitory activity, lack of ability to induce hyperinsulinemia, lack of ability to induce hyperglycemia, and PNPLA3 gene expression inhibitory activity.

In an embodiment, a candidate compound lacks PI3K inhibitory activity. As shown in Example 31, compounds that inhibit mTOR and PI3K also induced higher insulin and serum glucose levels in mice. Thus, inhibition of PI3K to reduce PNPLA3 expression also resulted in adverse effects. In an embodiment, a candidate compound selected for further study may thus lack PI3K or PI3Kβ inhibitory activity.

In an embodiment, the activity is mTORC2 inhibitory activity. In an embodiment, the activity is lack of PI3K inhibitory activity. In an embodiment, the activity is lack of PI3Kβ inhibitory activity. In an embodiment, the activity is lack of DNA-PK inhibitory activity. In an embodiment, the activity is lack of PIP4K2C inhibitory activity. In an embodiment, the activity is lack of ability to induce hyperinsulinemia. In an embodiment, the activity is lack of ability to induce hyperglycemia. In an embodiment, the activity is PNPLA3 gene expression inhibitory activity.

In some embodiments, the activity is mTOR inhibitory activity. In some embodiments, the activity is mTORC2 inhibitory activity. In some embodiments, the activity is PNPLA3 gene expression inhibitory activity.

In some embodiments, the activity is lack of PI3K inhibitory activity. In some embodiments, the activity is lack of PI3Kβ inhibitory activity. In some embodiments, the activity is lack of DNA-PK inhibitory activity. In some embodiments, the activity is lack of PIP4K2C inhibitory activity. In some embodiments, the activity is lack of the ability to induce hyperinsulinemia. In some embodiments, the activity is lack of the ability to induce hyperglycemia.

In an embodiment, the activity is any two of mTOR inhibitory activity, mTORC2 inhibitory activity, lack of PI3K inhibitory activity, lack of PI3Kβ inhibitory activity, lack of DNA-PK inhibitory activity, lack of PIP4K2C inhibitory activity, lack of the ability to induce hyperinsulinemia, lack of the ability to induce hyperglycemia, and PNPLA3 gene expression inhibitory activity. In an embodiment, the activity is any three of mTOR inhibitory activity, mTORC2 inhibitory activity, lack of PI3K inhibitory activity, lack of PI3Kβ inhibitory activity, lack of DNA-PK inhibitory activity, lack of PIP4K2C inhibitory activity, lack of the ability to induce hyperinsulinemia, lack of the ability to induce hyperglycemia, and PNPLA3 gene expression inhibitory activity. In an embodiment, the activity is any four of mTOR inhibitory activity, mTORC2 inhibitory activity, lack of PI3K inhibitory activity, lack of PI3Kβ inhibitory activity, lack of DNA-PK inhibitory activity, lack of PIP4K2C inhibitory activity, lack of the ability to induce hyperinsulinemia, lack of the ability to induce hyperglycemia, and PNPLA3 gene expression inhibitory activity. In an embodiment, the activity is any five of mTOR inhibitory activity, mTORC2 inhibitory activity, lack of PI3K inhibitory activity, lack of PI3Kβ inhibitory activity, lack of DNA-PK inhibitory activity, lack of PIP4K2C inhibitory activity, lack of the ability to induce hyperinsulinemia, lack of the ability to induce hyperglycemia, and PNPLA3 gene expression inhibitory activity. In an embodiment, the activity is any six of mTOR inhibitory activity, mTORC2 inhibitory activity, lack of PI3K inhibitory activity, lack of PI3Kβ inhibitory activity, lack of DNA-PK inhibitory activity, lack of PIP4K2C inhibitory activity, lack of the ability to induce hyperinsulinemia, lack of the ability to induce hyperglycemia, and PNPLA3 gene expression inhibitory activity. In an embodiment, the activity is any seven of mTOR inhibitory activity, mTORC2 inhibitory activity, lack of PI3K inhibitory activity, lack of PI3Kβ inhibitory activity, lack of DNA-PK inhibitory activity, lack of PIP4K2C inhibitory activity, lack of the ability to induce hyperinsulinemia, lack of the ability to induce hyperglycemia, and PNPLA3 gene expression inhibitory activity. In an embodiment, the activity is any eight of mTOR inhibitory activity, mTORC2 inhibitory activity, lack of PI3K inhibitory activity, lack of PI3Kβ inhibitory activity, lack of DNA-PK inhibitory activity, lack of PIP4K2C inhibitory activity, lack of the ability to induce hyperinsulinemia, lack of the ability to induce hyperglycemia, and PNPLA3 gene expression inhibitory activity. In an embodiment, the activity is any nine of mTOR inhibitory activity, mTORC2 inhibitory activity, lack of PI3K inhibitory activity, lack of PI3Kβ inhibitory activity, lack of DNA-PK inhibitory activity, lack of PIP4K2C inhibitory activity, lack of the ability to induce hyperinsulinemia, lack of the ability to induce hyperglycemia, and PNPLA3 gene expression inhibitory activity.

### Assays

Inhibitory activity of the candidate compound can be determined via an appropriate method known in the art. Inhibition assays include enzymatic assay that measure changes in phosphorylation of kinase target proteins, or binding assays that measure binding of a candidate compound to the kinase target protein. In some embodiments, the assay is a biochemical assay. In some embodiments, the assay is in a cell. In some embodiments, the assay is in a cell lysate.

For enzymatic assays, any appropriate assay may be used, such as antibody assays including Western blots or ELISAs; or biochemical assays that measure incorporation of radioactive or fluorescent ATP into kinase substrates (Ma et al, Expert Opin Drug Discov, 2008 3(6):607-621 which is hereby incorporated by reference in its entirety).

Radiometric assays include biochemical assays using purified kinase proteins and substrates. The kinase reaction is performed in solution in the presence of ³²P-γ-ATP, ³³P-γ-ATP, or ³⁵S-thio-labeled ATP and the candidate inhibitory compound. The radioisotope labeled substrate products are column purified and/or bound to filters or membranes and the free ATP is washed away, allowing for quantification of only the phosphorylated substrate. The radioisotope labeled protein can be measured via autoradiography or phosphorimager techniques known in the art.

An alternative to columns or membranes is to use a scintillation proximity assay, in which the radiolabeled proteins of interest are bound to beads that contain a scintillant that can emit light after stimulation by beta particles or auger elements. The stimulation of the scintillant occurs only when radiolabeled molecules are bound to the beads. The emission of light can be measured via a scintillation analyzer or flow scintillation analyzer. Commercial radioisotope and scintillation kits are available from multiple vendors, including PerkinElmer and Reaction Biology.

Fluorescent and luminescent assays include biochemical assays using purified kinase proteins and substrates. Any appropriate fluorescent or luminescent assay, including but not limited to, fluorescence or luminescent intensity, fluorescence polarization, fluorescence resonance energy transfer (FRET), or time resolved fluorescence resonance energy transfer (TRF-FRET).

Luminescent assays measure the amount of ADP in a sample after a kinase has phosphorylated a substrate using ATP. The remaining ATP after the kinase reaction is depleted and removed, leaving only the newly made ADP in the solution. A detection reagent is added that simultaneously converts the ADP to ATP and the new ATP to light using a luciferase/luciferin reaction. Commercial luminescent kits are available from Promega (ADP-Glo) and kits specific to PI3 kinases are available as well (ADP-Glo Lipid Kinase Kit).

Fluorescence intensity assays measure the amount of ADP in a sample after a kinase has phosphorylated a substrate using ATP. The newly made ADP is converted to ADHP (10-Acetyl-3,7-dihydroxyphenoxazine) and linked to hydrogen peroxide, resulting in the synthesis of fluorescent Resorufin. The signal produced by the Resorufin is proportional to the amount of the ADP in the sample, and therefore the activity of the kinase. Compounds that inhibit kinase activity result in less fluorescence signal. Commercial FI kits are available from DiscovRx (ADP Hunter Kit).

FRET analysis is based on donor and acceptor fluorophores in proximity to each other. An excited donor fluorophore transfers non-radiative energy to a proximal acceptor fluorophore, resulting in excitation and photon emittance of the acceptor fluorophore. Various methods of utilizing FRET for kinase assays are known in the art. In one method, a kinase is mixed with a acceptor fluorophore-tagged substrate and ATP, and the kinase phosphorylates the labeled substrate. Next, a terbium-labeled antibody specific for the phosphorylated substrate is added. The terbium molecule acts a donor fluorophore and transfers energy to the acceptor fluorophore, which is then quantified. The amount of FRET signal is proportional to the amount of phosphorylated substrate and thus the activity of the kinase. Commercial FRET assays for Class I and Class II PI3 kinases are available, including the HTS Kit and HTRF Enzyme Assay Kits from MilliporeSigma. Additional FRET kinase kits are the LANCE Ultra or Classic kits from PerkinElmer, and the LanthaScreen and Z′-LYTE kinase assay kit from ThermoFisher Scientific.

Detection of phosphorylated substrates can also be accomplished via antibody binding assays, such as ELISAs or Western blots. These assays can be done on both biochemical samples and cell based samples. In the case of a biochemical assay, the substrate is incubated with a kinase, ATP, and optionally a candidate compound. In a cell based assay, the cell is incubated with a candidate compound and then lysed for protein analysis. Once the biochemical kinase reaction is complete or the cell is lysed, the substrate protein or lysate is capture to a membrane by filtration or gel electrophoresis and membrane blotting. An antibody specific to the phosphorylated substrate is added and detected via binding of a fluorescent or enzyme-linked secondary antibody. Total protein can also be measured via antibody detection of total protein, phosphorylated and unphosphorylated via use of a second antibody that is not specific to the phosphorylated substrate. ELISA kits for phosphorylated mTOR and PI3K substrates, including AKT, S6, NDRG1, SGK1, PKC, PIP3, p53 and CHK2 are available from a variety of manufacturers, including MilliporeSigma, Cell Signaling, and Abcam. Antibodies for phosphorylated mTOR, PI3K, DNA-Pk, and PIP4K2C substrates, including AKT, S6, NDRG1, SGK1, PKC, PIP3, p53 and CHK2 are available from a variety of manufacturers, including Cell Signaling, Abcam, and Santa Cruz Biotech.

For binding assays, any appropriate binding assay known in the art may be used, including but not limited to differential scanning fluorimetry, also known as thermostability shift assay; surface plasmon resonance; or any other appropriate method known in the art. In a differential scanning fluorimetry assay, a target protein is incubated with and without a candidate compound and a fluorescent dye such as SyproOrange. The mixture is heated over a temperature gradient and the thermal unfolding of the protein is assessed via the dye, which is fluorescent in a nonpolar environment and quenched in an aqueous environment. Thus, as the protein unfolds, dye binds to the exposed core of the protein, resulting in a quantifiable increase in the fluorescent intensity of the mixture. Binding of a compound to the target protein stabilizes the protein and shifts the melting temperature (Tm) of the protein. Kinase inhibitor screening using differential scanning fluorimetry is described in Rudolf AF et al, PLoS ONE June 2014, https://doi.org/10.1371/journal.pone.0098800, hereby incorporated by reference in its entirety. Kits for differential scanning fluorimetry or thermoshift assays are available from various vendors, including ThermoFisher Scientific (Protein Thermal Shift Starter Kit) and Biotium (GloMelt).

Surface plasmon resonance assays may also be used to assess candidate compound binding to kinases. Surface plasmon resonance is a commonly used technique in the protein and molecule binding field to measure the binding of molecules with high sensitivity. SPR has been used to measure binding of small molecules to various protein factors (see e.g, Kennedy A E et al, J. Bio Screen, 2016: 21(1) 96-100 doi:10.1 177/1087057/15607814, hereby incorporated by reference in its entirety). SPR systems and reagents are commercially available from GE Healthcare under the BIAcore brand.

### Thresholds

Inhibitory activity of the candidate compound includes quantifying the IC50 or EC50 of the compound to provide an inhibitory threshold. IC50 or EC50 levels can be the compound enzymatic inhibition level or the compound binding level. An inhibitory threshold to identify a candidate compound can be selected to identify a possible lead compound that is later refined via structure refinement and design informed by structure-activity studies, medicinal chemistry-based studies, or other studies know in the art. An inhibitory threshold can be at least about 100 μM, 95 μM, 90 μM, 85 μM, 80 μM, 75 μM, 70 μM, 65 μM, 60 μM, 55 μM, 50 μM, 45 μM, 40 μM, 35 μM, 30 μM, 25 μM, 20 μM, 15 μM, 10 μM, 9 μM, 8 μM, 7 μM, 6 μM, 5 μM, 4 μM, 3 μM, 2 μM, 1 μM, 95 nM, 90 nM, 85 nM, 80 nM, 75 nM, 70 nM, 65 nM, 60 nM, 55 nM, 50 nM, 45 nM, 40 nM, 35 nM, 30 nM, 25 nM, 20 nM, 15 nM, 10 nM, 9 nM, 8 nM, 7 nM, 6 nM, 5 nM, 4 nM, 3 nM, 2 nM, or 1 nM. An inhibitory threshold can be a range of at least 1-100 nM, 1-10 nM, 1-5 nM, 5-10 nM, 10-15 nM, 15-20 nM, 20-25 nM, 25-30 nM, 30-35 nM, 35-40 nM, 40-45 nM, 45-50 nM, 50-55 nM, 55-60 nM, 60-65 nM, 65-70 nM, 70-75 nM, 75-80 nM, 80-85 nM, 85-90 nM, 90-95 nM, 95-100 nM, 1-100 μM, 1-10 μM, 1-5 μM, 5-10 μM, 10-15 μM, 15-20 μM, 20-25 μM, 25-30 μM, 30-35 μM, 35-40 μM, 40-45 μM, 45-50 μM, 50-55 μM, 55-60 μM, 60-65 μM, 65-70 μM, 70-75 μM, 75-80 μM, 80-85 μM, 85-90 μM, 90-95 μM, or 95-100 μM.

### Compound Library

Candidate compounds can be selected from any available library or commercial vendor. Candidate compounds can also by synthesized by the applicant or a third party company using chemistry methods generally known in the art. Libraries of candidate Pi3K/mTOR/Akt small molecule inhibitors are available from various commercial vendors, including the 223 compound library PI3K/Akt/mTOR Compound Library from MedChemExpress, catalogue no. HY-L015 and the 145 compound DiscoveryProbe™ PI3K/Akt/MTOR Compound Library from ApexBio, catalogue no. L1034. General small molecule libraries are also available from commercial vendors, including the 1496 compound DiscoveryProbe™ FDA-Approved Drug Library from ApexBio, catalogue no. L1021; the 493 compound DiscoveryProbe™ Kinase Inhibitor Library from ApexBio, catalogue no. L1024, the 1983 compound DiscoveryProbe™ Inhibitor Library from ApexBio, catalogue no. L1048; and the 7853 compound Bioactive Compound Library Plus from MedChemExpress, catalogue no. HY-L001P.

### VII. Definitions

The term “analog”, as used herein, refers to a compound that is structurally related to the reference compound and shares a common functional activity with the reference compound.

The term “biologic”, as used herein, refers to a medical product made from a variety of natural sources such as micro-organism, plant, animal, or human cells.

The term “boundary”, as used herein, refers to a point, limit, or range indicating where a feature, element, or property ends or begins.

The term “compound”, as used herein, refers to a single agent or a pharmaceutically acceptable salt thereof, or a bioactive agent or drug.

The term “derivative”, as used herein, refers to a compound that differs in structure from the reference compound, but retains the essential properties of the reference molecule.

The term “downstream neighborhood gene”, as used herein, refers to a gene downstream of primary neighborhood gene that may be located within the same insulated neighborhood as the primary neighborhood gene.

The term “drug”, as used herein, refers to a substance other than food intended for use in the diagnosis, cure, alleviation, treatment, or prevention of disease and intended to affect the structure or any function of the body.

The term “enhancer”, as used herein, refers to regulatory DNA sequences that, when bound by transcription factors, enhance the transcription of an associated gene.

The term “gene”, as used herein, refers to a unit or segment of the genomic architecture of an organism, e.g., a chromosome. Genes may be coding or non-coding. Genes may be encoded as contiguous or non-contiguous polynucleotides. Genes may be DNA or RNA.

The term “genomic signaling center”, as used herein, refers to regions within insulated neighborhoods that include regions capable of binding context-specific combinatorial assemblies of signaling molecules that participate in the regulation of the genes within that insulated neighborhood.

The term “genomic system architecture”, as used herein, refers to the organization of an individual's genome and includes chromosomes, topologically associating domains (TADs), and insulated neighborhoods.

The term “herbal preparation”, as used herein, refers to herbal medicines that contain parts of plants, or other plant materials, or combinations as active ingredients.

The term “insulated neighborhood” (IN), as used herein, refers to chromosome structure formed by the looping of two interacting sites in the chromosome sequence that may comprise CCCTC-Binding factor (CTCF) co-occupied by cohesin and affect the expression of genes in the insulated neighborhood as well as those genes in the vicinity of the insulated neighborhoods.

The term “insulator”, as used herein, refers to regulatory elements that block the ability of an enhancer to activate a gene when located between them and contribute to specific enhancer-gene interactions.

The term “master transcription factor”, as used herein, refers to a signaling molecule which alter, whether to increase or decrease, the transcription of a target gene, e.g., a neighborhood gene and establish cell-type specific enhancers. Master transcription factors recruit additional signaling proteins, such as other transcription factors to enhancers to form signaling centers.

The term “minimal insulated neighborhood”, as used herein, refers to an insulated neighborhood having at least one neighborhood gene and associated regulatory sequence region or regions (RSRs) which facilitate the expression or repression of the neighborhood gene such as a promoter and/or enhancer and/or repressor region, and the like.

The term “modulate”, as used herein, refers to an alteration (e.g., increase or decrease) in the expression of the target gene and/or activity of the gene product.

The term “neighborhood gene”, as used herein, refers to a gene localized within an insulated neighborhood.

The term “penetrance”, as used herein, refers to the proportion of individuals carrying a particular variant of a gene (e.g., mutation, allele or generally a genotype, whether wild type or not) that also exhibits an associated trait (phenotype) of that variant gene and in some situations is measured as the proportion of individuals with the mutation who exhibit clinical symptoms thus existing on a continuum.

The term “polypeptide”, as used herein, refers to a polymer of amino acid residues (natural or unnatural) linked together most often by peptide bonds. The term, as used herein, refers to proteins, polypeptides, and peptides of any size, structure, or function. In some instances, the polypeptide encoded is smaller than about 50 amino acids and the polypeptide is then termed a peptide. If the polypeptide is a peptide, it will be at least about 2, 3, 4, or at least 5 amino acid residues long.

The term “primary neighborhood gene” as used herein, refers to a gene which is most commonly found within a specific insulated neighborhood along a chromosome.

The term “primary downstream boundary”, as used herein, refers to the insulated neighborhood boundary located downstream of a primary neighborhood gene.

The term “primary upstream boundary”, as used herein, refers to the insulated neighborhood boundary located upstream of a primary neighborhood gene.

The term “promoter” as used herein, refers to a DNA sequence that defines where transcription of a gene by RNA polymerase begins and defines the direction of transcription indicating which DNA strand will be transcribed.

The term “regulatory sequence regions”, as used herein, include but are not limited to regions, sections or zones along a chromosome whereby interactions with signaling molecules occur in order to alter expression of a neighborhood gene.

The term “repressor”, as used herein, refers to any protein that binds to DNA and therefore regulates the expression of genes by decreasing the rate of transcription.

The term “secondary downstream boundary”, as used herein, refers to the downstream boundary of a secondary loop within a primary insulated neighborhood.

The term “secondary upstream boundary”, as used herein, refers to the upstream boundary of a secondary loop within a primary insulated neighborhood.

The term “signaling center”, as used herein, refers to a defined region of a living organism that interacts with a defined set of biomolecules, such as signaling proteins or signaling molecules (e.g., transcription factors) to regulate gene expression in a context-specific manner.

The term “signaling molecule”, as used herein, refers to any entity, whether protein, nucleic acid (DNA or RNA), organic small molecule, lipid, sugar or other biomolecule, which interacts directly, or indirectly, with a regulatory sequence region on a chromosome.

The term “signaling transcription factor”, as used herein, refers to signaling molecules which alter, whether to increase or decrease, the transcription of a target gene, e.g., a neighborhood gene and also act as cell-cell signaling molecules.

The term “small molecule”, as used herein, refers to a low molecular weight drug, i.e. <900 Daltons organic compound with a size on the order of 10−9 m that may help regulate a biological process.

The terms “subject” and “patient” are used interchangeably herein and refer to an animal to whom treatment with the compositions according to the present invention is provided.

Exemplary mammals include humans, monkeys, dogs, cats, mice, rats, cows, horses, camels, goats, rabbits, and sheep. In certain embodiments, the subject is a human. In some embodiments the subject has a disease or condition that can be treated with a compound provided herein. In some aspects, the disease or condition is a liver disease. In some aspects, the disease or condition is a PNPLA3-related disorder. In some aspects, the disease or condition is a PNPLA3-related disease.

The term “in vitro” refers to processes that occur in a living cell growing separate from a living organism, e.g., growing in tissue culture.

The term “in vivo” refers to processes that occur in a living organism.

The term “super-enhancers”, as used herein, refers to are large clusters of transcriptional enhancers that drive expression of genes that define cell identity.

The term “therapeutic agent”, as used herein, refers to a substance that has the ability to cure a disease or ameliorate the symptoms of the disease.

The term “therapeutic or treatment outcome”, as used herein, refers to any result or effect (whether positive, negative or null) which arises as a consequence of the perturbation of a GSC or GSN. Examples of therapeutic outcomes include, but are not limited to, improvement or amelioration of the unwanted or negative conditions associated with a disease or disorder, lessening of side effects or symptoms, cure of a disease or disorder, or any improvement associated with the perturbation of a GSC or GSN.

The term “topologically associating domains” (TADs), as used herein, refers to structures that represent a modular organization of the chromatin and have boundaries that are shared by the different cell types of an organism.

The term “transcription factors”, as used herein, refers to signaling molecules which alter, whether to increase or decrease, the transcription of a target gene, e.g., a neighborhood gene.

The term “therapeutic or treatment liability”, as used herein, refers to a feature or characteristic associated with a treatment or treatment regime which is unwanted, harmful or which mitigates the therapies positive outcomes. Examples of treatment liabilities include for example toxicity, poor half-life, poor bioavailability, lack of or loss of efficacy or pharmacokinetic or pharmacodynamic risks.

The term “upstream neighborhood gene”, as used herein, refers to a gene upstream of a primary neighborhood gene that may be located within the same insulated neighborhood as the primary neighborhood gene.

The term “about” indicates and encompasses an indicated value and a range above and below that value. In certain embodiments, the term “about” indicates the designated value±10%, ±5%, or ±1%. In certain embodiments, where applicable, the term “about” indicates the designated value(s)±one standard deviation of that value(s).

Described herein are compositions and methods for perturbation of genomic signaling centers (GSCs) or entire gene signaling networks (GSNs) for the treatment of liver diseases (e.g., NASH). The details of one or more embodiments of the invention are set forth in the accompanying description below. Although any materials and methods similar or equivalent to those described herein can be used in the practice or testing of the present invention, the preferred materials and methods are now described. Other features, objects and advantages of the invention will be apparent from the description. In the description, the singular forms also include the plural unless the context clearly dictates otherwise. Unless defined otherwise, all technical and scientific terms used herein have the same meaning as commonly understood by one of ordinary skill in the art to which this invention belongs. In the case of conflict, the present description will control.

### VIII. Additional Embodiments

A method of identifying a subject as eligible for a PNPLA3-targeted therapy, comprising the steps of:
a. obtaining a biological sample from the subject;
b. isolating genomic DNA sample from the biological sample;
c. determining in the genomic DNA sample the presence or absence of a G allele at SNP rs738409; and
d. identifying the subject as eligible based on the presence of the G allele at SNP rs738409.

The method, wherein the determining step comprises detecting the allele using a method selected from the group consisting of: mass spectroscopy, oligonucleotide microarray analysis, allele-specific hybridization, allele-specific PCR, and sequencing.

A method of identifying a subject as eligible for a PNPLA3-targeted therapy, comprising the steps of:
a. obtaining a biological sample from the subject;
b. determining in the biological sample the presence or absence of a mutant PNPLA3 protein carrying the I148M mutation; and
c. identifying the subject as eligible based on the presence of the mutant PNPLA3 protein carrying the I148M mutation.

The method, wherein the determining step comprises the use of an antibody that binds specifically to the mutant PNPLA3 protein carrying the I148M mutation.

The method, wherein the biological sample is a biopsy sample.

The method, wherein the method further comprises assessing hepatic triglyceride in the subject. The method, wherein the assessing step comprises using a method selected from the group consisting of liver biopsy, liver ultrasonography, computer-aided tomography (CAT) and nuclear magnetic resonance (NMR). The method, wherein the assessing step comprises proton magnetic resonance spectroscopy (¹H-MRS). The method, wherein the subject is eligible based on a hepatic triglyceride content greater than 5.5% volume/volume. The method, wherein the method further comprising verifying the outcome from the determining step in silico.

The method, wherein the PNPLA3-targeted therapy comprises administering to the subject an effective amount of a compound capable of reducing the expression of the PNPLA3 gene.

The method, wherein the compound comprises Momelotinib (CYT387), or a derivative or an analog thereof.

The method, wherein the compound capable of reducing the expression of the PNPLA3 gene comprises at least one selected from the group consisting of OSI-027, PF-04691502, LY2157299, Momelotinib, Apitolisib, BML-275, DMH-1, Dorsomorphin, Dorsomorphin dihydrochloride, K 02288, LDN-193189, LDN-212854, ML347, SIS3, AZD8055, BGT226 (NVP-BGT226), CC-223, Chrysophanic Acid, CZ415, Dactolisib (BEZ235, NVP-BEZ235), Everolimus (RAD001), GDC-0349, Gedatolisib (PF-05212384, PKI-587), GSK1059615, INK 128 (MLN0128), KU-0063794, LY3023414, MHY1485, Omipalisib (GSK2126458, GSK458), Palomid 529 (P529), PI-103, PP121, Rapamycin (Sirolimus), Ridaforolimus (Deforolimus, MK-8669), SF2523, Tacrolimus (FK506), Temsirolimus (CCI-779, NSC 683864), Torin 1, Torin 2, Torkinib (PP242), Vistusertib (AZD2014), Voxtalisib (SAR245409, XL765) Analogue, Voxtalisib (XL765, SAR245409), WAY-600, WYE-125132 (WYE-132), WYE-354, WYE-687, XL388, Zotarolimus (ABT-578), R788, tamatinib (R406), entospletinib (GS-9973), nilvadipine, TAK-659, BAY-61-3606, MNS (3,4-Methylenedioxy-β-nitrostyrene, MDBN), Piceatannol, PRT-060318, PRT062607 (P505-15, BIIB057), PRT2761, R09021, cerdulatinib, ibrutinib, ONO-4059, ACP-196, idelalisib, duvelisib, pilaralisib, TGR-1202, GS-9820, ACP-319, SF2523, BIO, AZD2858, 1-Azakenpaullone, AR-A014418, AZD1080, Bikinin, BIO-acetoxime, CHIR-98014, CHIR-99021 (CT99021), IM-12, Indirubin, LY2090314, SB216763, SB415286, TDZD-8, Tideglusib, TWS119, ACHP, 10Z-Hymenialdisine, Amlexanox, Andrographolide, Arctigenin, Bay 11-7085, Bay 11-7821, Bengamide B, BI 605906, BMS 345541, Caffeic acid phenethyl ester, Cardamonin, C-DIM 12, Celastrol, CID 2858522, FPS ZM1, Gliotoxin, GSK 319347A, Honokiol, HU 211, IKK 16, IMD 0354, IP7e, IT 901, Luteolin, MG 132, ML 120B dihydrochloride, ML 130, Parthenolide, PF 184, Piceatannol, PR 39 (porcine), Pristimerin, PS 1145 dihydrochloride, PSI, Pyrrolidinedithiocarbamate ammonium, RAGE antagonist peptide, Ro 106-9920, SC 514, SP 100030, Sulfasalazine, Tanshinone IIA, TPCA-1, Withaferin A, Zoledronic Acid, Ruxolitinib, Oclacitinib, Baricitinib, Filgotinib, Gandotinib, Lestaurtinib, PF-04965842, Upadacitinib, Cucurbitacin I, CHZ868, Fedratinib, AC430, AT9283, ati-50001 and ati-50002, AZ 960, AZD1480, BMS-911543, CEP-33779, Cerdulatinib (PRT062070, PRT2070), Curcumol, Decernotinib (VX-509), Fedratinib (SAR302503, TG101348), FLLL32, FM-381, GLPG0634 analogue, Go6976, JANEX-1 (WHI-P131), NVP-BSK805, Pacritinib (SB1518), Peficitinib (ASP015K, JNJ-54781532), PF-06651600, PF-06700841, R256 (AZD0449), Solcitinib (GSK2586184 or GLPG0778), S-Ruxolitinib (INCB018424), TG101209, Tofacitinib (CP-690550), WHI-P154, WP1066, XL019, ZM 39923 HCl, Amuvatinib, BMS-754807, BMS-986094, LY294002, Pifithrin-μ, and XMU-MP-1, or a derivative or an analog thereof.

The method, wherein the compound comprises one or more small interfering RNA (siRNA) targeting one or more genes selected from the group consisting of JAK1, JAK2, mTOR, SYK, PDGFRA, PDGFRB, GSK3, ACVR1, SMAD3, SMAD4, NF-κB and HSD17B13.

The method, wherein the subject has a G allele at SNP rs738409. The method, wherein the subject is homozygous for the G allele at SNP rs738409. The method, wherein the subject is heterozygous for the G allele at SNP rs738409.

The method, wherein the subject has a mutant PNPLA3 protein carrying the I148M mutation. The method, wherein the subject is homozygous for the mutant PNPLA3 protein carrying the I148M mutation. The method, wherein the subject is heterozygous for the mutant PNPLA3 protein carrying the I148M mutation.

A method of treating a subject with a PNPLA3-targeted therapy, comprising the steps of:
a. identifying the subject as eligible for the PNPLA3-targeted treatment according to any one of claims 1-46; and
b. administering to the subject an effective amount of a compound capable of reducing the expression of the PNPLA3 gene.

The method, wherein the compound comprises Momelotinib (CYT387), or a derivative or an analog thereof. The method, wherein the compound comprises OSI-027, or a derivative or an analog thereof. The method, wherein the compound comprises PF-04691502, or a derivative or an analog thereof. The method, wherein the compound comprises LY2157299 (Galunisertib), or a derivative or an analog thereof.

The method, wherein the compound capable of reducing the expression of the PNPLA3 gene comprises at least one selected from the group consisting of OSI-027, PF-04691502, LY2157299, Momelotinib, Apitolisib, BML-275, DMH-1, Dorsomorphin, Dorsomorphin dihydrochloride, K 02288, LDN-193189, LDN-212854, ML347, SIS3, AZD8055, BGT226 (NVP-BGT226), CC-223, Chrysophanic Acid, CZ415, Dactolisib (BEZ235, NVP-BEZ235), Everolimus (RAD001), GDC-0349, Gedatolisib (PF-05212384, PKI-587), GSK1059615, INK 128 (MLN0128), KU-0063794, LY3023414, MHY1485, Omipalisib (GSK2126458, GSK458), Palomid 529 (P529), PI-103, PP121, Rapamycin (Sirolimus), Ridaforolimus (Deforolimus, MK-8669), SF2523, Tacrolimus (FK506), Temsirolimus (CCI-779, NSC 683864), Torin 1, Torin 2, Torkinib (PP242), Vistusertib (AZD2014), Voxtalisib (SAR245409, XL765) Analogue, Voxtalisib (XL765, SAR245409), WAY-600, WYE-125132 (WYE-132), WYE-354, WYE-687, XL388, Zotarolimus (ABT-578), R788, tamatinib (R406), entospletinib (GS-9973), nilvadipine, TAK-659, BAY-61-3606, MNS (3,4-Methylenedioxy-β-nitrostyrene, MDBN), Piceatannol, PRT-060318, PRT062607 (P505-15, BIIB057), PRT2761, RO9021, cerdulatinib, ibrutinib, ONO-4059, ACP-196, idelalisib, duvelisib, pilaralisib, TGR-1202, GS-9820, ACP-319, SF2523, BIO, AZD2858, 1-Azakenpaullone, AR-A014418, AZD1080, Bikinin, BIO-acetoxime, CHIR-98014, CHIR-99021 (CT99021), IM-12, Indirubin, LY2090314, SB216763, SB415286, TDZD-8, Tideglusib, TWS119, ACHP, 10Z-Hymenialdisine, Amlexanox, Andrographolide, Arctigenin, Bay 11-7085, Bay 11-7821, Bengamide B, BI 605906, BMS 345541, Caffeic acid phenethyl ester, Cardamonin, C-DIM 12, Celastrol, CID 2858522, FPS ZM1, Gliotoxin, GSK 319347A, Honokiol, HU 211, IKK 16, IMD 0354, IP7e, IT 901, Luteolin, MG 132, ML 120B dihydrochloride, ML 130, Parthenolide, PF 184, Piceatannol, PR 39 (porcine), Pristimerin, PS 1145 dihydrochloride, PSI, Pyrrolidinedithiocarbamate ammonium, RAGE antagonist peptide, Ro 106-9920, SC 514, SP 100030, Sulfasalazine, Tanshinone IIA, TPCA-1, Withaferin A, Zoledronic Acid, Ruxolitinib, Oclacitinib, Baricitinib, Filgotinib, Gandotinib, Lestaurtinib, PF-04965842, Upadacitinib, Cucurbitacin I, CHZ868, Fedratinib, AC430, AT9283, ati-50001 and ati-50002, AZ 960, AZD1480, BMS-911543, CEP-33779, Cerdulatinib (PRT062070, PRT2070), Curcumol, Decemotinib (VX-509), Fedratinib (SAR302503, TG101348), FLLL32, FM-381, GLPG0634 analogue, Go6976, JANEX-1 (WHI-P131), NVP-BSK805, Pacritinib (SB1518), Peficitinib (ASP015K, JNJ-54781532), PF-06651600, PF-06700841, R256 (AZD0449), Solcitinib (GSK2586184 or GLPG0778), S-Ruxolitinib (INCB018424), TG101209, Tofacitinib (CP-690550), WHI-P154, WP1066, XL019, ZM 39923 HCl, Amuvatinib, BMS-754807, BMS-986094, LY294002, Pifithrinμ, and XMU-MP-1, or a derivative or an analog thereof.

The method, wherein the compound comprises one or more small interfering RNA (siRNA) targeting one or more genes selected from the group consisting of JAK1, JAK2, mTOR, SYK, PDGFRA, PDGFRB, GSK3, ACVR1, SMAD3, SMAD4, NF-κB and HSD17B13.

The method, wherein the subject has a G allele at SNP rs738409. The method, wherein the subject is homozygous for the G allele at SNP rs738409. The method, wherein the subject is heterozygous for the G allele at SNP rs738409.

The method, wherein the subject has a mutant PNPLA3 protein carrying the I148M mutation. The method, wherein the subject is homozygous for the mutant PNPLA3 protein carrying the I148M mutation. The method, wherein the subject is heterozygous for the mutant PNPLA3 protein carrying the I148M mutation.

The method, wherein the expression of the PNPLA3 gene is reduced by at least about 30%. The method, wherein the expression of the PNPLA3 gene is reduced by at least about 50%. The method, wherein the expression of the PNPLA3 gene is reduced by at least about 70%. The method, wherein the expression of the PNPLA3 gene is reduced in the liver of the subject.

The method, wherein the expression of the PNPLA3 gene is reduced in the hepatocytes of the subject. The method, wherein the expression of the PNPLA3 gene is reduced in the hepatic stellate cells of the subject. The method, wherein the expression of the PNPLA3 gene is reduced in the hepatocytes and hepatic stellate cells of the subject.

A diagnostic kit for the detection of the genetic marker of PNPLA3-I148M.

A method of treating a subject in need thereof with a PNPLA3-targeted therapy, comprising administering to the subject an effective amount of a compound capable of reducing the expression of the PNPLA3 gene.

The method, further comprising a step of identifying or having identified the presence or absence of a G allele at SNP rs738409 in a biological sample from the subject prior to the administering step.

The method, further comprising a step of identifying or having identified the presence or absence of a mutant PNPLA3 protein carrying the I148M mutation in a biological sample from the subject prior to the administering step.

The method, wherein the determining step comprises detecting the marker using a method selected from the group consisting of: mass spectroscopy, oligonucleotide microarray analysis, allele-specific hybridization, allele-specific PCR, and sequencing.

The method, wherein the determining step comprises the use of an antibody that binds specifically to the mutant PNPLA3 protein carrying the I148M mutation.

The method, wherein the biological sample is a biopsy sample.

The method, wherein the method further comprises assessing hepatic triglyceride in the subject.

The method, wherein the assessing step comprises using a method selected from the group consisting of liver biopsy, liver ultrasonography, computer-aided tomography (CAT) and nuclear magnetic resonance (NMR). The method, wherein the assessing step comprises proton magnetic resonance spectroscopy (¹H-MRS). The method, wherein the subject is eligible based on a hepatic triglyceride content greater than 5.5% volume/volume.

The method, wherein the method further comprising verifying the outcome from the determining step in silico.

The method, wherein the compound comprises Momelotinib (CYT387), or a derivative or an analog thereof. The method, wherein the compound comprises OSI-027, or a derivative or an analog thereof. The method, wherein the compound comprises PF-04691502, or a derivative or an analog thereof. The method, wherein the compound comprises LY2157299 (Galunisertib), or a derivative or an analog thereof.

The method, wherein the compound capable of reducing the expression of the PNPLA3 gene comprises at least one selected from the group consisting of OSI-027, PF-04691502, LY2157299, Momelotinib, Apitolisib, BML-275, DMH-1, Dorsomorphin, Dorsomorphin dihydrochloride, K 02288, LDN-193189, LDN-212854, ML347, SIS3, AZD8055, BGT226 (NVP-BGT226), CC-223, Chrysophanic Acid, CZ415, Dactolisib (BEZ235, NVP-BEZ235), Everolimus (RAD001), GDC-0349, Gedatolisib (PF-05212384, PKI-587), GSK1059615, INK 128 (MLN0128), KU-0063794, LY3023414, MHY1485, Omipalisib (GSK2126458, GSK458), Palomid 529 (P529), PI-103, PP121, Rapamycin (Sirolimus), Ridaforolimus (Deforolimus, MK-8669), SF2523, Tacrolimus (FK506), Temsirolimus (CCI-779, NSC 683864), Torin 1, Torin 2, Torkinib (PP242), Vistusertib (AZD2014), Voxtalisib (SAR245409, XL765) Analogue, Voxtalisib (XL765, SAR245409), WAY-600, WYE-125132 (WYE-132), WYE-354, WYE-687, XL388, Zotarolimus (ABT-578), R788, tamatinib (R406), entospletinib (GS-9973), nilvadipine, TAK-659, BAY-61-3606, MNS (3,4-Methylenedioxy-β-nitrostyrene, MDBN), Piceatannol, PRT-060318, PRT062607 (P505-15, BIIB057), PRT2761, R09021, cerdulatinib, ibrutinib, ONO-4059, ACP-196, idelalisib, duvelisib, pilaralisib, TGR-1202, GS-9820, ACP-319, SF2523, BIO, AZD2858, 1-Azakenpaullone, AR-A014418, AZD1080, Bikinin, BIO-acetoxime, CHIR-98014, CHIR-99021 (CT99021), IM-12, Indirubin, LY2090314, SB216763, SB415286, TDZD-8, Tideglusib, TWS119, ACHP, 10Z-Hymenialdisine, Amlexanox, Andrographolide, Arctigenin, Bay 11-7085, Bay 11-7821, Bengamide B, BI 605906, BMS 345541, Caffeic acid phenethyl ester, Cardamonin, C-DIM 12, Celastrol, CID 2858522, FPS ZM1, Gliotoxin, GSK 319347A, Honokiol, HU 211, IKK 16, IMD 0354, IP7e, IT 901, Luteolin, MG 132, ML 120B dihydrochloride, ML 130, Parthenolide, PF 184, Piceatannol, PR 39 (porcine), Pristimerin, PS 1145 dihydrochloride, PSI, Pyrrolidinedithiocarbamate ammonium, RAGE antagonist peptide, Ro 106-9920, SC 514, SP 100030, Sulfasalazine, Tanshinone IIA, TPCA-1, Withaferin A, Zoledronic Acid, Ruxolitinib, Oclacitinib, Baricitinib, Filgotinib, Gandotinib, Lestaurtinib, PF-04965842, Upadacitinib, Cucurbitacin I, CHZ868, Fedratinib, AC430, AT9283, ati-50001 and ati-50002, AZ 960, AZD1480, BMS-911543, CEP-33779, Cerdulatinib (PRT062070, PRT2070), Curcumol, Decemotinib (VX-509), Fedratinib (SAR302503, TG101348), FLLL32, FM-381, GLPG0634 analogue, Go6976, JANEX-1 (WHI-P131), NVP-BSK805, Pacritinib (SB1518), Peficitinib (ASP015K, JNJ-54781532), PF-06651600, PF-06700841, R256 (AZD0449), Solcitinib (GSK2586184 or GLPG0778), S-Ruxolitinib (INCB018424), TG101209, Tofacitinib (CP-690550), WHI-P154, WP1066, XL019, ZM 39923 HCl, Amuvatinib, BMS-754807, BMS-986094, LY294002, Pifithrin-μ, and XMU-MP-1, or a derivative or an analog thereof.

The method, wherein the compound comprises one or more small interfering RNA (siRNA) targeting one or more genes selected from the group consisting of JAK1, JAK2, mTOR, SYK, PDGFRA, PDGFRB, GSK3, ACVR1, SMAD3, SMAD4, NF-κB and HSD17B13.

The method, wherein the subject has a G allele at SNP rs738409. The method, wherein the subject is homozygous at the G allele at SNP rs738409. The method, wherein the subject is heterozygous at the G allele at SNP rs738409.

The method, wherein the subject has a mutant PNPLA3 protein carrying the I148M mutation. The method, wherein the subject is homozygous for the mutant PNPLA3 protein carrying the I148M mutation. The method, wherein the subject is heterozygous for the mutant PNPLA3 protein carrying the I148M mutation.

The method, wherein the expression of the PNPLA3 gene is reduced by at least about 30%. The method, wherein the expression of the PNPLA3 gene is reduced by at least about 50%. The method, wherein the expression of the PNPLA3 gene is reduced by at least about 70%. The method, wherein the expression of the PNPLA3 gene is reduced in the liver of the subject.

The method, wherein the expression of the PNPLA3 gene is reduced in the hepatocytes of the subject. The method, wherein the expression of the PNPLA3 gene is reduced in the hepatic stellate cells of the subject. The method, wherein the expression of the PNPLA3 gene is reduced in the hepatocytes and hepatic stellate cells of the subject.

A method of reducing the accumulation of PNPLA3 protein on lipid droplets in cells in a subject, comprising the steps of:
a. obtaining a biological sample from the subject;
b. determining in the biological sample the amount of accumulation of PNPLA3 protein on lipid droplets in cells; and
c. administering an effective amount of a compound capable of reducing the expression of the PNPLA3 gene.

The method, wherein the method further comprising assessing the hepatic triglyceride in the subject. The method, wherein the assessing step comprises using a method selected from the group consisting of liver biopsy, liver ultrasonography, computer-aided tomography (CAT) and nuclear magnetic resonance (NMR).

The method, wherein the PNPLA3 protein accumulation is in hepatocytes. The method, wherein the PNPLA3 protein accumulation is in hepatic stellate cells. The method, wherein the PNPLA3 protein accumulation is in a population of hepatocytes and hepatic stellate cells.

The method, wherein the compound comprises Momelotinib (CYT387), or a derivative or an analog thereof. The method, wherein the compound comprises OSI-027, or a derivative or an analog thereof. The method, wherein the compound comprises PF-04691502, or a derivative or an analog thereof. The method, wherein the compound comprises LY2157299 (Galunisertib), or a derivative or an analog thereof.

The method, wherein the compound capable of reducing the expression of the PNPLA3 gene comprises at least one selected from the group consisting of OSI-027, PF-04691502, LY2157299, Momelotinib, Apitolisib, BML-275, DMH-1, Dorsomorphin, Dorsomorphin dihydrochloride, K 02288, LDN-193189, LDN-212854, ML347, SIS3, AZD8055, BGT226 (NVP-BGT226), CC-223, Chrysophanic Acid, CZ415, Dactolisib (BEZ235, NVP-BEZ235), Everolimus (RAD001), GDC-0349, Gedatolisib (PF-05212384, PKI-587), GSK1059615, INK 128 (MLN0128), KU-0063794, LY3023414, MHY1485, Omipalisib (GSK2126458, GSK458), Palomid 529 (P529), PI-103, PP121, Rapamycin (Sirolimus), Ridaforolimus (Deforolimus, MK-8669), SF2523, Tacrolimus (FK506), Temsirolimus (CCI-779, NSC 683864), Torin 1, Torin 2, Torkinib (PP242), Vistusertib (AZD2014), Voxtalisib (SAR245409, XL765) Analogue, Voxtalisib (XL765, SAR245409), WAY-600, WYE-125132 (WYE-132), WYE-354, WYE-687, XL388, Zotarolimus (ABT-578), R788, tamatinib (R406), entospletinib (GS-9973), nilvadipine, TAK-659, BAY-61-3606, MNS (3,4-Methylenedioxy-β-nitrostyrene, MDBN), Piceatannol, PRT-060318, PRT062607 (P505-15, BIIB057), PRT2761, R09021, cerdulatinib, ibrutinib, ONO-4059, ACP-196, idelalisib, duvelisib, pilaralisib, TGR-1202, GS-9820, ACP-319, SF2523, BIO, AZD2858, 1-Azakenpaullone, AR-A014418, AZD1080, Bikinin, BIO-acetoxime, CHIR-98014, CHIR-99021 (CT99021), IM-12, Indirubin, LY2090314, SB216763, SB415286, TDZD-8, Tideglusib, TWS119, ACHP, 10Z-Hymenialdisine, Amlexanox, Andrographolide, Arctigenin, Bay 11-7085, Bay 11-7821, Bengamide B, BI 605906, BMS 345541, Caffeic acid phenethyl ester, Cardamonin, C-DIM 12, Celastrol, CID 2858522, FPS ZM1, Gliotoxin, GSK 319347A, Honokiol, HU 211, IKK 16, IMD 0354, IP7e, IT 901, Luteolin, MG 132, ML 120B dihydrochloride, ML 130, Parthenolide, PF 184, Piceatannol, PR 39 (porcine), Pristimerin, PS 1145 dihydrochloride, PSI, Pyrrolidinedithiocarbamate ammonium, RAGE antagonist peptide, Ro 106-9920, SC 514, SP 100030, Sulfasalazine, Tanshinone IIA, TPCA-1, Withaferin A, Zoledronic Acid, Ruxolitinib, Oclacitinib, Baricitinib, Filgotinib, Gandotinib, Lestaurtinib, PF-04965842, Upadacitinib, Cucurbitacin I, CHZ868, Fedratinib, AC430, AT9283, ati-50001 and ati-50002, AZ 960, AZD1480, BMS-911543, CEP-33779, Cerdulatinib (PRT062070, PRT2070), Curcumol, Decemotinib (VX-509), Fedratinib (SAR302503, TG101348), FLLL32, FM-381, GLPG0634 analogue, Go6976, JANEX-1 (WHI-P131), NVP-BSK805, Pacritinib (SB1518), Peficitinib (ASP015K, JNJ-54781532), PF-06651600, PF-06700841, R256 (AZD0449), Solcitinib (GSK2586184 or GLPG0778), S-Ruxolitinib (INCB018424), TG101209, Tofacitinib (CP-690550), WHI-P154, WP1066, XL019, ZM 39923 HCl, Amuvatinib, BMS-754807, BMS-986094, LY294002, Pifithrin-μ, and XMU-MP-1, or a derivative or an analog thereof.

The method, wherein the compound comprises one or more small interfering RNA (siRNA) targeting one or more genes selected from the group consisting of JAK1, JAK2, mTOR, SYK, PDGFRA, PDGFRB, GSK3, ACVR1, SMAD3, SMAD4, NF-κB and HSD17B13.

The method, wherein the expression of the PNPLA3 gene is reduced by at least about 30%. The method, wherein the expression of the PNPLA3 gene is reduced by at least about 50%. The method, wherein the expression of the PNPLA3 gene is reduced by at least about 70%.

### EXAMPLES

### Example 1

### Experimental Procedures

A. Human Hepatocyte Cell Culture

Human hepatocytes were obtained from two donors from Massachusetts General Hospital, namely MGH54 and MGH63, and one donor from Lonza, namely HUM4111B. Cryopreserved hepatocytes were cultured in plating media for 16 hours, transferred to maintenance media for 4 hours. Cultured on serum-free media for 2 hours, then a compound was added. The hepatocytes were maintained on the serum-free media for 16 hours prior to gene expression analysis. Primary Human Hepatocytes were stored in the vapor phase of a liquid nitrogen freezer (about −130° C.).

To seed the primary human hepatocytes, vials of cells were retrieved from the LN₂ freezer, thawed in a 37° C. water bath, and swirled gently until only a sliver of ice remains. Using a 10 ml serological pipet, cells were gently pipetted out of the vial and gently pipetted down the side of 50 mL conical tube containing 20 mL cold thaw medium. The vial was rinsed with about 1 mL of thaw medium, and the rinse was added to the conical tube. Up to 2 vials may be added to one tube of 20 mL thaw medium.

The conical tube(s) were gently inverted 2-3 times and centrifuged at 100 g for 10 minutes at 4° C. with reduced braking (e.g. 4 out of 9). The thaw medium slowly was slowly aspirated to avoid the pellet. 4 mL cold plating medium was added slowly down the side (8 mL if combined 2 vials to 1 tube), and the vial was inverted gently several times to resuspend cells.

Cells were kept on ice until 100 μl of well-mixed cells were added to 400 μl diluted Trypan blue and mixed by gentle inversion. They were counted using a hemocytometer (or Cellometer), and viability and viable cells/mL were noted. Cells were diluted to a desired concentration and seeded on collagen I-coated plates. Cells were pipetted slowly and gently onto plate, only 1-2 wells at a time. The remaining cells were mixed in the tubes frequently by gentle inversion. Cells were seeded at about 8.5×10⁶ cells per plate in 6 mL cold plating medium (10 cm). Alternatively, 1.5×10⁶ per well for a 6-well plate (1 mL medium/well); 7×10⁵ per well for 12-well plate (0.5 mL/well); or 3.75×10⁵ per well for a 24-well plate (0.5 mL/well)

After all cells and medium were added to the plate, the plate was transferred to an incubator (37° C., 5% CO₂, about 90% humidity) and rocked forwards and backwards, then side to side several times each to distribute cells evenly across the plate or wells. The plate(s) were rocked again every 15 minutes for the first hour post-plating. About 4 hours post-plating (or first thing the morning if cells were plated in the evening), cells were washed once with PBS and complete maintenance medium was added. The primary human hepatocytes were maintained in the maintenance medium and transferred to fresh medium daily.

B. Starvation and Compound Treatment of Human Hepatocytes

Human hepatocytes cultured as described above were plated in 24-well format, adding 375,000 cells per well in a volume of 500 μl plating medium. Four hours before treatment, cells were washed with PBS and the medium was changed to either: fresh maintenance medium (complete) or modified maintenance medium.

Compound stocks were prepared at 1000× final concentration and added in a 2-step dilution to the medium to reduce risk of a compound precipitating out of solution when added to the cells, and to ensure reasonable pipetting volumes. One at a time, each compound was first diluted 10-fold in warm (about 37° C.) modified maintenance medium (initial dilution=ID), mixed by vortexing, and the ID was diluted 100-fold into the cell culture (e.g. 5.1 μl into 1 well of a 24-well plate containing 0.5 mL medium). The plate was mixed by carefully swirling and after all wells were treated and returned to the incubator overnight. If desired, separate plates/wells were treated with vehicle-only controls and/or positive controls. If using multi-well plates, controls were included on each plate. After about 18 hours, cells were harvested for further analysis, e.g., ChIP-seq, RNA-seq, ATAC-seq, etc.

C. Mouse Hepatocyte Cell Culture and Compound Treatment

Female C57BL/6 mouse hepatocytes (F005152-cryopreserved) were purchased from BioreclamationIVT as a pool of 45 donors. Cells were plated in InvitroGRO CP Rodent Medium (Z990028) and Torpedo Rodent Antibiotic Mix (Z99027) on Collagen-coated 24-well plates for 24 hours at 200K cells/well in 0.5 mL media. Compound stocks in 10 mM DMSO, were diluted to 10 uM (with final concentration of 1% DMSO), and applied on cells in biological triplicates. Medium was removed after 20 hours and cells processed for further analysis, e.g. qRT-PCR.

D. Stellate Cell Culture and Compound Treatment

Human Primary Stellate cells (HSC) (ScienceCell Cat #5300) were originally isolated from the liver of a 15-year-old female donor. Cells were plated in Stellate Cell Medium (SteCM) (ScienCell Cat#5301) on black clear bottom plates (GREINER BIO-ONE:82050-730) coated with 2 μg/cm² PolyLLysine (PLL) (ScienceCell Cat #0413). Cells were plated at a density of 17000 cells/well in a 96-well plate and allowed to adhere overnight. The following day cell culture media was replenished with the indicated concentration(s) of compound for 18 hours. All wells possessed 1% DMSO. Medium was removed after 18 hours and cells were processed for further analysis, e.g. qRT-PCR.

E. HepG2 Cell Culture and Compound Treatment

HepG2 cells were plated in 24 well format at 100,000 cells per well in 500 μl DMEM. After 48 hours, the medium was removed and replaced with fresh medium containing 10 μM Momelotinib or DMSO. The following morning, the cells were harvested for RNA extraction.

F. Media Composition

The thaw medium contained 6 mL isotonic percoll and 14 mL high glucose DMEM (Invitrogen #11965 or similar). The plating medium contained 100 mL Williams E medium (Invitrogen #A1217601, without phenol red) and the supplement pack #CM3000 from ThermoFisher Plating medium containing 5 mL FBS, 10 μl dexamethasone, and 3.6 mL plating/maintenance cocktail. Stock trypan blue (0.4%, Invitrogen #15250) was diluted 1:5 in PBS. Normocin was added at 1:500 to both the thaw medium and the plating medium.

The ThermoFisher complete maintenance medium contained supplement pack #CM4000 (1 μl dexamethasone and 4 mL maintenance cocktail) and 100 mL Williams E (Invitrogen #A1217601, without phenol red).

The modified maintenance media had no stimulating factors (dexamethasone, insulin, etc.), and contained100 mL Williams E (Invitrogen #A1217601, without phenol red), 1 mL L-Glutamine (Sigma #G7513) to 2 mM, 1.5 mL HEPES (VWR #J848) to 15 mM, and 0.5 mL penicillin/streptomycin (Invitrogen #15140) to a final concentration of 50U/mL each.

G. DNA Purification

DNA purification was conducted as described in Ji et al., PNAS 112(12):3841-3846 (2015) Supporting Information, which is hereby incorporated by reference in its entirety. One milliliter of 2.5 M glycine was added to each plate of fixed cells and incubated for 5 minutes to quench the formaldehyde. The cells were washed twice with PBS. The cells were pelleted at 1,300 g for 5 minutes at 4° C. Then, 4×10⁷ cells were collected in each tube. The cells were lysed gently with 1 mL of ice-cold Nonidet P-40 lysis buffer containing protease inhibitor on ice for 5 minutes (buffer recipes are provided below). The cell lysate was layered on top of 2.5 volumes of sucrose cushion made up of 24% (wt/vol) sucrose in Nonidet P-40 lysis buffer. This sample was centrifuged at 18,000 g for 10 minutes at 4° C. to isolate the nuclei pellet (the supernatant represented the cytoplasmic fraction). The nuclei pellet was washed once with PBS/1 mM EDTA. The nuclei pellet was resuspended gently with 0.5 mL glycerol buffer followed by incubation for 2 minutes on ice with an equal volume of nuclei lysis buffer. The sample was centrifuged at 16,000 g for 2 minutes at 4° C. to isolate the chromatin pellet (the supernatant represented the nuclear soluble fraction). The chromatin pellet was washed twice with PBS/1 mM EDTA. The chromatin pellet was stored at −80° C.

The Nonidet P-40 lysis buffer contained 10 mM Tris.HCl (pH 7.5), 150 mM NaCl, and 0.05% Nonidet P-40. The glycerol buffer contained 20 mM Tris.HCl (pH 7.9), 75 mM NaCl, 0.5 mM EDTA, 0.85 mM DTT, and 50% (vol/vol) glycerol. The nuclei lysis buffer contained 10 mM Hepes (pH 7.6), 1 mM DTT, 7.5 mM MgCl₂, 0.2 mM EDTA, 0.3 M NaCl, 1 M urea, and 1% Nonidet P-40.

H. Chromatin Immunoprecipitation Sequencing (ChIP-seq)

ChIP-seq was performed using the following protocol for primary hepatocytes and HepG2 cells to determine the composition and confirm the location of signaling centers.

i. Cell Cross-Linking

2×10⁷ cells were used for each run of ChIP-seq. Two ml of fresh 11% formaldehyde (FA) solution was added to 20 ml media on 15 cm plates to reach a 1.1% final concentration. Plates were swirled briefly and incubated at room temperature (RT) for 15 minutes. At the end of incubation, the FA was quenched by adding 1 ml of 2.5M Glycine to plates and incubating for 5 minutes at RT. The media was discarded to a 1 L beaker, and cells were washed twice with 20 ml ice-cold PBS. PBS (10 ml) was added to plates, and cells were scraped off the plate. The cells were transferred to 15 ml conical tubes, and the tubes were placed on ice. Plates were washed with an additional 4 ml of PBS and combined with cells in 15 ml tubes. Tubes were centrifuged for 5 minutes at 1,500 rpm at 4° C. in a tabletop centrifuge. PBS was aspirated, and the cells were flash frozen in liquid nitrogen. Pellets were stored at −80° C. until ready to use.

ii. Pre-Block Magnetic Beads

Thirty μl Protein G beads (per reaction) were added to a 1.5 ml Protein LoBind Eppendorf tube. The beads were collected by magnet separation at RT for 30 seconds. Beads were washed 3 times with 1 ml of blocking solution by incubating beads on a rotator at 4° C. for 10 minutes and collecting the beads with the magnet. Five μg of an antibody was added to the 250 μl of beads in block solution. The mix was transferred to a clean tube, and rotated overnight at 4° C. On the next day, buffer containing antibodies was removed, and beads were washed 3 times with 1.1 ml blocking solution by incubating beads on a rotator at 4° C. for 10 minutes and collecting the beads with the magnet. Beads were resuspended in 50 μl of block solution and kept on ice until ready to use.

iii. Cell Lysis, Genomic Fragmentation, and Chromatin Immunoprecipitation

COMPLETE® protease inhibitor cocktail was added to lysis buffer 1 (LB1) before use. One tablet was dissolved in 1 ml of H₂O for a 50× solution. The cocktail was stored in aliquots at −20° C. Cells were resuspended in each tube in 8 ml of LB1 and incubated on a rotator at 4° C. for 10 minutes. Nuclei were spun down at 1,350 g for 5 minutes at 4° C. LB1 was aspirated, and cells were resuspended in each tube in 8 ml of LB2 and incubated on a rotator at 4° C. for 10 minutes.

A COVARIS® E220EVOLUTION™ ultrasonicator was programmed per the manufacturer's recommendations for high cell numbers. HepG2 cells were sonicated for 12 minutes, and primary hepatocyte samples were sonicated for 10 minutes. Lysates were transferred to clean 1.5 ml Eppendorf tubes, and the tubes were centrifuged at 20,000 g for 10 minutes at 4° C. to pellet debris. The supernatant was transferred to a 2 ml Protein LoBind Eppendorf tube containing pre-blocked Protein G beads with pre-bound antibodies. Fifty μl of the supernatant was saved as input. Input material was kept at −80° C. until ready to use. Tubes were rotated with beads overnight at 4° C.

iv. Wash, Elution, and Cross-Link Reversal

All washing steps were performed by rotating tubes for 5 minutes at 4° C. The beads were transferred to clean Protein LoBind Eppendorf tubes with every washing step. Beads were collected in 1.5 ml Eppendorf tube using a magnet. Beads were washed twice with 1.1 ml of sonication buffer. The magnetic stand was used to collect magnetic beads. Beads were washed twice with 1.1 ml of wash buffer 2, and the magnetic stand was used again to collect magnetic beads. Beads were washed twice with 1.1 ml of wash buffer 3. All residual Wash buffer 3 was removed, and beads were washed once with 1.1 ml TE+0.2% Triton X-100 buffer. Residual TE+0.2% Triton X-100 buffer was removed, and beads were washed twice with TE buffer for 30 seconds each time. Residual TE buffer was removed, and beads were resuspended in 300 μl of ChIP elution buffer. Two hundred fifty μl of ChIP elution buffer was added to 50 μl of input, and the tubes were rotated with beads 1 hour at 65° C. Input sample was incubated overnight at 65° C. oven without rotation. Tubes with beads were placed on a magnet, and the eluate was transferred to a fresh DNA LoBind Eppendorf tube. The eluate was incubated overnight at 65° C. oven without rotation

v. Chromatin Extraction and Precipitation

Input and immunoprecipitant (IP) samples were transferred to fresh tubes, and 300 μl of TE buffer was added to IP and Input samples to dilute SDS. RNase A (20 mg/ml) was added to the tubes, and the tubes were incubated at 37° C. for 30 minutes. Following incubation, 3 μl of 1M CaCl₂ and 7 μl of 20 mg/ml Proteinase K were added, and incubated 1.5 hours at 55° C. MaXtract High Density 2 ml gel tubes (Qiagen) were prepared by centrifugation at full speed for 30 seconds at RT. Six hundred μl of phenol/chloroform/isoamyl alcohol was added to each proteinase K reaction and transferred in about 1.2 ml mixtures to the MaXtract tubes. Tubes were spun at 16,000 g for 5 minutes at RT. The aqueous phase was transferred to two clean DNA LoBind tubes (300 μl in each tube), and 1.5 μl glycogen, 30 μl of 3M sodium acetate, and 900 μl ethanol were added. The mixture was precipitated overnight at −20° C. or for 1 hour at −80° C., and spun down at maximum speed for 20 minutes at 4° C. The ethanol was removed, and pellets were washed with 1 ml of 75% ethanol by spinning tubes down at maximum speed for 5 minutes at 4° C. Remnants of ethanol were removed, and pellets were dried for 5 min at RT. Twenty-five μl of H₂O was added to each immunoprecipitant (IP) and input pellet, left standing for 5 minutes, and vortexed briefly. DNA from both tubes was combined to obtain 50 μl of IP and 50 μl of input DNA for each sample. One μl of this DNA was used to measure the amount of pulled down DNA using Qubit dsDNA HS assay (ThermoFisher, #Q32854). The total amount of immunoprecipitated material ranged from several ng (for TFs) to several hundred ng (for chromatin modifications). Six μl of DNA was analyzed using qRT-PCR to determine enrichment. The DNA was diluted if necessary. If enrichment was satisfactory, the rest was used for library preparation for DNA sequencing.

vi. Library Preparation for DNA Sequencing

Libraries were prepared using NEBNext Ultra II DNA library prep kit for Illumina (NEB, #E7645) using NEBNext Multiplex Oligos for Illumina (NEB, #6609S) according to manufacturer's instructions with the following modifications. The remaining ChIP sample (about 43 μl) and 1 μg of input samples for library preparations were brought up the volume of 50 μl before the End Repair portion of the protocol. End Repair reactions were run in a PCR machine with a heated lid in a 96-well semi-skirted PCR plate (ThermoFisher, #AB1400) sealed with adhesive plate seals (ThermoFisher, #AB0558) leaving at least one empty well in-between different samples. Undiluted adapters were used for input samples, 1:10 diluted adapters for 5-100 ng of ChIP material, and 1:25 diluted adapters for less than 5 ng of ChIP material. Ligation reactions were run in a PCR machine with the heated lid off. Adapter ligated DNA was transferred to clean DNA LoBind Eppendorf tubes, and the volume was brought to 96.5 μl using H₂O.

200-600 bp ChIP fragments were selected using SPRIselect magnetic beads (Beckman Coulter, #B23317). Thirty μl of the beads were added to 96.5 μl of ChIP sample to bind fragments that are longer than 600 bp. The shorter fragments were transferred to a fresh DNA LoBind Eppendorf tube. Fifteen μl of beads were added to bind the DNA longer than 200 bp, and beads were washed with DNA twice using freshly prepared 75% ethanol. DNA was eluted using 17 μl of 0.1× TE buffer. About 15 μl was collected.

Three μl of size-selected Input sample and all (15 μl) of the ChIP sample was used for PCR. The amount of size-selected DNA was measured using a Qubit dsDNA HS assay. PCR was run for 7 cycles of for Input and ChIP samples with about 5-10 ng of size-selected DNA, and 12 cycles with less than 5 ng of size-selected DNA. One-half of the PCR product (25 μl) was purified with 22.5 μl of AMPure XP beads (Beckman Coulter, #A63880) according to the manufacturer's instructions. PCR product was eluted with 17 μl of 0.1× TE buffer, and the amount of PCT product was measured using Qubit dsDNA HS assay. An additional 4 cycles of PCR were run for the second half of samples with less than 5 ng of PCR product, DNA was purified using 22.5 μl of AMPure XP beads. The concentration was measured to determine whether there was an increased yield. Both halves were combined, and the volume was brought up to 50 μl using H₂O.

A second round of purifications of DNA was run using 45 μl of AMPure XP beads in 17 μl of 0.1× TE, and the final yield was measured using Qubit dsDNA HS assay. This protocol produces from 20 ng to 1 mg of PCR product. The quality of the libraries was verified by diluting 1 μl of each sample with H₂O if necessary using the High Sensitivity BioAnalyzer DNA kit (Agilent, #5067-4626) based on manufacturer's recommendations.

vii. Reagents

11% Formaldehyde Solution (50 mL) contained 14.9 ml of 37% formaldehyde (final conc. 11%), 1 ml of 5M NaCl (final conc. 0.1 M), 100 μl of 0.5M EDTA (pH 8) (final conc. 1 mM), 50 μl of 0.5M EGTA (pH 8) (final conc. 0.5 mM), and 2.5 ml 1M Hepes (pH 7.5) (final conc. 50 mM).

Block Solution contained 0.5% BSA (w/v) in PBS and 500 mg BSA in 100 ml PBS. Block solution may be prepared up to about 4 days prior to use.

Lysis buffer 1 (LB1) (500 ml) contained 25 ml of 1 M Hepes-KOH, pH 7.5; 14 ml of 5M NaCl; 1 ml of 0.5M EDTA, pH 8.0; 50 ml of 100% Glycerol solution; 25 ml of 10% NP-40; and 12.5 ml of 10% Triton X-100. The pH was adjusted to 7.5. The buffer was sterile-filtered, and stored at 4° C. The pH was re-checked immediately prior to use.

Lysis buffer 2 (LB2) (1000 ml) contained 10 ml of 1 M Tris-HCL, pH 8.0; 40 ml of 5 M NaCl; 2 ml of 0.5M EDTA, pH 8.0; and 2 ml of 0.5M EGTA, pH 8.0. The pH was adjusted to 8.0. The buffer was sterile-filtered, and stored at 4° C. The pH was re-checked immediately prior to use.

Sonication buffer (500 ml) contained 25 ml of 1M Hepes-KOH, pH 7.5; 14 ml of 5M NaCl; 1 ml of 0.5M EDTA, pH 8.0; 50 ml of 10% Triton X-100; 10 ml of 5% Na-deoxycholate; and 5 ml of 10% SDS. The pH was adjusted to 7.5. The buffer was sterile-filtered, and stored at 4° C. The pH was re-checked immediately prior to use.

Proteinase inhibitors were included in the LB1, LB2, and Sonication buffer.

Wash Buffer 2 (500 ml) contained 25 ml of 1M Hepes-KOH, pH 7.5; 35 ml of 5M NaCl; 1 ml of 0.5M EDTA, pH 8.0; 50 ml of 10% Triton X-100; 10 ml of 5% Na-deoxycholate; and 5 ml of 10% SDS. The pH was adjusted to 7.5. The buffer was sterile-filtered, and stored at 4° C. The pH was re-checked immediately prior to use.

Wash Buffer 3 (500 ml) contained 10 ml of 1M Tris-HCL, pH 8.0; 1 ml of 0.5M EDTA, pH 8.0; 125 ml of 1M LiCl solution; 25 ml of 10% NP-40; and 50 ml of 5% Na-deoxycholate. The pH was adjusted to 8.0. The buffer was sterile-filtered, and stored at 4° C. The pH was re-checked immediately prior to use.

ChIP elution Buffer (500 ml) contained 25 ml of 1 M Tris-HCL, pH 8.0; 10 ml of 0.5M EDTA, pH 8.0; 50 ml of 10% SDS; and 415 ml of ddH₂O. The pH was adjusted to 7.5. The buffer was sterile-filtered, and stored at 4° C. The pH was re-checked immediately prior to use.

I. Analysis of ChIP-Seq Results

All obtained reads from each sample were trimmed using trim_galore 0.4.1 requiring a Phred score≥20 and a read length≥30. The trimmed reads were mapped against the human genome (hg19 build) using Bowtie (version 1.1.2) with the parameters: -v 2 -m 1 -S -t. All unmapped reads, non-uniquely mapped reads and PCR duplicates were removed. All the ChIP-seq peaks were identified using MACS2 with the parameters: -q 0.01—SPMR. The ChIP-seq signal was visualized in the UCSC genome browser. ChIP-seq peaks that are at least 2 kb away from annotated promoters (RefSeq, Ensemble and UCSC Known Gene databases combined) were selected as distal ChIP-seq peaks.

J. RNA-Seq

This protocol is a modified version of the following protocols: MagMAX mirVana Total RNA Isolation Kit User Guide (Applied Biosystems #MAN0011131 Rev B.0), NEBNext Poly(A) mRNA Magnetic Isolation Module (E7490), and NEBNext Ultra Directional RNA Library Prep Kit for Illumina (E7420) (New England Biosystems #E74901).

The MagMAX mirVana kit instructions (the section titled “Isolate RNA from cells” on pages 14-17) were used for isolation of total RNA from cells in culture. Two hundred μl of Lysis Binding Mix was used per well of the multiwell plate containing adherent cells (usually a 24-well plate).

For mRNA isolation and library prep, the NEBNext Poly(A) mRNA Magnetic Isolation Module and Directional Prep kit was used. RNA isolated from cells above was quantified, and prepared in 500 μg of each sample in 50 μl of nuclease-free water. This protocol may be run in microfuge tubes or in a 96-well plate.

The 80% ethanol was prepared fresh, and all elutions are done in 0.1× TE Buffer. For steps requiring Ampure XP beads, beads were at room temperature before use. Sample volumes were measured first and beads were pipetted. Section 1.9B (not 1.9A) was used for NEBNext Multiplex Oligos for Illumina (#E6609). Before starting the PCR enrichment, cDNA was quantified using the Qubit (DNA High Sensitivity Kit, ThermoFisher #Q32854). The PCR reaction was run for 12 cycles.

After purification of the PCR Reaction (Step 1.10), the libraries were quantified using the Qubit DNA High Sensitivity Kit. 1 μl of each sample were diluted to 1-2 ng/μl to run on the Bioanalyzer (DNA High Sensitivity Kit, Agilent #5067-4626). If Bioanalyzer peaks were not clean (one narrow peak around 300 bp), the AMPure XP bead cleanup step was repeated using a 0.9× or 1.0× beads:sample ratio. Then, the samples were quantified again with the Qubit, and run again on the Bioanalyzer (1-2 ng/μl).

Nuclear RNA from INTACT-purified nuclei or whole neocortical nuclei was converted to cDNA and amplified with the Nugen Ovation RNA-seq System V2. Libraries were sequenced using the Illumina HiSeq 2500.

K. RNA-Seq Data Analysis

All obtained reads from each sample were mapped against the human genome (hg19 build) using STAR_2.5.2b, which allows mapping across splice sites by reads segmentation (Dobin et al., Bioinformatics (2012) 29 (1): 15-21, which is hereby incorporated by reference in its entirety). The uniquely mapped reads were subsequently assembled into transcripts guided by reference annotation (RefSeq gene models) (Pruitt et al., Nucleic Acids Res. 2012 January; 40(Database issue): D130-D135, which is incorporated by reference in its entirety) with Cuffnorm v2.2.1 (Trapnell et al., Nature Protocols 7, 562-578 (2012), which is hereby incorporated by reference in its entirety). The expression level of each gene was quantified with normalized FPKM (fragments per kilobase of exon per million mapped fragments). The differentially expressed genes were called using Cuffdiff v2.2.1 with q value<0.01 and log2 fold change>=1 or <=−1.

L. ATAC-Seq

Hepatocytes were seeded overnight, then the serum and other factors were removed. After 2-3 hours, the cells were treated with the compound and incubated overnight. The cells were harvested and the nuclei were prepared for the transposition reaction. 50,000 bead bound nuclei were transposed using Tn5 transposase (Illumina FC-121-1030) as described in Mo et al., 2015, Neuron 86, 1369-1384, which is hereby incorporated by reference in its entirety. After 9-12 cycles of PCR amplification, libraries were sequenced on an Illumina HiSeq 2000. PCR was performed using barcoded primers with extension at 72° C. for 5 minutes, PCR, then the final PCR product was sequenced.

All obtained reads from each sample were trimmed using trim_galore 0.4.1 requiring Phred score≥20 and read length≥30 for data analysis. The trimmed reads were mapped against the human genome (hg19 build) using Bowtie2 (version 2.2.9) with the parameters: -t -q -N 1 -L 25 -X 2000 no-mixed no-discordant. All unmapped reads, non-uniquely mapped reads and PCR duplicates were removed. All the ATAC-seq peaks were called using MACS2 with the parameters -nolambda -nomodel -q 0.01 -SPMR. The ATAC-seq signal was visualized in the UCSC genome browser. ATAC-seq peaks that were at least 2 kb away from annotated promoters (RefSeq, Ensemble and UCSC Known Gene databases combined) were selected as distal ATAC-seq peaks.

M. qRT-PCR

qRT-PCR was performed as described in North et al., PNAS, 107(40) 17315-17320 (2010), which is hereby incorporated by reference in its entirety. Prior to qRT-PCR analysis, cell medium was removed and replaced with RLT Buffer for RNA extraction (Qiagen RNeasy 96 QIAcube HT Kit Cat #74171). Cells were processed for RNA extraction using RNeasy 96 kit (Qiagen Cat #74182). For Taqman qPCR analysis, cDNA was synthesized using High-Capacity cDNA Reverse Transcription Kit (ThermoFisher Scientific cat:4368813 or 4368814) according to manufacturer instructions. qRT-PCR was performed with cDNA using the iQ5 Multicolor rtPCR Detection system from BioRad with 60° C. annealing. Samples were amplified using the following Taqman probes from ThermoFisher for each target: Hs01552217_m1 (human PNPLA3), Mm00504420_m1 (mouse PNPLA3); Hs00164004_m1 (COL1A1); Hs01078136_m1 (JAK2); Hs00895377_m1 (SYK); Hs00234508_m1 (mTOR); Hs00998018_m1 (PDGFRA); Hs00909233_m1 (GFAP); 4352341E (ACTB); 4326320E (GUSB); 4326319E (B2M); and 4326317E (GAPDH).

Analysis of the fold changes in expression as measured by qRT-PCR were performed using the technique below. The control was DMSO, and the treatment was the selected compound (CPD). The internal control was GAPDH or B-Actin (or otherwise indicated), and the gene of interest is the target. First, the averages of the 4 conditions were calculated for normalization: DMSO:GAPDH, DMSO:Target, CPD: GAPDH, and CPD:Target. Next, the ΔCT of both control and treatment were calculated to normalize to internal control (GAPDH) using (DMSO:Target)−(DMSO:GAPDH)=ΔCT control and (CPD:Target)−(CPD: GAPDH)=ΔCT experimental. Then, the ΔΔCT was calculated by ΔCT experimental−ΔCT control. The Expression Fold Change (or Relative Quantification, abbreviated as RQ) was calculated by 2−(ΔΔCT) (2-fold expression change was shown by RNA-Seq results provided herein).

In some examples, RQ Min and RQ Max values are also reported. RQ Min and RQ Max are the minimum and maximum relative levels of gene expression in the test samples, respectively. They were calculated using the confidence level set in the analysis settings and the confidence level was set to one standard deviation (SD). These values were calculated using standard deviation as follows: RQ Min=2−(ΔΔCT-SD); and RQ Max=2−(ΔΔCT+SD).

N. Chromatin Interaction Analysis by Paired-End Tag Sequencing (ChIA-PET)

ChIA-PET is performed as previously described in Chepelev et al. (2012) Cell Res. 22, 490-503; Fullwood et al. (2009) Nature 462, 58-64; Goh et al. (2012) J. Vis. Exp., http://dx.doi.org/10.3791/3770; Li et al. (2012) Cell 148, 84-98; and Dowen et al. (2014) Cell 159, 374-387, which are each hereby incorporated by reference in their entireties. Briefly, embryonic stem (ES) cells (up to 1×10⁸ cells) are treated with 1% formaldehyde at room temperature for 20 minutes and then neutralized using 0.2M glycine. The crosslinked chromatin is fragmented by sonication to size lengths of 300-700 bp. The anti-SMC1 antibody (Bethyl, A300-055A) is used to enrich SMC1-bound chromatin fragments. A portion of ChIP DNA is eluted from antibody-coated beads for concentration quantification and for enrichment analysis using quantitative PCR. For ChIA-PET library construction ChIP DNA fragments are end-repaired using T4 DNA polymerase (NEB). ChIP DNA fragments are divided into two aliquots and either linker A or linker B is ligated to the fragment ends. The two linkers differ by two nucleotides which are used as a nucleotide barcode (Linker A with CG; Linker B with AT). After linker ligation, the two samples are combined and prepared for proximity ligation by diluting in a 20 ml volume to minimize ligations between different DNA-protein complexes. The proximity ligation reaction is performed with T4 DNA ligase (Fermentas) and incubated without rocking at 22° C. for 20 hours. During the proximity ligation DNA fragments with the same linker sequence are ligated within the same chromatin complex, which generated the ligation products with homodimeric linker composition. However, chimeric ligations between DNA fragments from different chromatin complexes could also occur, thus producing ligation products with heterodimeric linker composition. These heterodimeric linker products are used to assess the frequency of nonspecific ligations and were then removed.

i. Day 1

The cells are crosslinked as described for ChIP. Frozen cell pellets are stored in the −80° C. freezer until ready to use. This protocol requires at least 3×10⁸ cells frozen in six 15 ml Falcon tubes (50 million cells per tube). Six 100 μl Protein G Dynabeads (for each ChIA-PET sample) are added to six 1.5 ml Eppendorf tubes on ice. Beads are washed three times with 1.5 ml Block solution, and incubated end over end at 4° C. for 10 minutes between each washing step to allow for efficient blocking. Protein G Dynabeads are resuspended in 250 μl of Block solution in each of six tubes and 10 μg of SMC1 antibody (Bethyl A300-055A) is added to each tube. The bead-antibody mixes are incubated at 4° C. end-over-end overnight.

ii. Day 2

Beads are washed three times with 1.5 ml Block solution to remove unbound IgG and incubated end-over-end at 4° C. for 10 minutes each time. Smc1-bound beads are resuspended in 100 μl of Block solution and stored at 4° C. Final lysis buffer 1 (8 ml per sample) is prepared by adding 50× Protease inhibitor cocktail solution to Lysis buffer 1 (LB1) (1:50). Eight ml of Final lysis buffer 1 was added to each frozen cell pellet (8 ml per sample×6). The cells are thoroughly resuspended and thawed on ice by pipetting up and down. The cell suspension is incubated again end-over-end for 10 minutes at 4° C. The suspension is centrifuged at 1,350 g for 5 minutes at 4° C. Concurrently, Final lysis buffer 2 (8 ml per sample) is prepared by adding 50× Protease inhibitor cocktail solution to lysis buffer 2 (LB2) (1:50)

After centrifugation, the supernatant is discarded, and the nuclei are thoroughly resuspended in 8 ml Final lysis buffer 2 by pipetting up and down. The cell suspension is incubated end-over-end for 10 minutes at 4° C. The suspension is centrifuged at 1,350 g for 5 minutes at 4° C. During incubation and centrifugation, the Final sonication buffer (15 ml per sample) is prepared by adding 50× Protease inhibitor cocktail solution to sonication buffer (1:50). The supernatant is discarded, and the nuclei are fully resuspended in 15 ml Final sonication buffer by pipetting up and down. The nuclear extract is extracted to fifteen 1 ml Covaris Evolution E220 sonication tubes on ice. An aliquot of 10 μl is used to check the size of unsonicated chromatin on a gel.

A Covaris sonicator is programmed according to manufacturer's instructions (12 minutes per 20 million cells=12×15=3 hours). The samples are sequentially sequenced as described above. The goal is to break chromatin DNA to 200-600 bp. If sonication fragments are too big, false positives become more frequent. The sonicated nuclear extract is dispensed into 1.5 ml Eppendorf tubes. 1.5 ml samples are centrifuged at full speed at 4° C. for 10 minutes. Supernatant (SNE) is pooled into a new pre-cooled 50 ml Falcon tube, and brought to a volume of 18 ml with sonication buffer. Two tubes of 50 μl were taken as input and to check the size of fragments. 250 μl of ChIP elution buffer is added and reverse crosslinking occurs at 65° C. overnight in the oven After reversal of crosslinking, the size of sonication fragments is determined on a gel.

Three ml of sonicated extract is added to 100 μl Protein G beads with SMC1 antibodies in each of six clean 15 ml Falcon tubes. The tubes containing SNE-bead mix are incubated end-over-end at 4° C. overnight (14 to 18 hours)

iii. Day 3

Half the volume (1.5 ml) of the SNE-bead mix is added to each of six pre-chilled tubes and SNE is removed using a magnet. The tubes are sequentially washed as follows: 1) 1.5 ml of Sonication buffer is added, the beads are resuspended and rotated for 5 minutes at 4° C. for binding, then the liquid was removed (step performed twice); 2) 1.5 ml of high-salt sonication buffer is added, and the beads are resuspended and rotated for 5 minutes at 4° C. for binding, then the liquid is removed (step performed twice); 3) 1.5 ml of high-salt sonication buffer is added, and the beads are resuspended and rotated for 5 minutes at 4° C. for binding, then the liquid is removed (step performed twice); 4) 1.5 ml of LiCl buffer is added, and the cells are resuspended and incubated end-over-end for 5 minutes for binding, then the liquid is removed (step performed twice); 5) 1.5 ml of 1× TE+0.2% Triton X-100 is used to wash the cells for 5 minutes for binding, then the liquid is removed; and 1.5 ml of ice-cold TE Buffer is used to wash the cells for 30 seconds for binding, then the liquid is removed (step performed twice). Beads from all six tubes are sequentially resuspended in beads in one 1,000 ul tube of 1× ice-cold TE buffer.

ChIP-DNA is quantified using the following protocol. Ten percent of beads (by volume), or 100 μl, are transferred into a new 1.5 ml tube, using a magnet. Beads are resuspended in 300 μl of ChIP elution buffer and the tube is rotated with beads for 1 hour at 65° C. The tube with beads is placed on a magnet and the eluate was transferred to a fresh DNA LoBind Eppendorf tube. The eluate is incubated overnight at 65° C. oven without rotating. Immuno-precipitated samples are transferred to fresh tubes, and 300 μl of TE buffer is added to the immuno-precipitants and Input samples to dilute. Five μl of RNase A (20 mg/ml) is added, and the tube is incubated at 37° C. for 30 minutes.

Following incubation, 3 μl of 1M CaCl₂ and 7 μl of 20 mg/ml Proteinase K is added to the tube and incubated 1.5 hours at 55° C. MaXtract High Density 2 ml gel tubes (Qiagen) were prepared by centrifuging them at full speed for 30 seconds at RT. 600 μl of phenol/chloroform/isoamyl alcohol is added to each proteinase K reaction. About 1.2 ml of the mixtures is transferred to the MaXtract tubes. Tubes are spun at 16,000 g for 5 minutes at RT. The aqueous phase is transferred to two clean DNA LoBind tubes (300 μl in each tube), and 1 μl glycogen, 30 μl of 3M sodium acetate, and 900 μl ethanol is added. The mixture is allowed to precipitate overnight at −20° C. or for 1 hour at −80° C.

The mixture is spun down at maximum speed for 20 minutes at 4° C., ethanol is removed, and the pellets are washed with 1 ml of 75% ethanol by spinning tubes down at maximum speed for 5 minutes at 4° C. All remnants of ethanol are removed, and pellets are dried for 5 minutes at RT. H₂O is added to each tube. Each tube is allowed to stand for 5 minutes, and vortexed briefly. DNA from both tubes is combined to obtain 50 μl of IP and 100 μl of Input DNA.

The amount of DNA collected is quantitated by ChIP using Qubit (Invitrogen #Q32856). One μl intercalating dye is combined with each measure 1 μl of sample. Two standards that come with the kit are used. DNA from only 10% of the beads is being measured. About 400 ng of chromatin in 900 μl of bead suspension is obtained with a good enrichment at enhancers and promoters as measured by qPCR.

iv. Day 3 or 4

End-blunting of ChIP-DNA is performed on the beads using the following protocol. The remaining chromatin/beads are split by pipetting, and 450 μl of bead suspension is aliquoted into 2 tubes. Beads are collected on a magnet. Supernatant is removed, and then the beads are resuspended in the following reaction mix: 70 μl 10× NEB buffer 2.1 (NEB, M0203L), 7 μl 10 mM dNTPs, 615.8 μl dH₂0, and 7.41 of 3U/μl T4 DNA Polymerase (NEB, M0203L). The beads are incubated at 37° C. with rotation for 40 minutes. Beads are collected with a magnet, then the beads are washed 3 times with 1 ml ice-cold ChIA-PET Wash Buffer (30 seconds per each wash).

On-Bead A-tailing was performed by preparing Klenow (3′ to 5′exo-) master mix as stated below: 70 μl 10× NEB buffer 2, 7 μl 10 mM dATP, 616 μl dH20, and 7 μl of 3U/μl Klenow (3′ to 5′exo-) (NEB, M0212L). The mixture is incubated at 37° C. with rotation for 50 minutes. Beads are collected with a magnet, then beads are washed 3 times with 1 ml of ice-cold ChIA-PET Wash Buffer (30 seconds per each wash).

Linkers are thawed gently on ice. Linkers are mixed well with water gently by pipetting, then with PEG buffer, then gently vortexed. Then, 1394 μl of master mix and 6 μl of ligase is added per tube and mixed by inversion. Parafilm is put on the tube, and the tube is incubated at 16° C. with rotation overnight (at least 16 hours). The biotinylated linker was ligated to ChIP-DNA on beads by setting up the following reaction mix and adding reagents in order: 1110 μl dH₂0, 4 μl 200 ng/μl biotinylated bridge linker, 280 μl 5× T4 DNA ligase buffer with PEG (Invitrogen), and 6 μl 30 U/μl T4 DNA ligase (Fermentas).

v. Day 5

Exonuclease lambda/Exonuclease I On-Bead digestion was performed using the following protocol. Beads were collected with a magnet and washed 3 times with 1 ml of ice-cold ChIA-PET Wash Buffer (30 seconds per each wash). The Wash buffer is removed from beads, then resuspended in the following reaction mix: 70 μl 10× lambda nuclease buffer (NEB, M0262L), 618 μl nuclease-free dH20, 6 μl 5 U/μl Lambda Exonuclease (NEB, M0262L), and 6 μl Exonuclease I (NEB, M0293L). The reaction is incubated at 37° C. with rotation for 1 hour. Beads are collected with a magnet, and beads are washed 3 times with 1 ml ice-cold ChIA-PET Wash Buffer (30 seconds per each wash).

Chromatin complexes are eluted off the beads by removing all residual buffer and resuspending the beads in 300 μl of ChIP elution buffer. The tube with beads is rotated 1 hour at 65° C. The tube is placed on a magnet and the eluate is transferred to a fresh DNA LoBind Eppendorf tube. The eluate is incubated overnight at 65° C. in an oven without rotating.

vi. Day 6

The eluted sample is transferred to a fresh tube and 300 μl of TE buffer is added to dilute the SDS. Three μl of RNase A (30 mg/ml) is added to the tube, and the mixture is incubated at 37° C. for 30 minutes. Following incubation, 3 μl of 1M CaCl₂ and 7 μl of 20 mg/ml Proteinase K is added, and the tube is incubated again for 1.5 hours at 55° C. MaXtract High Density 2 ml gel tubes (Qiagen) are precipitated by centrifuging them at full speed for 30 seconds at RT. Six hundred μl of phenol/chloroform/isoamyl alcohol is added to each proteinase K reaction, and about 1.2 ml of the mixture is transferred to the MaXtract tubes. Tubes are spun at 16,000 g for 5 minutes at RT.

The aqueous phase is transferred to two clean DNA LoBind tubes (300 μl in each tube), and 1 μl glycogen, 30 μl of 3M sodium acetate, and 900 μl ethanol is added. The mixture is precipitated for 1 hour at −80° C. The tubes are spun down at maximum speed for 30 minutes at 4° C., and the ethanol is removed. The pellets are washed with 1 ml of 75% ethanol by spinning tubes down at maximum speed for 5 minutes at 4° C. Remnants of ethanol are removed, and the pellets are dried for 5 minutes at RT. Thirty μl of H₂O is added to the pellet and allowed to stand for 5 minutes. The pellet mixture is vortexed briefly, and spun down to collect the DNA.

Qubit and DNA High Sensitivity ChIP are performed to quantify and assess the quality of proximity ligated DNA products. About 120 ng of the product is obtained.

vii. Day 7

Components for Nextera tagmentation are then prepared. One hundred ng of DNA is divided into four 25 μl reactions containing 12.5 μl 2× Tagmentation buffer (Nextera), 1 μl nuclease-free dH₂0, 2.5 μl Tn5 enzyme(Nextera), and 9 μl DNA (25 ng). Fragments of each of the reactions are analyzed on a Bioanalyzer for quality control.

The reactions are incubated at 55° C. for 5 minutes, then at 10° C. for 10 minutes. Twenty-five μl of H₂O is added, and tagmented DNA is purified using Zymo columns. Three hundred fifty μl of Binding Buffer is added to the sample, and the mixture is loaded into a column and spun at 13,000 rpm for 30 seconds. The flow through is re-applied and the columns are spun again. The columns are washed twice with 200 μl of wash buffer and spun for 1 minute to dry the membrane. The column is transferred to a clean Eppendorf tube and 25 μl of Elution buffer is added. The tube is spun down for 1 minute. This step is repeated with another 25 μl of elution buffer. All tagmented DNA is combined into one tube.

ChIA-PETs are immobilized on Streptavidin beads using the following steps. 2× B&W Buffer (40 ml) is prepared as follows for coupling of nucleic acids: 400 μl 1M Tris-HCl pH 8.0 (10 mM final), 80 μl 1M EDTA (1 mM final), 16 ml 5M NaCl (2M final), and 23.52 ml dH₂O. 1× B&W Buffer (40 ml total) is prepared by adding 20 ml dH₂O to 20 ml of the 2× B&W Buffer.

MyOne Streptavidin Dynabeads M-280 are allowed to come to room temperature for 30 minutes, and 30 μl of beads are transferred to a new 1.5 ml tube. Beads are washed with 150 μl of 2× B&W Buffer twice. Beads are resuspended in 100 μl of iBlock buffer (Applied Biosystems), and mixed. The mixture is incubated at RT for 45 minutes on a rotator.

I-BLOCK Reagent is prepared to contain: 0.2% I-Block reagent (0.2 g), 1× PBS or 1× TBS (10 ml 10× PBS or 10× TBS), 0.05% Tween-20 (50 μl), and H₂O to 100 ml. 10× PBS and I-BLOCK reagent is added to H₂O, and the mixture is microwaved for 40 seconds (not allowed to boil), then stirred. Tween-20 is added after the solution is cooled. The solution remains opaque, but particles are dissolved. The solution is cooled to RT for use.

During incubation of beads, 500 ng of sheared genomic DNA is added to 50 μl of H₂O and 50 μl of 2× B&W Buffer. When the beads finish incubating with the iBLOCK buffer, they are washed twice with 200 μl of 1× B&W buffer. The wash buffer is discarded, and 100 μl of the sheared genomic DNA is added. The mixture is incubated with rotation for 30 minutes at RT. The beads are washed twice with 200 μl of 1× B&W buffer. Tagmented DNA is added to the beads with an equal volume of 2× B&W buffer and incubated for 45 minutes at RT with rotation. The beads are washed 5 times with 500 μl of 2×SSC/0.5% SDS buffer (30 seconds each time) followed by 2 washes with 500 ml of 1× B&W Buffer and incubating each after wash for 5 minutes at RT with rotation. The beads are washed once with 100 μl elution buffer (EB) from a Qiagen Kit by resuspending beads gently and putting the tube on a magnet. The supernatant is removed from the beads, and they were resuspended in 30 μl of EB.

A paired end sequencing library is constructed on beads using the following protocol. Ten μl of beads are tested by PCR with 10 cycles of amplification. The 50 μl of the PCR mixture contains: 10 μl of bead DNA, 15 μl NPM mix (from Illumina Nextera kit), 5 μl of PPC PCR primer, 5 μl of Index Primer 1 (i7), 5 μl of Index Primer 2 (i5), and 10 μl of H₂O. PCR is performed using the following cycle conditions: denaturing the DNA at 72° C. for 3 minutes, then 10-12 cycles of 98° C. for 10 seconds, 63° C. for 30 seconds, and 72° C. for 50 seconds, and a final extension of 72° C. for 5 minutes. The number of cycles is adjusted to obtain about 300 ng of DNA total with four 25 μl reactions. The PCR product may be held at 4° C. for an indefinite amount of time.

The PCR product was cleaned-up using AMPure beads. Beads are allowed to come to RT for 30 minutes before using. Fifty μl of the PCR reaction is transferred to a new Low-Bind Tube and (1.8× volume) 90 μl of AMPure beads is added. The mixture is pipetted well and incubated at RT for 5 minutes. A magnet is used for 3 minutes to collect beads and remove the supernatant. Three hundred μl of freshly prepared 80% ethanol is added to the beads on the magnet, and the ethanol is carefully dicarded. The wash is repeated, and then all ethanol is removed. The beads are dried on the magnet rack for 10 minutes. Ten μl EB is added to the beads, mixed well, and incubated for 5 minutes at RT. The eluate is collected, and 1 μl of eluate is used for Qubit and Bioanalyzer.

The library is cloned to verify complexity using the following protocol. One μl of the library is diluted at 1:10. A PCR reaction is performed as described below. Primers that anneal to Illumina adapters are chosen (Tm=52.2° C.). The PCR reaction mixture (total volume: 50 μl) contains the following: 10 μl of 5× GoTaq buffer, 1 μl of 10 mM dNTP, 5 μl of 10 μM primer mix, 0.25 μl of GoTaq polymerase, 1 μl of diluted template DNA, and 32.75 μl of H₂O. PCR is performed using the following cycle conditions: denaturing the DNA at 95° C. for 2 minutes and 20 cycles at the following conditions: 95° C. for 60 seconds, 50° C. for 60 seconds, and 72° C. for 30 seconds with a final extension at 72° C. for 5 minutes. The PCR product may be held at 4° C. for an indefinite amount of time.

The PCR product is ligated with the pGEM® T-Easy vector (Promega) protocol. Five μl of 2× T4 Quick ligase buffer, 1 μl of pGEM® T-Easy vector, 1 μl of T4 ligase, 1 μl of PCR product, and 2 μl of H₂O are combined to a total volume of 10 μl. The product is incubated for 1 hour at RT and 2 μl is used to transform Stellar competent cells. Two hundred μl of 500 μl of cells are plated in SOC media. The next day, 20 colonies are selected for Sanger sequencing using a T7 promoter primer. 60% clones had a full adapter, and 15% had a partial adapter.

viii. Reagents

Protein G Dynabeads for 10 samples are from Invitrogen Dynal, Cat #10003D. Block solution (50 ml) contains 0.25 g BSA dissolved in 50 ml of ddH2O (0.5% BSA, w/v), and is stored at 4° C. for 2 days before use.

Lysis buffer 1 (LB1) (500 ml) contains 25 ml of 1M Hepes-KOH, pH 7.5; 14 ml of 5M NaCl; 1 ml of 0.5 M EDTA, pH 8.0; 50 ml of 100% Glycerol solution; 25 ml of 10% NP-40; and 12.5 ml of 10% Triton X-100. The pH is adjusted to 7.5. The buffer is sterile-filtered, and stored at 4° C. The pH is re-checked immediately prior to use. Lysis buffer 2 (LB2) (1000 ml) contains 10 ml of 1M Tris-HCL, pH 8.0; 40 ml of 5 M NaCl; 2 ml of 0.5 M EDTA, pH 8.0; and 2 ml of 0.5 M EGTA, pH 8.0. The pH is adjusted to 8.0. The buffer is sterile-filtered, and stored at 4° C. The pH is re-checked immediately prior to use.

Sonication buffer (500 ml) contains 25 ml of 1M Hepes-KOH, pH 7.5; 14 ml of 5M NaCl; 1 ml of 0.5 M EDTA, pH 8.0; 50 ml of 10% Triton X-100; 10 ml of 5% Na-deoxycholate; and 5 ml of 10% SDS. The buffer is sterile-filtered, and stored at 4° C. The pH is re-checked immediately prior to use. High-salt sonication buffer (500 ml) contains 25 ml of 1M Hepes-KOH, pH 7.5; 35 ml of 5M NaCl; 1 ml of 0.5 M EDTA, pH 8.0; 50 ml of 10% Triton X-100; 10 ml of 5% Na-deoxycholate; and 5 ml of 10% SDS. The buffer is sterile-filtered, and stored at 4° C. The pH is re-checked immediately prior to use.

LiCl wash buffer (500 ml) contains 10 ml of 1M Tris-HCL, pH 8.0; 1 ml of 0.5M EDTA, pH 8.0; 125 ml of 1M LiCl solution; 25 ml of 10% NP-40; and 50 ml of 5% Na-deoxycholate. The pH is adjusted to 8.0. The buffer is sterile-filtered, and stored at 4° C. The pH is re-checked immediately prior to use.

Elution buffer (500 ml) used to quantify the amount of ChIP DNA contains 25 ml of 1M Tris-HCL, pH 8.0; 10 ml of 0.5M EDTA, pH 8.0; 50 ml of 10% SDS; and 415 ml of ddH₂O. The pH is adjusted to 8.0. The buffer is sterile-filtered, and stored at 4° C. The pH is re-checked immediately prior to use.

ChIA-PET Wash Buffer (50 ml) contains 500 μl of 1M Tris-HCl, pH 8.0 (final 10 mM); 100 μl of 0.5M EDTA, pH 8.0 (final 1 mM); 5 ml of 5M NaCl (final 500 mM); and 44.4 ml of dH₂0.

O. HiChIP

Alternatively to ChIA-PET, HiChIP was used to analyze chromatin interactions and conformation. HiChIP requires fewer cells than ChIA-PET.

i. Cell Crosslinking

Cells were cross-linked as described in the ChIP protocol above. Crosslinked cells were either stored as pellets at −80° C. or used for HiChIP immediately after flash-freezing the cells.

ii. Lysis and Restriction

Fifteen million cross-linked cells were resuspended in 500 μL of ice-cold Hi-C Lysis Buffer and rotated at 4° C. for 30 minutes. For cell amounts greater than 15 million, the pellet was split in half for contact generation and then recombined for sonication. Cells were spun down at 2500 g for 5 minutes, and the supernatant was discarded. The pelleted nuclei were washed once with 500 μL of ice-cold Hi-C Lysis Buffer. The supernatant was removed, and the pellet was resuspended in 100 μL of 0.5% SDS. The resuspension was incubated at 62° C. for 10 minutes, and then 285 μL of H₂O and 50 μL of 10% Triton X-100 were added to quench the SDS. The resuspension was mixed well, and incubated at 37° C. for 15 minutes. Fifty μL of 10× NEB Buffer 2 and 375 U of Mbol restriction enzyme (NEB, R0147) was added to the mixture to digest chromatin for 2 hours at 37° C. with rotation. For lower starting material, less restriction enzyme is used: 15 μL was used for 10-15 million cells, 8 μL for 5 million cells, and 4 μL for 1 million cells. Heat (62° C. for 20 minutes) was used to inactivate MboI.

iii. Biotin Incorporation and Proximity Ligation

To fill in the restriction fragment overhangs and mark the DNA ends with biotin, 52 μL of fill-in master mix was reacted by combining 37.5 μL of 0.4 mM biotin-dATP (Thermo 19524016); 1.5 μL of 10 mM dCTP, dGTP, and dTTP; and 10 μL of 5 U/μL DNA Polymerase I, Large (Klenow) Fragment (NEB, M0210). The mixture was incubated at 37° C. for 1 hour with rotation.

948 μL of ligation master mix was added. Ligation Master Mix contains 150 μL of 10× NEB T4 DNA ligase buffer with 10 mM ATP (NEB, B0202); 125 μL of 10% Triton X-100; 3 μL of 50 mg/mL BSA; 10 μL of 400 U/μL T4 DNA Ligase (NEB, M0202); and 660 μL of water. The mixture was incubated at room temperature for 4 hours with rotation. The nuclei were pelleted at 2500 g for 5 minutes, and the supernatant was removed.

iv. Sonication

For sonication, the pellet was brought up to 1000 μL in Nuclear Lysis Buffer. The sample was transferred to a Covaris millitube, and the DNA was sheared using a Covaris® E220Evolution™ with the manufacturer recommended parameters. Each tube (15 million cells) was sonicated for 4 minutes under the following conditions: Fill Level 5; Duty Cycle 5%; PIP 140; and Cycles/Burst 200.

v. Preclearing, Immunoprecipitation, IP Bead Capture, and Washes

The sample was clarified for 15 minutes at 16,100 g at 4° C. The sample is split into 2 tubes of about 400 μL each and 750 μL of ChIP Dilution Buffer is added. For the Smc1a antibody (Bethyl A300-055A), the sample is diluted 1:2 in ChIP Dilution Buffer to achieve an SDS concentration of 0.33%. 60 μL of Protein G beads were washed for every 10 million cells in ChIP Dilution Buffer. Amounts of beads (for preclearing and capture) and antibodies were adjusted linearly for different amounts of cell starting material. Protein G beads were resuspended in 50 μL of Dilution Buffer per tube (1004, per HiChIP). The sample was rotated at 4° C. for 1 hour. The samples were put on a magnet, and the supernatant was transferred into new tubes. 7.5 μg of antibody was added for every 10 million cells, and the mixture was incubated at 4° C. overnight with rotation. Another 60 μL of Protein G beads for every 10 million cells in ChIP Dilution Buffer was added. Protein G beads were resuspended in 50 μL of Dilution Buffer (100 μL per HiChIP), added to the sample, and rotated at 4° C. for 2 hours. The beads were washed three times each with Low Salt Wash Buffer, High Salt Wash Buffer, and LiCl Wash Buffer. Washing was performed at room temperature on a magnet by adding 500 μL of a wash buffer, swishing the beads back and forth twice by moving the sample relative to the magnet, and then removing the supernatant

vi. ChIP DNA Elution

ChIP sample beads were resuspended in 100 μL of fresh DNA Elution Buffer. The sample beads were incubated at RT for 10 minutes with rotation, followed by 3 minutes at 37° C. with shaking. ChIP samples were placed on a magnet, and the supernatant was removed to a fresh tube. Another 100 μL of DNA Elution Buffer was added to ChIP samples and incubations were repeated. ChIP sample supernatants were removed again and transferred to a new tube. There was about 200 μL of ChIP sample. Ten μL of Proteinase K (20 mg/ml) was added to each sample and incubated at 55° C. for 45 minutes with shaking. The temperature was increased to 67° C., and the samples were incubated for at least 1.5 hours with shaking. The DNA was Zymo-purified (Zymo Research, #D4014) and eluted into 10 μL of water. Post-ChIP DNA was quantified to estimate the amount of Tn5 needed to generate libraries at the correct size distribution. This assumed that contact libraries were generated properly, samples were not over sonicated, and that material was robustly captured on streptavidin beads. SMC1 HiChIP with 10 million cells had an expected yield of post-ChIP DNA from 15 ng-50 ng. For libraries with greater than 150 ng of post-ChIP DNA, materials were set aside and a maximum of 150 ng was taken into the biotin capture step.

vii. Biotin Pull-Down and Preparation for Illumina Sequencing

To prepare for biotin pull-down, 5 μL of Streptavidin C-1 beads were washed with Tween Wash Buffer. The beads were resuspended in 10 μL of 2× Biotin Binding Buffer and added to the samples. The beads were incubated at RT for 15 minutes with rotation. The beads were separated on a magnet, and the supernatant was discarded. The beads were washed twice by adding 500 μL of Tween Wash Buffer and incubated at 55° C. for 2 minutes while shaking. The beads were washed in 100 μL of 1× (diluted from 2×) TD Buffer. The beads were resuspended in 25 μL of 2× TD Buffer, 2.5 μL of Tn5 for each 50 ng of post-ChIP DNA, and water to a volume of 50 μL.

The Tn5 had a maximum amount of 4 μL. For example, for 25 ng of DNA transpose, 1.25 μL of Tn5 was added, while for 125 ng of DNA transpose, 4 μL of Tn5 was used. Using the correct amount of Tn5 resulted in proper size distribution. An over-transposed sample had shorter fragments and exhibited lower alignment rates (when the junction was close to a fragment end). An undertransposed sample has fragments that are too large to cluster properly on an Illumina sequencer. The library was amplified in 5 cycles and had enough complexity to be sequenced deeply and achieve proper size distribution regardless of the level of transposition of the library.

The beads were incubated at 55° C. with interval shaking for 10 minutes. Samples were placed on a magnet, and the supernatant was removed. Fifty mM EDTA was added to samples and incubated at 50° C. for 30 minutes. The samples were then quickly placed on a magnet, and the supernatant was removed. The samples were washed twice with 50 mM EDTA at 50° C. for 3 minutes, then were removed quickly from the magnet. Samples were washed twice in Tween Wash Buffer for 2 minutes at 55° C., then were removed quickly from the magnet. The samples were washed with 10 mM Tris-HCl, pH 8.0.

viii. PCR and Post-PCR Size Selection

The beads were resuspended in 50 μL of PCR master mix (use Nextera XT DNA library preparation kit from Illumina, #15028212 with dual-Index adapters #15055289). PCR was performed using the following program. The cycle number was estimated using one of two methods: (1) A first run of 5 cycles (72° C. for 5 minutes, 98° C. for 1 minute, 98° C. for 15 seconds, 63° C. for 30 seconds, 72° C. for 1 minute) is performed on a regular PCR and then the product is removed from the beads. Then, 0.25× SYBR green is added, and the sample is run on a qPCR. Samples are pulled out at the beginning of exponential amplification; or (2) Reactions are run on a PCR and the cycle number is estimated based on the amount of material from the post-ChIP Qubit (greater than 50 ng is run in 5 cycles, while approximately 50 ng is run in 6 cycles, 25 ng is run in 7 cycles, 12.5 ng is run in 8 cycles, etc.).

Libraries were placed on a magnet and eluted into new tubes. The libraries were purified using a kit form Zymo Research and eluted into 10 μL of water. A two-sided size selection was performed with AMPure XP beads. After PCR, the libraries were placed on a magnet and eluted into new tubes. Then, 25 μL of AMPure XP beads were added, and the supernatant was kept to capture fragments less than 700 bp. The supernatant was transferred to a new tube, and 15 μL of fresh beads were added to capture fragments greater than 300 bp. A final elution was performed from the Ampure XP beads into 10 μL of water. The library quality was verified using a Bioanalyzer.

ix. Buffers

Hi-C Lysis Buffer (10 mL) contains 100 μL of 1M Tris-HCl pH 8.0; 20 μL of 5M NaCl; 200 μL of 10% NP-40; 200 μL of 50× protease inhibitors; and 9.68 mL of water. Nuclear Lysis Buffer (10 mL) contains 500 μL of 1M Tris-HCl pH 7.5; 200 μL of 0.5M EDTA; 1 mL of 10% SDS; 200 μL of 50× Protease Inhibitor; and 8.3 mL of water. ChIP Dilution Buffer (10 mL) contains 10 μL of 10% SDS; 1.1 mL of 10% Triton X-100; 24 μL of 500 mM EDTA; 167 μL of 1M Tris pH 7.5; 334 μL of 5M NaCl; and 8.365 mL of water. Low Salt Wash Buffer (10 mL) contains 100 μL of 10% SDS; 1 mL of 10% Triton X-100; 40 μL of 0.5M EDTA; 200 μL of 1M Tris-HCl pH 7.5; 300 μL of 5M NaCl; and 8.36 mL of water. High Salt Wash Buffer (10 mL) contains 100 μL of 10% SDS; 1 mL of 10% Triton X-100; 40 μL of 0.5M EDTA; 200 μL of 1M Tris-HCl pH 7.5; 1 mL of 5M NaCl; and 7.66 mL of water. LiCl Wash Buffer (10 mL) contains 100 μL of 1M Tris pH 7.5; 500 μL of 5M LiCl; 1 mL of 10% NP-40; 1 mL of 10% Na-deoxycholate; 20 μL of 0.5M EDTA; and 7.38 mL of water.

DNA Elution Buffer (5 mL) contains 250 μL of fresh 1M NaHCO₃; 500 μL of 10% SDS; and 4.25 mL of water. Tween Wash Buffer (50 mL) contains 250 μL of 1M Tris-HCl pH 7.5; 50 μL of 0.5M EDTA; 10 mL of 5M NaCl; 250 μL of 10% Tween-20; and 39.45 mL of water. 2× Biotin Binding Buffer (10 mL) contains 100 μL 1M Tris-HCl pH 7.5; 20 μL of 0.5M; 4 mL of 5M NaCl; and 5.88 mL of water. 2× TD Buffer (1 mL) contains 20 μL of 1M Tris-HCl pH 7.5; 10 μL of 1M MgCl₂; 200 μL of 100% Dimethylformamide; and 770 μL of water.

P. Drug Dilutions for Administration to Hepatocytes

Prior to compound treatment of hepatocytes, 100 mM stock drugs in DMSO were diluted to 10 mM by mixing 0.1 mM of the stock drug in DMSO with 0.9 ml of DMSO to a final volume of 1.0 ml. Five μl of the diluted drug was added to each well, and 0.5 ml of media was added per well of drug. Each drug was analyzed in triplicate. Dilution to 1000× was performed by adding 5 μl of drug into 45 μl of media, and the 50 μl being added to 450 μl of media on cells.

Bioactive compounds were also administered to hepatocytes. To obtain 1000× stock of the bioactive compounds in 1 ml DMSO, 0.1 ml of 10,000× stock was combined with 0.9 ml DMSO.

Q. siRNA Knockdown

Primary human hepatocytes were reverse transfected with siRNA with 6 pmol siRNA using RNAiMAX Reagent (ThermoFisher Cat #13778030) in 24 well format, 1 μl per well. The following morning, the medium was removed and replaced with modified maintenance medium for an additional 24 hours. The entire treatment lasted 48 hours, at which point the medium was removed and replaced with RLT Buffer for RNA extraction (Qiagen RNeasy 96 QIAcube HT Kit Cat #74171). Cells were processed for qRT-PCR analysis and then levels of target mRNA were measured.

siRNAs were obtained from Dharmacon and are a pool of four siRNA duplex all designed to target distinct sites within the specific gene of interest (known as “SMARTpool”). The following siRNAs were used: D-001206-13-05 (non-targeting); M-003145-02-0005 (JAK1); M-003146-02-0005 (JAK2); M-003176-03-0005 (SYK); M-003008-03-0005 (mTOR); M-003162-04-0005 (PDGFRA), M-012723-01-0005 (SMAD1); M-003561-01-0005 (SMAD2); M-020067-00-0005 (SMAD3); M-003902-01-0005 (SMAD4); M-015791-00-0005 (SMADS); and M-016192-02-0005 (SMAD9); M-004924-02-0005 (ACVR1); and M-003520-01-0005 (NF-κB).

R. Mice Studies

A group of 6 mice (C57BL/6J strain), 3 male and 3 female, were administered with a candidate compound once daily via oral gavage for four consecutive days. Mice were sacrificed 4 hours post-last dose on the fourth day. Organs including liver, spleen, kidney, adipose, plasma were collected. Mouse liver tissues were pulverized in liquid nitrogen and aliquoted into small microtubes. TRIzol (Invitrogen Cat #15596026) was added to the tubes to facilitate cell lysis from tissue samples. The TRIzol solution containing the disrupted tissue was then centrifuged and the supernatant phase was collected. Total RNA was extracted from the supernatant using Qiagen RNA Extraction Kit (Qiagen Cat #74182) and the target mRNA levels were analyzed using qRT-PCR.

### Example 2

### RNA-Seq Study for Stimulated Hepatocytes

To identify small molecules that modulate PNPLA3, primary human hepatocytes were prepared as a monoculture, and at least one small molecule compound was applied to the cells.

RNA-seq was performed to determine the effects of the compounds on PNPLA3 expression in hepatocytes. Fold change was calculated by dividing the level of expression in the cell system that had been perturbed by the level of expression in an unperturbed system. Changes in expression having a p-value≤0.05 were considered significant.

Compounds used to perturb the signaling centers of hepatocytes include at least one compound listed in Table 1. In the table, compounds are listed with their ID, target, pathway, and pharmaceutical action. Most compounds chosen as perturbation signals are known in the art to modulate at least one canonical cellular pathway. Some compounds were selected from compounds that failed in Phase III clinical evaluation due to lack of efficacy.

TABLE 1
Compounds used in RNA-seq

| ID | Compound Name | CAS Number | Target | Pathway | Action |
|---|---|---|---|---|---|
| 1 | Simvastatin | 79902-63-9 | HMG-CoA reductase | Metabolic | Inhibitor |
| 2 | Adapin (doxepin) | 1229-29-4 | H₁ histamine, | Histamine receptor | Antagonist |
| | | | α-adrenoreceptors | signaling | |
| 3 | Methapyrilene | 91-80-5 | | | |
| 4 | Danazol | 17230-88-5 | ER, AR, Progesteron | Estrogen signaling | Agonist |
| | | | receptor | | |
| 5 | Nefazodone | 83366-66-9 | HTR2A | Calcium signaling | Antagonist |
| 6 | Rosiglitazone maleate | 155141-29-0 | PPARg | PPAR signaling | Agonist |
| 7 | Sulpiride | 15676-16-1 | D₂ dopamine | cAMP signaling | Antagonist |
| 8 | Captopril | 62571-86-2 | MMP2 | Estrogen signaling | Inhibitor |
| 9 | atenolol | 29122-68-7 | ADRB1 | Adrenergic signaling | Antagonist |
| 10 | Ranitidine | 66357-59-3 | H₂ histamine receptor | Histamine receptor | Antagonist |
| | | | | signaling | |
| 11 | Metformin | 1115-70-4 | AMPK | Insulin & AMPK | Activator |
| | | | | signaling | |
| 12 | imatinib | 220127-57-1 | RTK, Bcr-Abl | PDGFR, ABL signaling | Inhibitor |
| 13 | Papaverine | 61-25-6 | phosphodiesterase | AMPK signaling | Inhibitor |
| 14 | Amiodarone | 19774-82-4 | Adrenergic receptor | Adrenergic signaling | Antagonist |
| | | | β, CYP | | |
| 15 | Nitrofurantoin | 67-20-9 | pyruvate-flavodoxin | antibiotic | Activator |
| | | | oxidoreductase | | |
| 16 | prednisone | 53-03-2 | GR | GR signaling | Agonist |
| 17 | Penicillamine(D-) | 52-67-5 | copper | copper chelation | Chelator |
| 18 | Disopyramide | 3737-09-5 | SCN5A | Adrenergic signaling | Inhibitor |
| 19 | Rifampicin | 13292-46-1 | PXR | PXR | Inhibitor |
| 20 | Benzbromarone | 3562-84-3 | xanthine oxidase, | uric acid formation | Inhibitor |
| | | | CYP2C9 | | |
| 21 | isoniazid | 54-85-3 | CYP2C19, CYP3A4 | unknown | Inhibitor |
| 22 | Acetaminophen | 103-90-2 | COX1/2 | COX | Inhibitor |
| | (paracetamol) | | | | |
| 23 | Ritonavir | 155213-67-5 | CYP3A4, Pol polyprotein | HIV Transcription | Inhibitor |
| 24 | SGI-1776 | 1025065-69-3 | PIM | JAK/STAT signaling | Inhibitor |
| 25 | Valproate | 1069-66-5 | HDAC9, glucuronyl | unknown | Inhibitor |
| | | | transferase, | | |
| | | | epoxide hydrolase | | |
| 26 | Ibuprofen | 15687-27-1 | COX, PTGS2 | COX | Inhibitor |
| 27 | Propylthiouracil | 51-52-5 | thyroperoxidase | Thyroid hormone | Inhibitor |
| | | | | synthesis | |
| 28 | rapamycin | 53123-88-9 | mTOR | mTOR signaling | Inhibitor |
| 29 | BIO | 667463-62-9 | GSK-3 | WNT, TGF beta signaling | Inhibitor |
| 30 | ATRA | 302-79-4 | RXRb, RXRg, RARg | RAR signaling | Agonist |
| 31 | Xav939 | 284028-89-3 | tankyrase | WNT & PARP pathway | Inhibitor |
| 32 | bms189453 | 166977-43-1 | RARB | Nuclear Receptor | Agonist |
| | | | | transcription | |
| 33 | dorsomorphin | 866405-64-3 | ALK | TGF beta signaling | Inhibitor |
| 34 | BMP2 | P12643 | BMPR1A | TGF beta signaling | Agonist |
| | | (Uniprot) | | | |
| 35 | BMS777607 | 1025720-94-8 | Met | Ras signaling | Inhibitor |
| 36 | bms833923 | 1059734-66-5 | SMO | Hedgehog signaling | Antagonist |
| 37 | dmPGE2 | 39746-25-3 | EPR, PGDH | EP receptor signaling | Agonist |
| 38 | MK-0752 | 471905-41-6 | y-secretase | NOTCH signaling | Inhibitor |
| 39 | N-Acetylpurinomycin | 22852-13-7 | SnoN, SKI, SKIL | TGF beta signaling | Modulator |
| 40 | LY 364947 | 396129-53-6 | TGF-β RI, TGFR-I, | TGF beta signaling | Inhibitor |
| | | | TβR-I, ALK-5 | | |
| 41 | Enzastaurin | 170364-57-5 | PKC | Epigenetics; | Inhibitor |
| | | | | TGF-beta/Smad | |
| 42 | DMXAA | 117570-53-3 | Unclear | Tumor necrosis | Inhibitor |
| 43 | BSI-201 | 160003-66-7 | PARP | Cell Cycle/DNA | Inhibitor |
| | | | | Damage; Epigenetics | |
| 44 | Darapladib | 356057-34-6 | Phospholipase | Others | Inhibitor |
| 45 | Selumetinib | 606143-52-6 | MEK | MAPK/ERK Pathway | Inhibitor |
| 46 | Peramivir (trihydrate) | 1041434-82-5 | Influenza Virus | Anti-infection | Inhibitor |
| 47 | Palifosfamide | 31645-39-3 | DNA alkylator/crosslinker | Cell Cycle/DNA Damage | |
| 48 | Evacetrapib | 1186486-62-3 | CETP | Others | Inhibitor |
| 49 | Cediranib | 288383-20-0 | VEGFR | Protein Tyrosine | Inhibitor |
| | | | | Kinase/RTK | |
| 50 | R788 (fostamatinib, | 914295-16-2 | Syk | Protein Tyrosine | Inhibitor |
| | disodium hexahydrate) | | | Kinase/RTK | |
| 51 | Torcetrapib | 262352-17-0 | CETP | Others | Inhibitor |
| 52 | Tivozanib | 475108-18-0 | VEGFR | Protein Tyrosine | Inhibitor |
| | | | | Kinase/RTK | |
| 53 | 17-AAG (Tanespimycin) | 75747-14-7 | HSP | Cell Cycle/DNA Damage | Inhibitor |
| | | | | Metabolic | |
| | | | | Enzyme/Protease | |
| 54 | Zibotentan | 186497-07-4 | Endothelin Receptor | GPCR/G protein | Antagonist |
| 55 | Semagacestat | 425386-60-3 | y-secretase | Neuronal Signaling Stem | Inhibitor |
| | | | | Cells/Wnt | |
| 56 | Dalcetrapib | 211513-37-0 | CETP | Others | Inhibitor |
| 57 | Latrepirdine | 97657-92-6 | AMPK | Epigenetics; | Activator |
| | (dihydrochloride) | | | PI3K/Akt/mTOR | |
| 58 | CMX001 (Brincidofovir) | 444805-28-1 | CMV | Anti-infection | NA |
| 59 | Vicriviroc (maleate) | 599179-03-0 | CCR | GPCR/G protein; | Antagonist |
| | | | | Immunology/ | |
| | | | | Inflammation | |
| 60 | Temsirolimus | 162635-04-3 | mTOR | PI3K/Akt/mTOR | Inhibitor |
| 61 | Preladenant | 377727-87-2 | Adenosine Receptor | GPCR/G protein | Antagonist |
| 62 | EVP-6124 | 550999-74-1 | nAChR | Membrane | Activator |
| | (hydrochloride) | | | Transporter/Ion | |
| | (encenicline) | | | Channel | |
| 63 | Bitopertin | 845614-11-1 | GlyT1 | Membrane Transporter/ | Inhibitor |
| | | | | Ion Channel | |
| 64 | Latrepirdine | 97657-92-6 | AMPK | Epigenetics; | Inhibitor |
| | | | | PI3K/Akt/mTOR | |
| 65 | Vanoxerine | 67469-78-7 | Dopamine Reuptake | Neuronal Signaling | Inhibitor |
| | (dihydrochloride) | | Inhibitor | | |
| 66 | CO-1686 (Rociletinib) | 1374640-70-6 | EGFR | JAK/STAT | Inhibitor |
| | | | | Signaling Protein | |
| | | | | Tyrosine Kinase/RTK | |
| 67 | Laropiprant (tredaptive) | 571170-77-9 | Prostaglandin Receptor | GPCR/G protein | Antagonist |
| 68 | Bardoxolone | 218600-44-3 | Keap1-Nrf2 | NF-κB | Activator |
| 69 | VX-661 (tezacaptor) | 1152311-62-0 | CFTR | Membrane transporter/ion | Corrector |
| | | | | channel | |
| 70 | INNO-206 | 1361644-26-9 | Topoisomerase | Cell Cycle/DNA Damage | NA |
| | (aldoxorubicin) | | | | |
| 71 | LY404039 | 635318-11-5 | mGluR | GPCR/G protein | Inhibitor |
| | (pomaglumetad | | | | |
| | methionil (mGlu2/3)) | | | | |
| 72 | Perifosine (KRX-0401) | 157716-52-4 | AKT | PI3K/AKT | Inhibitor |
| 73 | Cabozantinib (XL184, | 849217-68-1 | VEGFR2, MET, | MET | Inhibitor |
| | BMS-907351) | | Ret, Kit, Flt-1/3/4, | | |
| | | | Tie2, and AXL | | |
| 74 | Dacomitinib (PF299804, | 1110813-31-4 | EGFR, ErbB2, ErbB4 | AKT/ERK, HER | Inhibitor |
| | PF299) | | | | |
| 75 | Pacritinib (SB1518) | 937272-79-2 | FLT3, JAK2, TYK2, | JAK-STAT | Inhibitor |
| | | | JAK3, JAK1 | | |
| 76 | TH-302 (Evofosfamide) | 918633-87-1 | hypoxic regions | Unclear | NA |
| 77 | α-PHP | 13415-59-3 | Unclear | Unclear | NA |
| 78 | LY 2140023 | 635318-55-7 | mGlu₂ & mGlu₃ | Gαi/o protein-dependent | Activator |
| | (Pomaglumetad | | | | |
| | methionil-LY404039) | | | | |
| 79 | TP-434 (Eravacycline) | 1207283-85-9 | Antibiotic resistance | Tetracycline-specific | Inhibitor |
| | | | mechanisms | efflux | |
| 80 | TC-5214 (S-(+)- | 107596-30-5 | Nicotinic acetylcholine | Base excision repair and | Antagonist |
| | MecaMylaMine | | receptors | homologous | |
| | Hydrochloride) | | | recombination repair | |
| 81 | Rolofylline (KW-3902) | 136199-02-5 | Al adenosine receptor | Unclear | Antagonist |
| 82 | Amigal | 75172-81-5 | a-galactosidase | Stress signaling | Inhibitor |
| | (Deoxygalactonojirimycin | | | | |
| | hydrochloride) | | | | |
| 83 | NOV-002 (oxidized L- | 103239-24-3 | gamma-glutamyl- | Glutathione pathway | NA |
| | Glutathione) | | transpeptidase (GGT) | | |
| 84 | bms-986094 (inx-189) | 1234490-83-5 | NS5B | Unclear | Inhibitor |
| 85 | TC-5214 (R- | 826-39-1 | Nicotinic receptors | Base excision repair and | Antagonist |
| | Mecamylamine | | | homologous | |
| | hydrochloride) | | | recombination repair | |
| 86 | Ganaxolone | 38398-32-2 | GBAA receptors | Unclear | Modulator |
| 87 | Irinotecan Hydrochloride | 136572-09-3 | DNA Topo I | Unclear | Inhibitor |
| | Trihydrate | | | | |
| 88 | TFP | 117-89-5 | D2R, Calmodulin | Calmodulin | Inhibitor |
| 89 | Perphenazine | 58-39-9 | D2R, Calmodulin | Calmodulin | Inhibitor |
| 90 | A3-HCI | 78957-85-4 | CKI, CKII, PKC, PKA | WNT, Hedgehog, | Inhibitor |
| | | | | PKC, PKA | |
| 91 | FICZ | 172922-91-7 | Aryl hydrocarbon | Aryl hydrocarbon | Agonist |
| | | | receptor | receptor | |
| 92 | Pifithrin-a | 63208-82-2 | p53 | p53 | Inhibitor |
| 93 | Deferoxamine mesylate | 138-14-7 | HIF | Hypoxia activated | Inhibitor |
| 94 | Insulin | 11061-68-0 | InsR | IGF-1R/InsR | Activator |
| 95 | Phorbol 12,13-dibutyrate | 37558-16-0 | PKC | PKC | Activator |
| 96 | RU 28318 | 76676-34-1 | MR | Mineralcorticoid receptor | Antagonist |
| 97 | Bryostatin1 | 83314-01-6 | PKC | PKC | Activator |
| 98 | DY 268 | 1609564-75-1 | FXR | FXR | Antagonist |
| 99 | GW 7647 | 265129-71-3 | PPARa | PPAR | Agonist |
| 100 | CI-4AS-1 | 188589-66-4 | AR | Androgen receptor | Agonist |
| 101 | T0901317 | 293754-55-9 | LXR | LXR | Agonist |
| 102 | BMP2 | P12643 | BMPR1A | TGF-B | Activator |
| | | (Uniprot) | | | |
| 103 | 22S-Hydroxycholesterol | 22348-64-7 | LXR | LXR | Inhibitor |
| 104 | CALP1 | 145224-99-3 | Calmodulin | Calmodulin | Activator |
| 105 | CALP3 | 261969-05-5 | Calmodulin | Calmodulin | Activator |
| 106 | Forskolin | 66575-29-9 | Adenylyl cyclase | cAMP related | Activator |
| 107 | Dexamethasone | 50-02-2 | GR | Glucocorticoid receptor | Activator |
| 108 | IFN-y | 98059-61-1 | IFNGR1/IFNGR2 | JAK/STAT | Activator |
| 109 | TGF-b | P01579 | TGF-beta Receptor | TGF-B | Activator |
| | | (uniprot) | | | |
| 110 | TNF-a | P01375 | TNF-R1/TNF-R2 | NF-κB, MAPK, | Activator |
| | | (uniprot) | | Apoptosis | |
| 111 | PDGF | | Pan-PDGFR | PDGFR | Activator |
| 112 | IGF-1 | P05019 | IGF-1R | IGF-1R/InsR | Activator |
| | | (uniprot) | | | |
| 113 | FGF | P05230 | FGFR | FGFR | Activator |
| | | (uniprot) | | | |
| 114 | EGF | P01133 | Pan-ErbB | EGFR | Activator |
| | | (uniprot) | | | |
| 115 | HGF/SF | P14210 | c-Met | c-MET | Activator |
| | | (uniprot) | | | |
| 116 | TCS 359 | 301305-73-7 | FLT3 | Protein Tyrosine | Inhibitor |
| | | | | Kinase/RTK | |
| 117 | Cobalt chloride | 7646-79-9 | HIF1 | Hypoxia activated | Inducer |
| 118 | CH223191 | 301326-22-7 | AhR | Aryl hydrocarbon | Antagonist |
| | | | | receptor | |
| 119 | Echinomycin | 512-64-1 | HIF | Hypoxia activated | Inhibitor |
| 120 | PAF C-16 | 74389-68-7 | MEK | MAPK | Activator |
| 121 | Bexarotene | 153559-49-0 | RXR | RXR | Agonist |
| 122 | CD 2665 | 170355-78-9 | RAR | RAR | Antagonist |
| 123 | Pifithrin-μ | 64984-31-2 | p53 | p53 | Inhibitor |
| 124 | EB1089 | 134404-52-7 | VDR | Vitamin D Receptor | Agonist |
| 125 | BMP4 | P12644 | TGF-beta | TGF-B | Activator |
| | | (uniprot) | | | |
| 126 | IWP-2 | 686770-61-6 | Wnt | WNT | Inhibitor |
| 127 | RITA (NSC 652287) | 213261-59-7 | p53 | p53 | Inhibitor |
| 128 | Calcitriol | 32222-06-3 | VDR | Vitamin D Receptor | Agonist |
| 129 | ACEA | 220556-69-4 | CB1 | Cannabinoid receptor | Agonist |
| 130 | Rimonabant | 158681-13-1 | CB1 | Cannabinoid receptor | Antagonist |
| 131 | Otenabant | 686344-29-6 | CB1 | Cannabinoid receptor | Antagonist |
| 132 | DLPC | 18194-25-7 | LRH-1/NR5A2 | LHR-1 | Agonist |
| 133 | LRH-1 antagonist | | LRH-1/NR5A3 | LHR-1 | Antagonist |
| 134 | Wnt3a | | FRIZZLED | WNT | Activator |
| 135 | Activin | | TGF-beta | TGF-B | Activator |
| 136 | Nodal | | TGF-beta | TGF-B | Activator |
| 137 | Anti mullerian hormone | | TGF-beta | TGF-B | Activator |
| 138 | GDF2 (BMP9) | | TGF-beta | TGF-B | Activator |
| 139 | GDF10 (BMP3b) | | TGF-beta | TGF-B | Activator |
| 140 | Oxoglaucine | 5574-24-3 | PI3K/Akt | PI3K/AKT | Activator |
| 141 | BMS 195614 | 182135-66-6 | RAR | RAR | Antagonist |
| 142 | LDNI93189 | 1062368-24-4 | ALK2/3 | TGF-B | Inhibitor |
| 143 | Varenicline Tartrate | 375815-87-5 | AchR | Acetylcholine receptor | Agonist |
| 144 | Histamine | 51-74-1 | Histamine receptor | Histamine receptor | Activator |
| 145 | Chloroquine phosphate | 50-63-5 | ATM/ATR | ATM/ATR | Activator |
| 146 | LJI308 | 1627709-94-7 | RSK1/2/3 | S6K | Inhibitor |
| 147 | GSK621 | 1346607-05-3 | AMPK | AMPK | Activator |
| 148 | STA-21 | 111540-00-2 | STAT3 | JAK/STAT | Inhibitor |
| 149 | SMI-4a | 438190-29-5 | Pim1 | PIM | Inhibitor |
| 150 | AMG 337 | 1173699-31-4 | c-Met | c-MET | Inhibitor |
| 151 | Wnt agonist 1 | 853220-52- | Wnt | WNT | Activator |
| | | 7(free-base) | | | |
| 152 | PRI-724 | 847591-62-2 | Wnt | WNT | Inhibitor |
| 153 | ABT-263 | 923564-51-6 | Pan-Bcl-2 | BCL2 | Inhibitor |
| 154 | Axitinib | 319460-85-0 | Pan-VEGFR | VEGFR | Inhibitor |
| 155 | Afatinib | 439081-18-2 | Pan-ErbB | EGFR | Inhibitor |
| 156 | Bosutinib | 380843-75-4 | Src | Src | Inhibitor |
| 157 | Dasatinib | 302962-49-8 | Bcr-Abl | ABL | Inhibitor |
| 158 | Masitinib | 790299-79-5 | c-Kit | c-KIT | Inhibitor |
| 159 | Crizotinib | 877399-52-5, | c-Met | c-MET | Inhibitor |
| | | 877399-53-6 | | | |
| | | (acetate) | | | |
| 160 | PHA-665752 | 477575-56-7 | c-Met | c-MET | Inhibitor |
| 161 | GSK1904529A | 1089283-49-7 | IGF-1R/InsR | IGF-1R/InsR | Inhibitor |
| 162 | GDC-0879 | 905281-76-7 | Raf | MAPK | Inhibitor |
| 163 | LY294002 | 154447-36-6 | Pan-PI3K | PI3K/AKT | Inhibitor |
| 164 | OSU-03012 | 742112-33-0 | PDK-1 | PDK-1 | Inhibitor |
| 165 | JNJ-38877605 | 943540-75-8 | c-Met | c-MET | Inhibitor |
| 166 | BMS-754807 | 1001350-96-4 | IGF-1R/InsR | IGF-1R/InsR | Inhibitor |
| 167 | TGX-221 | 663619-89-4 | p110b | PI3K/AKT | Inhibitor |
| 168 | Regorafenib | 755037-03-7 | Pan-VEGFR | VEGFR | Inhibitor |
| 169 | Thalidomide | 50-35-1 | AR | NF-κB | Antagonist |
| 170 | Amuvatinib | 850879-09-3 | PDGFRA | PDGFR | Inhibitor |
| 171 | Etomidate | 33125-97-2 | GABA | GABAergic receptor | Inhibitor |
| 172 | Glimepiride | 93479-97-1 | Potassium channel | Potassium channel | Inhibitor |
| 173 | Omeprazole | 73590-58-6 | Proton pump | Proton pump | Agonist |
| 174 | Tipifarnib | 192185-72-1 | Ras | RAS | Inhibitor |
| 175 | SP600125 | 129-56-6 | ink | MAPK | Inhibitor |
| 176 | Quizartinib | 950769-58-1 | FLT3 | FLT3 | Inhibitor |
| 177 | CP-673451 | 343787-29-1 | Pan-PDGFR | PDGFR | Inhibitor |
| 178 | Pomalidomide | 19171-19-8 | TNF-a | NF-κB | Inhibitor |
| 179 | KU-60019 | 925701-49-1 | ATM kinase | DNA Damage | Inhibitor |
| 180 | BIRB 796 | 285983-48-4 | p38 | MAPK | Inhibitor |
| 181 | R04929097 | 847925-91-1 | Gamma-secretase | NOTCH | Inhibitor |
| 182 | Hydrocortisone | 50-23-7 | GR | Glucocorticoid receptor | Agonist |
| 183 | AICAR | 2627-69-2 | AMPK | AMPK | Activator |
| 184 | Amlodipine Besylate | 111470-99-6 | Calcium channel | Calcium channel | Inhibitor |
| 185 | DPH | 147-24-0 | Bcr-Abl | ABL | Activator |
| 186 | Taladegib | 1258861-20-9 | Hedgehog/Smoothened | Hedgehog/Smoothened | Inhibitor |
| 187 | AZD1480 | 935666-88-9, | JAK2 | JAK/STAT | Inhibitor |
| | | 1260222-79-4 | | | |
| | | (TFA) | | | |
| 188 | AST-1306 | 1050500-29-2 | Pan-ErbB | EGFR | Inhibitor |
| 189 | AZD8931 | 848942-61-0 | Pan-ErbB | EGFR | Inhibitor |
| 190 | Momelotinib | 1056634-68-4 | Pan-Jak | JAK/STAT | Inhibitor |
| 191 | Cryptotanshinone | 35825-57-1 | STAT3 | JAK/STAT | Inhibitor |
| 192 | Bethanechol chloride | 590-63-6 | AchR | Acetylcholine receptor | Activator |
| 193 | Clozapine | 5786-21-0 | 5-HT | 5-HT | Antagonist |
| 194 | Dopamine | 62-31-7 | Dopamine | Dopamine receptor | Agonist |
| 195 | Phenformin | 834-28-6 | AMPK | AMPK | Activator |
| 196 | Mifepristone | 84371-65-3 | GR | Glucocorticoid receptor | Antagonist |
| 197 | GW3965 | 405911-17-3 | LXR | LXR | Agonist |
| 198 | WYE-125132 | 1144068-46-1 | mTOR | mTOR | Inhibitor |
| | (WYE-132) | | | | |
| 199 | Crenolanib | 670220-88-9 | Pan-PDGFR | PDGFR | Inhibitor |
| 200 | PF-04691502 | 1013101-36-4 | Pan-Akt | PI3K/AKT | Inhibitor |
| 201 | GW4064 | 278779-30-9 | FXR | FXR | Agonist |
| 202 | Sotrastaurin | 425637-18-9 | PKC | PKC | Inhibitor |
| 203 | Ipatasertib | 1001264-89-6 | Pan-Akt | PI3K/AKT | Inhibitor |
| 204 | ARN-509 | 956104-40-8 | AR | Androgen receptor | Inhibitor |
| 205 | T0070907 | 313516-66-4 | PPARg | PPAR | Antagonist |
| 206 | GO6983 | 133053-19-7 | PKC | PKC | Inhibitor |
| 207 | Epinephrine | 55-31-2 | Adrenergic | Adrenergic receptor | Agonist |
| 208 | Eletriptan | 177834-92-3 | 5-HT | 5-HT | Agonist |
| 209 | Trifluoperazine | 440-17-5 | Dopamine | Dopamine receptor | Inhibitor |
| 210 | Fexofenadine | 153439-40-8 | Histamine | Histamine receptor | Inhibitor |
| 211 | Corticosterone | 56-47-3 | MR | Mineralcorticoid receptor | Agonist |
| 212 | Tamibarotene | 94497-51-5 | RAR | RAR | Agonist |
| 213 | Leucine | 99-15-0 | mTOR | mTOR | Activator |
| 214 | Glycopyrrolate | 596-51-0 | AchR | Acetylcholine receptor | Antagonist |
| 215 | Tiagabine | 115103-54-3 | GABA | GABAergic receptor | Inhibitor |
| 216 | Fluoxymesterone | 76-43-7 | AR | Androgen receptor | Agonist |
| 217 | Tamsulosin | 106463-17-6 | Adrenergic | Adrenergic receptor | Antagonist |
| | hydrochloride | | | | |
| 218 | Ceritinib | 1032900-25-6 | ALK | ALK | Inhibitor |
| 219 | GSK2334470 | 1227911-45-6 | PDK-1 | PDK-1 | Inhibitor |
| 220 | AZD1208 | 1204144-28-4 | Pan-PIM | PIM | Inhibitor |
| 221 | CGK733 | 905973-89-9 | ATM/ATR | DNA Damage | Inhibitor |
| 222 | LDN-212854 | 1432597-26-6 | Pan-TGFB | TGF-B | Inhibitor |
| 223 | GZD824 Dimesylate | 1421783-64-3 | Bcr-Abl | ABL | Inhibitor |
| 224 | AZD2858 | 486424-20-8 | Pan-GSK-3 | GSK-3 | Inhibitor |
| 225 | FRAX597 | 1286739-19-2 | PAK | PAK | Inhibitor |
| 226 | SC75741 | 913822-46-5 | NF-κB | NF-κB | Inhibitor |
| 227 | SH-4-54 | 1456632-40-8 | Pan-STAT | JAK/STAT | Inhibitor |
| 228 | HS-173 | 1276110-06-5 | p110a | PI3K/AKT | Inhibitor |
| 229 | K02288 | 1431985-92-0 | Pan-TGFB | TGF-B | Inhibitor |
| 230 | EW-7197 | 1352608-82-2 | Pan-TGFB | TGF-B | Inhibitor |
| 231 | Decernotinib | 944842-54-0 | Pan-Jak | JAK/STAT | Inhibitor |
| 232 | MI-773 | 1303607-60-4 | p53 | p53 | Inhibitor |
| 233 | PND-1186 | 1061353-68-1 | FAK | FAK | Activator |
| 234 | Kartogenin | 4727-31-5 | SMAD4/5 | TGF-B | Activator |
| 235 | Picropodophyllin | 477-47-4 | IGF-1R | IGF-1R/InsR | Inhibitor |
| 236 | AZD6738 | 1352226-88-0 | ATR | ATM/ATR | Inhibitor |
| 237 | Smoothened Agonist | 912545-86-9 | Hedgehog/Smoothened | Hedgehog/Smoothened | Agonist |
| 238 | Erlotinib | 183321-74-6 | EGFR/ErbB1 | EGFR | Inhibitor |
| 239 | MHY1485 | 326914-06-1 | mTOR | mTOR | Activator |
| 240 | SC79 | 305834-79-1 | Pan-Akt | PI3K/AKT | Activator |
| 241 | meBIO | 667463-95-8 | AhR | Aryl hydrocarbon | Agonist |
| | | | | receptor | |
| 242 | Huperzine | 102518-79-6 | AchE | Acetylcholine receptor | Inhibitor |
| 243 | BGJ398 | 872511-34-7 | Pan-FGFR | FGFR | Inhibitor |
| 244 | Netarsudil | 1253952-02-1 | ROCK | ROCK | Inhibitor |
| 245 | Acetycholine | 2260-50-6 | AchR | Acetylcholine receptor | Agonist |
| 246 | Purmorphamine | 483367-10-8 | Hedgehog/Smoothened | Hedgehog/Smoothened | Agonist |
| 247 | LY2584702 | 1082949-67-4 | p70 S6K | S6K | Inhibitor |
| 248 | Dorsomorphin | 866405-64-3 | AMPK | AMPK | Inhibitor |
| 249 | Glasdegib | 1095173-27-5 | Hedgehog/Smoothened | Hedgehog/Smoothened | Inhibitor |
| | (PF-04449913) | | | | |
| 250 | LDN193189 | 1062368-24-4 | Pan-TGFB | TGF-B | Inhibitor |
| 251 | Oligomycin A | 579-13-5 | ATPase | ATP channel | Inhibitor |
| 252 | BAY 87-2243 | 1227158-85-1 | HIFI | Hypoxia activated | Inhibitor |
| 253 | SIS3 | 521984-48-5 | SMAD3 | TGF-B | Inhibitor |
| 254 | BDA-366 | 1909226-00-1 | Bcl-2 | BCL2 | Antagonist |
| 255 | XMU-MP-1 | 2061980-01-4 | MST1/2 | Hippo | Inhibitor |
| 256 | Semaxinib | 1080622-86-1 | Pan-VEGFR | VEGFR | Inhibitor |
| 257 | BAM7 | 331244-89-4 | Bcl-2 | BCL2 | Activator |
| 258 | GDC-0994 | 1453848-26-4 | Erk | MAPK | Inhibitor |
| 259 | SKL2001 | 909089-13-0 | Wnt | WNT | Agonist |
| 260 | Merestinib | 1206799-15-6 | c-Met | c-MET | Inhibitor |
| 261 | APS-2-79 | 2002381-31-7 | MEK | MAPK | Antagonist |
| 262 | NSC228155 | 113104-25-9 | Pan-ErbB | EGFR | Activator |
| 263 | 740 Y-P | 1236188-16-1 | Pan-PI3K | PI3K/AKT | Activator |
| 264 | b-Estradiol | 50-28-2 | ER | ER | Activator |
| 265 | Glucose | 50-99-7 | GLUTs | metabolic/glycolysis | Activator |
| 266 | Transferrin | 11096-37-0 | Transferrin Receptor | Iron transport | Activator |
| 267 | AM 580 | 102121-60-8 | RAR | RAR | Activator |

### Example 3

### Identification of Compounds that Modulate PNPLA3 Expression

Analysis of RNA-seq data revealed 23 compounds that caused significant changes in the expression of PNPLA3 (p<0.01). Among these compounds, 9 compounds were observed to result in reduction in PNPLA3 expression with a minimum log2 fold change of −0.5. The results are presented in Table 2.

TABLE 2
PNPLA3 expression modulated by compounds

| | | Fold change (Log 2) |
|---|---|---|
| ID | Compound | vs untreated |
| 50 | R788 (fostamatinib, disodium | −1.38 |
| | hexahydrate) | |
| 75 | Pacritinib | −1.32 |
| 84 | BMS-986094 | −0.69 |
| 123 | Pifithrin-μ | −0.68 |
| 163 | LY294002 | −0.76 |
| 166 | BMS-754807 | −0.53 |
| 170 | Amuvatinib | −0.52 |
| 190 | Momelotinib | −0.78 |
| 198 | WYE-125132 (WYE-132) | −0.86 |
| 255 | XMU-MP-1 | −0.66 |

Two identified compounds, Pacritinib and Momelotinib, are known inhibitors of the JAK/STAT pathway. Pacritinib mainly inhibits Janus kinase 2 (JAK2) and Fms-like tyrosine kinase 3 (FLT3). Momelotinib is an ATP competitor that specifically inhibits Janus kinases JAK1 and JAK2. This finding strongly suggests that PNPLA3 expression may be regulated by the JAK/STAT pathway. Inhibiting signaling molecules, particularly JAK1 and JAK2, in the JAK/STAT pathway may potentially downregulate PNPLA3.

The results also suggest that PNPLA3 expression may be associated with other signaling pathways. R788 (fostamatinib, disodium hexahydrate) is an inhibitor of spleen tyrosine kinase (Syk), which selectively inhibits Syk-dependent signaling. BMS-986094 is a guanosine nucleotide analog that inhibits the nucleotide polymerase nonstructural protein 5B (NSSB) from Hepatitis C virus. Pifithrin-μ inhibits p53 binding to mitochondria by reducing its affinity for antiapoptotic proteins Bcl-2 and Bcl-XL, thereby inhibiting p53-dependent apoptosis. LY294002 is a potent inhibitor of many proteins and a strong phosphoinositide 3-kinases (PI3Ks) inhibitor. BMS-754807 is a potent and reversible inhibitor of insulin-like growth factor 1 receptor (IGF-1R)/insulin receptor family kinases (InsR). Amuvatinib is a multi-targeted inhibitor of c-Kit, Platelet-derived growth factor receptor alpha (PDGFRα) and FLT3. WYE-125132 (WYE-132) is a highly potent, ATP-competitive mammalian Target Of Rapamycin (mTOR) inhibitor. XMU-MP-1 is an inhibitor of Mammalian sterile 20-like kinases 1 and 2 (MST1 and MST2), which are kinases involved in the Hippo signaling pathway. Targeting these targets and/or associated pathways may be potentially effective to reduce PNPLA3 expression in hepatocytes.

### Example 4

### Determining Genomic Position and Composition of Signaling Centers

A multilayered approach was used herein to identify locations or the “footprint” of signaling centers. The linear proximity of genes and enhancers is not always instructive to determine the 3D conformation of the signaling centers.

ChIP-seq was used to determine the genomic position and composition of signaling centers. The ChIP-seq experiments and analysis were performed according to Example 1. Antibodies specific to 67 targets, including transcription factors, signaling proteins, and chromatin modifications or chromatin-associated proteins, were used in ChIP-seq studies. These antibody targets are shown in Table 3. In the signaling proteins column, the associated canonical pathway is included after the “-”.

TABLE 3
ChIP-seq targets for primary human hepatocytes

| Chromatin | Transcription factors | | Signaling proteins | |
|---|---|---|---|---|
| H3K4me3 | HNFlA | RNA Pol II | STAT1-JAK/STAT | NR3C1 (glucocorticoid receptor, |
| | | | | GR)-nuclear receptor signaling |
| H3K27ac | FOXA1 | ONECUT2 | STAT3-JAK/STAT | AR (androgen receptor)-nuclear |
| | | | | receptor signaling |
| H3K4me1 | HNF4A | PROX1 | TP53-p53, mTOR, AMPK | ESR1 (estrogen receptor, ERa)- |
| | | | | nuclear receptor signaling |
| H3K27me3 | NROB2 | YY1 | TEAD 1/2-Hippo | NR1H3 (liver X receptor alpha, |
| | | | | LXRa)-nuclear receptor signaling |
| p300 | FOXA2 | CTCF | NF-κB (RelA/p65)-NF-κB | NR1H4 (farnesoid X receptor, FXR)- |
| | (HNF3b) | | | nuclear receptor signaling |
| BRD4 | CUX2 | ONECUT1 | CREB1-MAPK | AHR (aryl hydrocarbon receptor)- |
| | | (HNF6) | | aryl hydrocarbon signaling |
| SMC1 | HHEX | MYC | CREB2-MAPK | NR1I2 (pregnane X receptor, PXR)- |
| | | | | nuclear receptor signaling |
| | ZGPAT | ATF5 | JUN-TLR, MAPK | HIF1a (hypoxia inducible factor)- |
| | | | | hypoxia activated signaling |
| | NR113 | | FOS-TLR, MAPK | TCF7L2 (TCF4)-WNT |
| | | | ELK1-MAPK | CTNNB1-WNT |
| | | | SMAD2/3-TGF-β | RBPJ-NOTCH |
| | | | SMAD4-TGF-β | SREBP1-cholesterol biosynthesis |
| | | | SMAD1/5/8-TGF-β | SREBP2-cholesterol biosynthesis |
| | | | ETV4-ERK MAPK | RXR (RA pathway)-nuclear |
| | | | | receptor signaling |
| | | | RARA-nuclear receptor signaling | NR3C2 (Mineralocorticoid receptor)- |
| | | | | nuclear receptor signaling |
| | | | NR1I1 (Vitamin D receptor, VDR)- | STAT5-JAK/STAT |
| | | | nuclear receptor signaling | |
| | | | NR5A2 (liver receptor homolog 1, | PPARG-nuclear receptor signaling |
| | | | LRH-1)-nuclear receptor | |
| | | | signaling | |
| | | | YAP1-Hippo signaling | PPARA-nuclear receptor signaling |
| | | | TAZ-Hippo signaling | mTOR-mTOR signaling |
| | | | MLXIPL-carbohydrate response | GLI3-Hedgehog signaling |
| | | | signaling | |
| | | | GLI1-Hedgehog signaling | ATR-DNA damage response |
| | | | | signaling |
| | | | WWTR1-Hippo signaling | |

In primary human hepatocytes, the insulated neighborhood that contains the PNPLA3 gene was identified to be on chromosome 22 at position 43,782,676-45,023,137 with a size of approximately 1,240 kb. 12 signaling centers were found within the insulated neighborhood. The chromatin marks or chromatin-associated proteins, transcription factors and signaling proteins that were found in the insulated neighborhood are presented in Table 4.

TABLE 4
Insulated neighborhood containing PNPLA3

| Chromatin | Transcription factors | Signaling proteins |
|---|---|---|
| H3k27ac | HNF3b | TCF4 |
| BRD4 | HNF4a | HIF1a |
| p300 | HNF4 | HNF1 |
| H3K4me1 | HNF6 | ERa |
| H3K4me3 | MYC | GR |
| | ONECUT2 | JUN |
| | YY1 | RXR |
| | | STAT3 |
| | | VDR |
| | | NF-κB |
| | | SMAD2/3 |
| | | STAT1 |
| | | TEAD1 |
| | | p53 |
| | | SMAD4 |
| | | FOS |

The ChIP-seq profile suggests that the insulated neighborhood containing PNPLA3 may be regulated by JAK/STAT signaling, TGF-beta/SMAD signaling, BMP signaling, nuclear receptor signaling, VDR signaling, NF-κB signaling, MAPK signaling, and/or Hippo signaling pathways. STAT1 and STAT3, both associated with the JAK/STAT pathway, were observed to bind to the signaling centers within the neighborhood, which coincides with the finding that disrupting the JAK/STAT pathway with compounds altered PNPLA3 expression. Moreover, the insulated neighborhood is also enriched with NF-κB, which is a transcription factor regulated by the mTOR pathway. Targeting one or more of these pathways may be effective in downregulating PNPLA3 expression.

### Example 5

### Determining Genome Architecture in Hepatocytes

HI-ChIP was performed as described in Example 1 to decipher genome architecture. In some cases, ChIA-PET for SMC1 structural protein was used for the same purpose. These techniques identify portions of the chromatin that interact to form 3D structures, such as insulated neighborhood and gene loops.

The insulated neighborhood containing the PNPLA3 gene was identified to be on chromosome 22 at position 43,782,676-45,023,137 with a size of approximately 1,240 kb. The insulated neighborhood contains PNPLA3 and 7 other genes, with four genes upstream of PNPLA3, namely MPPED1, EFCAB6, SULT4A1, and PNPLA5, and three genes downstream of PNPLA3, namely SAMM50, PARVB, and PARVG.

### Example 6

### Validating Compounds and Pathways in Human Hepatocytes

Initial RNA-seq screen and ChIP-seq profile identified compounds and pathways that may be utilized to downregulate PNPLA3 expression. The aim of the validation studies was to test the identified compounds from key pathways, and expand the compound franchise to identify other potential hits. Candidate compounds were subjected to validation with qRT-PCR in human hepatocytes. qRT-PCR was performed on samples of primary human hepatocytes from a second donor treated with the candidate compounds. Compounds were tested at concentrations ranging from 0.01 μM to 50 μM, with the majority tested at 10 μM. Fold change in PNPLA3 expression observed via qRT-PCR was analyzed as described in Example 1. Compounds that caused robust reduction of PNPLA3 expression were selected for further characterization.

Initial RNA-seq screen and ChIP-seq data suggested that the JAK/STAT pathway may play a role in controlling PNPLA3 expression. The two JAK inhibitors identified from the RNA-seq screen, Momelotinib and Pacritinib, and an additional panel of JAK inhibitors were tested in human hepatocytes. As expected, both Momelotinib and Pacritinib induced a substantial decrease in PNPLA3 expression in human hepatocytes. Two other JAK inhibitors, Oclacitinib and AZD1480, also showed efficient downregulation of PNPLA3. This confirms JAK inhibitors reduce PNPLA3 expression. qRT-PCR results from human hepatocytes treated with 10 μM of selected JAK inhibitors are shown in Table 5. Each value is the mean of three replicates±standard deviation.

TABLE 5
JAK inhibitors in human hepatocytes

| | Relative PNPLA3 |
|---|---|
| Compound | mRNA levels |
| DMSO | 1.10 ± 0.09 |
| Momelotinib | 0.38 ± 0.01 |
| Pacritinib | 0.23 ± 0.02 |
| Oclacitinib | 0.56 ± 0.03 |
| AZD1480 | 0.69 ± 0.10 |
| Ruxolitinib | 0.89 ± 0.17 |
| Solcitinib | 1.10 ± 0.01 |
| Gandotinib | 0.76 ± 0.13 |
| Upadacitinib | 0.93 ± 0.15 |
| JANEX-1 | 0.78 ± 0.11 |
| Filgotinib | 1.07 ± 0.14 |
| Cerdulatinib | 0.82 ± 0.00 |

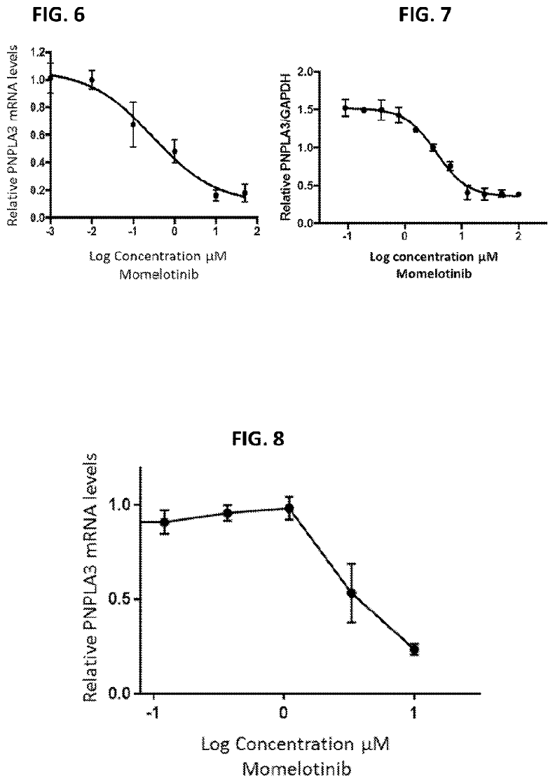

PNPLA3 expression in human hepatocytes exhibited a dose-dependent response to Momelotinib (see FIG. 6), indicating a drug-specific action. Furthermore, no cytotoxicity was observed with Momelotinib at any tested concentration (0.01˜50 μM).

An mTOR inhibitor, WYE-125132 (WYE-132), was identified in the initial RNA-seq experiment. In addition, Momelotinib is also known to inhibit a spectrum of kinases, including TANK-binding kinase 1 (TBK1), which has been linked to the mTOR pathway. Therefore, a number of mTOR inhibitors were tested in human hepatocytes. Several mTOR inhibitors showed inhibition of PNPLA3 expression in human hepatocytes, reaffirming the role of mTOR signaling in PNPLA3 gene expression control. qRT-PCR results from human hepatocytes treated with 1 μM of WYE-125132 or 10 μM of selected mTOR pathway inhibitors are presented in Table 6. Each value is the mean of three replicates±standard deviation.

TABLE 6
mTOR inhibitors in human hepatocytes

| | Relative PNPLA3 |
|---|---|
| Compound | mRNA levels |
| DMSO | 1.00 ± 0.12 |
| WYE-125132 | 0.67 ± 0.09 |
| CZ415 | 0.62 ± 0.07 |
| AZD-8055 | 0.52 ± 0.16 |
| PP242 | 0.50 ± 0.02 |
| OSI-027 | 0.21 ± 0.00 |
| PF-04691502 | 0.35 ± 0.01 |
| Everolimus | 0.98 ± 0.17 |

One TBK1 inhibitor, BX795, was also tested. Relative PNPLA3 mRNA levels in human hepatocytes after BX795 treatment were 0.51±0.06.

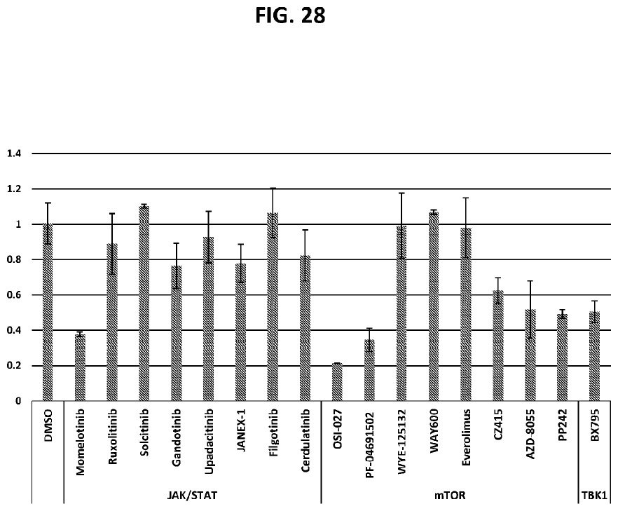

A selection of the PNPLA3 mRNA levels from Tables 5 and 6 are shown in FIG. 28.

Initial RNA-seq screen also demonstrated downregulation of PNPLA3 expression by R788 (fostamatinib, disodium hexahydrate), which is a Syk inhibitor. R788 and an additional panel of Syk pathway inhibitors were thus tested in human hepatocytes. At 10 μM, R788 and 6 other Syk pathway inhibitors reduced PNPLA3 expression from about 22% to 55% in human hepatocytes. This shows that targeting the Syk pathway can also effectively downregulate PNPLA3. qRT-PCR results from human hepatocytes treated with 10 μM of selected Syk pathway inhibitors are presented in Table 7. Relative PNPLA3 mRNA levels were normalized to B2M. Each value is the mean of three replicates±standard deviation.

TABLE 7
Syk inhibitors in human hepatocytes

| | Relative PNPLA3 |
|---|---|
| Compound | mRNA levels |
| DMSO | 1.00 ± 0.10 |
| R788 | 0.77 ± 0.02 |
| tamatinib | 0.63 ± 0.06 |
| entospletinib | 0.67 ± 0.05 |
| nilvadipine | 0.61 ± 0.08 |
| ibrutinib | 0.50 ± 0.06 |
| idelalisib | 0.65 ± 0.01 |
| TAK-659 | 0.44 ± 0.11 |

### Example 7

### Interrogating Pathways of Interest via siRNA

The aim of this experiment was to confirm relative roles of the identified signaling pathways (e.g., JAK/STAT, Syk, mTOR and PDGFR) that are controlling PNPLA3 expression. The end component of each pathway was targeted via siRNA-mediated knock-down. Primary human hepatocytes were reverse transfected with 10 nM siRNA targeting one or more of the following mRNAs: JAK1, JAK2, SYK, mTOR and/or PDGFRA. After 48 hours of treatment, levels of the target mRNA were measured via qRT-PCR and compared with a non-targeting siRNA control to evaluate the known-down efficiency (reported as percent decrease). PNPLA3 mRNA levels were then assayed via qRT-PCR and normalized to the geometric mean of two internal controls, GAPDH and B2M.

The knock-down efficiency of the siRNA experiments ranged from 50%˜95%. The knock-down was also highly specific. Knocking down JAK1, JAK2, SYK, mTOR or PDGFRA each led to a decrease of PNPLA3 mRNA levels, consistent with previous observations. However, the data also suggest that inhibition of a single kinase is not sufficient to decrease PNPLA3. This indicates that PNPLA3 expression is well regulated through a signaling network including functions from at least the JAK/STAT, Syk, mTOR and/or PDGFR pathways. Results of the siRNA experiments are presented in Table 8.

TABLE 8
Knock-down of signaling proteins via siRNA

| | Target mRNA | |
|---|---|---|
| | knock-down | Relative PNPLA3 |
| mRNA targeted | efficiency | mRNA levels |
| Non-targeting | / | 1.00 ± 0.06 |
| JAK1 | 0.95 ± 0.01 | 0.87 ± 0.05 |
| JAK2 | 0.77 ± 0.05 | 0.72 ± 0.06 |
| JAK1 + JAK2 | JAK1: 0.93 ± 0.01 | 0.90 ± 0.06 |
| | JAK2: 0.76 ± 0.02 | |
| SYK | 0.52 ± 0.10 | 0.69 ± 0.03 |
| mTOR | 0.88 ± 0.01 | 0.81 ± 0.03 |
| PDGFRA | 0.93 ± 0.05 | 0.64 ± 0.02 |

### Example 8

### Compound Validation in Mouse Hepatocytes

Selected compounds were tested in mouse hepatocytes to confirm their ability to downregulate PNPLA3. qRT-PCR was performed on samples of mouse hepatocytes treated with the candidate compounds. Compounds were tested at concentrations ranging from 0.01 μM to 50 μM. Fold change in PNPLA3 expression observed via qRT-PCR was analyzed as described in Example 1. PNPLA3 levels were normalized to the level of a house keeping gene ACTB. Compounds that caused robust reduction of PNPLA3 expression were selected for further characterization.

The effect of Momelotinib and Pacritinib on PNPLA3 expression was validated in mouse hepatocytes. Both Momelotinib and Pacritinib induced significant reduction of PNPLA3 mRNA levels in mouse hepatocytes, with respective fold changes of 10% and 13% relative to the control. While slight cytotoxicity was observed with Pacritinib at 10 μM, Momelotinib was well tolerated at 10 μM by mouse hepatocytes.

Downregulation of PNPLA3 expression by mTOR pathway inhibitors was also observed in mouse hepatocytes, consistent with the data in human primary hepatocytes. qRT-PCR results from mouse hepatocytes treated with selected mTOR pathway inhibitors are presented in Table 9. In the table, all compounds were tested at 1 μM, except for Torin 1, which was at 10 μM.

TABLE 9
mTOR inhibitors in mouse hepatocytes

| | Relative PNPLA3 |
|---|---|
| Compound | mRNA levels |
| Everolimus | 0.31 ± 0.10 |
| Torin 1 | 0.53 ± 0.25 |
| PP242 | 0.44 ± 0.10 |
| WAY600 | 0.67 ± 0.21 |
| CZ415 | 0.20 ± 0.11 |
| INK128 | 0.30 ± 0.14 |
| TAK659 | 0.31 ± 0.12 |
| AZD-8055 | 0.21 ± 0.11 |
| PF-04691502 | 0.21 ± 0.10 |
| Voxtalisib | 0.31 ± 0.08 |
| Deforolimus | 0.30 ± 0.10 |
| OSI-027 | 0.24 ± 0.12 |

### Example 9

### Compound Testing in Hepatic Stellate Cells

Hepatic stellate cells (HSCs, also called perisinusoidal cells or Ito cells) are contractile cells that wrap around the endothelial cells. In normal liver, they are present in a quiescent state and make about 10% of the liver. When liver is damaged, they change to activated state and play a major role in liver fibrosis. PNPLA3 is expressed in stellate cells as well as hepatocytes. Emerging evidence suggests that PNPLA3 is involved in HSC activation and its genetic variant I148M potentiates pro-fibrogenic features such as increased pro-inflammatory cytokine secretion. Therefore, candidate compounds were tested for their effect on PNPLA3 expression in stellate cells. Besides PNPLA3, compound effect on collagen 1a1 (Col1a1, encoded by the COL1A1 gene) expression was also evaluated in stellate cells as Col1a1 plays a major role in fibrosis and decreasing Col1a1 levels are predicted to improve fibrosis. The COL1A1 gene is not typically expressed in hepatocytes, but is expressed at a much higher level in HSCs. Reduction of PNPLA3 has been reported to affect the fibrotic phenotype in HSCs including Col1a1 levels. Therefore, compounds that are capable of decreasing levels of both PNPLA3 and Col1a1 may provide additional benefits for treating NASH.

Candidate compounds were tested in stellate cells for their ability to modulate PNPLA3 and COL1A1. Stellate cells were treated with serial dilutions of the compounds, ranging from 0.1 μM to 100 μM. Changes in PNPLA3 (or COL1A1) mRNA levels in stellate cells were analyzed with qRT-PCR. Once compounds capable of downregulating PNPLA3 and/or COL1A1 were identified, additional compounds that are known to act in the same pathways were also tested. Transforming growth factor beta (TGF-beta) is known to induce fibrotic genes including COL1A1 in vitro, and was thus chosen as a positive control (i.e., positively regulate COL1A1 expression).

Momelotinib reduced PNPLA3 mRNA levels in stellate cells in a dose-dependent manner (see FIG. 7), consistent with previous observations in human and mouse hepatocytes. However, at the tested concentrations (0.01 μM, 0.1 μM, 1 μM and 10 μM), Momelotinib did not alter COL1A1 expression.

Encouragingly, the mTOR inhibitor WYE-125132 (WYE-132) decreased both PNPLA3 and COL1A1 in HSCs in a dose-dependent manner (see Table 10). Additional mTOR compounds were then tested, including everolimus, Torin 1, PP242, CZ415, INK-128, and AZD-8055. Serial dilutions of the mTOR compounds had robust effects on PNPLA3 and COL1A1 gene expression in HSCs. All tested mTOR inhibitors decreased PNPLA3 levels and all tested mTOR inhibitors, with the exception of everolimus, decreased COL1A1 levels. Results of mTOR compound treatments in HSCs are presented in Table 10. Fold change, expressed as Relative Quantification (RQ), RQ Min, and RQ Max values were calculated as described in Example 1. These results were obtained from four technical replicates.

TABLE 10
mTOR inhibitors in hepatic stellate cells

| | |
|---|---|
| Relative PNPLA3 mRNA levels | Relative COL1A1 mRNA levels |

| Compound | Concentration | RQ | RQ Min | RQ Max | RQ | RQ Min | RQ Max |
|---|---|---|---|---|---|---|---|
| DMSO | / | 1.00 | 0.90 | 1.11 | 1.00 | 0.95 | 1.05 |
| TGF-beta | 0.1 ng/ml | 1.66 | 1.57 | 1.76 | 1.59 | 1.50 | 1.69 |
| | 1 ng/ml | 1.97 | 1.83 | 2.11 | 2.03 | 1.94 | 2.14 |
| (positive | 10 ng/ml | 1.88 | 1.71 | 2.05 | 1.80 | 1.70 | 1.90 |
| control) | 100 ng/ml | 3.06 | 2.71 | 3.45 | 1.82 | 1.80 | 1.84 |
| WYE-125132 | 0.01 μM | 0.44 | 0.38 | 0.50 | 0.92 | 0.88 | 0.97 |
| | 0.1 μM | 0.36 | 0.32 | 0.40 | 0.42 | 0.38 | 0.46 |
| | 1 μM | 0.42 | 0.38 | 0.46 | 0.26 | 0.25 | 0.27 |
| | 10 μM | 0.42 | 0.40 | 0.45 | 0.29 | 0.28 | 0.31 |
| everolimus | 0.01 μM | 0.64 | 0.60 | 0.68 | 1.07 | 0.92 | 1.24 |
| | 0.1 μM | 0.47 | 0.41 | 0.55 | 1.01 | 0.94 | 1.08 |
| | 1 μM | 0.56 | 0.52 | 0.59 | 1.12 | 1.04 | 1.21 |
| | 10 μM | 0.44 | 0.34 | 0.57 | 1.19 | 1.16 | 1.22 |
| Torin 1 | 0.01 μM | 0.34 | 0.29 | 0.40 | 0.29 | 0.28 | 0.29 |
| | 0.1 μM | 0.65 | 0.60 | 0.70 | 0.41 | 0.39 | 0.43 |
| | 1 μM | 0.99 | 0.91 | 1.07 | 0.43 | 0.42 | 0.44 |
| | 10 μM | 2.39 | 2.14 | 2.67 | 0.36 | 0.35 | 0.37 |
| PP242 | 0.01 μM | 1.07 | 0.97 | 1.18 | 1.09 | 1.02 | 1.15 |
| | 0.1 μM | 0.74 | 0.67 | 0.82 | 1.02 | 0.99 | 1.05 |
| | 1 μM | 0.39 | 0.36 | 0.41 | 0.40 | 0.38 | 0.41 |
| | 10 μM | 0.63 | 0.60 | 0.67 | 0.18 | 0.17 | 0.18 |
| CZ415 | 0.01 μM | 0.87 | 0.74 | 1.02 | 1.01 | 0.94 | 1.08 |
| | 0.1 μM | 0.47 | 0.42 | 0.53 | 0.86 | 0.84 | 0.89 |
| | 1 μM | 0.31 | 0.24 | 0.39 | 0.28 | 0.26 | 0.30 |
| | 10 μM | 0.35 | 0.32 | 0.38 | 0.27 | 0.26 | 0.28 |
| INK-128 | 0.01 μM | 0.40 | 0.31 | 0.52 | 1.05 | 1.02 | 1.07 |
| | 0.1 μM | 0.28 | 0.26 | 0.30 | 0.27 | 0.26 | 0.28 |
| | 1 μM | 0.58 | 0.49 | 0.69 | 0.32 | 0.30 | 0.35 |
| | 10 μM | 0.58 | 0.52 | 0.64 | 0.21 | 0.21 | 0.22 |
| AZD-8055 | 0.01 μM | 0.38 | 0.36 | 0.40 | 0.71 | 0.68 | 0.73 |
| | 0.1 μM | 0.44 | 0.42 | 0.47 | 0.58 | 0.56 | 0.60 |
| | 1 μM | 0.45 | 0.27 | 0.57 | 0.35 | 0.34 | 0.37 |
| | 10 μM | 0.39 | 0.27 | 0.57 | 0.27 | 0.26 | 0.28 |

Surprisingly, compound screening in HSCs also identified two additional compounds, BIO and AZD2858, which modestly decreased both PNPLA3 and COL1A1 in a dose dependent manner. BIO and AZD2858 are inhibitors of Glycogen synthase kinase 3 (GSK3). Results of GSK3 inhibitors in HSCs are presented in Table 11. Fold change, expressed as Relative Quantification (RQ), RQ Min, and RQ Max values were calculated as described in Example 1. These results were obtained from four technical replicates.

TABLE 11
GSK3 inhibitors in hepatic stellate cells

| | |
|---|---|
| Relative PNPLA3 mRNA levels | Relative COL1A1 mRNA levels |

| Compound | Concentration | RQ | RQ Min | RQ Max | RQ | RQ Min | RQ Max |
|---|---|---|---|---|---|---|---|
| DMSO | / | 1.00 | 0.90 | 1.11 | 1.00 | 0.86 | 1.16 |
| BIO | 1 μM | 1.09 | 0.96 | 1.23 | 0.74 | 0.68 | 0.79 |
| | 10 μM | 0.54 | 0.43 | 0.67 | 0.59 | 0.56 | 0.62 |
| AZD2858 | 1 μM | 0.48 | 0.38 | 0.60 | 0.83 | 0.78 | 0.89 |
| | 10 μM | 0.72 | 0.65 | 0.79 | 0.72 | 0.66 | 0.79 |

### Example 10

### Compound Testing in PNPLA3 Mutant Cell Line HepG2

Candidate compounds were evaluated in a PNPLA3 mutant cell line HepG2 to test their effects on mutant PNPLA3 expression. The HepG2 cells have the I148M mutation in PNPLA3. Changes in PNPLA3 expression in HepG2 cells were analyzed with qRT-PCR. PNPLA3 mRNA levels were normalized to the geometric mean of two internal controls, GUSB and B2M.

Momelotinib showed consistent downregulation of PNPLA3 in HepG2 cells. At 10 μM, Momelotinib treatment caused an approximately 85% drop in PNPLA3 mRNA level compared to the DMSO control. The effect is compatible with results from other tested cells. Moreover, mutant PNPLA3 mRNA levels in HepG2 cells responded to Momelotinib in a dose-dependent manner (see FIG. 8). These experiments demonstrated that Momelotinib can decrease mutant PNPLA3 expression as well.

### Example 11

### Momelotinib Mechanism of Action Studies

As Momelotinib consistently exhibited downregulation of PNPLA3 in multiple experiments, its mechanism of action was further investigated. The siRNA knock-down experiments (see Example 7) demonstrated that knocking down JAK1 or JAK2, whether alone or jointly, failed to fully recapitulate the effect of Momelotinib on PNPLA3, which prompted the hypothesis that Momelotinib may exert its activities through additional pathways. In fact, Momelotinib is known to inhibit a spectrum of kinases with submicromolar affinity in addition to JAK1 and JAK2 (Tyner J W et al., Blood, 2010, 115(25), 5232-5240, which is hereby incorporated by reference in its entirety). Among the list of Momelotinib targets, TBK1 and ACVR1 (Activin A receptor, type I) were of particular interest. TBK1, also known as the NF-κB-activating kinase, can mediate NF-κB activation in response to certain growth factors. ACVR1 is a member of the TGF-beta family subgroup of receptors and can activate SMAD transcriptional regulators upon ligand binding. This coincides with the ChIP-seq data (described in Example 4) which showed that the insulated neighborhood of PNPLA3 is bound by a number of signaling proteins including NF-κB, SMAD2/3 and SMAD4. This is further supported by the observation that Activin and bone morphogenic proteins (BMPs), such as BMP2 and GDF2, were the best upregulators of PNPLA3 and PNPLA5 in the RNA-seq study. Therefore, signaling proteins in the NF-κB pathway and ACVR1/SMAD pathway were targeted via siRNA to test their effect on PNPLA3. Additionally, as PNPLA5 is located in the same insulated neighborhood as PNPLA3 and has been observed to respond similarly to compound treatments as PNPLA3, PNPLA5 expression was included in the analysis as a second readout.

Primary human hepatocytes were reverse transfected with 10 nM siRNA specific for each of the six SMAD proteins: SMAD1, SMAD2, SMAD3, SMAD4, SMAD5, and SMAD9. The knock-down treatment was performed in the presence of either BMP2 (220 nM) or TGF-beta (100 ng/mL) to stimulate SMAD activation. After 72 hours of treatment, levels of target mRNAs were evaluated for knock-down efficiency and the effect of each knock-down on PNPLA3 and PNPLA5 expression was examined. Each target mRNA was efficiently knocked down by the siRNA. The result of the SMAD protein knock-down experiments are presented in Table 12. The data showed that PNPLA3 and PNPLA5 expression can be reduced by SMAD3 or SMAD4 knock-down, consistent with the ChIP-seq data.

TABLE 12
Knock-down of SMAD proteins via siRNA

| | | Relative | Relative |
|---|---|---|---|
| | Ligand | PNPLA3 | PNPLA5 |
| mRNA targeted | treatment | mRNA levels | mRNA levels |
| Non-targeting | BMP2 | 1.00 ± 0.09 | 1.05 ± 0.38 |
| Non-targeting | TGF-beta | 1.00 ± 0.05 | 1.01 ± 0.20 |
| SMAD1 | BMP2 | 0.92 ± 0.06 | 0.61 ± 0.03 |
| SMAD2 | TGF-beta | 1.19 ± 0.14 | 1.47 ± 0.09 |
| SMAD3 | TGF-beta | 0.44 ± 0.02 | 0.23 ± 0.04 |
| SMAD4 | TGF-beta | 0.50 ± 0.10 | 0.17 ± 0.03 |
| SMAD5 | BMP2 | 1.01 ± 0.05 | 0.98 ± 0.05 |
| SMAD9 | BMP2 | 0.99 ± 0.04 | 1.15 ± 0.06 |

The experiment was repeated for a longer siRNA treatment time of 36 hours in the absence of BMP2 or TGF-beta stimulation. Additional targets, ACVR1 and NF-κB, were targeted via siRNA-mediated knock-down. Relative PNPLA3 or PNPLA5 mRNA levels were normalized to GUSB. The results are presented in Table 13.

TABLE 13
Knock-down of SMAD proteins, ACVR1
and NF-κB via siRNA

| | Relative | Relative |
|---|---|---|
| | PNPLA3 | PNPLA5 |
| mRNA targeted | mRNA levels | mRNA levels |
| Non-targeting | 1.00 ± 0.03 | 1.01 ± 0.17 |
| SMAD3 | 0.79 ± 0.03 | 0.58 ± 0.09 |
| SMAD4 | 0.63 ± 0.05 | 0.40 ± 0.05 |
| SMAD5 | 0.83 ± 0.04 | 1.89 ± 0.17 |
| ACVR1 | 0.82 ± 0.05 | 0.44 ± 0.04 |
| NF-κB | 0.72 ± 0.04 | 0.37 ± 0.03 |

The above experiments confirmed that ACVR1, SMAD3, SMAD4, and NF-κB contribute to the regulation of PNPLA3 expression. It is likely that Momelotinib acts through inhibiting the TGF-beta/SMAD and NF-κB pathways in addition to JAK/STAT inhibition to downregulate PNPLA3.

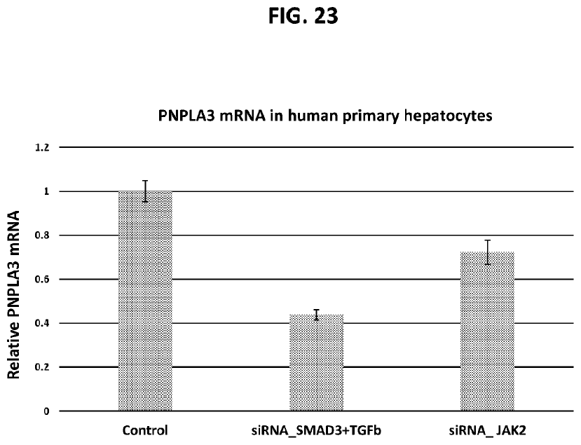

Next, primary human hepatocytes were reverse transfected with 10 nM siRNA specific for JAK2 as previously described. After 72 hours of treatment, levels of JAK2 mRNA was evaluated for knock-down efficiency and the effect of the knock-down on PNPLA3 expression was examined. The relative PNPLA3 expression for cells treated with JAK2 siRNA and for SMAD3 siRNA and TGF-beta ligand are shown in FIG. 23.

### Example 12

### In Vivo Compound Testing in Mice

Compounds that showed effective downregulation in ex vivo validation studies were chosen for in vivo testing in mice. Candidate compounds were administered at an appropriate dose once daily to a group of wild-type mice consisting of 3 male and 3 female mice. Mice were sacrificed on the fourth day and liver tissue was collected and analyzed for PNPLA3 (or COL1A1) expression by qRT-PCR. PNPLA3 expression was observed to be higher and more variable in females than in males, and therefore the data was analyzed separately for each gender. When COL1A1 was analyzed, a stellate cell specific gene GFAP was used as a house-keeping control.

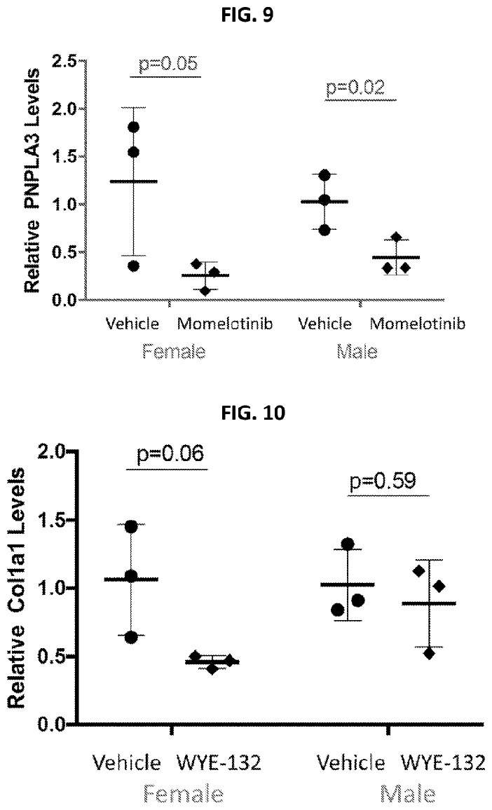

Momelotinib was dosed at 50 mg/kg and treatment of Momelotinib reduced PNPLA3 significantly in mouse liver. Albeit different baseline PNPLA3 levels, both male and female mice responded to Momelotinib treatment (see FIG. 9). No change was observed in animal body weight, organ weight or in many other liver genes such as albumin, ASGR1, and HAMP1.

WYE-125132 (WYE-132) was dosed at 50 mg/kg and treatment of WYE-125132 reduced COL1A1 expression in mouse liver (see FIG. 10), more predominantly in female mice. This is consistent with the observation that WYE-125132 decreased COL1A1 mRNA in HSCs. The reduction of COL1A1 expression levels indicates conserved mechanism between in vitro and in vivo animals.

### Example 13

### Compound Testing in Patient Cells

Candidate compounds are evaluated in patient derived induced pluripotent stem (iPS)-hepatoblast cells to confirm their efficacy. Selected patients have the I148M mutation in the PNPLA3 gene. Changes in PNPLA3 expression in hepatoblast cells are analyzed with qRT-PCR. Results are used to confirm if the pathway is similarly functional in patient cells and if the compounds have the same impact.

### Example 14

### Compound Testing in a Mouse Model

Candidate compounds are evaluated in a mouse model of PNPLA3-mediated liver disease (e.g., NASH) for in vivo activity and safety.

### Example 15

### PNPLA3 Downregulation by Momelotinib in Hepatocytes from Different Donors

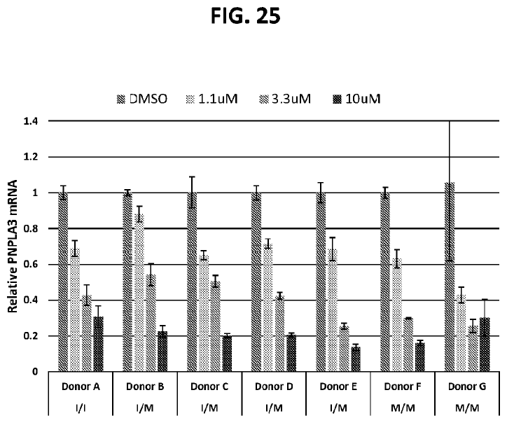

Momelotinib was tested in human hepatocytes from seven different donors at three concentrations. The donors were genotyped for the presence of the marker PNPLA3 I148M, SNP rs738409 c.444 C-G. The seven donors consisted of one homozygous WT (I/I), four heterozygous (I/M) and two homozygous mutants (M/M). The hepatocytes were treated with Momelotinib as described in Example 1 and the mRNA levels were determined by qRT-PCR. The results are presented in the Table 14 and FIG. 25. Momelotinib effectively decreased PNPLA3 expression in a dose-dependent manner in the hepatocytes from all seven donors regardless of the PNPLA3 allele status and the gender of the donor.

TABLE 14
PNPLA3 downregulation in hepatocytes from different donors

| | | | Relative PNPLA3 mRNA level vs Untreated | | |
|---|---|---|---|---|---|
| | | PNPLA3 | (±Standard Deviation) | | |
| | | allele | 1.1 μM | 3.3 μM | 10 μM |
| Donor ID | Sex | status | Momelotinib | Momelotinib | Momelotinib |
| HH1045 | M | I/I | 0.69 ± 0.04 | 0.43 ± 0.06 | 0.31 ± 0.06 |
| HH1086 | F | I/M | 0.88 ± 0.04 | 0.54 ± 0.06 | 0.23 ± 0.03 |
| HH1113 | M | I/M | 0.65 ± 0.03 | 0.51 ± 0.03 | 0.20 ± 0.01 |
| HH1121 | F | I/M | 0.72 ± 0.03 | 0.42 ± 0.02 | 0.21 ± 0.01 |
| HH1131 | F | I/M | 0.69 ± 0.06 | 0.26 ± 0.02 | 0.14 ± 0.02 |
| HH1043 | M | M/M | 0.63 ± 0.05 | 0.30 ± 0.00 | 0.16 ± 0.01 |
| HH1110 | M | M/M | 0.43 ± 0.04 | 0.26 ± 0.04 | 0.30 ± 0.10 |

### Example 16

### PNPLA3 Downregulation by Momelotinib in Stellate Cells from Different Donors

Momelotinib was tested in stellate cells from two donors across 8 concentrations. The donors were genotyped for the presence of the marker PNPLA3 I148M, SNP rs738409 c.444 C-G. The two donors were a homozygous WT (I/I) and a homozygous mutant (M/M). The stellate cells were treated with Momelotinib as described in Example 1 and the mRNA levels were determined by qRT-PCR. The results are presented in the Table 15. Momelotinib effectively decreased PNPLA3 expression in a dose-dependent manner in the stellate cells from both the WT donor and homozygous mutant donor.

TABLE 15
PNPLA3 downregulation in stellate cells from different donors

| Momelotinib | Relative PNPLA3 level vs Untreated | |
|---|---|---|
| Concentration | (±Standard Deviation) | |
| (μM) | Donor 1 (I/I) | Donor 2 (M/M) |
| 0.1 | 1.02 ± 0.14 | 1.00 ± 0.03 |
| 1.56 | 0.94 ± 0.21 | 0.85 ± 0.14 |
| 3.12 | 0.87 ± 0.08 | 0.72 ± 0.12 |
| 6.25 | 0.67 ± 0.25 | 0.69 ± 0.03 |
| 12.5 | 0.26 ± 0.14 | 0.37 ± 0.05 |
| 25 | 0.41 ± 0.10 | 0.30 ± 0.04 |
| 50 | 0.39 ± 0.07 | 0.23 ± 0.10 |
| 100 | 0.29 ± 0.03 | 0.25 ± 0.05 |

### Example 17

### PNPLA3 Downregulation Human Hepatocytes, Mouse Hepatocytes, and Stellate Cells

Additional compounds targeting various pathways were tested in human hepatocytes from five donors, mouse hepatocytes, and human stellate cells at two concentrations. The human hepatocytes, mouse hepatocytes, and stellate cells were treated with the indicated compounds as described in Example 1, and the mRNA levels were determined by qRT-PCR. The results are presented in Table 16. The numbers indicate the amount of PNPLA3 mRNA remaining after treatment with the indicated compound compared to untreated cells.

TABLE 16
PNPLA3 downregulation in human hepatocytes,
human stellate cells, and mouse hepatocytes

| | | H Hep | H Hep | H Hep | | H Hep | | H Hep | | Human | | |
|---|---|---|---|---|---|---|---|---|---|---|---|---|
| Compound | | Donor-1 | Donor-2 | Donor-3 | | Donor-4 | | Donor-5 | | Stellate | | Mouse |
| Name | Target | 10 uM | 10 uM | 10 uM | 1 uM | 10 uM | 1 uM | 10 uM | 1 uM | 10 uM | 1 uM | 10 uM |
| Momelotinib | JAK/multiple | 0.5 | 0.2 | 0.4 | 1.1 | 0.35 | 0.75 | 0.55 | 1 | 0.6 | ND | 0.24 |
| PF-00562271 | FAK | ND | 0.34 | 0.2 | 1.1 | 0.5 | 1 | 0.25 | 1 | 0.18 | 1.15 | 0.27 |
| Mubritinib | HER2 (Tyr | ND | 0.39 | 0.2 | 0.55 | 0.4 | 0.6 | 0.25 | 0.2 | 0.78 | 1.12 | 0.09 |
| (TAK 165) | Kin) | | | | | | | | | | | |
| PF-04691502 | PI3K/mTOR | 0.2 (1 uM) | 0.3 (1 uM) | 0.25 | 0.45 | 0.38 | 0.38 | 0.35 | 0.2 | 0.35 | 0.43 | 0.2 |
| XL228 | IGF1R/ | 0.4 (1 uM) | ND | 0.3 | 0.55 | 0.55 | 0.55 | 0.25 | 0.8 | 0.7 | 0.4 | 0.12 |
| | SRC-ABL | | | | | | | | | | | |
| OSI-027/ | mTORC1/2 | 0.3 (1 uM) | 0.4 (1 uM) | 0.55 | 0.6 | 0.38 | 0.5 | 0.2 | 0.2 | 0.65 | 0.3 | 0.35 |
| ASP7486 | | | | | | | | | | | | |
| LY2157299 | Alk5/TgfbRI | 0.50 | 0.7 | 0.8 | 0.75 | 0.5 | 0.55 | 0.5 | 0.65 | 1 | ND | 1.02 |
| Galunisertib | | | | | | | | | | | | |
| SIS3 | SMAD3 i/tool | 0.2 | 0.55 | ND | ND | ND | ND | ND | ND | ND | ND | 0.50 |

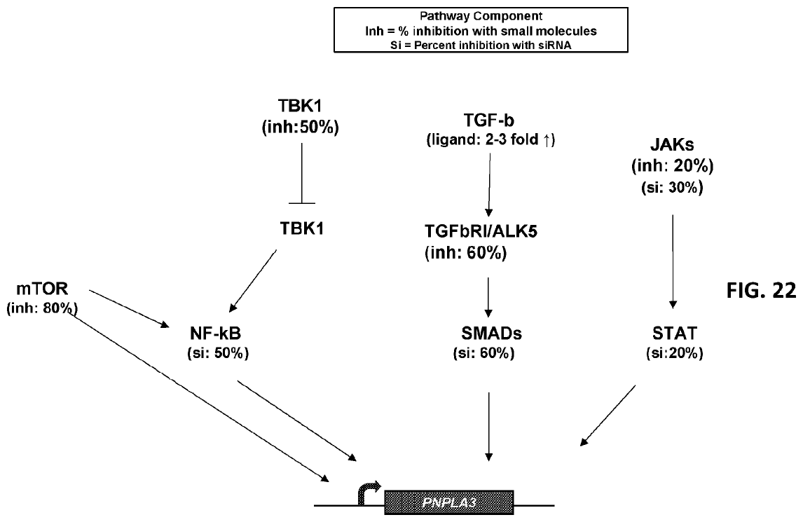

A diagram of the signaling pathways that affect PNPLA3 expression is shown in FIG. 22. Also shown is a comparison of the inhibition of the signaling pathways with small molecules or siRNA.

### Example 18

### OSI-027 and PF-04691502 Downregulate PNPLA3 in Human Hepatocytes and Stellate Cells

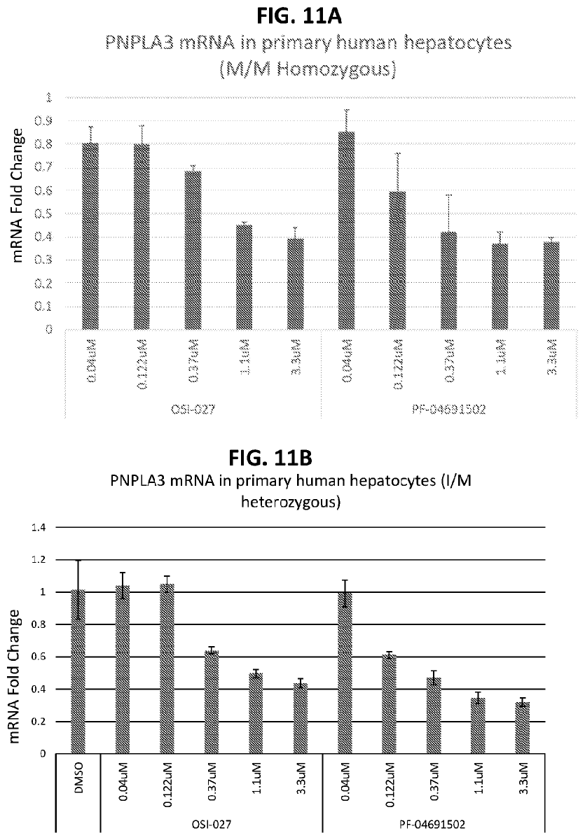

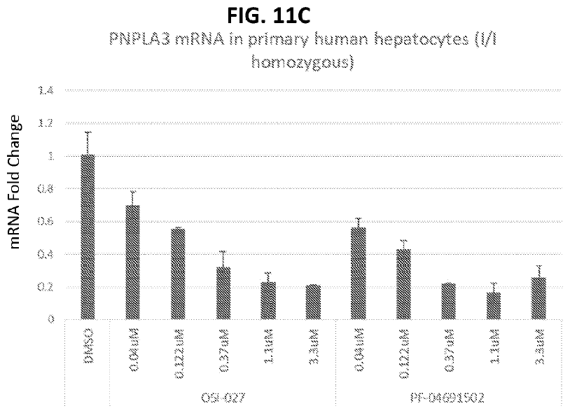

OSI-027 and PF-04691502 were tested in human hepatocytes from 5 different donors at five concentrations. The donors were genotyped for the presence of the marker PNPLA3 I148M, SNP rs738409 c.444 C-G. The 5 donors consisted of 1 homozygous WT (I/I), 2 heterozygous (I/M) and 2 homozygous mutants (M/M). The hepatocytes were treated with OSI-027 and PF-04691502 as described in Example 1 and the mRNA levels were determined by qRT-PCR. PNPLA3 mRNA levels were normalized to GUSB. The homozygous (M/M) results are presented in FIG. 11A and Table 17, the heterozygous (I/M) results are presented in FIG. 11B and Table 18, and the homozygous (I/I) results are presented in FIG. 11C and Table 18. OSI-027 and PF-04691502 effectively decreased PNPLA3 expression in a dose-dependent manner in the hepatocytes from all donors regardless of the PNPLA3 allele status of the donor.

TABLE 17
PNPLA3 downregulation in (M/M) homozygous
human hepatocytes from different donors

| | | Relative PNPLA3 |
|---|---|---|
| | Concentration, | level vs Untreated |
| Compound | μM | (±Standard Deviation) |
| DMSO | | 1.00 ± 0.14 |
| OSI-027 | 0.04 uM | 0.70 ± 0.082 |
| | 0.122 uM | 0.56 ± .0071 |
| | 0.37 uM | 0.32 ± 0.096 |
| | 1.1 uM | 0.23 ± 0.0523 |
| | 3.3 uM | 0.21 ± 0.0024 |
| PF-04691502 | 0.04 uM | 0.56 ± 0.056 |
| | 0.122 uM | 0.43 ± 0.052 |
| | 0.37 uM | 0.22 ± 0.0015 |
| | 1.1 uM | 0.17 ± 0.053 |
| | 3.3 uM | 0.26 ± 0.072 |

TABLE 18
PNPLA3 downregulation in (I/M) heterozygous
human hepatocytes from different donors

| | | Relative PNPLA3 |
|---|---|---|
| | Concentration, | level vs Untreated |
| Compound | μM | (±Standard Deviation) |
| DMSO | | 1.01 ± 0.18 |
| OSI-027 | 0.04 uM | 1.04 ± 0.081 |
| | 0.122 uM | 1.05 ± 0.051 |
| | 0.37 uM | 0.64 ± 0.022 |
| | 1.1 uM | 0.50 ± 0.026 |
| | 3.3 uM | 0.44 ± 0.029 |
| PF-04691502 | 0.04 uM | 1.0 ± 0.083 |
| | 0.122 uM | 0.61 ± 0.021 |
| | 0.37 uM | 0.47 ± 0.043 |
| | 1.1 uM | 0.35 ± 0.035 |
| | 3.3 uM | 0.32 ± 0.026 |

TABLE 19
PNPLA3 downregulation in (I/I) homozygous
human hepatocytes from different donors

| | | Relative PNPLA3 |
|---|---|---|
| | Concentration, | level vs Untreated |
| Compound | μM | (±Standard Deviation) |
| OSI-027 | 0.04 uM | 0.80 ± 0.070 |
| | 0.122 uM | 0.80 ± 0.080 |
| | 0.37 uM | 0.68 ± 0.023 |
| | 1.1 uM | 0.45 ± 0.015 |
| | 3.3 uM | 0.39 ± 0.048 |
| PF-04691502 | 0.04 uM | 0.85 ± 0.094 |
| | 0.122 uM | 0.60 ± 0.16 |
| | 0.37 uM | 0.42 ± 0.16 |
| | 1.1 uM | 0.37 ± 0.050 |
| | 3.3 uM | 0.38 ± 0.020 |

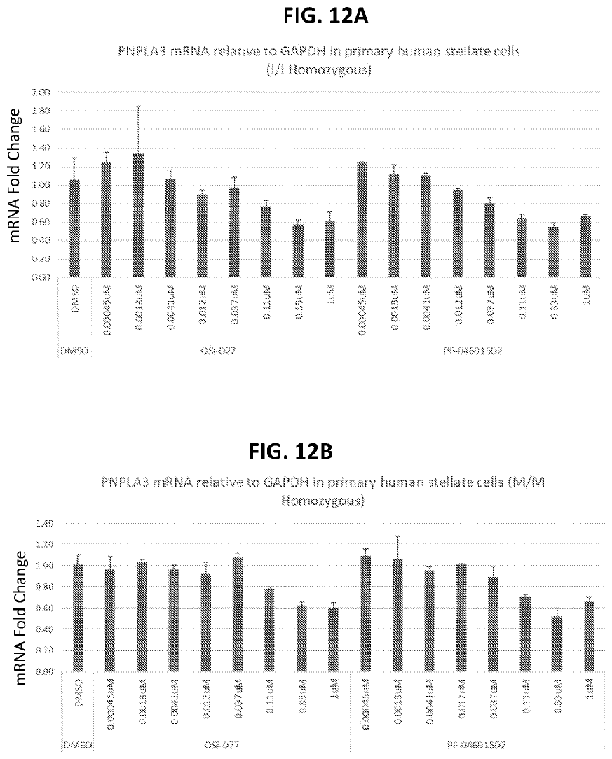

OSI-027 and PF-04691502 were also tested in PNPLA3 1148 (I/I) or (M/M) homozygous human stellate cells at eight concentrations. The stellate cells were treated with the indicated compounds as described in Example 1, and the mRNA levels were determined by qRT-PCR. PNPLA3 mRNA levels were normalized to GAPDH. The homozygous (I/I) results are presented in FIG. 12A and Table 20, and the homozygous (M/M) results are presented in FIG. 12B and Table 21. OSI-027 and PF-04691502 effectively decreased PNPLA3 expression in a dose-dependent manner in the hepatocytes from all donors regardless of the PNPLA3 allele status of the donor.

TABLE 20
PNPLA3 downregulation in (I/I) homozygous
human stellate cells

| | | Relative PNPLA3 |
|---|---|---|
| | Concentration, | level vs Untreated |
| Compound | μM | (±Standard Deviation) |
| DMSO | | 1.06 ± 0.23 |
| OSI-027 | 0.00045 uM | 1.25 ± 0.10 |
| | 0.0013 uM | 1.34 ± 0.51 |
| | 0.0041 uM | 1.06 ± 0.10 |
| | 0.012 uM | 0.89 ± 0.05 |
| | 0.037 uM | 0.97 ± 0.12 |
| | 0.11 uM | 0.77 ± 0.07 |
| | 0.33 uM | 0.57 ± 0.05 |
| | 1 uM | 0.62 ± 0.09 |
| PF-04691502 | 0.00045 uM | 1.24 ± 0.01 |
| | 0.0013 uM | 1.12 ± 0.09 |
| | 0.0041 uM | 1.10 ± 0.03 |
| | 0.012 uM | 0.95 ± 0.01 |
| | 0.037 uM | 0.80 ± 0.06 |
| | 0.11 uM | 0.64 ± 0.05 |
| | 0.33 uM | 0.55 ± 0.04 |
| | 1 uM | 0.66 ± 0.02 |

TABLE 21
PNPLA3 downregulation in (M/M) homozygous
human stellate cells

| | | Relative PNPLA3 |
|---|---|---|
| | Concentration, | level vs Untreated |
| Compound | μM | (±Standard Deviation) |
| DMSO | | 1.01 ± 0.09 |
| OSI-027 | 0.00045 uM | 0.96 ± 0.12 |
| | 0.0013 uM | 1.04 ± 0.02 |
| | 0.0041 uM | 0.96 ± 0.04 |
| | 0.012 uM | 0.92 ± 0.12 |
| | 0.037 uM | 1.08 ± 0.04 |
| | 0.11 uM | 0.78 ± 0.01 |
| | 0.33 uM | 0.63 ± 0.03 |
| | 1 uM | 0.60 ± 0.05 |
| PF-04691502 | 0.00045 uM | 1.09 ± 0.07 |
| | 0.0013 uM | 1.06 ± 0.21 |
| | 0.0041 uM | 0.96 ± 0.03 |
| | 0.012 uM | 1.01 ± 0.01 |
| | 0.037 uM | 0.89 ± 0.10 |
| | 0.11 uM | 0.71 ± 0.02 |
| | 0.33 uM | 0.52 ± 0.08 |
| | 1 uM | 0.66 ± 0.04 |

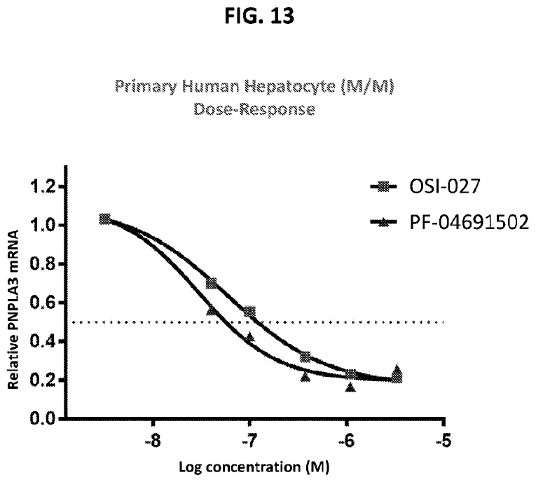

The EC50 of both OSI-027 and PF-04691502 in primary hepatocytes and stellate cells in shown in FIG. 13 and Table 22.

TABLE 22
PNPLA3 Dose-Response Summary and EC50

| Cell type | Compound | EC50 |
|---|---|---|
| Primary hep (M/M) | OSI-027 | 60 nM |
| | PF-0469152 | 30 nM |
| Stellate cell (M/M) | OSI-027 | 62 nM |
| | PF-0469152 | 88 nM |

### Example 19

### OSI-027 and PF-04691502 Reduce Lipid Content in Primary Human Hepatocytes and HepG2 Cells

The ability of OSI-027 and PF-04691502 to reduce lipid content in hepatocytes or HepG2 cells was next assessed.

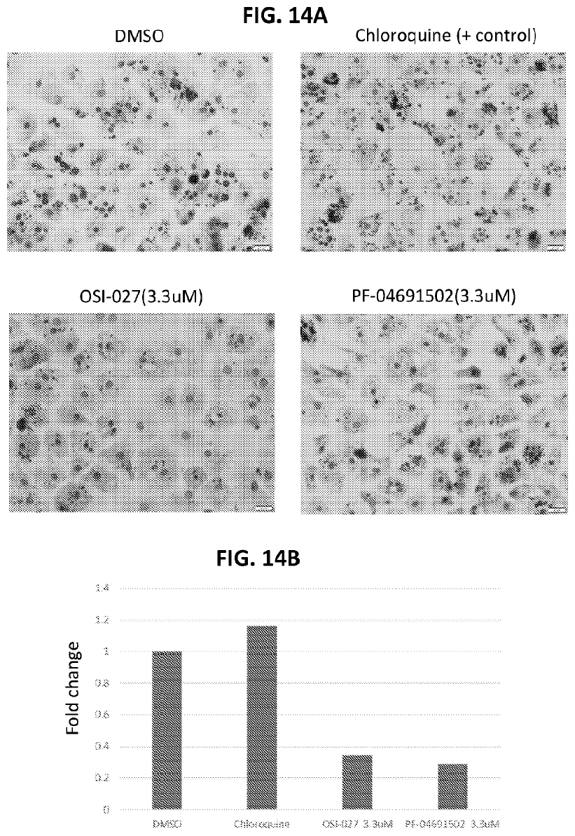

Primary human hepatocytes (M/M homozygous) were treated with 3.3 μM OSI-027 or PF-04691502 as described in Example 1. DMSO and chloroquine were used as controls. After treatment, the cells were fixed and stained with ORO using the BioVision Lipid (Oil Red O) Staining Kit (cat #K580-24) according to the manufacturer's instructions. Parallel treatment samples were processed for qRT-PCR as previously described. Representative light microscopy images of each treatment are shown in FIG. 14A and a quantification of the PNPLA3 mRNA levels are shown in FIG. 14B. Both OSI-027 and PF-04691502 treatment resulted in reduced lipid content in primary human hepatocytes.

HepG2 cells were treated with OSI-027 as described in Example 1. DMSO was used as a control. After treatment, the cells were stained with the AdipoRed™ Assay Reagent (cat #PT-7009) according to the manufacturer's instructions. Parallel treatment samples were processed for triglyceride quantification. For triglyceride quantification, HepG2 cells were treated with OSI-027 for 72 hours. Cells were collected and the lipid droplet (LD) fraction of the cell lysate enriched using the lipid droplet isolation kit (Cell Biolabs Inc., #MET-5011) per the manufacturer's instructions. The triglyceride content of the LD-enriched fraction measured using a Triglyceride Quantification Kit (Biovision Inc., #K622) per manufacturer's protocol, with a fluorimetric read-out.

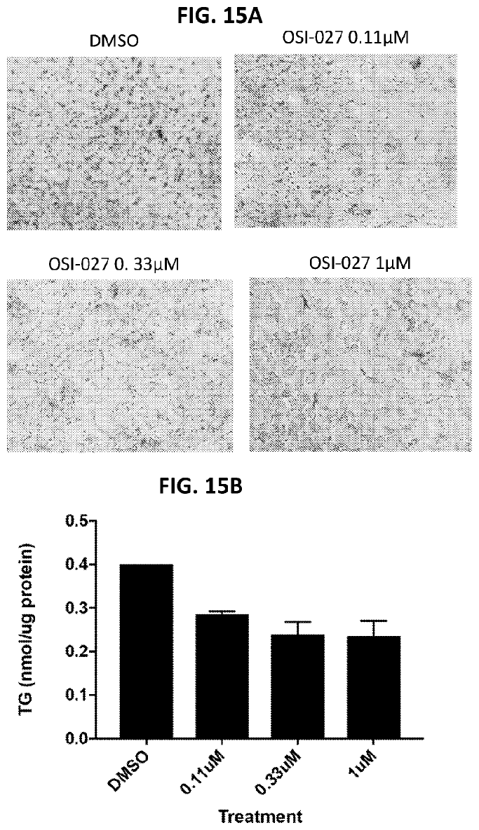

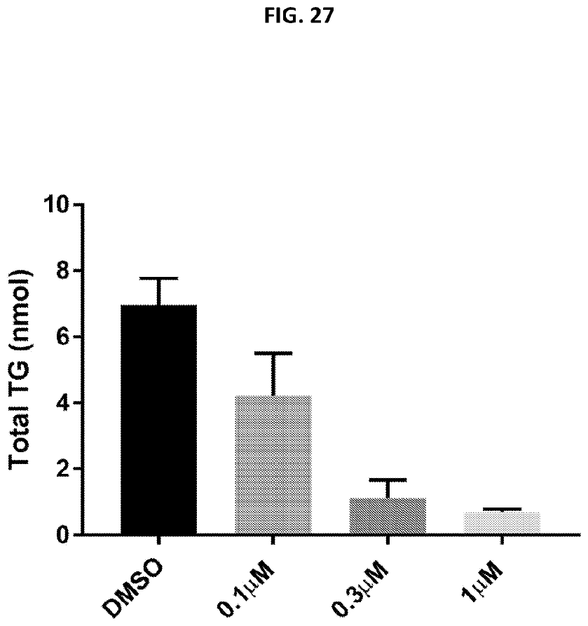

Representative light microscopy images of each treatment are shown in FIG. 15A and a quantification of the triglyceride levels are shown in FIGS. 15B, 27 and Table 22. FIG. 15B shows the relative amount (nmol/ug protein) of trigyceride in each sample after OSI-027 treatment, while FIG. 27 provides the total triglyceride (nmol) in each sample after OSI-027 treatment. OSI-027 treatment resulted in reduced triglyceride content in HepG2 cells.

TABLE 22
OSI-027 reduces triglyceride content in HepG2 cells

| | | TG (nmol/ug |
|---|---|---|
| | | protein) |
| | Concentration, | (±Standard |
| Compound | μM | Deviation) |
| DMSO | | 0.4 ± 0 |
| OSI-027 | 0.11 μM | 0.285 ± 0.00707 |
| | 0.33 μM | 0.24 ± 0.028284 |
| | 1 μM | 0.235 ± 0.035355 |

### Example 20

### Murine In Vivo OSI-027, PF-04691502, and LY2157299 Pharmacology Study

OSI-027 and PF-04691502 showed effective downregulation in ex vivo validation studies and were next tested in vivo in mice. LY2157299 was also tested in vivo. C57BL/6J mice were divided into 12 groups. Each group had 6 male mice. All mice were given a high sucrose (HS) diet at night on a synchronized schedule. The diet regimen was initiated 6 days prior to dosing. Starting Day 7, mice were administered with a single concentration of a candidate compound four times QD daily via oral gavage for four consecutive days. OSI-027 was administered at 50 mg/kg, PF-04691502 was administered at 10 mg/kg, and LY2157299 was administered at 75 mg/kg. Groups 1-10 received food throughout the dosing period. Groups 11-14 received food for three out of the four nights of the dosing and were fed the following morning along with the final dose of the drug. Mice in groups 1-10 were sacrificed 12 hours post-last dose on Day 11, and mice in groups 11-14 were sacrificed 6 hours post-last dose on Day 11. Organs including liver, spleen, kidney, adipose, plasma, and muscle were collected. Eyes were also collected for groups 11-14.

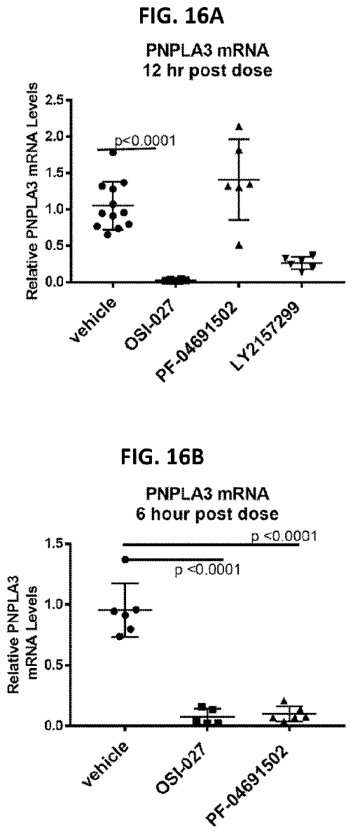

Mouse liver tissues were pulverized in liquid nitrogen and aliquoted into small microtubes. TRIzol (Invitrogen Cat #15596026) was added to the tubes to facilitate cell lysis from tissue samples. The TRIzol solution containing the disrupted tissue was then centrifuged and the supernatant phase was collected. Total RNA was extracted from the supernatant using Qiagen RNA Extraction Kit (Qiagen Cat#74182) and the PNPLA3 mRNA levels were analyzed using qRT-PCR. PNPLA3 mRNA levels in the 6 and 12 hr post dose groups is shown in FIGS. 16A and 16B.

Treatment with OSI-027 reduced PNPLA3 mRNA at both 12 hours post-dose and 6 hours post-dose, as shown in FIGS. 16A and 16B. However, animals showed toxicity at the 50 mg/kg dose.

Treatment with PF-04691502 reduced PNPLA3 mRNA at 6 hours post-dose but not at 12 hours post-dose, as shown in FIGS. 16A and 16B. However, animals showed toxicity at the 10 mg/kg dose.

Treatment with LY2157299 reduced PNPLA3 mRNA at 12 hours post-dose, as shown in FIG. 16B. In addition, animals did not show toxicity at the 75 mg/kg dose.

### Example 21

### Murine In Vivo OSI-027, PF-04691502, and LY2157299 Dose Response Pharmacology Study

As OSI-027 and PF-04691502 both showed toxicity in mice, an in vivo dose titration study was completed. C57BL/6J mice were divided into 14 groups. Each group had 6 male mice. All mice were given an HS diet at night on a synchronized schedule. The diet regimen was initiated 6 days prior to dosing. Starting Day 7, different mice groups were administered decreasing amounts of a candidate compound four times QD daily via oral gavage for four consecutive days. Table 23 shows the treatment and dose for each animal group. The animals received no food at night on Day 10. Animals were sacrificed 6 hours post-last dose on Day 11. Organs including liver, spleen, kidney, adipose, plasma, and muscle were collected.

Mouse liver tissues were pulverized in liquid nitrogen and aliquoted into small microtubes. TRIzol (Invitrogen Cat #15596026) was added to the tubes to facilitate cell lysis from tissue samples. The TRIzol solution containing the disrupted tissue was then centrifuged and the supernatant phase was collected. Total RNA was extracted from the supernatant using Qiagen RNA Extraction Kit (Qiagen Cat #74182) and the target mRNA levels were analyzed using qRT-PCR. mRNA levels for PNPLA3, PNPLA5, COL1A1, and PCSK9 were assessed.

TABLE 23
Mouse Groups and Compound Doses

| Group | Compound Name | Dose | Termination |
|---|---|---|---|
| 1 | Vehicle | — | Day 11, 1-2 pm |
| 2 | OSI-027 | 50 mg/kg QD | Day 11, 1-2 pm |
| 3 | OSI-027 | 25 mg/kg QD | Day 11, 1-2 pm |
| 4 | OSI-027 | 10 mg/kg QD | Day 11, 1-2 pm |
| 5 | OSI-027 | 5 mg/kg QD | Day 11, 1-2 pm |
| 6 | OSI-027 | 2 mg/kg | Day 11, 1-2 pm |
| 7 | PF-04691502 | 10 mg/kg QD | Day 11, 1-2 pm |
| 8 | PF-04691502 | 5 mg/kg QD | Day 11, 1-2 pm |
| 9 | PF-04691502 | 2 mg/kg QD | Day 11, 1-2 pm |
| 10 | PF-04691502 | 1 mg/kg QD | Day 11, 1-2 pm |
| 11 | LY2157299 | 75 mg/kg QD | Day 11, 1-2 pm |
| 12 | LY2157299 | 50 mg/kg QD | Day 11, 1-2 pm |
| 13 | LY2157299 | 25 mg/kg QD | Day 11, 1-2 pm |
| 14 | LY2157299 | 10 mg/kg QD | Day 11, 1-2 pm |

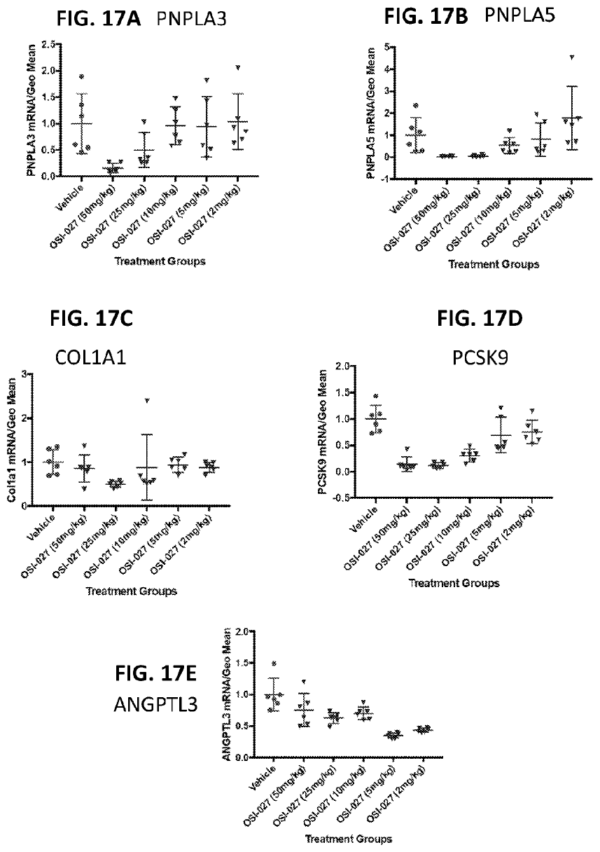

Mice in groups 2-6 treated with OSI-027 had a dose dependent decrease in PNPLA3, PNPLA5, PSCK9, and ANGLPTL3 mRNA at 6 hours post dose (FIGS. 17A, 17B, 17C, and 17E). Treatment with OS1-027 did not result in a significant decrease in COL1A1 mRNA at 6 hours post dose (FIG. 17C), similar to the result seen with Momelotinib treatment in Example 9.

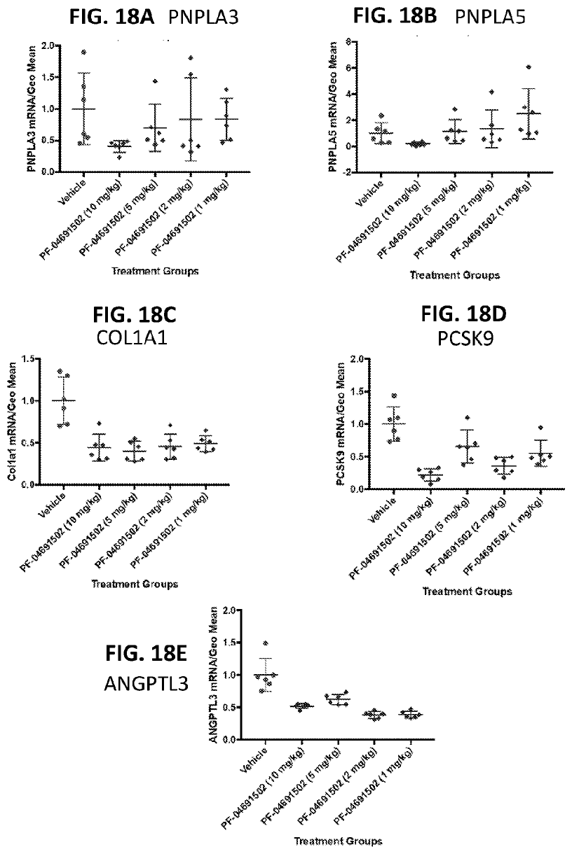

Mice in groups 7-10 treated with PF-04691502 had a dose dependent decrease in PNPLA3 and PNPLA5 mRNA 6 hours post dose (FIGS. 18A and 18B). All concentrations of PF-04691502 tested resulted in a decrease in COL1A1, ANGLPTL3 and PCSK9 mRNA at 6 hours post dose (FIGS. 18C, 18D, and 18E).

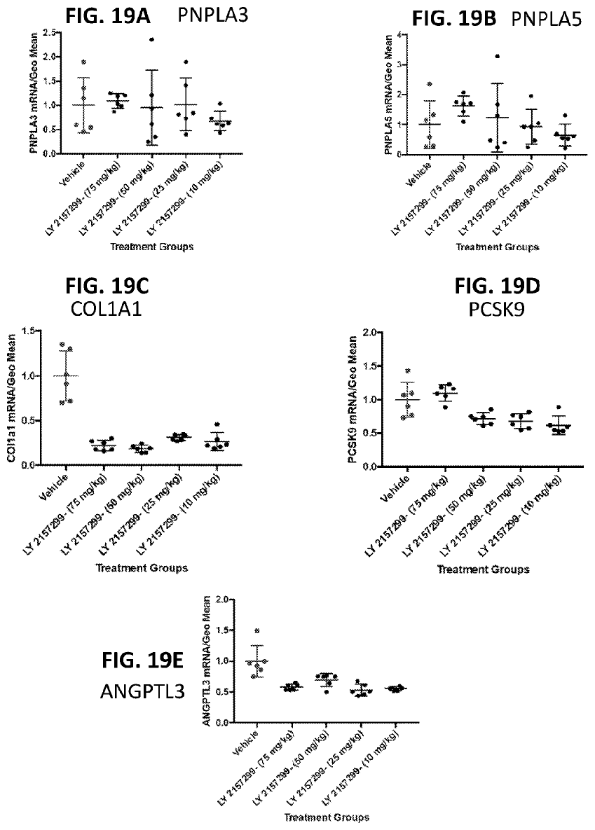

Mice in groups 11-14 treated with LY 2157299 did not show a significant decrease in PNPLA3 or PNPLA5 mRNA at 6 hours post dose (FIGS. 19A and 19B). However, all concentrations of LY 2157299 tested resulted in a decrease in COL1A1 and ANGLPTL3 mRNA at 6 hours post dose (FIGS. 19C and 19E), and the three lower doses (50 mg/kg, 25 mg/kg, and 10 mg/kg) resulted in a decrease in PCSK9 mRNA at 6 hours post dose (FIG. 19D).

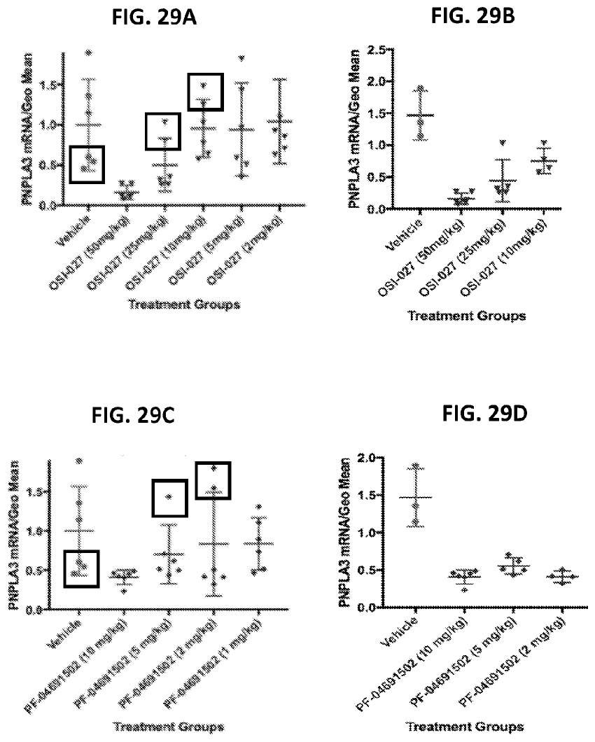

Further characterization of the lower outlier mice in the OSI-027 control treatment groups showed that the control mice with the lowest PNPLA3 mRNA expression also had low pS6 and/or pAKT expression and thus low mTOR pathway activation (data not shown), while the mice in the 25 mg/kg OSI-027 treatment group with the highest amount of PNPLA3 mRNA after treatment had high pS6 and pAKT and thus high mTOR pathway activation. Exclusion of these outliers and re-analysis of the data showed that OSI-027 had a more significant dose dependent decrease in PNPLA3 mRNA at 6 hours post dose (FIG. 29A, boxed groups, and FIG. 29B). Similar characterization and reanalysis of the PF-04691502 treated mice showed that PF-04691502 treatment resulted in a greater decrease in PNPLA3 mRNA at 6 hours post dose (FIG. 29C, boxed groups, and FIG. 29D). These data confirm the role of mTOR in regulation of PNPLA3 expression.

### Example 22

### Human Treatment Using PNPLA3 Inhibitors

A human subject is administered an effect amount of any of the compounds in the forgoing examples and Table 1, such as OSI-027, PF-04691502, LY2157299, Momelotinib, Apitolisib, BML-275, DMH-1, Dorsomorphin, Dorsomorphin dihydrochloride, K 02288, LDN-193189, LDN-212854, ML347, SIS3, AZD8055, BGT226 (NVP-BGT226), CC-223, Chrysophanic Acid, CZ415, Dactolisib (BEZ235, NVP-BEZ235), Everolimus (RAD001), GDC-0349, Gedatolisib (PF-05212384, PKI-587), GSK1059615, INK 128 (MLN0128), KU-0063794, LY3023414, MHY1485, Omipalisib (GSK2126458, GSK458), Palomid 529 (P529), PI-103, PP121, Rapamycin (Sirolimus), Ridaforolimus (Deforolimus, MK-8669), SF2523, Tacrolimus (FK506), Temsirolimus (CCI-779, NSC 683864), Torin 1, Torin 2, Torkinib (PP242), Vistusertib (AZD2014), Voxtalisib (SAR245409, XL765) Analogue, Voxtalisib (XL765, SAR245409), WAY-600, WYE-125132 (WYE-132), WYE-354, WYE-687, XL388, Zotarolimus (ABT-578), R788, tamatinib (R406), entospletinib (GS-9973), nilvadipine, TAK-659, BAY-61-3606, MNS (3,4-Methylenedioxy-β-nitrostyrene, MDBN), Piceatannol, PRT-060318, PRT062607 (P505-15, BIIB057), PRT2761, R09021, cerdulatinib, ibrutinib, ONO-4059, ACP-196, idelalisib, duvelisib, pilaralisib, TGR-1202, GS-9820, ACP-319, SF2523, BIO, AZD2858, 1-Azakenpaullone, AR-A014418, AZD1080, Bikinin, BIO-acetoxime, CHIR-98014, CHIR-99021 (CT99021), IM-12, Indirubin, LY2090314, SB216763, SB415286, TDZD-8, Tideglusib, TWS119, ACHP, 10Z-Hymenialdisine, Amlexanox, Andrographolide, Arctigenin, Bay 11-7085, Bay 11-7821, Bengamide B, BI 605906, BMS 345541, Caffeic acid phenethyl ester, Cardamonin, C-DIM 12, Celastrol, CID 2858522, FPS ZM1, Gliotoxin, GSK 319347A, Honokiol, HU 211, IKK 16, IMD 0354, IP7e, IT 901, Luteolin, MG 132, ML 120B dihydrochloride, ML 130, Parthenolide, PF 184, Piceatannol, PR 39 (porcine), Pristimerin, PS 1145 dihydrochloride, PSI, Pyrrolidinedithiocarbamate ammonium, RAGE antagonist peptide, Ro 106-9920, SC 514, SP 100030, Sulfasalazine, Tanshinone IIA, TPCA-1, Withaferin A, Zoledronic Acid, Ruxolitinib, Oclacitinib, Baricitinib, Filgotinib, Gandotinib, Lestaurtinib, PF-04965842, Upadacitinib, Cucurbitacin I, CHZ868, Fedratinib, AC430, AT9283, ati-50001 and ati-50002, AZ 960, AZD1480, BMS-911543, CEP-33779, Cerdulatinib (PRT062070, PRT2070), Curcumol, Decernotinib (VX-509), Fedratinib (SAR302503, TG101348), FLLL32, FM-381, GLPG0634 analogue, Go6976, JANEX-1 (WHI-P131), NVP-BSK805, Pacritinib (SB1518), Peficitinib (ASP015K, JNJ-54781532), PF-06651600, PF-06700841, R256 (AZD0449), Solcitinib (GSK2586184 or GLPG0778), S-Ruxolitinib (INCB018424), TG101209, Tofacitinib (CP-690550), WHI-P154, WP1066, XL019, ZM 39923 HCl, Amuvatinib, BMS-754807, BMS-986094, LY294002, Pifithrin-μ, and XMU-MP-1, or a derivative or an analog thereof.

A reduction in the expression of the PNPLA3 gene is observed in the subject after the treatment.

### Example 23

### Momelotinib Reduces Chromatin Accessibility and PNPLA3 mRNA Levels

Human primary hepatocytes were treated with 10 uM Momelotinib for 1 hr or 16 hrs. Untreated hepatocytes were used as a control (0 hr timepoint). Calles were processed for ATAC-Seq PCR as previously described in Example 1.

PCR primers used for the ATAC-Seq are shown in Table 24.

TABLR 24

| # | Target | Forward Primer | Reverse Primer |
|---|---|---|---|
| 1 | PNPLA3 super-enhancer | CCCAAACCCCTTTCCCCACAT | GGTCAGAGGAGGAGACTGGCA |
| | (chr22: 43, 897, 991-43, | | |
| | 898, 239) | | |
| 2 | PNPLA3 super-enhancer | AAGAGTCATCTCCTCCGGGCA | TCCAGCTGCACAGCTCAATCT |
| | (chr22: 43, 883, 098-43, | | |
| | 883, 380) | | |
| 3 | PNPLA3 super-enhancer | CATTGGGTAGGAGCAGTGGGC | TCCACAGGCCACCTTGGGATA |
| | (chr22: 43, 925, 416-43, | | |
| | 926, 009) | | |
| 4 | PNPLA3 super-enhancer | TCAGGAGGTGAGGTGCTTGGA | GCACAACAGGGCCTCCTGAAA |
| | (chr22: 43, 899, 201-43, | | |
| | 899, 638) | | |
| 5 | PNPLA3 super-enhancer | GTGTGGGGGAAACTGATAGGC | CCAGTAGAGGGCACCACACAC |
| | (chr22: 43, 945, 868-43, | | |
| | 946, 068) | | |

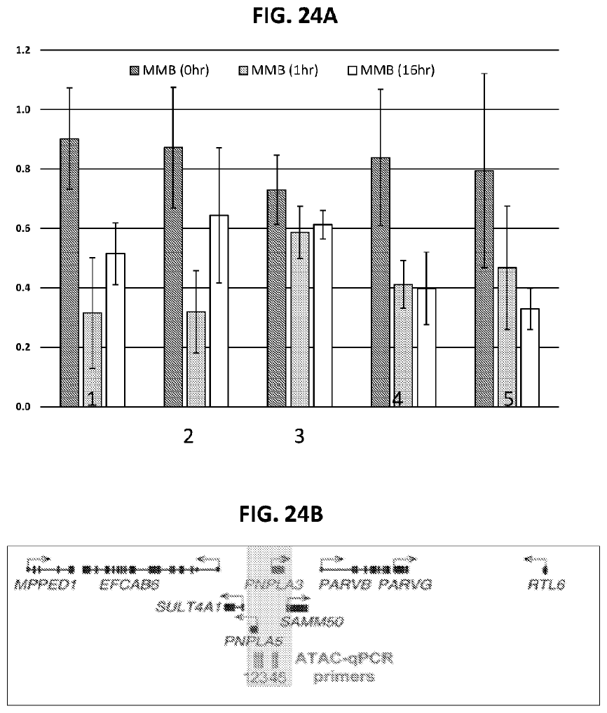

The chromatin accessibility of the PNPLA3 enhancer at 1 hr and 16 hrs post Momelotinib treatment is shown in FIGS. 24A and B. FIG. 24A provides a quantification of the relative fold change in enrichment of accessible chromatin in the indicated samples, the numbers refer to the primer pair used in the ATAC-seq, while FIG. 24B provides a diagram of the chromosomal region and primer locations. As shown, Momelotinib reduces the chromatin associability in the PNPLA3 region.

### Example 24

### Murine In Vivo Momelotinib Dose Response Pharmacology Study

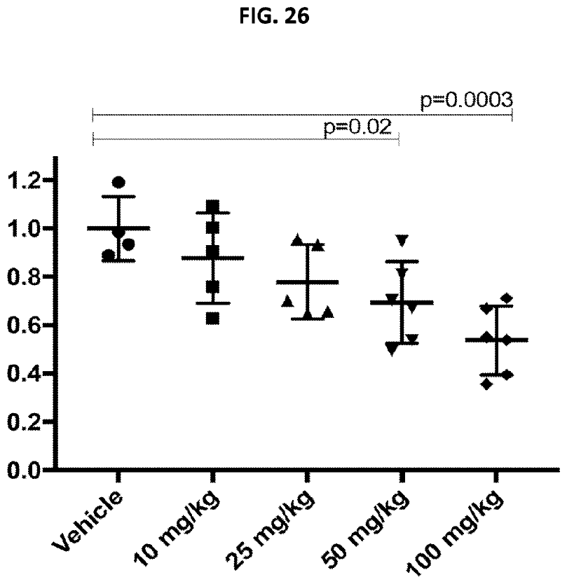

An in vivo dose response pharmacology study was also performed with Momelotinib. Mice were dosed with indicated 10, 25, 50, or 100 mg/kg Momelotinib as described in Example 12. Mouse liver tissues were pulverized in liquid nitrogen and aliquoted into small microtubes. TRIzol (Invitrogen Cat #15596026) was added to the tubes to facilitate cell lysis from tissue samples. The TRIzol solution containing the disrupted tissue was then centrifuged and the supernatant phase was collected. Total RNA was extracted from the supernatant using Qiagen RNA Extraction Kit (Qiagen Cat #74182) and the PNPLA3 mRNA levels were analyzed using qRT-PCR. PNPLA3 mRNA levels are shown in FIG. 26.

Treatment with Momelotinib reduced PNPLA3 mRNA in a dose dependent manner as shown in FIG. 26. No cytotoxicity was shown at any concentration.

### Example 25

### Momelotinib Metabolites Reduce PNPLA3 Expression

Momelotinib metabolite M21 was synthesized and tested in human hepatocytes and stellate cells in parallel with momelotinib, according to methods previously described. M21 metabolite synthesis is described in Zheng et al, Drug Metab Dispos, 2018:237. Cells were treated with Momelotinib or M21 for 16 hours. Two different human hepatocyte lines were used. Yecuris RMG and Lonza HU4282 hepatocytes and PNPLA3 mRNA fold change was determined relative to GUSB. Stellate cells form two different donors were used, ST1 and ST8, and PNPLA3 mRNA fold change was determined relative to GAPDH.

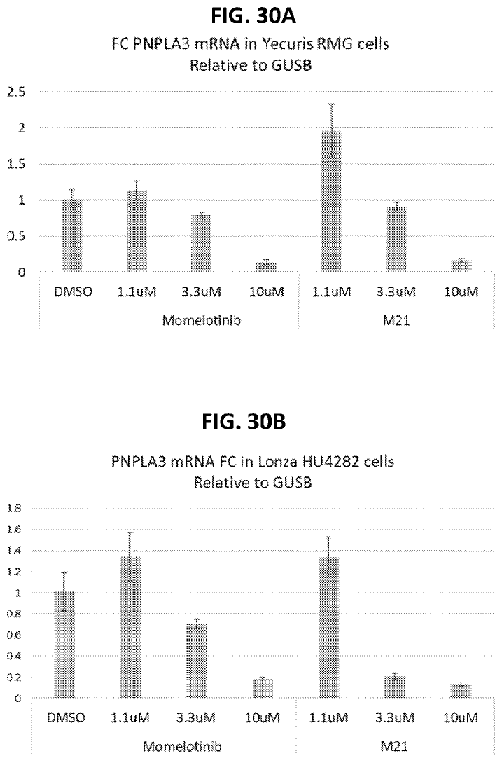

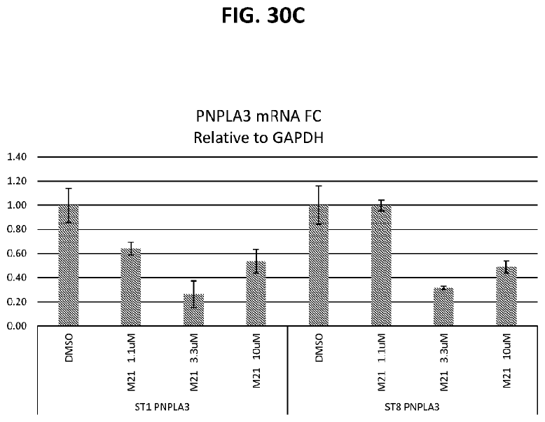

As shown in FIGS. 30A and 30B, treatment of hepatocyte cells lines with the momelotinib metabolite M21 reduced PNPLA3 mRNA expression in a dose dependent manner. FIG. 30A and Table 25 show the PNPLA3 expression in Yecuris RMG cells, while FIG. 30B and Table 26 show PNPLA3 expression in HU4282 cells.

TABLE 25

| | | Relative |
|---|---|---|
| | | PNPLA3 |
| | | mRNA |
| | | expression |
| | | in Yecuris |
| | | RMG cells |
| | DMSO | 1.008657 ± 0.13748 |
| Momelotinib | 1.1 uM | 1.138 ± 0.128 |
| | 3.3 uM | 0.802 ± 0.032 |
| | 10 uM | 0.137 ± 0.038 |
| M21 | 1.1 uM | 1.958 ± 0.368 |
| | 3.3 uM | 0.910 ± 0.068 |
| | 10 uM | 0.165 ± 0.018 |

TABLE 26

| | | Relative PNLPA3 |
|---|---|---|
| | | mRNA expression |
| | | in HU4282 cells |
| | DMSO | 1.014 ± 0.182 |
| Momelotinib | 1.1 uM | 1.342 ± 0.231 |
| | 3.3 uM | 0.705 ± 0.048 |
| | 10 uM | 0.185 ± 0.014 |
| M21 | 1.1 uM | 1.340 ± 0.188 |
| | 3.3 uM | 0.213 ± 0.028 |
| | 10 uM | 0.138 ± 0.015 |

As shown in FIG. 30C and Table 27, treatment of stellate cells with the Momelotinib metabolite M21 reduced PNPLA3 mRNA expression, with the most significant decrease after treatment with 3.3 μM M21.

TABLE 27

| | | Relative |
|---|---|---|
| | | PNPLA3 nRNA |
| | | expression in |
| | | Stellate cells |
| ST1 PNPLA3 | DMSO | 1.00 ± 0.14 |
| | M21 1.1 uM | 0.64 ± 0.05 |
| | M21 3.3 uM | 0.26 ± 0.11 |
| | M21 10 uM | 0.54 ± 0.10 |
| ST8 PNPLA3 | DMSO | 1.00 ± 0.16 |
| | M21 1.1 uM | 1.00 ± 0.04 |
| | M21 3.3 uM | 0.31 ± 0.01 |
| | M21 10 uM | 0.49 ± 0.05 |

### Example 26

### Additional Inhibitor Compound Testing

Additional compounds to reduce PNPLA3 expression were tested in human and mouse primary hepatocytes and human primary stellate cells as previously described. Additional compounds tested are shown in Table 28.

TABLE 28

| Compound Name | Synonyms | CAS Number | Target |
|---|---|---|---|
| Apitolisib (GDC-0980, | GDG-0980; GNE 390; | 1032754-93-0 | PI3K (α/β/δ/γ)/mTOR |
| RG7422) | RG 7422 | | |
| PF-04691502 | | 1013101-36-4 | PI3K (α/β/δ/γ)/mTOR |
| GDC-0980 | Apitolisib | 1032754-93-0 | PI3K (α/β/δ/γ)/mTOR |
| VS-5584 (SB2343) | | 1246560-33-7 | PI3K (α/β/δ/γ)/mTOR |
| Buparlisib (BKM120, | NVP-BKM120; | 944396-07-0 | PI3K (α/β/δ/γ) |
| NVP-BKM120) | BKM120 | | |
| CC-223 | | 1228013-30-6 | mTORC1/2 |
| CH5132799 | | 1007207-67-1 | PI3Ka/b |
| WYE-125132 | WYE-125132 | 1144068-46-1 | mTORC1 and 2 |
| (WYE-132) | | | |
| CZ415 | | 1429639-50-8 | mTORC1 and 2 |
| AZD-8055 | | 1009298-09-2 | mTORC1 and 2 |
| NVP-BKM120 (Hydrochloride) | | 1312445-63-8 | PI3K (α/β/δ/γ |
| NVP-BKM120 | Buparlisib | 944396-07-0 | PI3K (α/β/δ/γ |
| AZD2014 | Vistusertib | 1009298-59-2 | PI3K (α/β/δ/γ)/mTOR |
| GDC-0032 | Taselisib | 1282512-48-4 | PI3Kα/β/δ/γ |
| PQR309 | Bimiralisib | 1225037-39-7 | PI3K (α/β/δ/γ)/mTOR, |
| | | | brain penetrant |
| everolimus | RAD001 | 159351-69-6 | mTORC1, cell type |
| | | | spfc mtorc2, FKBP12 |
| OSI-027 | ASP4786 | 936890-98-1 | mTORC1/2 |
| BYL-719 | Alpelisib | 1217486-61-7 | PI3K |
| ZSTK474 | | 475110-96-4 | PI3K (δ) and pan PI3K |
| Taselisib (GDC 0032) | GDC-0032; RG-7604 | 1282512-48-4 | PI3Kα/β/δ/γ |
| IPI-549 | | 1693758-51-8 | PI3Kγ, 100 fold less for other |
| CUDC-907 | | 1339928-25-4 | PI3K and HDAC |
| AZD6482 | KIN 193 | 1173900-33-8 | PI3Kβ |
| Alpelisib (BYL-719) | | 1217486-61-7 | PI3Kα |
| AZD8186 | | 1627494-13-6 | PI3Kβ and PI3Kδ |
| GSK2636771 | | 1372540-25-4 | PI3Kβ |
| AMG319 | 1608125-21-8 385.40 PI3K | 1608125-21-8 | PI3Kδ |
| GSK2126458 | Omipalisib | 1086062-66-9 | mTOR/PI3K |
| LY3023414 | | 1386874-06-1 | PI3K/DNA-PK/mTOR |
| Palomid 529 (P529) | P529 | 914913-88-5 | mTORC1 and 2 |
| umbralisib (TGR-1202) | TGR-1202; RP5264 | 1532533-67-7 | PI3Kδ |
| SC79 | | 305834-79-1 | Akt activator |
| torin1 | | 1222998-36-8 | mTORC1/2 |
| Acalisib | GS-9820; CAL-120 | 870281-34-8 | PI3Kδ |
| TG100-115 | | 677297-51-7 | PI3Kγ/δ |
| 3BDO | | 890405-51-3 | mTOR activator |
| Nemiralisib | | 1254036-71-9 | PI3Kδ |
| (GSK2269557) | | | |
| Rapamycin | Sirolimus | 53123-88-9 | mTORC-1 |
| MHY1485 | | 326914-06-1 | mTOR activator |
| Quercetin | Quercetin | 117-39-5 | flavonol |
| MLN1117 | Scrabelisib; TAK-117 | 1268454-23-4 | PI3Kα |
| INK-128 | Sapanisertib | 1224844-38-5 | mTORC1/2 |
| CC-115 | | 1228013-15-7 | DNA-PK, mTOR |
| CC-115 (hydrochloride) | | 1300118-55-1 | DNA-PK, mTOR |
| GSK1059615 | | 958852-01-2 | dual inhibitor of PI3Kα/β/δ/γ |
| | | | (reversible) and mTOR |
| Pilaralisib (XL147) | | 934526-89-3 | PI3K |
| MI-773 (SAR405838) | | 1303607-60-4 | MDM2 antagonist |
| GDC-0349 | RG-7603 | 1207360-89-1 | mTOR |
| Voxtalisib (XL765, | | 934493-76-2 | mTOR/PI3K |
| SAR245409) | | | |
| GSK2126458 | | 1086062-66-9 | (PI3K) |
| BMS-214662 farnesyl | | 195987-41-8 | (CDK2, JAK2, and FLT3) |
| transferase | | | |
| SB1317 | | 937270-47-8 | (JAK1) |
| Filgotinib | | 1206161-97-8 | |
| PF-06459988 | | 1428774-45-1 | (EGFR) |
| GDC-0980 | | 1032754-93-0 | |
| Buparlisib (BKM120, | | 944396-07-0 | |
| NVP-BKM120) | | | |

Provided in Table 29 are fold changes in PNPLA3 and PCSK9 mRNA expression in primary hepatocytes from two different donors (4178 and 4282) relative to GUSB, and fold changes in PNPLA3 and COL1A1 mRNA expression in primary stellate cells relative to GAPDH. All compounds were tested at 3 μM concentrations.

TABLE 29

| | Primary Hepatocytes | | Primary Hepatocytes | Primary Stellate | |
|---|---|---|---|---|---|
| | 4178 | | 4282 | cells | |
| Compound | PNPLA3 | PCSK9 | PNPLA3 | PNPLA3 | COL1A1 |
| Apitolisib (GDC-0980, | 0.32 | 0.31 | 0.46 | 0.56 | 0.54 |
| RG7422) | | | | | |
| PF-04691502 | 0.36 | 0.44 | 0.47 | 0.54 | 0.48 |
| GDC-0980 | 0.37 | 0.40 | 0.40 | 0.67 | 0.61 |
| VS-5584 (SB2343) | 0.50 | 0.47 | 0.18 | 0.63 | 0.58 |
| Buparlisib (BKN120, | 0.52 | 0.46 | 0.60 | 0.50 | 0.81 |
| NVE-BKM120) | | | | | |
| CC-223 | 0.51 | 0.72 | 0.80 | 0.33 | 0.50 |
| CH5132799 | 0.49 | 0.51 | 0.60 | 0.56 | 0.81 |
| WYE-125132 (WYE-132) | 0.43 | 0.43 | 0.70 | 0.54 | 0.46 |
| CZ415 | 0.60 | 0.66 | 0.70 | 0.37 | 0.59 |
| AZD-8055 | 0.63 | 0.68 | 0.69 | 0.36 | 0.46 |
| NVP-BKM120 | 0.56 | 0.50 | 0.70 | 0.43 | 0.70 |
| (Hydrochloride) | | | | | |
| NVP-BKM120 | 0.60 | 0.75 | 0.70 | 0.43 | 0.66 |
| AZD2014 | 0.43 | 0.36 | 0.80 | 0.56 | 0.55 |
| GDC-0032 | 0.58 | 0.47 | 0.60 | 0.61 | 1.05 |
| PQR309 | 0.47 | 0.50 | 0.60 | 0.88 | 0.81 |
| everolimus | 0.60 | 0.66 | 1.00 | 0.36 | 0.93 |
| OSI-027 | 0.60 | 0.69 | 0.80 | 0.57 | 0.51 |
| BYL-719 | 0.69 | 0.71 | 0.60 | 0.70 | 1.24 |
| ZSTK474 | 0.60 | 0.66 | 0.80 | 0.68 | 0.71 |
| Taselisib (GDC 0032) | 0.72 | 0.67 | 0.62 | 0.77 | 1.32 |
| IPI-549 | 0.68 | 0.74 | 0.68 | 0.75 | 1.00 |
| CUDC-907 | 0.50 | 0.83 | 0.50 | 1.33 | 0.41 |
| AZD6482 | 0.72 | 0.56 | 0.78 | 0.87 | 1.20 |
| Alpelisib (BYL719) | 0.80 | 0.67 | 0.69 | 0.94 | 1.16 |
| AZD8186 | 0.80 | 0.70 | 0.80 | 0.86 | 1.22 |
| GSK2636771 | 0.75 | 0.59 | 0.68 | 1.06 | 1.04 |
| AMG319 | 0.73 | 0.74 | 0.85 | 0.99 | 0.99 |
| GSK2126458 | 0.92 | 0.91 | 0.80 | 0.90 | 0.42 |
| LY3023414 | 0.80 | 0.65 | 1.00 | 0.94 | 0.49 |
| Palomid 529 (P529) | 1.00 | 1.05 | 0.80 | 0.97 | 1.10 |
| umbralisib (TGR-1202) | 0.90 | 0.80 | 0.80 | 1.10 | 1.15 |
| SC79 | 1.10 | 1.11 | 0.94 | 0.85 | 0.98 |
| torin1 | 1.10 | 0.89 | 0.91 | 0.88 | 0.61 |
| Acalisib | 1.00 | 0.99 | 1.00 | 0.91 | 0.96 |
| TG100-115 | 1.00 | 0.69 | 0.90 | 1.07 | 1.11 |
| 3BDO | 1.10 | 1.01 | 1.00 | 0.91 | 1.03 |
| Nemiralisib | 1.00 | 0.84 | 0.95 | 1.08 | 1.00 |
| (GSK2269557) | | | | | |
| Rapamycin | 1.50 | 1.37 | 1.10 | 0.49 | 1.43 |
| MHY1485 | 1.10 | 1.04 | 1.20 | 0.86 | 0.87 |
| Quercetin | 1.16 | 0.95 | 0.93 | 1.11 | 0.94 |
| MLN1117 | 1.57 | 0.69 | 0.97 | 0.70 | 1.14 |
| INK-128 | 1.50 | 0.98 | 1.63 | 0.56 | 0.47 |
| CC-115 | 1.30 | 0.62 | 3.00 | 1.43 | 0.43 |
| CC-115 (hydrochloride) | 2.00 | 1.19 | 2.60 | 1.18 | 0.35 |

Additional compounds were tested in hepatocytes and PNPLA3 mRNA levels were assessed via qRT-PCR. Table 30 provides a summary of the relative PNPLA3 mRNA and standard deviation (SD) for each compound and concentration tested.

TABLE 30

| | | Relative | |
|---|---|---|---|
| | | PINPLA3 | |
| | | mRNA | SD |
| DMSO (vehicle control) | DMSO | 0.950 | 0.233 |
| GSK2126458 (PI3K) | 1 uM | 0.395 | 0.047 |
| | 10 uM | 0.800 | 0.060 |
| BMS-214662 | 1 uM | 0.760 | 0.199 |
| farnesyl transferase | 10 uM | 0.417 | 0.099 |
| SB1317 (CDK2, JAK2, | 1 uM | 0.400 | 0.028 |
| and FLT3) | 10 uM | 0.413 | 0.068 |
| Filgotinib (JAK1) | 1 uM | 0.781 | 0.001 |
| | 10 uM | 0.365 | 0.088 |
| PF-06459988 (EGFR) | 1 uM | 0.965 | 0.085 |
| | 10 uM | 0.398 | 0.067 |
| GDC-0980 | 0.1 uM | 0.893 | 0.048 |
| | 0.3 uM | 0.669 | 0.067 |
| | 1 uM | 0.420 | 0.026 |
| | 3 uM | 0.365 | 0.037 |
| Buparlisib (BKM120, | 0.1 uM | 1.049 | 0.207 |
| NVP-BKM120) | 0.3 uM | 0.729 | 0.135 |
| | 1 uM | 0.659 | 0.027 |
| | 3 uM | 0.515 | 0.059 |
| CH5132799 | 0.1 uM | 0.940 | 0.166 |
| | 0.3 uM | 0.870 | 0.252 |
| | 1 uM | 0.743 | 0.088 |
| | 3 uM | 0.491 | 0.153 |
| PQR309 | 0.1 uM | 1.35287 | 0.078677 |
| | 0.3 uM | 0.823563 | 0.207695 |
| | 1 uM | 0.526692 | 0.06262 |
| | 3 uM | 0.465688 | 0.054363 |
| VS-5584 (SB2343) | 0.1 uM | 0.862628 | 0.143622 |
| | 0.3 uM | 0.720309 | 0.100062 |
| | 1 uM | 0.386546 | 0.039667 |
| | 3 uM | 0.498318 | 0.059621 |
| Pamidronate | 10 uM | 0.2 | |
| Fedratinib (SAR302503, | 10 uM | 0.25 | |
| TG101348) | | | |
| Romidepsin (FK228, | 10 uM | 0.3 | |
| Depsipeptide) | | | |
| BI 2536 | 10 uM | 0.38 | |
| Hydralazine HCI | 10 uM | 0.25 | |

### Example 27

### mTOR Pathway Inhibitors

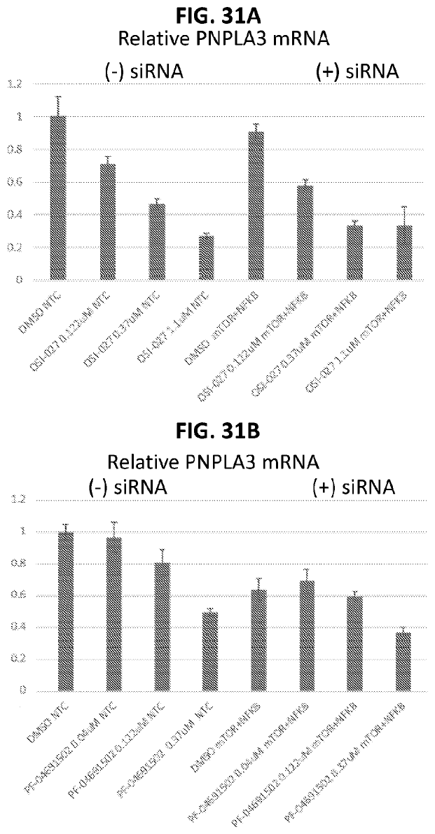

Primary human hepatocytes were treated with mTOR siRNA for 72 hours and then treated with OSI-027 or PF-04691502, and assayed for PNPLA3 expression, as previously described. PNPLA3 mRNA was normalized to GUSB. The combination of siRNA knockdown of mTOR and treatment with the chemical inhibitors did not provide additional benefit in decreasing PNPLA3 mRNA, indicating that the compounds affected PNPLA3 expression via the mTOR pathway. FIG. 31A shows PNPLA3 expression after treatment with OSI-027 with and without mTOR siRNA knockdown and FIG. 31B shows PNPLA3 expression after treatment with PF-04691502 with and without mTOR siRNA knockdown, PNPLA3 mRNA quantification for both experiments is shown in Table 31.

TABLE 31

| | Relative |
|---|---|
| | PNPLA3 |
| | mRNA |
| | expression |
| DMSO NTC | 1.005 ± 0.120 |
| OSI-027 0.1.22 uM NTC | 0.709 ± 0.049 |
| OSI-027 0.37 uM NTC | 0.467 ± 0.032 |
| OSI-027 1.1 uM NTC | 0.272 ± 0.016 |
| DMSO OSI-027 mTOR + NFKB | 0.905 ± 0.048 |
| OSI-027 0.122 uM mTOR + NFKB | 0.581 ± 0.033 |
| OSI-027 0.37 uM mTOR + NFKB | 0.334 ± 0.028 |
| OSI-027 1.1 uM mTOR + NFKB | 0.335 ± 0.115 |
| DMSO PF-04691502 NTC | 1.001 ± 0.047 |
| PF-04691502 0.04 uM NTC | 0.966 ± 0.094 |
| PF-04691502 0.122 uM NTC | 0.807 ± 0.086 |
| PF-04691502 0.37 uM NTC | 0.496 ± 0.024 |
| DMSO PF-04691502 mTOR +NFKB | 0.639 ± 0.072 |
| PF-04691502 0.04 uM mTOR +NFKB | 0.692 ± 0.074 |
| PF-04691502 0.122 uM mTOR +NFKB | 0.596 ± 0.030 |
| PF-04691502 0.37 uM mTOR +NFKB | 0.370 ± 0.032 |

To further characterize the role of the mTOR pathway in liver fibrosis, a selection of mTOR inhibitors (TORIN1, INK-128, and WYE-132) were used to treat stellate cells and determine the effects on fibrosis related genes. Stellate cells P7 were treated with 0.5 μM of each of the indicated compounds for 18 hours. Cells were processed for RNA extraction and qRT-PCR as previously described. The assay was repeated in triplicate.

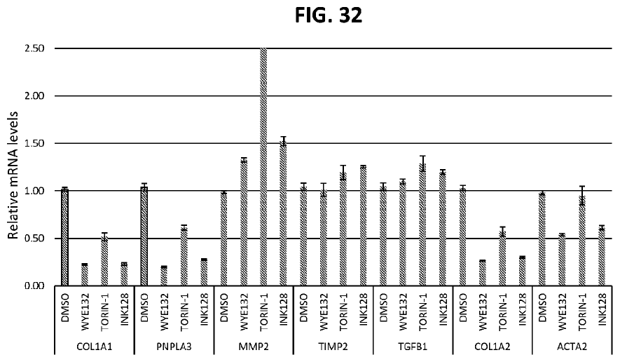

Results of the compounds on COL1A1, PNPLA3, MMP2, TIM2, TGFB1, COL1A2, and ACTA2 are shown in FIG. 32 and Table 32.

TABLE 32

| Gene | Compound | Relative mRNA |
|---|---|---|
| COL1A1 | DMSO | 1.02 ± 0.02 |
| | WYE-132 | 0.22 ± 0.01 |
| | TORIN1 | 0.52 ± 0.04 |
| | INK-128 | 0.23 ± 0.01 |
| PNPLA3 | DMSO | 1.04 ± 0.04 |
| | WYE-132 | 0.20 ± 0.01 |
| | TORIN1 | 0.61 ± 0.03 |
| | INK-128 | 0.28 ± 0.01 |
| MMP2 | DMSO | 0.98 ± 0.01 |
| | WYE-132 | 1.33 ± 0.02 |
| | TORIN1 | 3.44 ± 0.26 |
| | INK-128 | 1.53 ± 0.05 |
| TIMP2 | DMSO | 1.04 ± 0.04 |
| | WYE-132 | 1.01 ± 0.07 |
| | TORIN1 | 1.19 ± 0.08 |
| | INK-128 | 1.26 ± 0.01 |
| TGFB1 | DMSO | 1.05 ± 0.04 |
| | WYE-132 | 1.10 ± 0.03 |
| | TORIN1 | 1.29 ± 0.08 |
| | INK-128 | 1.20 ± 0.02 |
| COL1A2 | DMSO | 1.03 ± 0.03 |
| | WYE-132 | 0.27 ± 0.00 |
| | TORIN1 | 0.57 ± 0.05 |
| | INK-128 | 0.30 ± 0.01 |
| ACTA2 | DMSO | 0.98 ± 0.02 |
| | WYE-132 | 0.54 ± 0.01 |
| | TORIN1 | 0.95 ± 0.10 |
| | INK-128 | 0.61 ± 0.02 |

A further analysis of the compounds that provided the best PNPLA3 mRNA response showed that compounds inhibiting both mTOR and PI3K comprised the majority of the best results, In contrast, most of the compounds that did not reduce PNPLA3 expression targeted PI3K only, A summary of the data is provided below in Table 33.

TABLE 33

| | | | PI3K | mTOR | mTOR + |
|---|---|---|---|---|---|
| | Compound Name | Target | only | only | PI3K |
| Best hits | OSI-027 | mTORC1/2 | | x | |
| ( >50% | PF-04691502 | PI3K (α/β/δ/γ)/mTOR | | | x |
| reduction + | Apitolisib (GDC-0980, | PI3K (α/β/δ/γ)/mTOR | | | x |
| dose- | RG7422) | | | | |
| response | WYE-125132 | mTORC1/2 | | x | |
| in 2 or | (WYE-132) | | | | |
| more hep | PQR309 | PI3K (α/β/δ/γ)/mTOR | | | x |
| donors) | CH5132799 | PI3Ka/b | x | | |
| | VS-5584 (SB2343) | PI3K (α/β/δ/γ)/mTOR | | | x |
| | Buparlisib (BKM120, | PI3K (α/β/δ/γ | x | | |
| | NVP-BKM120) | | | | |
| Hit | CC-223 | mTORC1 2 | | x | |
| (any reduction | CZ415 | mTOR c1/2 | | x | |
| that is dose- | NVP-BKM120 | PI3K | x | | |
| dependent in 1 | ZSTK474 | PI3K (δ) | x | | |
| hep donor +1 | | and pan PI3K | | | |
| stellate donor) | AZD-8055 | mTORC1/2 | | x | |
| | Taselisib (GDC 0032) | PI3Kα/β/γ | x | | |
| | GSK2636771 | PI3Kβ | x | | |
| No | AZD2014 | PI3K (α/β/δ/γ)/mTOR | | | x |
| reduction | CUDC-907 | PI3K and HDAC | x | | |
| of PNPLA3 | NVP-BKM120 | PI3K (α/β/δ/γ | x | | |
| 16% of | (Hydrochloride) | | | | |
| non-hits | GDC-0032 | PI3Kα/δ/γ | x | | |
| target | everolimus | mTORC1 | | x | |
| mTOR + | IPI-549 | PI3Kγ, 100 fold | x | | |
| PI3K | | less for other | | | |
| | BYL-719 | PI3K | x | | |
| | AZD6482 | PI3Kβ | x | | |
| | AMG319 | PI3Kδ | x | | |
| | Alpelisib (BYL719) | PI3Kα | x | | |
| | AZD8186 | PI3Kβ and PI3Kδ | x | | |
| | LY3023414 | PI3K/DNA- | | | x |
| | | PK/mTOR | | | |
| | umbralisib (TGR-1202) | PI3Kδ | x | | |
| | GSK2126458 | mTOR/PI3K | | | x |
| | Acalisib | PI3Kδ | x | | |
| | Nemiralisib | PI3Kδ | x | | |
| | (GSK2269557) | | | | |
| | Palomid 529 (P529) | mTORC1 and 2 | | x | |
| | TG100-115 | PI3Kγ/δ | x | | |
| | torin1 | mTORC1/2, | | x | |
| | | DNA-PK | | | |
| | Rapamycin | mTORC-1 | | x | |
| | MLN1117 | PI3Kα | x | | |
| | GSK1059615 | dual inhibitor of | | | x |
| | | PI3Kα/β/δ/γ and | | | |
| | | mTOR | | | |
| | Pilaralisib (XL147) | PI3K | x | | |
| | Voxtalisib (XL765, | mTOR/PI3K | | | x |
| | SAR245409) | | | | |
| | GDC-0349 | mTORC1/2 | | x | |
| | Rapamycin | mTORC-1 | | x | |

Based on the reported compound specificity, compounds that target mTORC1 only, such as rapamycin, did not decrease PNPLA3 expression, while compounds that target both mTORC1 and mTORC2, such as OSI-027 and WYE-125132, did decrease PNPLA3 expression. Thus, mTORC1 may not play a role in PNPLA3 expression.

PI3Kα, PI3Kγ, and PI3Kδ inhibitors did not decrease PNPLA3 expression. Notably, PI3Kα and PI3Kγ have low expression in hepatocytes. PI3Kβ inhibitors did result in decreased PNPLA3 expression, but the inhibitors with the most robust PNPLA3 inhibition also inhibited the mTOR pathway, for example PF-04691502, Apitolisib, PQR309, and VS-5584.

### Example 28

### TGFβ Pathway Inhibitors

Primary human hepatocytes and stellate cells were also incubated with inhibitors of the TGFβ pathway and PNPLA3 gene expression changes assessed via qRT-PCR as previously described. In addition, primary hepatocytes and stellate cells were treated with TGFβ-ligand alone or with selected the small molecule inhibitors for 18 hours and subsequently harvested. for gene expression assays.

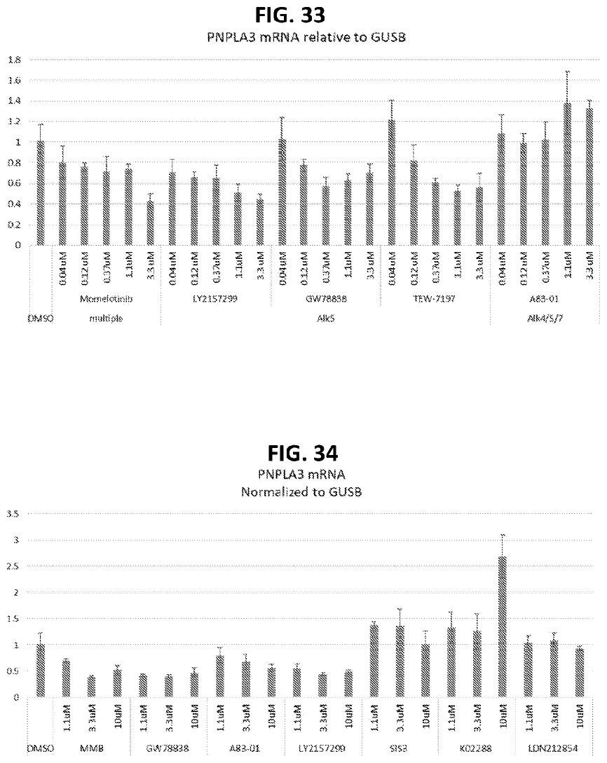

As shown in FIG. 33 and Table 34 TGF-β pathway inhibitors decrease PNPLA3 mRNA primary human hepatocytes.

TABLE 34

| | | | Relative |
|---|---|---|---|
| | | | PNPLA3 |
| Target | | | mRNA |
| Pathway | | | expression |
| | DMSO | | 1.011 ± 0.163 |
| multiple | Momelotinib | 0.04 uM | 0.807 ± 0.160 |
| | | 0.12 uM | 0.761 ± 0.038 |
| | | 0.37 uM | 0.719 ± 0.145 |
| | | 1.1 uM | 0.741 ± 0.050 |
| | | 3.3 uM | 0.430 ± 0.071 |
| Alk5 | LY2157299 | 0.04 uM | 0.707 ± 0.125 |
| | | 0.12 uM | 0.658 ± 0.054 |
| | | 0.37 uM | 0.654 ± 0.126 |
| | | 1.1 uM | 0.510 ± 0.082 |
| | | 3.3 uM | 0.448 ± 0.047 |
| | GW78838 | 0.04 uM | 1.031 ± 0.210 |
| | | 0.12 uM | 0.777 ± 0.061 |
| | | 0.37 uM | 0.574 ± 0.085 |
| | | 1.1 uM | 0.631 ± 0.059 |
| | | 3.3 uM | 0.701 ± 0.090 |
| | TEW-7197 | 0.04 uM | 1.219 ± 0.186 |
| | | 0.12 uM | 0.822 ± 0.152 |
| | | 0.37 uM | 0.613 ± 0.035 |
| | | 1.1 uM | 0.527 ± 0.054 |
| | | 3.3 uM | 0.560 ± 0.134 |
| Alk4/5/7 | A83-01 | 0.04 uM | 1.084 ± 0.184 |
| | | 0.12 uM | 0.993 ± 0.091 |
| | | 0.37 uM | 1.022 ± 0.177 |
| | | 1.1 uM | 1.380 ± 0.303 |
| | | 3.3 uM | 1.326 ± 0.077 |

However, BMP pathway inhibitors K02288 and LDN212854, did not decrease PNPLA3 mRNA in primary human hepatocytes (FIG. 34 and Table 35).

TABLE 35

| | | Relative PNPLA3 |
|---|---|---|
| | | mRNA expression |
| DMSO | | 1.01702 ± 0.204018 |
| Momelotinib | 1.1 uM | 0.703361 ± 0.031598 |
| | 3.3 uM | 0.38926 ± 0.02341 |
| | 10 uM | 0.52548 ± 0.084162 |
| GW78838 | 1.1 uM | 0.417738 ± 0.029149 |
| | 3.3 uM | 0.399023 ± 0.033823 |
| | 10 uM | 0.479321 ± 0.0927 |
| A83-01 | 1.1 uM | 0.801925 ± 0.155823 |
| | 3.3 uM | 0.683958 ± 0.149173 |
| | 10 uM | 0.563366 ± 0.068538 |
| LY2157299 | 1.1 uM | 0.553742 ± 0.081115 |
| | 3.3 uM | 0.450262 ± 0.024608 |
| | 10 uM | 0.493695 ± 0.031934 |
| SIS3 | 1.1 uM | 1.381939 ± 0.060339 |
| | 3.3 uM | 1.358315 ± 0.330258 |
| | 10 uM | 1.010613 ± 0.257414 |
| K02288 | 1.1 uM | 1.341947 ± 0.289934 |
| | 3.3 uM | 1.259226 ± 0.326774 |
| | 10 uM | 2.686821 ± 0.414619 |
| LDN212854 | 1.1 uM | 1.046279 ± 0.128556 |
| | 3.3 uM | 1.094531 ± 0.133199 |
| | 10 uM | 0.933889 ± 0.040933 |
| Pacritinib | 1.1 uM | 0.722913 ± 0.083112 |
| | 3.3 uM | 0.820353 ± 0.027184 |
| | 10 uM | 1.951342 ± 0.247959 |

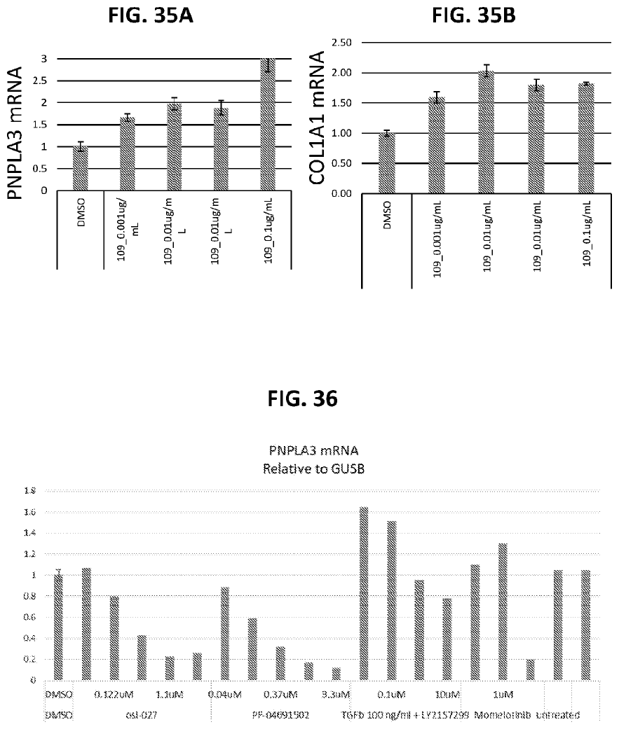

Incubation of stellate cells with TGFβ-ligand alone induced expression of PNPLA3 and COL1A1 in a dose dependent manner (FIG. 35A and FIG. 35B)

However, even with the TGFb-ligand-induced expression, PNPLA3 expression was reduced with co-treatment of LY2157299 in a dose dependent manner (FIG. 36 and Table 36).

TABLE 36

| | | Relative |
|---|---|---|
| | | PNPLA3 |
| | | mRNA |
| | | expression |
| DMSO | | 1.001 |
| OSI-027 | 0.04 uM | 1.071 |
| | 0.122 uM | 0.795 |
| | 0.37 uM | 0.427 |
| | 1.1 uM | 0.228 |
| | 3.3 uM | 0.262 |
| PF-04691502 | 0.04 uM | 0.880 |
| | 0.122 uM | 0.590 |
| | 0.37 uM | 0.322 |
| | 1.1 uM | 0.170 |
| | 3.3 uM | 0.119 |
| TGFb ligand + | 0 uM | 1.646 |
| LY2157299 | 0.1 uM | 1.511 |
| | 1 uM | 0.951 |
| | 10 uM | 0.784 |
| Momelotinib | 1 uM | 1.101 |
| | 10 uM | 1.299 |
| | 0.1 uM | 0.200 |
| untreated | | 1.048 |
| | | 1.044 |

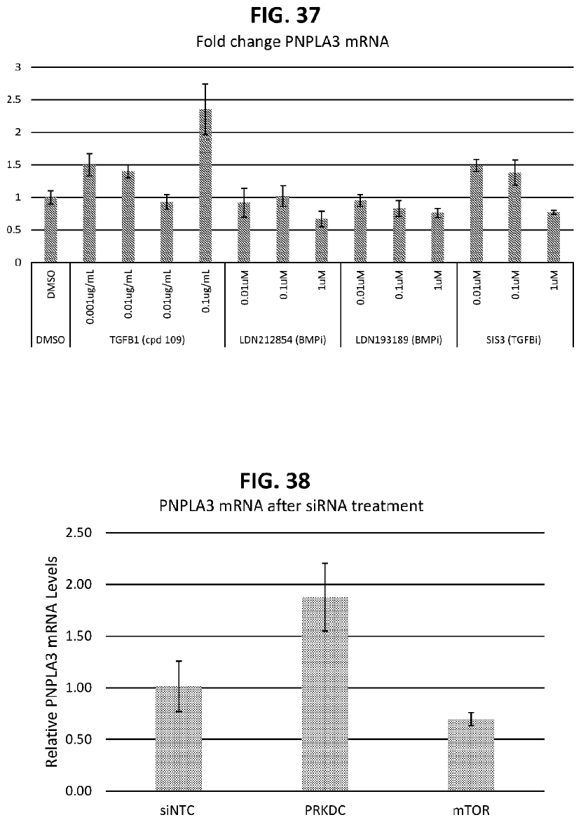

However, in stellate cells, TGFβ ligand stimulated PNPLA3 expression most at the highest concentration tested (0.1 μg/ml) but the TGFβ superfamily inhibitors had only a modest effect on reducing PNPLA3 expression (FIG. 37 and Table 37). However, this experiment did not include LY2157299, which was previously shown to still inhibit PNPLA3 expression after TGFβ-ligand induced expression.

TABLE 37

| | | Relative |
|---|---|---|
| | | PNPLA3 |
| | | mRNA |
| | | expression |
| DMSO | DMSO_ | 1.00 ± 0.10 |
| TGFB1 (TGFB) | 0.001 ug/ML | 1.50 ± 0.17 |
| | 0.01 ug/ML | 1.40 ± 0.10 |
| | 0.001 ug/ML | 0.93 ± 0.11 |
| | 0.01 ug/ML | 2.35 ± 0.39 |
| LDN212854 | 0.01 uM | 0.92 ± 0.22 |
| (BMP) | 0.1 uM | 1.02 ± 0.16 |
| | 1 uM | 0.67 ± 0.12 |
| | 10 uM | 7.43 ± 3.91 |
| LDN193189 | 0.01 uM | 0.95 ± 0.09 |
| (BMP) | 0.1 uM | 0.83 ± 0.12 |
| | 1 uM | 0.76 ± 0.07 |
| | 10 uM | 9.25 ± nd |
| SIS3 (TGFB) | 0.01 uM | 1.49 ± 0.09 |
| | 0.1 uM | 1.38 ± 0.19 |
| | 1 uM | 0.77 ± 0.03 |
| | 10 uM | 7.95 ± 2.29 |

### Example 29

### siRNA Knockdown of mTOR and PI3K Pathways

To further interrogate the pathways that control PNPLA3 expression, hepatocytes were treated with siRNAs against specific members of the mTOR and PI3K pathways. Cells were treated with siRNA and mRNA harvested as previously described in Example 1. siRNA for mTOR, PRKDC, PI3Kα, PI3Kβ, AKT3, and RICTOR were purchased from Dharmacon (catalogue numbers siNTC D-001206-13-05, DNA-PK M-005030-01-0005, mTOR M-003008-03-0005, PI3Kα M-003018-03-0005, PI3Kβ M-003019-02-0005, AKT3 M-003002-02-0005, RICTOR M-016984-02-0005).

The siRNA results are shown in Table 38. PNPLA3 expression after each siRNA knockdown is shown relative to the geometric mean (GeoMean) of housekeeping genes GUSB, B2M, and HPRT.

TABLE 38
PNPLA3 relative to GeoMean

| siRNA | FC in PNPLA3 | Std Dev |
|---|---|---|
| siNTC (Non Targeting) | 1.01 | 0.24 |
| PRKDC (DNA-PK) | 1.88 | 0.33 |
| mTOR | 0.60 | 0.06 |
| mTOR + PI3K α | 1.27 | 0.02 |
| mTOR + PI3K β | 0.58 | 0.20 |
| mTOR + AKT3* | 0.60 | 0.20 |
| RICTOR (part of | 0.70 | 0.2 |
| mTORC2) | | |

FIG. 38 provides a comparison of the PNPLA3 gene expression after control siRNA treatment, mTOR siRNA treatment, or PRKDC (DNA-PK) siRNA treatment for 3 replicates.

siRNA knockdown of mTOR or the mTORC2 subunit RICTOR resulted in a decrease in PNPLA3 expression (0.60 fold change and 0.70 fold change, respectively). Knockdown of mTOR and AKT3 also resulted in PNPLA3 decrease, but since AKT3 is expressed at very low levels in hepatocytes, the effect may be due in more part to the knockdown of mTOR than AKT3. Knockdown of both mTOR. and PI3Kβ also resulted in PNPLA3 expression decrease. Conversely, knockdown of both mTOR and DNA-PK resulted in an increase in PNPLA3 expression, as did knockdown of mTOR and PI3Kα. Thus, inhibition of DNA-PK or PI3Kα resulted in adverse effects, i.e. an increase in PNPLA3 expression.

Based on the siRNA data, the mTOR signaling pathway, specifically the mTORC2 pathway, plays a significant roles in modulating PNPLA3 expression, while the PI3Kα signaling pathway does not. Furthermore, knocking down the mTOR and PI3Kβ pathway did not result in significant changes in PNPLA3 expression (see e.g. a 0.60 fold change for mTOR alone vs a 0.58 fold change for both mTOR and PI3Kβ), suggesting that combination mTOR and PI3Kβ inhibition does not have a synergistic or additive effect on PNPLA3 expression.

Next, hepatocytes were treated with both mTOR siRNA and either mTOR or mTOR/PI3K small molecule inhibitors. Hepatocytes were treated with siRNA against mTOR and AKT3 or control siRNA (siNTC) as previously described. The hepatocytes were then treated with various concentrations of OSI-027 or PF-04691502 for 16 hours. Cells were collected after and mRNA harvested for qRT-PCR as previously described. PNPLA3 expression was normalized to GUSB.

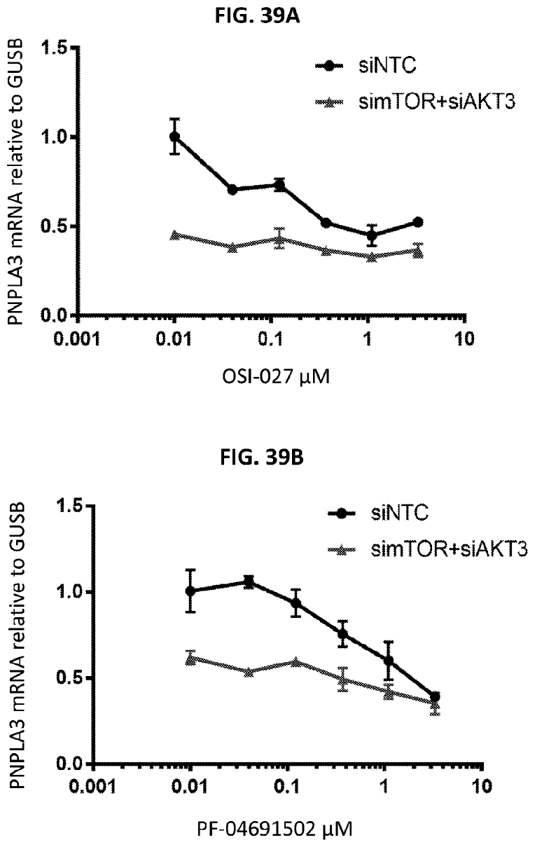

FIG. 39A shows the relative amount of PNPLA3 mRNA normalized to GUSB after OSI-027 treatment in cells that were pretreated with mTOR and AKT3 siRNA (triangles) or control siRNA (siNTC, circles). FIG. 398 shows the relative amount of PNPLA3 mRNA normalized to GUSB after PF-04691502 treatment in cells that were pretreated with mTOR and AKT3 siRNA (triangles) or control siRNA (siNTC, circles). Both OSI-027 and reduced PNPLA3 in a dose dependent manner in the absence of siRNA treatment. However, the combination of OSI-027 and mTOR+AKT3 siRNA knockdown did not result in a dose dependent decrease of PNPLA3 expression, indicating that OSI-027 treatment is not additive to mTOR and AKT3 siRNA treatment for PNPLA3 reduction. Thus, mTOR knockdown alone is sufficient to downregulate PNPLA3 expression, In contrast, the combination of PF-04691502 and mTOR+AKT3 siRNA knockdown did result in a slight dose dependent decrease of PNPLA3 expression at the higher concentrations of PF-04691502 used, indicating that PF-04691502 treatment is slightly additive to mTOR and AKT3 siRNA treatment for PNPLA3 reduction.

### Example 30

### OSI-027 and PF-04691502 Inhibit Activation of mTOR and PI3Kβ Pathway Proteins

Next, activation of protein members of the mTOR and/or PI3K pathway were assessed after OSI-027, PF-04691502, CH5132799, rapamycin, or Alpelisib (BYL719) treatment. CH5132799 is a PI3kα/β inhibitor, rapamycin is an mTORC1 specific inhibitor, and Alpelisib is a PI3kα specific inhibitor.

Parallel samples of human hepatocytes were treated with 3 μM each of OSI-027, PF-04691502, CH5132799, rapamycin, or Alpelisib (BYL719) for 35 min, 1 hr, 2 hrs, 3 hrs, 4.5 hrs, or 20 hrs. One set of samples were harvested for Western Blots using Laemmli buffer (2% SDS, 10% glycerol, 75 mM Tris-Cl, pH 6.8, 5% beta-mercaptoethanol, bromphenol blue). The other set was harvest for mRNA processing as previously described. Hepatocyte cell lysates were loaded onto 4-12% Bis-Tris gels with 35,000 cells/15 μL per lane. Blots were incubated with primary antibodies overnight in Odyssey blocking buffer. Antibodies used were pAKT (Ser473) Rabbit mAb 4060 (Cell Signaling (1:1000)), pS6 Ser235/236 Rabbit mAb 4858 (Cell Signaling (1:1000)), pNDRG1 T346 Rabbit mAb 5482 (Cell Signaling (1:1000)), p4EBP1c (Thr37/46) Rabbit mAb 2855 (Cell Signaling (1:1000)), AKT (pan) Mouse mAh 2920 (Cell Signaling (1:1000)), Ribosomal Protein S6 (C-8) se-74459 Mouse mAb (Santa Cruz Biotech (1:2000)), NDRG1 A-5 sc-398823 Mouse mAh (Santa Cruz Biotech (1:200)) and 4EBP1 (53H11) Rabbit mAb 9644 (Cell Signaling (1:1000)). Blots were incubated with secondary antibodies IRDye® 800CW Donkey anti-Rabbit IgG (H+L) 926-32213 or Donkey Anti-Mouse IgG Polyclonal Antibody (IRDye® 680LT) 926-68022 at 1:10,000 in Odyssey blocking buffer for 1 hour, and imaged using Odyssey Licor Scanner. Image Studio software was used to quantify phosphorylated protein abundance to total protein abundance, relative to DMSO control from each timepoint.

Levels of phosphorylated S6, AKT, and NDRG1 proteins were determined as compared to total S6, AKT, and NDRG1 protein. PNPLA3 mRNA expression was quantified and normalized to housekeeping gene GUSB.

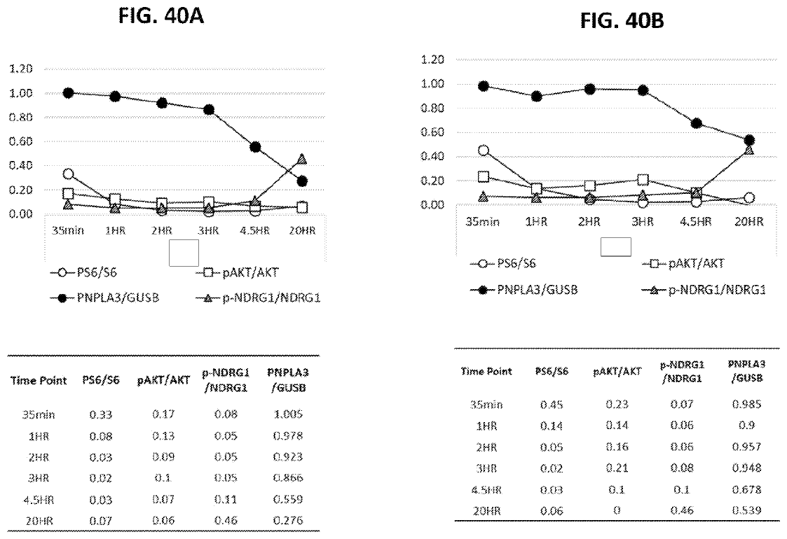

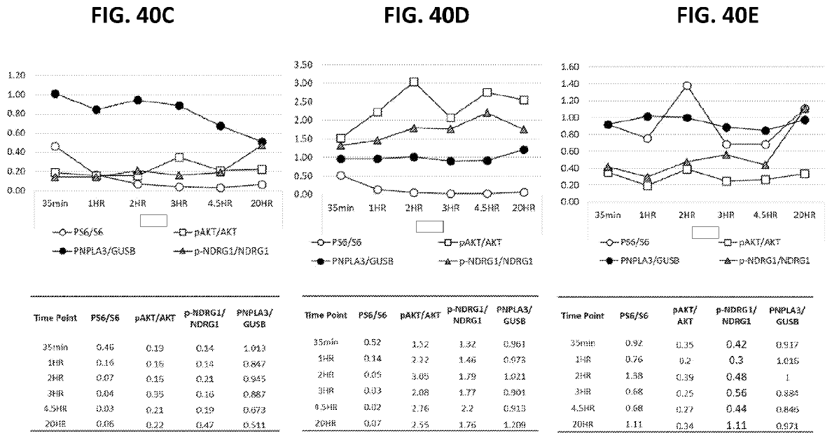

FIG. 40A show the PNPLA3 mRNA expression level, phosphorylated S6 (pS6/S6), phosphorylated AKT (pAKT/AKT), and phosphorylated NDRG1 (pNDRG1/NDRG1) after treatment with PF-04691502. Treatment resulted in decreased levels of PNPLA3 expression after 3 hours. Phosphorylated S6 decreased at 1 hour. Phosphorylated AKT also decreased at 20 hours.

FIG. 40B shows the PNPLA3 mRNA expression level, phosphorylated S6 (pS6/S6), phosphorylated AKT (pAKT/AKT), and phosphorylated NDRG1 (pNDRG1/NDRG1) after treatment with OSI-027. Treatment resulted in decreased levels of PNPLA3 expression after 3 hours. Phosphorylated S6 decreased at 1 hour. Phosphorylated AKT decreased at 4.5 hours.

FIG. 40C shows the PNPLA3 mRNA expression level, phosphorylated S6 (pS6/S6), phosphorylated AKT (pAKT/AKT), and phosphorylated NDRG1 (pNDRG1/NDRG1) after treatment with CH5132799. Treatment resulted in decreased levels of PNPLA3 expression after 3 hours. Phosphorylated S6 decreased at 1 hour. Phosphorylated AKT increased at 3 hours and decreased to pre-treatment levels at 4.5 hours.

FIG. 40D shows the PNPLA3 mRNA expression level, phosphorylated S6 (pS6/S6), phosphorylated AKT (pAKT/AKT), and phosphorylated NDRG1 (pNDRG1/NDRG1) after treatment with rapamycin. PNPLA3 expression did not decrease, and in fact slightly increased at 20 hours. Phosphorylated S6 decreased at 1 hour. Phosphorylated AKT increased at 1 hour and remained high.

FIG. 40E shows the PNPLA3 mRNA expression level, phosphorylated S6 (pS6/S6), phosphorylated AKT (pAKT/AKT), and phosphorylated NDRG1 (pNDRG1/NDRG1) after treatment with Alpelisib (BYL719). PNPLA3 expression largely did not change. Phosphorylated S6 increased at 2 hours, then decreased. Phosphorylated AKT largely did not change.

Table 39 provides quantitation of the phosphorylated proteins shown in FIGS. 40A-E.

TABLE 39

| | | mTORC2 pathway | | mTORC1 pathway | | |
|---|---|---|---|---|---|---|
| Compound | | pAKT | p-NDRG1 | p4EBP1c (37/46)#/ | | |
| Name | Target | (473)/AKT | /NDRG1 | 4EBP1c | pS6/S6 | PNPLA3 mRNA |
| PF-04691502 | PI3K/mTOR | 0.06 | 0.46 | 0.45 | 0.06 | 0.276 |
| OSI-027 | mTORC1/2 | 0.06 | 0.46 | 0.43 | 0.06 | 0.539 |
| CH51332799 | PI3Kα/β | 0.22 | 0.47 | 0.40 | 0.06 | 0.511 |
| Rapamycin | mTORC1 | 2.55 | 1.76 | 0.49 | 0.07 | 1.2 |
| Alpenisib | PI3Kα | 0.34 | 1.11 | 0.61 | 1.11 | 0.971 |

These results show that compounds that inhibit both mTORC1 and mTORC2 (see e.g. cells treated with OSI-027 or PF-04691502), and/or PI3Kβ (see e.g cells treated with PF-04691502 and CH51332799) down regulated PNPLA3 gene expression. In contrast, compounds that inhibit mTORC1 only (see e.g. cells treated with rapamycin) or PI3Kα (see e.g. cells treated with Alpenisib) did not lead to decreased PNPLA3 expression. Thus, compounds that target mTORC2, in addition to mTORC1, and/or PI3Kβ are the most efficient at reducing PNPLA3 gene expression.

### Example 31

### In Vivo Glucose and Insulin Quantification After Inhibitor Treatment

Next, the effect of mTOR and mTOR/PI3K inhibitors on in vivo insulin and glucose levels were assessed.

### In Vivo Dosing Materials and Methods

Mouse 7-8 week old C57BL/6J mice were divided into 9 groups. Each group had 8 male mice. All mice were given a high sucrose diet for 10 days (Diet no. 901683; 74% kCal from sucrose. MP Biomedicals, Santa Ana, Calif.) at the start of the dark cycle, about 7 pm. Food was removed at the start of the light cycle, about 7 am, except on the last day, when food was left in the cage until termination. On day 7-10, mice were administered daily (QD) via oral gavage, candidate compounds at a volume of 10 mL/kg with the compound in vehicle solution (0.5% methylcellulose/0.2% tween20). Vehicle alone was administered to control group 1. OSI-027 was administered at 25 mg/kg, 10 mg/kg, 5 mg/kg, and 2 mg/kg to groups 2-5. PF-04691502 was administered at 10 mg/kg, 5 mg/kg, 2 mg/kg, and 1 mg/kg to groups 6-9. The treatment was administered in the evening on Days 7 to 10 and in the morning on Day 11, starting at 5 am. On Day 11, mice were terminated 4 hours post last dose at 9 am, for a total of 5 doses of each candidate compound. Mice were weighed 2×/week until Day 11. Liver and blood samples were collected after mice were terminated. Liver samples were process for mRNA extraction as previously described. Blood samples were processed for serum collection. The geometric mean for the mRNA analysis was calculated by averaging the PCR CTs from the housekeepering genes ACTB, GAPDH, GUSB, HPRT, and B2M from the same cDNA sample.

### Serum Glucose Quantification

Serum glucose levels were measured in a single-reagent coupled-enzyme assay, against a glucose standard curve, colorimetrically. The glucose assay reagent was prepared as follows: one capsule of glucose oxidase/peroxidase (Sigma, cat #G3660-1CAP) was dissolved in 19.6 ml of deionized water. Separately, one vial of O-Dianisidine reagent (Sigma, cat #D2679) was dissolved in 0.5 ml of deionized water, 0.4 ml of the O-Dianisidine reagent was added into the enzyme mix to make 20 ml of 2× Glucose assay reagent. The glucose assay reagent was made fresh prior to running the assay.

A glucose standard curve was prepared by serially diluting D-glucose two-fold from 200 ug/ml to 12.5 ug/ml in 1× PBS. A no glucose control was included as a reagent blank.

Mouse serum samples were diluted 30-fold in 1× PBS. 50 μl of the sample (or standard) was combined with 50 μl of the glucose assay reagent in a 96-well microplate. The reaction was incubated at 37° C. for 30 min. 100 μl of 2N sulfuric acid was then added to quench the reaction. The color developed was read spectrophotometrically at 540 nm. The amount of glucose in the samples were determined based on the parameters of the linear fit obtained from the glucose standard curve.

### Serum Insulin Quantification

Serum insulin levels in mouse samples were quantified using an ELISA kit purchased from Crystal Chem (Catalog #90080), per the manufacturer's instructions.

### Results

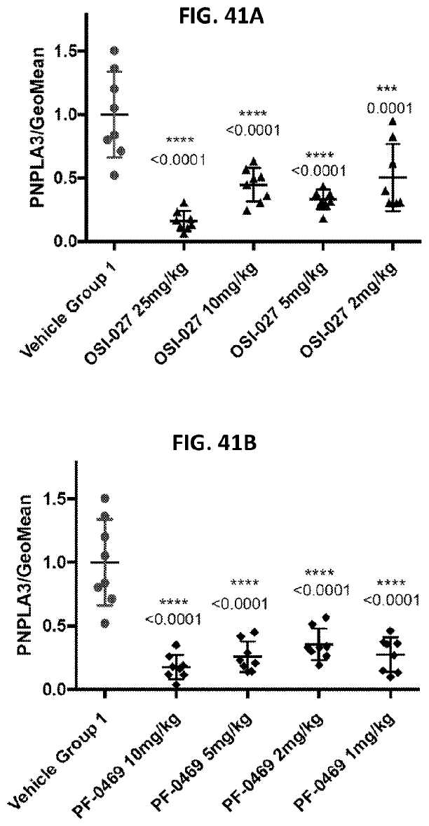

FIG. 41A shows the relative PNPLA3 mRNA in mouse livers as normalized against the geometric mean of the housekeeping genes after treatment with OSI-027. FIG. 41B shows the relative PNPLA3 mRNA in mouse livers as normalized against the geometric mean of the housekeeping genes after treatment with PF-04691502. Both OSI-027 and PF-04691502 treatment resulted in decreased PNPLA3 at each dose tested, with p<0.00001 as compared to untreated control mice. Statistical analysis was performed with one way ANOVA, followed by Dunnett test for multiple comparisions, ns=not significant.

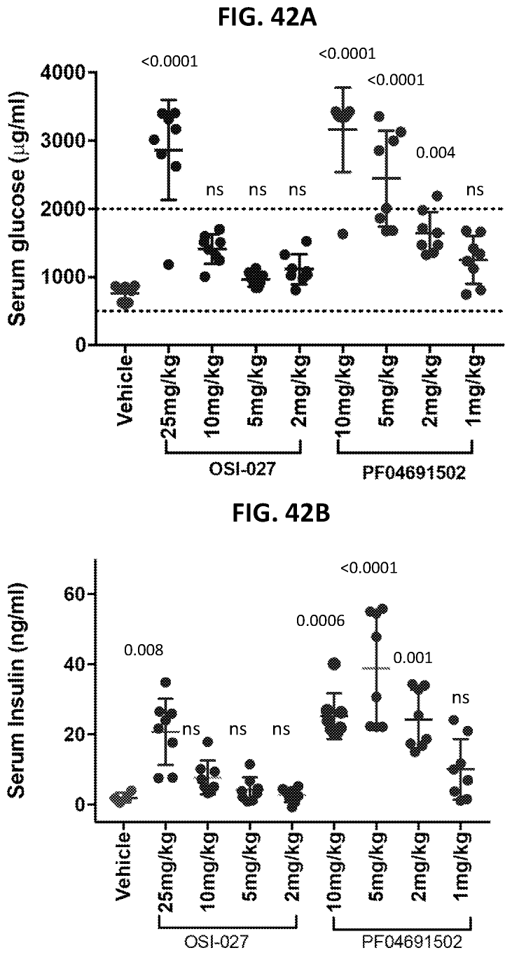

However, treatment of mice with OSI-027 and PF-04691502 resulted in different serum glucose and serum insulin outcomes. Only the highest dose of OSI-027 treatment, 25 mg/kg, resulted in significant increased serum glucose (FIG. 42A) and serum insulin (FIG. 42B), while the three lower doses, 10 mg/kg, 5 mg/kg, and 2 mg/kg, resulted in no significant changes in serum glucose or serum insulin levels in the mice. In contrast, PF-04691502 treatment resulted in statisically significant increases in the insulin and glucose levels at the 10 mg/kg, 5 mg/kg, and 2 mg/kg doses. Thus, OSI-027 treatment at three different concentrations lead to a 50% reduction of PNPLA3 expression in viva without an adverse increase in serum insulin or glucose. Statiscial analysis was performed with one way ANOVA, followed by Dunnett test for multiple comparisions, ns=not significant.

In contrast, mice treated with PF-04691502 experienced significant serum glucose and serum insulin increases at the three highest doses, 10 mg/kg, 5 mg/kg, and 2 mg/kg (FIG. 42A and FIG. 42B), and moderate increases in serum insulin at the lowest dose, 1 mg/kg (FIG. 42B). The lowest dose of PF-0469150, 1 mg/kg, lead to a 50% decrease in PNPLA3, but still induced moderate increased insulin and glucose levels in the mice.

The dual PI3kα/β and mTORC1/C2 inhibitor PF-0469150 decreased PNPLA3 expression in vivo but also induced increased levels of serum glucose and insulin, while the mTOR only inhibitor OSI-027 decreased PNPLA3 expression with minimum adverse side effects. Based on these results, inhibition of the PI3kα/β pathway leads to adverse in vivo results, e.g. increased serum glucose and insulin levels. Increased levels of serum insulin, or hyperinsulinernia, is associated with pre-diabetes, hypertension, obesity, dyslipidemia, and glucose intolerance. High blood sugar, or hyperglycemia, can lead to nerve damage, blood vessel damage, or organ damage, as well as decreased healing, increased skin and mucosal infections, vision problems, or gastrointestinal issues such as constipation or diarrhea.

Therefore inhibition of only the mTOR pathway to reduce PNPLA3 expression is preferable, due to the adverse effects induced by inhibition of the PI3K pathway.

### Example 32

### mTOR Inhibitory Activity

A candidate compound is tested for mTOR inhibitory activity via an antibody binding assay.

Human hepatocytes are treated with various concentrations of the candidate compound for 35 min, 1 hr, 2 hrs, 3 hrs, 4.5 hrs, or 20 hrs. Cells are harvested for protein immunoblots using Laemmli buffer (2% SDS, 10% glycerol, 75 mM Tris-Cl, pH 6.8, 5% beta-mercaptoethanol, bromphenol blue). Hepatocyte cell lysates are loaded onto 4-12% Bis-Tris gels with 35,000 cells/15 uL per lane. Blots are incubated with primary antibodies overnight in Odyssey blocking buffer. Antibodies used include pAKT (Ser473) Rabbit mAb 4060 (Cell Signaling (1:1000)), pS6 Ser235/236 Rabbit mAb 4858 (Cell Signaling (1:1000)), pNDRG1 T346 Rabbit mAb 5482 (Cell Signaling (1:1000)), p4EBP1c (Thr37/46) Rabbit mAb 2855 (Cell Signaling (1:1000)), AKT (pan) Mouse mAb 2920 (Cell Signaling (1:1000)), Ribosomal Protein S6 (C-8) sc-74459 Mouse mAb (Santa Cruz Biotech (1:2000)), NDRG1 A-5 sc-398823 Mouse mAb (Santa Cruz Biotech (1:200)) and 4EBP1 (53H11) Rabbit mAb 9644 (Cell Signaling (1:1000)), pSGK1 (Ser78) rabbit mAB 5599 (Cell Signaling), SGK1 rabbit mAb 12103 (Cell Signaling), pPKC (Thr410) rabbit mAb 2060 (Cell Signaling), PKC rabbit mAb 9960 (Cell Signaling). Blots are incubated with secondary antibodies IRDye® 800CW Donkey anti-Rabbit IgG (H+L) 926-32213 or Donkey Anti-Mouse IgG Polyclonal Antibody (IRDye® 680LT) 926-68022 at 1:10,000 in Odyssey blocking buffer for 1 hour, and are imaged using Odyssey Licor Scanner. Image Studio software is used to quantify phosphorylated protein abundance to total protein abundance, relative to DMSO control from each timepoint.

Levels of at least one of phosphorylated S6, AKT, SGK1, PKC, NDRG1, and 4EBP1c proteins are determined as compared to total S6, AKT, SGK1, PKC, NDRG1, and 4EBP1c protein levels.

Cells treated with candidate compounds that have mTORC1/C2 inhibitory activity show a decrease in the relative amount of phosphorylated S6, AKT, SGK1, PKC, NDRG1, and/or 4EBP1c. mTORC2 specific inhibitors show decreased levels of phosphorylated AKT, SGK1, PKC, and/or NDRG1 but not S6 and/or 4EBP1c. mTORC1 specific inhibitors show decreased levels of phosphorylated S6 and/or 4EBP1c but not AKT, SGK1, PKC, and/or NDRG1. mTORC1/C2 inhibitors show decreased levels of both phosphorylated S6 and/or 4EBP1c and AKT, SGK1, PKC, and/or NDRG1.

### Example 33

### PI3K Inhibitory Activity

Compounds identified in Example 32 as mTOR inhibitors are assessed for PI3K inhibitory activity in a biochemical assay.

Purified PI3Kα or PI3Kβ is purchased from Promega (catalogue no. V1721 or V1751). An ADP-Glo kit with PIP2 is purchased from Promega (catalogue no. V1791). Alternatively, an ADP-Glo kit with PI is purchased from Promega (catalogue no. V1781).

A standard curve of the kinase substrate is prepared according to the manufactures instructions. A working solution of the PI3K kinase in reaction buffer with the substrate is prepared. Serial dilutions of the candidate compound are made in buffer. The candidate compound samples are added to the kinase and substrate mixture and incubated to allow binding of the kinase to the substrate. Control sample with no enzyme (background control) or no candidate compound (negative control) are run. A known PI3K inhibitor, such as CH51332799, is used as a positive control. The reaction is started by adding ATP to a final concentration of 25 μM and incubated for 1 hr. The reaction is halted by adding ADP-Glo Reagent. Kinase Detection Reagent is added to the samples to convert the ADP to ATP, and the luciferase and luciferin to detect the new ATP. The luminescence of the samples is quantified with a luminescent plate reader. The IC50 of a candidate compound is determined from the serial dilution curve, as compared to the luminescence of the sample with no candidate compound (100% activity).

Inhibition of the PI3K kinase reaction results in reduced luminescence of the samples. Thus, samples treated with compounds with PI3K inhibitory activity show decreased luminescence, while samples treated with compounds that do not inhibit PI3K do not show decreased luminescence in the assay.

Candidate compounds selected for further analysis and development are those that have mTORC1/2 or mTORC2 inhibitory activity and do not inhibit the activity of PI3K, including PI3Kβ.

### Example 34

### DNA-PK Inhibitory Activity

Compounds identified in Example 32 as mTOR inhibitors are assessed for DNA-PK inhibitory activity in a biochemical assay.

Purified DNA-PK and the DNA-PK substrate is purchased from Promega in a kit (catalogue no. V4106). An ADP-Glo kit is purchased from Promega (catalogue no. V9101, or V4107 when purchased with the DNA-PK kit).

A dose response curve of the DNA-PK kinase substrate is prepared according to the manufactures instructions to determine the optimal kinase and ATP concentration. A working solution of the DNA-PK kinase in reaction buffer with the substrate is prepared. Serial dilutions of the candidate compound are made in buffer. The candidate compound samples are added to the kinase and substrate mixture and incubated to allow binding of the kinase to the substrate. Control sample with no enzyme (background control) or no candidate compound (negative control) are run. A known DNA-PK inhibitor, such as LY3023414 or CC-115, is used as a positive control. The reaction is started by adding ATP to a final concentration as previously determined and incubated for 1 hr. The reaction is halted by adding ADP-Glo Reagent. Kinase Detection Reagent is added to the samples to convert the ADP to ATP, and the luciferase and luciferin to detect the new ATP. The luminescence of the samples is quantified with a luminescent plate reader. The IC50 of a candidate compound is determined from the serial dilution curve, as compared to the luminescence of the sample with no candidate compound (100% activity).

Inhibition of the DNA-PK kinase reaction results in reduced luminescence of the samples. Thus, samples treated with compounds with DNA-PK inhibitory activity show decreased luminescence, while samples treated with compounds that do not inhibit DNA-PK do not show decreased luminescence in the assay.

Candidate compounds selected for further analysis and development are those that have mTORC1/2 or mTORC2 inhibitory activity and do not inhibit the activity of DNA-PK.

### Example 35

### Insulin and Glucose Assays

Compounds identified in Example 32 as mTOR inhibitors are assessed for the ability to increase insulin and glucose levels in vivo.

Mice are dosed with candidate compounds and serum is collected for glucose and insulin quantification as described in Example 31. Increased levels of serum insulin or glucose are observed in mice treated with compounds that increase insulin or glucose.

Candidate compounds selected for further analysis and development are those that have mTORC1/2 or mTORC2 inhibitory activity and do not increase insulin or glucose.

### Example 36

### PNPLA3 Gene Expression Assays

Compounds identified in Example 32 as mTOR inhibitors are assessed for the ability to decrease PNPLA3 expression. Hepatocytes are treated with a candidate compound and PNPLA3 expression is quantified as described in Examples 3, 6, and 18. Decreased PNPLA3 mRNA is observed in cells treated with compounds that reduce PNPLA3 gene expression.

Candidate compounds selected for further analysis and development are those that have mTORC1/2 or mTORC2 inhibitory activity and decrease PNPLA3 gene expression.

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

1. A method of treating a subject in need thereof with a Patatin-like phospholipase domain-containing protein 3 (PNPLA3)-targeted therapy comprising
a. obtaining or having obtained a dataset comprising genomic data from a biological sample obtained from the subject;
b. determining or having determined the presence or absence of a G allele at SNP rs738409 in the dataset;
c. identifying or having identified the subject as eligible for the PNPLA3-targeted treatment based on the presence of the G allele at SNP rs738409; and
d. administering to the subject an effective amount of a compound capable of reducing the expression of the PNPLA3 gene, wherein the compound capable of reducing the expression of the PNPLA3 gene comprises an mTOR inhibitor that does not inhibit the PI3K pathway.

2. The method of claim 1, wherein the determining step comprises detecting the allele using a method selected from the group consisting of: mass spectroscopy, oligonucleotide microarray analysis, allele-specific hybridization, allele-specific PCR, and nucleic acid sequencing.

3. A method of treating a subject in need thereof with a PNPLA3-targeted therapy comprising
a. obtaining or having obtained a dataset comprising proteomic data from a biological sample obtained from the subject;
b. determining or having determined the presence or absence of a mutant PNPLA3 protein carrying the I148M mutation in the dataset;
c. identifying or having identified the subject as eligible for the PNPLA3-targeted treatment based on the presence of the mutant PNPLA3 protein carrying the I148M mutation; and
d. administering to the subject an effective amount of a compound capable of reducing the expression of the PNPLA3 gene, wherein the compound capable of reducing the expression of the PNPLA3 gene comprises an mTOR inhibitor that does not inhibit the PI3K pathway.

4. The method of claim 3, wherein the determining step comprises detecting the mutant protein using mass spectroscopy.

5. The method of any one of claims 1-4, wherein the biological sample is a biopsy sample.

6. The method of any one of claims 1-5, wherein the mTOR inhibitor does not inhibit PI3Kβ activity.

7. The method of any one of claims 1-5, wherein the mTOR inhibitor does not inhibit DNA-PK.

8. The method of any one of claims 1-7, wherein the mTOR inhibitor is OSI-027.

9. The method of any one of claims 1-7, wherein the mTOR inhibitor comprises an mTORC2 inhibitor.

10. The method of claim 9, wherein the mTORC2 inhibitor comprises a RICTOR inhibitor.

11. The method of claim 10, wherein the RICTOR inhibitor is JR-AB2-011.

12. The method of any one of claims 1-11, wherein the administration of the compound capable of reducing the expression of the PNPLA3 gene does not induce hyperinsulinemia in the subject.

13. The method of any one of claims 1-11, wherein the administration of the compound capable of reducing the expression of the PNPLA3 gene does not induce hyperglycemia in the subject.

14. The method of any one of claims 1-5, wherein the compound capable of reducing the expression of the PNPLA3 gene is selected from the group consisting of OSI-027, WYE-125132, CC-223, Everolimus, Palomid 529 (P529), GDC-0349, Torin 1, PP242, WAY600, CZ415, INK128, TAK659, AZD-8055, and JR-AB2-011.

15. The method of any one of claims 1-7, wherein the compound comprises one or more small interfering RNA (siRNA) targeting one or more genes selected from the group consisting of RICTOR, mTOR, Deptor, AKT, mLST8, mSIN1, and Protor.

16. The method of claim 15, wherein the one or more small interfering RNA (siRNA) targets RICTOR.

17. The method of any one of claims 1-16, wherein the subject is homozygous for the G allele at SNP rs738409.

18. The method of any one of claims 1-16, wherein the subject is heterozygous for the G allele at SNP rs738409.

19. The method of any one of claims 1-16, wherein the subject is homozygous for the mutant PNPLA3 protein carrying the I148M mutation.

20. The method of any one of claims 1-16, wherein the subject is heterozygous for the mutant PNPLA3 protein carrying the I148M mutation.

21. The method of any one of claims 1-20, wherein the expression of the PNPLA3 gene is reduced by at least about 30%.

22. The method of any one of claims 1-20, wherein the expression of the PNPLA3 gene is reduced by at least about 50%.

23. The method of any one of claims 1-20, wherein the expression of the PNPLA3 gene is reduced by at least about 70%.

24. The method of any one of claims 21-23, wherein the reduction is determined in a population of test subjects and the amount of reduction is determined by reference to a matched control population.

25. The method of any one of claims 1-24, wherein the expression of the PNPLA3 gene is reduced in the liver of the subject.

26. The method of claim 25, wherein the expression of the PNPLA3 gene is reduced in the hepatocytes of the subject.

27. The method of claim 25, wherein the expression of the PNPLA3 gene is reduced in the hepatic stellate cells of the subject.

28. The method of claim 25, wherein the expression of the PNPLA3 gene is reduced in the hepatocytes and hepatic stellate cells of the subject.

29. The method of any one of the preceding claims, wherein the method further comprises assessing or having assessed a hepatic triglyceride content in the subject.

30. The method of claim 29, wherein the assessing or having assessed step comprises using a method selected from the group consisting of liver biopsy, liver ultrasonography, computer-aided tomography (CAT) and nuclear magnetic resonance (NMR).

31. The method of claim 30, wherein the assessing or having assessed step comprises proton magnetic resonance spectroscopy (¹H-MRS).

32. The method of claim 29, wherein the subject is eligible for treatment based on a hepatic triglyceride content greater than 5.5% volume/volume.

33. A method of reducing the lipid content in cells in a subject, comprising the steps of:
a. obtaining or having obtained a biological sample from the subject;
b. determining or having determined in the biological sample the amount of lipid content; and
c. administering an effective amount of a compound capable of reducing the expression of the PNPLA3 gene.

34. The method of claim 33, wherein the method further comprising assessing the hepatic triglyceride in the subject.

35. The method of claim 34, wherein the assessing step comprises using a method selected from the group consisting of liver biopsy, liver ultrasonography, computer-aided tomography (CAT) and nuclear magnetic resonance (NMR).

36. The method of any one of claims 33-35, wherein the lipid content is in hepatocytes.

37. The method of claim 33-35, wherein the lipid content is in hepatic stellate cells.

38. The method of claim 33-35, wherein the lipid content is in a population of hepatocytes and hepatic stellate cells.

39. The method of any one of claims 33-38, wherein the compound comprises an mTOR inhibitor.

40. The method of any one of claims 33-38, wherein the compound comprises OSI-027.

41. The method of any one of claims 39-40, wherein the mTOR inhibitor comprises an mTORC2 inhibitor.

42. The method of claim 41, wherein the mTORC2 inhibitor comprises a RICTOR inhibitor.

43. The method of claim 42, wherein the RICTOR inhibitor is JR-AB2-011.

44. The method of any one of claims 33-38, wherein the compound comprises PF-04691502.

45. The method of any one of claims 33-38, wherein the compound capable of reducing the expression of the PNPLA3 gene comprises at least one selected from the group consisting of OSI-027, PF-04691502, Momelotinib, WYE-125132, CC-223, Everolimus, Palomid 529 (P529), GDC-0349, Torin 1, PP242, WAY600, CZ415, INK128, TAK659, AZD-8055, Deforolimus, and JR-AB2-011.

46. The method of any one of claims 33-38, wherein the compound comprises one or more small interfering RNA (siRNA) targeting one or more genes selected from the group consisting of JAK1, JAK2, mTOR, RICTOR, Deptor, AKT, mLST8, mSIN1, and Protor.

47. The method of claim 46, wherein the one or more small interfering RNA (siRNA) targets RICTOR.

48. The method of claim 46, wherein the one or more small interfering RNA (siRNA) targets mTOR.

49. The method of any one of claims 33-48, wherein the expression of the PNPLA3 gene is reduced by at least about 30%.

50. The method of any one of claims 33-48, wherein the expression of the PNPLA3 gene is reduced by at least about 50%.

51. The method of any one of claims 33-48, wherein the expression of the PNPLA3 gene is reduced by at least about 70%.

52. The method of any one of claims 49-51, wherein the reduction is determined in a population of test subjects and the amount of reduction is determined by reference to a matched control population.

53. A method for identifying a compound that reduces PNPLA3 gene expression comprising
a. providing a candidate compound;
b. assaying the candidate compound for at least two of the activities selected from the group consisting of: mTOR inhibitory activity, mTORC2 inhibitory activity, PI3K inhibitory activity, PI3Kβ inhibitory activity, DNA-PK inhibitory activity, ability to induce hyperinsulinemia, ability to induce hyperglycemia, and PNPLA3 gene expression inhibitory activity; and
c. identifying the candidate compound as the compound based on results of the two or more assays that indicate the candidate compound has two or more desirable properties.

54. The method of claim 53, wherein the desirable properties are selected from the group consisting of: mTOR inhibitory activity, lack of PI3K inhibitory activity, lack of PI3Kβ inhibitory activity, lack of DNA-PK inhibitory activity, lack of ability to induce hyperinsulinemia, lack of ability to induce hyperglycemia, and PNPLA3 gene expression inhibitory activity.

55. The method of claim 54, wherein mTOR inhibitory activity comprises inhibition of mTORC2 activity.

56. The method of claim 54, wherein the mTOR inhibitory activity is mTORC1 and mTOR2 inhibitory activity.

57. The method of claim 54, wherein the PI3K inhibitory activity is PI3Kβ inhibitory activity.

58. The method of any of claims 53-57, wherein the assaying step comprises assaying for at least three of the activities.

59. The method of any of claims 53-57, wherein the assaying step comprises assaying for at least four of the activities.

60. The method of any of claims 53-57, wherein the assaying step comprises assaying for at least five of the activities.

61. The method any of claims 53-57, wherein the at least two assays of step (b) comprise assays for mTOR inhibitory activity and PI3K inhibitory activity.

62. The method any of claims 53-57, wherein the at least two assays of step (b) comprise assays for mTORC2 inhibitory activity and PI3Kβ inhibitory activity.

63. The method of claim 58, wherein the at least three assays of step (b) comprise assays for mTOR inhibitory activity, PI3K inhibitory activity, and ability to induce hyperinsulinemia.

64. The method of claim 59, wherein the at least four assays of step (b) comprise mTOR inhibitory activity, PI3K inhibitory activity, ability to induce hyperinsulinemia, and DNA-PK inhibitory activity.

65. The method of any one of claims 53-64, wherein the assay is a biochemical assay.

66. The method of any one of claims 53-64, wherein the assay is in a cell.

67. The method of claim 66, wherein the cell is an animal cell or a human cell.

68. The method of claim 66 or 67, wherein the cell is a wild type cell.

69. The method of claim 66 or 67, wherein the cell comprises the G allele at SNP rs738409 of the PNPLA3 gene or a mutant I148M PNPLA3 protein.

70. The method of claim 69, wherein the cell is homozygous for the G allele at SNP rs738409.

71. The method of claim 69, wherein the cell is heterozygous for the G allele at SNP rs738409.

72. The method of claim 69, wherein the cell is homozygous for the mutant PNPLA3 protein carrying the I148M mutation.

73. The method of claim 69, wherein the cell is heterozygous for the mutant PNPLA3 protein carrying the I148M mutation.

74. The method of claim 53, wherein assaying the PNPLA3 gene expression comprises a method selected from the group consisting of: mass spectroscopy, oligonucleotide microarray analysis, allele-specific hybridization, allele-specific PCR, and nucleic acid sequencing.

75. The method of claim 74, wherein the expression of the PNPLA3 gene is reduced by at least about 30%.

76. The method of claim 74, wherein the expression of the PNPLA3 gene is reduced by at least about 50%.

77. The method of claim 74, wherein the expression of the PNPLA3 gene is reduced by at least about 70%.

78. The method of any one of claims 75-77, wherein the reduction is determined in a population of cells and the amount of reduction is determined by reference to a matched control cell population.


SEQUENCE LISTING (1 sequence)

SEQ ID NO: 1 - DNA, 23830 bp, Homo sapiens
atggtccgaggggggcggggctgacgtcgcgctgggaatgccctggccgagacactgaggcagggtagagagcgcttgcgggcgccgggcggagctgctgcggatcaggacccgagccgattcccgatcccgacccagatcctaacccgcgcccccgccccgccgccgccgccatgtacgacgcagagcgcggctggagcttgtccttcgcgggctgcggcttcctgggcttctaccacgtcggggcgacccgctgcctgagcgagcacgccccgcacctcctccgcgacgcgcgcatgttgttcggcgcttcggccggggcgttgcactgcgtcggcgtcctctccggtatcccgctgggtgcgtctggggacgctgcccgggctccacgtgcggagtgggtgccccctaggccggggagcgggggatccccaggggtcgcggggccctggaggagcgggcatcggacgcggacacggcggggtgcatcccgagggccccctccgaggcagatgcttcctgcgggggcgctgttcctgggcccgggaagggggcgttggaaccccgagcggtccgggccgaagcctgggactctcgtgcgtccccacccctacccccatcaggcgcccgtgcatgaagggagaccctcacctccggactgagagtcggagcgtctcggagcgacggggagtagggagcgggacccggggcggagggtagtgctggcccctgcggactccgggtcccctgtgtcctctcgggaggggctggacgggctgagctgccgaggggccgatttgccctgggccggacaaagagtggggctttggccggtcccccacggtgggctccttccctctggggattgagggactcaagacaccccgcgcctgcgcttttcttttctttttttctttttttttttttgagacggagtttcgctcagtcgcccaggctggagtgcagtggcgtgatctcaactcactgcaagctccacctcccaggttcacgccattctcctgcctcagcctcccgagtagctgggactacaggcgccagccaccaagcccggctaattttttgtattttttagtagagacggggtttcaccgtgttagccaggatggtctcgatctcctgacctcgtgatctgcccacctcggcctcccagaatgctggggttacaggcgtgagccactgctccctgctgcctacgctctctgggtcgcagcccagccttctgggggctgggtagcctcccagaagggcaaccctgggcatcctccagggcaggctaactggagtctagtggggaggggtaccttgaaagaggaaagttgtttcctcctcctcctcctcctccagtgtttgggacccttcctgggggctggagtgcatccctggacaccccccaatcccatcctcttctctagtttccactgacctaggcccaccctcccctctccggctcagtactcctggaaatgagattccgtacatttgaatcttgtcctaatgaaatatttgtccatgtgggtacctgtgtgtgtgtggtgggggtgcagacggagggtttgtttctcactagctggaactactggggtgtggtatgcttcctgggaatttgtgtgccacagtcctggaggcgaggagggggttgtgagccagtaggcaggggctggggcaagtagcattgtgaagctattgacacccagacgtccccaggcaggagattatgcccccattagcccccttttatctgggcttccttaacaatggactctttgccctgcctgccagagccagcagggagtgactgttcagtggtgaggaagcgggcagaggaagccctgccattgggtaggagcagtgggcagcccctgggctgactgggaggtggggattagggattagacagtcctggctgtctgccttcccctaagccagggggagaggagcaaagggcacgaaatgtggcctccaggaggattagaccgccacatgatcatttgcacaccctggggtttagcaacaataaaagtcagcttttttgtatcccaaggtggcctgtggacacccacatggacaaatgtttacactgggacagaattcaaatgcagaggtcccaggagcctaaagtacactcactctggtatagaaaggattccttactgggcagaggacaggtgcagcctggggctttcccaggcaggacacagggaggctcaggaaccaccaagtccctggaaggtggatctggaggcgttggcaggagccactccctgggttccagggctccaggttcctgctttaaccccctgtctcacagagggctgtgcacttgggggctgctgagcatgtcccagaggctgcatcctggacacagcacctcagtgcatctgagctgaggctaacttggcaggagggacaggcagaacctgccagccacgtgcaattccacccctctggccactcagggaaggagagctgtgagtcaagatcagatttgggtcaggacaggctggggcctgcctgtccctgtgcatcccaagatttatggctggccaggggttgggctgggaggggtggtcttgcatgccaggagagtgcagatcagcctgagaggccaggccagtaagtgaggtcagatctcctgcacctgatagcattaaggccatctacaccaaagctctaatgctgatatgttcctggcctctatgtggggcatggaggtggggcatggaggtgaggcctgctcgcctgggcttctggaagtgggagactcattcctgtggctgaggcctacagcagtgctgtgtggtaggaatacactggaagccatgatgtcattgtgcattttctagaagccacattgaataaagtaaaagacacaggtagaattaatttcattgagcccaatatatccaaaataatatcattttcacatctattcaatataaaaatttactaatgagatatttcatactaagccactgaaatccagtttgtatcttacacatctcagttttgacgagccacatttcaagggcgtgatagccacatgtggctcccatagtagacagtactggtctagagaaatgttggtggcatccttgctgtctggtttctggccttgccaaaagtattaccatcccagtgtggtacattctttcatgtatttgtctcctgtccccagagcagactctgcaggtcctctcagatcttgtgcggaaggccaggagtcggaacattggcatcttccatccatccttcaacttaagcaagttcctccgacagggtctctgcaaatgcctcccggccaatgtccaccagctcatctccggcaaaataggcatctctcttaccagagtgtctgatggggaaaacgttctggtgtctgactttcggtccaaagacgaagtcgtggatgtaagcagtttgcttatctggacgttgtcaagttagaaaagctgttttgggatgggtgtggtggctcatgcctgtcatcccggcactttgggaggccgaagcgggtgggttgcttgagcccaggagctcgagaccaacatgatgaaacccagtctctacaaaaattacagaaaaattagctaggcatggtgttgtgggcccatagtcccagctactagggaggctgaggcaggagaattgcttgagcctgggaggtggaggttgcagtaagtcatgatcatgccactgtactccagcccgggtgacagtgagatgctgtctggaaaaaaaaaaaaaagaaagactgttttgttttggaagcaacacaggcagttgtaggccccctgtgccagagtgacataaactctgtacacctccagtgatttggtccatgtttgtaaaccctgaatgttccagggcagtttcttttcttcactttttatctcttttttttgggtgggggggcggggtacagagtcttgctctgtctcccaggctggagtgcagtggcgcaatctcaacctcccgaggagctgggactacaggcacaggccatcacaccttgctaatgtttgtactttttgtagagacggggttttgccctgttgcccaggctggtcccaaactcctgcacccaagtaatctgcccacctctgcctggcagttacaatttcaaataattcctccctttccttcaacacttggctcatgaccgtccagtccaaggaacctgtcctgcaggtgtgcctctcccgagcttcctctatgcatcttccataatgaagatgccttctcactggaaaccctacaagggtgggaacgtgccttatttgcctgtatcctcagggtctagcagagagaagataatttgtaataccaaaacaccattaaattcagctgatgctttcataagcgctccttggaggaaggactccatttacttgacagatctgtgcaagacagcagcctggcgcgtctaacctgcagccagttgcatcctctgtttaaccttgtttgcggaagctttctctaaacagccagcacttgtctgttcccacatgggtccgttctcccagtgaatcaccgtggtgcctgctgactgctctgtagcacagtgcttcgcaaagtgtgatcctgggaccagcagagcagcagctcctttgagcttattggaatggcagaccctcaggtcccacctctgacctgctgcatgggaattctggggagggacgcagaatctctggttccacaggctctccggtgatgctaatgaataccggcatttgaacagcaccgatctagcccctttcagtccatgagccaacaacccttggtcctgtctgtggtgacccagtgtgactctcatggggagcaaggagaggaagttgaagttcactgacagggttgttaaggggattatgcaatagatgagacccatgggcctgaagtccgagggtgtatgttagttccccgttcttttgacccatggattaacctactctgtgcaaagggcattttcaagtttgttgccctgctcacttggagaaagcttatgaaggatcaggaaaattaaaagggtgctctcgcctataacttctctctcctttgctttcacaggccttggtatgttcctgcttcatccccttctacagtggccttatccctccttccttcagaggcgtggtaagtcggctttctctgctagcgctgagtcctgggggcctctgaagtgtgctcacacatctcctgcctgcagggcactggtgtcgggcacctcagggtctgtcccatggtggagccccatgcctcactgcctttcagacagagtagccacagctggccctatttccaggctacccgggcagcaaaacttactgcatgtgtaattaattatttggctatctgtaaggtaaactggctggttcacttaatctgcaccttaagcatcagatagcttctcagtgatctagttaaactatatgatgttggccaggcgcggtggctcatgtctgtaatcccagcactttgggagcctgaagcaggcagatcacttgaggtcaggagttcgagaccagcctggccaacagtgtgaaactctgtctctcctaaaaatacaaaaattagctgggcatggtggtgtgcacctgtaatcccagctgctcgggaggctgaggcaggagaattgcttgaacttgggaggcggaagttgcagtgagccaagatcgcaccactgcactccatcctgggtgacagagcgagactctatctcaaaaagaaaaaaaaaaaaaaggtaaataaagtatatgacactgaagaatctgttacccctggaaggtggagctttactcttagggggaactataacagtcatatatatatatttttttcttttctttttttttttttttgagatggagtctggctctgtcgcccaggctggagtgcagtggtgcaatctcggctcactgcaacctccacctcacaggttcaggcaattctcctgcctcaacctcccgagtagctgggattacaggtgcctgccgtcacgccaagctaatttttgtatttttagtagagacagggtttcatcatattggccaggctggtctccaactcctgacctcaggtgatccgcccgccttggcctcccaaagtgctgagattacaggcgtgagccatggtgcccggccaacaatcacatgtgttgtaaacaacaacaaaaatctgtcagcctggtctaacctagatttgtgctttgttttgttttgccactttgtgatgcacaggaggaagtttaggctgtaaaatactagccttttagggtaatttttgaactcacaagagcagcagcggaacctttgatgcaatcctgtatgtagcaccagcagagccacgtggcagagggactcgcattaggagcctcccattacagactacgtgctcctgtgcgttatcttatagggtccccacaaccaaggggagatgtgattattcatcctgtgtggctgtggggaacttgagagtcatacttgcccaaagagcacggccagcgagcttgcacccaggtcactctctgctcctctgtcagaacagggcatgtcttggttcactgcagggcggctcttctcattctctgtagtttggggtccaggatagtggtccacggagccactggagtgcccagctactgagtgaccaaagcatattttggatttccgacattgccacagcatggttgggcatcagcaggaccccaaccccttgttatgctggtggctttatgtggttatttgatcttccccagaactcagcaggagtgcacccagcagcaccgtagtgatgctctctggctccccagtgcacggttctggctttccttcctggtcgagagtttcaagccctctgggtcctactctgtccttttcagcccatagctttgttcaaaagctgctggcagtgttcagatttggctgagttcagtgaatatgtgcattggctgatttctgagccatgccagggggatggagaagccgaagcaggagtgtttgttctgcaggctctggagtaggcattgggtctgtgccggctcacttgctagtcttgcatccttccccaaccccctctggggatgtctggccacatcagaagacagtttgggttgtcagaactgggggagtaccaggccgaggtgggtggatcatgaggtcaggagatcgagaccatcctggctaacacagtgaaacctcatctctactaaacatacgaaaaaaattagctgggcgtggtggcgggcgcctgtagtcccagctactcgggaggctgaggcaggagaatggtgtgaacccggggggcggagcttgcagtgagctgagatcctgccactgcactccagcctgggcaacaaagcgagactccgtctcacaaacaaaacaaaacaaaacaaaacaaaatctgggggagtgccactggcatctgatgtatagaggcccgagatgctgtgtcatcacccgttgagtgcgctcataggcatcttcctgacaattagaacccattattcttcaaattcaatgcaagcaaattcaaagcattactgtgtacataccgcatgctaatcaattgcaccactggagctcctaaattcaaaacattactataaaaaagttcaaaatgcatggaaaagttgtacatggcaggagaatatttgggcttctgactaccccttgaatgaagatgatccaccagccgccttcctccttggtcttcactccagattcctagcatttcattctgtgtctctttatgcagtgaggtttttgtttgttttttgagacagagtctcactgtatcacctaggcctggagtgcagtggcgcgatctcagctcactgcaaccctcggctcctgggtttaagcgattctcctgcctcagcctcccgagcagctgagattacaagcacacatccccatgcccagctaatttttgtatttttagcagagacagggtttcaccatgttgcccaggctggtctcgaactcctggcctcaagtgatccatgtgcctcagccttccaaagtgctgggattacaggcgtgagccaccatgcccagctcctagtgaggtttttgatgccttgctacatctgccctagaaattgtgtgactacgattttggaaatgttgctgtgtaaacttgtgatcatttctggactccaggcaagaatcttgatggctaaggtgtggctgaacatgtctgattctctcctggacctgttttaggccaaactctgctctgaaattcctccgtgtggaagggcgggctggggagagcctcccagctggaatcttttggatgcctttctctgtgggtatctgatggctggctctgatggctggctgtgatggctgtggctggaaatcattgttgacatgagtttcacagatgcaggctctgtccaaattgtagcaaaagctgcctgccccagccgagctatgggcaataaggtggtttaaggatatagatgaaggaaaactcacccttagaataatttatccaaaatgctgctgtgttgtgggttagaggacattttctgaggtcccaggttcattgtttcatttaagtctcaaaagtccctccaggtgttggttctaattgtcaaagcatggggggagatgggctcatgggttaaaggtcttatcccagatttctgtatcctccttgcaagcagcaaaggggtctggatttgaatccatgaccatgtttctcctttgggtttccatcacactctgtccccgtgcactgagcaccctttagttcatatgacccccttaggcatgttacatgggcactcctataggtgcccatctggccctaggacttggccaacacaacatggactccagtttccatctgcctctttgccaggcacttttgtgcagtgcacacactgtacaacagtagacggcaaccctgagagccagagtagagcctgtcctagcaccggaatgctcggtaaggatttgtcgcaggagtgattccaaagccaatgtcctccctccatatcagcctgtttgtggctctgagaagctctgcccacatgtgaaagcttgttaagcacttaagcactaacccagagcttcagacagtgccagtcctttttccccttctttaaaagcgatatgtggatggaggagtgagtgacaacgtacccttcattgatgccaaaacaaccatcaccgtgtcccccttctatggggagtacgacatctgccctaaagtcaagtccacgaactttcttcatgtggacatcaccaagctcagtctacgcctctgcacagggaacctctaccttctctcgagagcttttgtccccccggatctcaaggtgagttggtggtgagggggcaggtgttctggggtgcagctcttctttgcctccctgattgccaggagctaccagttactgtctgcacaatcaaacagaaatagacctgtccttgatggttaacggaaataaaaggcgcttgtcccagaagctcaggtgaggcaccaccctgattatgggaatcacctgggaacatatacccagacctaaaactcagatccacttcccaggctgtggttatatagtcaggggggtgcagtatgggtattaggattttttattttttagttataaagatttttttttggtttgtttttgagacagggtcttgctctgccgcttaggctggagtgcagtggtgcaatcatagctcactgaagcctcagactcctgggttcaagcagtcctcccacctcagcctcctaaggagctgggacccacaggcatgcagcaccacacctggctaatttttaaaaattttgtggagtgttgcccaggctggtctcacactcctggcctcaagcgatcctcccaccccagcctcccaatgtgttgggattacaggcatgagccattgtacccagccactaagatgattcttatttggaaacacggtcaagaacaactgcgttcggtagtttaaccttttttgattgtggtggttttagtatgccttaccactctaccatagtaagaaatttgcagaccatgtacaccaacctttggtgctcctggggagaaagaaagaaggctatgcaatgcaatgcatgctcacagtccaagggagagggaaagctgtctaacaggattggttttcccgtgtgctttataagcagatgagtagaggagacagctcttattgtcctagtggcaattgggataggctgcaaagtttgttagggtggaggcttattccgggaccaagggagcccaaagaaacaagctcctgccaggcgcggtggctcacgcctgtaatcccagcactttgggaggctgaggcaggtggatcacctgaggtcaggagtttgagaccagcctggccaacatggtgaaaccccgtctccatgaaaaatacaaaaattacccgggcatggtggcgggcacctgtaatcccagctactagggaggctgaggcaggaaaatggcttgaacctcggaagtggaggtggccgttagccgagatcacgccactgcactccagcctgggcaacagagcaagactctgccttaaaaaaaaaaaaaaaaaaaagaaaagtaaaaggaaaaaaaagaggctctggcctgctggggtgcctgcaaagtctccgtggaagggtgacattcaagccgagacctccagggaactgtctcctgggagcacagagccctttgctcagcccccaggtggctcagtgcccccagccagcagactcagagcttgcatgattctttggtgctctctgcggtcttccaatgatgctgaaataaatggtgcttggtgtctccctgctgtagtccccttgcttgctttgctcacaggtgctgggagagatatgccttcgaggatatttggatgcattcaggttcttggaagagaagggtatgtatgggctgggaggatcagccatgcccttttgacaagcatttactagcggtcttggtaaagacttgagatttgccttagttctaacacttagtgcccaacgccttccttgtgttgctcaacctactcatgagcccaggagataggaaatctccgtcccattgtacagatggggaaacagaattttggaaaggagagccaagcagcacacacccctccctgaggggcagagccgagatttgaactgggatgtcatgactccagggccctctccctccccagggtccccttatctgaaggcggtttttctttccagctcgacctcttgtgacccttagtttaacaagggccgaagttaaagagtttctgcgcctggaccccaaatgaagcaatcagatttctcatctccagtcaggtgtgggtccaagcccactagacaagtttgctcttcccagagcacatttctgccttcaagtcatcctggcttgtcagggctgggggagttctgctgtagaaatattagagtggaaggaaaaagatgtgttgggagctatttttctttaatactaaaagttggttgatgaatttgtcgttggccaagaccaaggagactgcatttttaaggacatatgtgtatttatctgctcagaaaatgttcattgctgtgtgctagggatactgcagtgaacacagaggtgtgacccttgccagccttgtgagagaagtgagcagataagtaagcagaagggtgatgctgtgtcgatgggaaagtacaggtgccaatgagaaggcacaggtgtcaaggagaagacacaggatgctggaggctcatgcaggatggatctccaaggcccaggggaagaagggcctctcggaggacgtgaatccacattaagactttggggataagtaggagcgccttaggcatggggacccatggatgcgaggcctgtaggacacagacaggatggcatgaaggcctgtgcaactggaggggtggggatggggacactaagagatggctggaagtgtgggggtggggacactaagagatgactggagaagagggggtcaggagtggtgaaaaatgggagaggagggcaggctgggccttttggatacagggggattgcatcctgcagtggtagggagccactgagggctgctgcagtaggagtgaggggatcagaggagagctttggaagccccctggatgcgggacaggaagcgagataccagtgtctaggaggccagtgaggcagccacaggctccaccaggatcagggctgcgagggtcatgaggaggaaaccaatttgaaggagtccaggggaataggacttggaaatgaccgatgggacatttgggaagaggaagacagaagagcgcagtcccggcttctggctttagcagttgggcaaggggagatggggagatgtgcccatgggttgagggttgaggacattaggagggagccggtatggcaggaagagctggtgtgccagagatgctggaagcagcatctgcctgagaacagatacctggcaatattcctaagggaaagtgacatctcggagggtgaggagggcatctgatagggcctggaaagagccggggcaagcatgaatgtgaggttatcttggggggcaaggctcaggcgttgaggagcagcccctggtctcttcagcctgaagttggaagccagagttgggccaggtgcagctgtggttgtctgaagtccccctcccccagcccagtgtgccaatgctgtaagagcaagggccgctcactggtgctggtggctgagtcccagcacccaggacagggcctggcacatactggtgcccaatcctcccttctgggtgcttcttccaaggccttgtgatggaagtgagtaccctcttcgacatcagacccagcttcaaatcctggctctgctatgtattggctgcgtggctttagacaagtcttttaaccttgctgtgcttctgatttctcagctgaaaaatggagatgatgatagtggtttctgtaaggccttatggtgaagcacctagctcagggcctggaaggcaggtgtaaccagtggttcagttgttataaaccaacactaaccctcgcctttgcacctcatgaaaccagatatgtagatggagcccacaaagctagcaggagccaagctcacgtgtgtcctgctttaaagccccatacccctttctccgggtgacaaacacctgtgctcgttctcttcccttcccctcttccccttgcatttggctaataacaggccagctgcctgcctccctgcagtttggtagatgggtgggtaacgaccaccactcccacgttcgcctgatgggcttgttttccgtgcccttcacaggcatctgcaacaggccccagccaggcctgaagtcatcctcagaagggatggatcctgaggtcgccatgcccagctgggcaaacatgagtctggattcttccccggagtcggctgccttggctgtgaggctggagggagatgagctgctagaccacctgcgtctcagcatcctgccctgggatgagagcatcctggacaccctctcgcccaggctcgctacaggtacccactcctcggggtgagcacgggcagcaccttgttttctttcttgtgcattatggaggaagatggtactgccacatgggagcgatagggtgaggcaaccatgacaggtggttgggaacatctccttccatgtgtacagcctgggctgctgccatcactcccagcacagcccccaacccccccaatcctggaaccttgccaagtctcccttcccatggggtcatgaccaggaggaaaacaaactccagctgagccccttggggttccccatataggctcctgcctgtggcagctgggccctctgtacccctttccaactctgtctccctaacatggcacctgagctcctgccatcctggatttcatggaccccaaggatgggggtcctgcatctgggacttggcctattactcggagctccttttcagccgcctccctccacctgtccacccacctcaaggctcctttcttgagacctctcctaatttctcccttcccctaaacccacaattttgaacctccatcgaatggtgctgtattttataatgtcatcaaatatcaaatggagacagtgctatggtccaaatgattgtgtaccccccagaatttgtcttttgaaatcctaacccccaacatgatggtcttaggaggtggggcctttgggaggagattaggtcatgaggaaagggctgtcatgaatgggattggtgcccttattaaacagacccaagagaggtcccttgtcccttctactgtgtgaggactcagaaggtggtgtctatgaagaagcaggccctcaccagacaccaacatgtctgctgccccttgatctgggaccttgcagcctctagaactctgaaaaatcgatgtttgttgttttataagccactcagttggtggcattttgttagagtagcctgaacacggactaagtcaaacagaagaacccacaaaccagctacagagttgggcatttggagaaattcaaaaatgagtcagacataactccttattcttgaggtgccctaagagatgggacacagcagctgcccaggtgcattagtttgttctcacattgctataaagaaatacctgagactgggtaactcataaagaaagaggttgaattggctcacagttgcacaggctggacaggaagcatggtgctggcatctgctcagcttctggggaggcctcaggaaacttacaatcatggcagaaggtgaacgggaagcatgcacatcccatgactggagcaggagtgagagagagagggaaatagagggaaggtgccatacacttttaaacaaccagatctcatgagaacacattcactatcaagagaacagcaccagtggggaaatccgcccccatgatccaatcacctcccatcaggctccgcctccaacactgggaattacaatttgacatgagatgtgggcagggacacagatccaaaccatatgaccagattaatacgatttgaggcatcacgaggtcattaaagagagggaataaaagactggggctccaggaagaaggctctggaatccagcagagggtcaaggaccagcttgtaaagctggtggtgcctgagaagtacctaggagaacatagatgctgtgacgtttgatgtagctgttttttgttttgtgttttggtttttgagacagagtctcactctgttgcccaggctggagtgtgcagtggcgtgatcttggctcactggagcctccatctcccaggttcaaatgatcctcatgcctcagcctcctgagttgctgggattacaggtgcacaccaccacgcctggctaatttttgtgttttcagtagagacagggtttcaccatgttggccaggctggtcttgaactcctgacctcaagtgatccaacaacttcagcctcccaaagtgctgggatgacaggcatgagccaccatgcccagcctgatgtagctgtttctgtgcacattatttgctgtggggtatattcagatttcttaatacaagatgattctttgcctcatgacttacacaccattttctatttaatttcagctatgatattggaaatggacatgtcttttcaaggaaaataaaagcaggctttctggaatggcgacttccaaacatatttgtcaatttaaaggagctgggagtggggaccctatgctccgtaagcactctcttagctgttcttggctgtgctccccgcttcagcttcacactgcccttgctgtgaagggagcagcctgggccgggcgcggtggcttacacctgtaatcctagcactttgggaggccgaggtgggtggatcacctgaggtcaggagttcaagaccagcctggccaacatggtgaaactccatctctactaaaaatacaaaaaattagctgggcatggtggcaggtgcctgtaatcccagctacttgggaggctgaggcagaagaatcgcttgaacccaggaggcggaggttgcagtgagccgagattgcgccattgcactccagcctgggggcaacaagagcaaaactctgtctggaaaaaaaagaaaggagcagcttggcaaaccccaccttgtcgcttttgtgagtgcctctgaccctttggctgccaggacgggcgtattttatggaaatgctaagcaccaacagagtaaagtggtttggtttttcacagtggtgggagataatagctccaaattgtctttttcagcactgagtgaagaaatgaaagacaaaggtggatacatgagcaagatttgcaacttgctacccattaggataatgtcttatgtaatgctgccctgtaccctgcctgtggaatctgccattgcgattgtccagaggtgagcattttaggtggctccgtgtcttcctcacagggttgatatgaggatgaaacaagatgatagatcatggtggcatgtagtctgggacctggattgtcgtgccacagatcacagctcacagtctatgtgcaatgcccctgaatgttgcccacctgtcctcaagccacacatgcacctgtaactcagtgcaagcccagaaactccccgtggggactcctagagctgtcagtggcctcacatagcagctggtccagtctcttgtgattgcccaaggaaactgaggcctggagagcttggggtcactgctctgaggccatagagatgcctagtagaagggccaggcctagaagcaggatccttgctgcccctctgagctgtttccatttaaaatcacatgaaggccggcgccgtggctcacggctgtaatcccagcattttgggaggccaaggtgggtggatcatgtgaggtcaggagtttgagaccagcttggccaacatggtgaaatgccatctgtactaaaaatacaaaaattagtggagcatggtggcacgtgcctgtactcccagctacttggaaggctggggcagaagaatcgcttgagcctgggaggcagaggttgtagtgagccaagattgtaccactgcactccagcctgggtgacaggagagaaaccctatctcaaaataaaatgaaaggtaatgaaatgaataaaataataaatcaagtcacggccgggcacggtggctcacacctgtaatcccagcgctttgggaggccgaggtgggtggataatgaggtcaggagttcaagaccagcctggccaacatggtgaaaccatgtctctactaaaaatacaaaaattagctgggcatggtggtgcatgcctgtaatcccagctactccggaggctaaggcaggagaattgcttgaagcaggacctaggaggcggaggttggttgcagtgagccgagatcatgccactgcactctagcctgggctacagagcgaaactccgactcaaaaaaaaaaaaaaaaaaaaatcaaatcacatgaaagtagaacatagggaattccatctttcgttctaggcatagtttgttaatatgattcagagccagcagttaggagaacacagtgtgactctcctagaacttcttgattgggcttcctctgattgggtttcctctgattgggcttcctctgaaagtgggggggatggggggtggggagcagaatggtcagagcttggctcagcagtcagactgctcttcttcaaatcctggctgcattgcttactacagctgtgtgactccagatgactgaatccacctctctgtgctgcagcttcccgtctagagagatcacctggagcagagggtggtcaggagactcaatctggttactgactcacagtgcaggagtactcatcccatagtaagcatccagctagagatgttgatttctattttcaggtaataatgatgatcgtaaaattagagacagataaaaggtatgggcattaggccagggcactgcaatttctaagctgtgtgacctcaggcaagttactcgacttctctgagcctcagcggtttcatccgcaatatatggataggaaaaccgacctcagtgggttgtctgacagtggagggcacttgattaaaaaaaaaaaaattaccctggtctgaatattaccctggactgaaagaaaaatattgagctaatacaggcatcaggaatggggctgcagggagtccagggaagggagaacgaagagcctgaaggtgtgaggaggtgcgagtgctgatctgtctgctacaaagaggctgctgagcctcctgtggatgtggccctggacttggcagtttaatacctgagctgttaaaataacctcagatgctgtgttctttaaggggtaggattcagattcctgctgaaatgcttctgaaagggagggaatgagccagcccatccccagttgctttttaagatcattgggaagttctggtcttgccatttgtccctggaccactcttaggtcctcctgccccacttccatctgggtgtgtgccctgggctgtccaccacacagctacatcctgccatcttccctcctggagccactgtgccatgcatggatctgtagcttcatttttcttggcttttccctggtttttctggagcagagtctctagtaaactcccaaggaagaaaacgtttgactttatgtgtgttgggaaacgtgctttttttctattacatctcagtgataggttggccatgtctagaattgcaggttgaaaatcatttcctctcagtatattggttagtgagaagcctgggactgagacagtcacattctcacttctttgcaggtgagtgctcttaggactgtctttttatcccttatactctgaaatgtcatatgtcttggtgtaagtccttatttcagttattgagctggacaagtactggagaccccttcagtcaaagccttctgtcattctccagctctaggaaattatcttctattgttatttctgttattccttcccttccattttcttttttctttttttttttttttttttgagacagggtcttactctggtgcccaggctggaatgcagtgacctgatcatggtacactgcagcctgaacctcccagactcaagtgatcctcccacctcaacctcctaagtagctgggactgcaagcacacatcaccacacccaacaaatattttttaaaaattttgtaagatgggatcttactatgttgcccagactttttcttcctcttcctggggctcttattaggaagatgtttgacttcctgggttggattcctgtctccgtgtctgactttctctctttgtcatatttttcatcactcgttgtctttttgcgtctgctctgacagatttcctcaaattttgtcttctagtcctatcctacagtttttactttcagcaaatataatttaatctccaagagtactctcttgttcttttttcttagcattctgttcttgttttatggatgtaacattctcttggaatatttgctgtcctctagatcatcccttctccatttcttcttgggctagtttttctgtttcttcatctttctcttttatgctacttattctgggcgtgttcttggtgggttttttcccatatagcaacagaggacttggagctcagggagaaaagggtaggtgcatcacctggcagagctcccagacagtgacaggcaggctgcgggaaggatgtctacttggcggtgctaccgctttcctagaaaccctttccctggagctggttgaactgttgggttttgccctggtggtgaacgctggctccccgtgctctgcctgtttcatcaccagccccctccccttctgcctggggtccagtaatctgttgaaatatatatcttgctcattggtgagctcctgctccttcctcgttgctcttgcagatttatcacttctcgtaaggctgcgcttgtacttcgggattttctctgtgccacactgggaaacatagggtggttgcatgctgcagtcctgagcacttatttcactcacatctttacacgaagatttggtgggtgtttactttgtttttagtaagttagtctgtcatgtcctttgatcctttttttttgttttttgagatggagtctctctgtgtcctccaggctggagtgcaatgtcgcgatctcagctcactgcaacctccacctcctgggctcaagagattctcctgcttcagtctcctgagtagctgggattacaggcatgtgccaccacacctggctaatttttgtatttttagtagaggtggggtttggcatgttggccagcctggtctcaaactcctgacctcctgacctgcctgccttggcctcccaaagtgctgggattacaggtgtgagccaccacacctggccctgattaatcttttaatgcccagtctctccttcaaaagccggctcctttctctccctcgccttcctagattccttctccactccccaggatcagcctcctcctccccaccccaccactgccggggggatgtctgtggtcaggcatttatcagagaccctgaggtgggggtcctttatgtgtctgggggatggagagtctagaggaggtagcgttcagacctctccatggtgcctctgctgggctcacatgtgaccaagcacagcaaaccatgaggcaggggatggtcttgaccatgagagcccttgcagcagctgccatgggcctcagctcctctccaagctgggaagagccctgaaaagccaaggtgtttttttttccctctttatttcagtgtaagtcccttgagctttcttgaaccagaagtgggctcattttgctttagagatttcaggtgggcttgtccttgtcctagcatcccagatccaccttctgggaagtcatcagattggaggtgatgttggcagcttttgtaaacaaagggtagtgttgtaagctgttgtgtctgcctatgtgtgtgtttgtgtacttggtctcatctctgcagactggtgacatggcttccagatatgcccgacgatgtcctgtggttgcagtgggtgacctcacaggtgttcactcgagtgctgatgtgtctgctccccgcctccaggtaaatactttggctgtgggtgtgtgggccggacgggcacctctctcatctgatgaggcctcacacgacattctagaaacagctggctgaacaccaagcaaggagcttgcccttgggtgtggggaccctgtctcatgggaggcagctgagtcagtcagaggtcctggcacacctgctgagagctgccacccaggccaacctgaaccggagcctgggaagacttcccgtcggatgagtctctttgagtgcagcattgatggtggaagagcagagaggccccagataagcagggaaaggtgcttcagacagagtggctgggatgaggactggggagtgtcagatagcgctggcgtgtctgagcgaaggagctctggcacccatggcacaggaaggaggtgggaccctggaggggcagggctagcagagctcctcggagcgtgtggctaggtgcctggtaatgcaagccccctgtcctccaccctctgttgtactgagtcacagtctccggggtgaagcctagcagtctgcgttgacaggccccaggggatgccgctacttcctgaattctgaattctggaaactgagccggagttcagggcctggctcccattaccagggttgggcgttatcctgaaaatcataggccttggtttcctcacttggctaacaggggtgatccccatcccctcaatgggtttccgtgagctcctgagagcccgtagcatggtacttggcacatgctgggcatcaggaggtatggcctctcttgctattgttgttattggtagacacagaaggatttaaaagtaggggaatgcaaagatccgatttgctagggaagagggcagtagtggccaagtagagggtggatcctgggccctggctggcagcaggcagcaaggggggctgccagggcccaggcagggacgatctgtagaccgagaggcttcctaaggctcttggacaggaggaggtgtcggttccaagcctaaggagtggggcagccctggtgactggtggtcagtggtgccaggcggtgggtggtaggacaccctggcaggcaagtaggtttgtgtgggggaaactgataggcccctccagggattcgttggtggacaacacctgtgatgtccagtgggaggtgtccaggtagctgggagggccacaggcttggaagacctaggtggtgacatcagcccagcactgagggctagaagaagctgtgtctctggctgtgacggcaccctagagtgtgtgtggtgccctctactggccggcaatgtgggtccaccgtagctcagactgcacactgcagcagcgggaacggcctctaagccaacttcctccatgtgtttcaggtcccaaatgccagtgagcagccaacaggcctccccatgcacacctgagcaggactggccctgctggactccctgctcccccaagggctgtccagcagagaccaaagcagaggccaccccgcggtccatcctcaggtccagcctgaacttcttcttgggcaataaagtacctgctggtgctgaggggctctccacctttcccagtttttcactagagaagagtctgtgagtcacttgaggaggcgagtctagcagattctttcagaggtgctaaagtttcccatctttgtgcagctacctccgcattgctgtgtagtgacccctgcctgtgacgtggaggatcccagcctctgagctgagttggttttatgaaaagctaggaagcaacctttcgcctgtgcagcggtccagcacttaactctaatacatcagcatgcgttaattcagctggttgggaaatgacaccaggaagcccagtgcagagggtcccttactgactgtttcgtggccctattaatggtcagactgttccagcatgaggttcttagaatgacaggtgtttggatgggtgggggccttgtgatggggggtaggctggcccatgtgtgatcttgtggggtggagggaagagaatagcatgatcccacttccccatgctgtgggaaggggtgcagttcgtccccaagaacgacactgcctgtcaggtggtctgcaaagatgataaccttgactactaaaaacgtctccatggcgggggtaacaagatgataatctacttaattttagaacacctttttcacctaactaaaataatgtttaaagagttttgtataaaaatgtaaggaagcgttgttacctgttgaattttgtattatgtgaatcagtgagatgttagtagaataagccttaaaaaaaaaaaaatcggttgggtgcagtggcacacggctgtaatcccagcactttgggaggccaaggttggcagatcacctgaggtcaggagttcaagaccagtctggccaacatagcaaaaccctgtctctactaaaaatacaaaaattatctgggcatggtggtgcatgcctgtaatcccagctattcggaaggctgaggcaggagaatcacttgaacccaggaggcggaggttgcggtgagctgagattgcaccatttcattccagcctgggcaacatgagtgaaagtctgactcaaaaaaaaaaaatttaaaaaacaaaataatctagtgtgcagggcattcacctcagccccccaggcaggagccaagcacagcaggagcttccgcctcctctccactggagcacacaacttgaacctggcttattttctgcagggaccagccccacatggtcagtgagtttctccccatgtgtggcgatgagagagtgtagaaataaagac